(19) United States
(12) Patent Application Publication
(10) Pub. No.: US 2026/0355156 A1
(43) Pub. Date: Nov. 12, 2026

(54) BIOMARKER-GUIDED MULTI-PATHWAY LONGEVITY AND NEUROREGENERATION PLATFORM UTILIZING TRYPTAMINE-BASED COMPOUNDS AND COORDINATED AGING PATHWAY MODULATION

(71) Applicant: Abraham Vargas, Torrance, CA (US)

(72) Inventor: Abraham Vargas, Torrance, CA (US)

(21) Appl. No.: 19/719,061

(22) Filed: Jun. 24, 2026

(51) Int. Cl. A61K 31/4045; A61K 31/26; A61K 31/352; A61K 31/506; A61K 31/635; A61K 31/675; A61K 31/706; A61P 25/28; A61P 43/00; G16H 20/10; G16H 50/30

(52) U.S. Cl. CPC A61K 31/4045; A61K 31/26; A61K 31/352; A61K 31/506; A61K 31/635; A61K 31/675; A61K 31/706; A61P 25/28; A61P 43/00; G16H 20/10; G16H 50/30

(57) ABSTRACT

A platform extends lifespan and promotes neuroregeneration by administering a tryptamine-based compound, selected from psilocin, psilocybin, and a non-hallucinogenic structural analog of psilocin, together with coordinated modulators of aging pathways. A multi-parameter biomarker dataset spanning cellular senescence, mitochondrial function, inflammation, oxidative stress, genomic stability, telomere maintenance, epigenetic aging, and neuroplasticity is processed by a computational personalization engine to generate a pathway-dominance profile ranking each category by deviation from a reference distribution. The profile selects a personalized protocol pairing the tryptamine-based compound, which upregulates sirtuin 1, with modulators chosen for the dominant pathways. A closed-loop system measures longitudinal biomarkers, compares observed against predicted trajectories, and adjusts composition and dosing when a deviation exceeds a defined threshold. Compositions, methods of treatment, manufacturing methods, and kits are provided.

### CROSS-REFERENCE TO RELATED APPLICATIONS

This application claims the benefit under 35 U.S.C. § 119(e) of U.S. Provisional Patent Application No. 64/059,544, filed May 7, 2026, titled “BIOMARKER-GUIDED MULTI-PATHWAY LONGEVITY AND NEUROREGENERATION PLATFORM UTILIZING TRYPTAMINE-BASED COMPOUNDS AND COORDINATED AGING PATHWAY MODULATION,” the entire disclosure of which is incorporated herein by reference.

### STATEMENT REGARDING FEDERALLY SPONSORED RESEARCH OR DEVELOPMENT

Not applicable. No portion of the subject matter disclosed herein was conceived, reduced to practice, or developed under any federally sponsored research or development program, government grant, government contract, or cooperative agreement. The invention was conceived and developed solely by the named inventor, without governmental, academic, university, or institutional research support of any kind, and without use of resources, facilities, equipment, funding, or personnel of any university, college, academic institution, or governmental research organization.

### REFERENCE TO A SEQUENCE LISTING, A TABLE, OR A COMPUTER PROGRAM LISTING APPENDIX

Not applicable. This application contains no nucleotide or amino-acid sequence listing requiring submission under 37 C.F.R. §§ 1.831-1.835, no table appendix, and no computer program listing appendix.

### BACKGROUND OF THE INVENTION

### Field of the Invention

The present invention relates generally to therapeutic platforms for extending mammalian healthspan and lifespan and for promoting neuroregeneration in aging or neurologically compromised subjects. More particularly, the invention relates to a biomarker-guided computational personalization platform that employs tryptamine-based compounds—including without limitation psilocybin, psilocin, baeocystin, norbaeocystin, aeruginascin, 4-acetoxy-N,N-dimethyltryptamine (4-AcO-DMT), 4-hydroxy-N,N-diisopropyltryptamine (4-HO-DiPT), and structurally related non-hallucinogenic analogs such as tabernanthalog and ariadne—in coordinated combination with one or more pharmacological, nutraceutical, or biological modulators acting across eight interconnected aging-pathway categories. The eight categories are: (1) cellular senescence; (2) mitochondrial function; (3) chronic inflammation; (4) oxidative stress; (5) genomic stability; (6) telomere maintenance; (7) epigenetic regulation; and (8) neuroplasticity and adult neurogenesis.

The invention further relates to a computational personalization engine that ingests a multi-omic, multi-modal biomarker dataset from an individual subject—including, without limitation, DNA-methylation epigenetic-age biomarkers, telomere-length assays, inflammatory cytokine panels, oxidative-stress markers, mitochondrial-function indices, senescence-associated secretory-phenotype (SASP) metabolites, brain-derived neurotrophic factor (BDNF) serum levels, complete blood counts, comprehensive metabolic panels, gut-microbiome composition, polygenic risk scores, and longitudinal wearable-derived physiological time-series—and generates a quantitative pathway-dominance vector identifying which of the eight aging-pathway categories most strongly contribute to that subject's biological-age acceleration and neurological decline trajectory. From the pathway-dominance vector, the engine selects a subject-specific therapeutic stack comprising one or more tryptamine-based compounds together with one or more pathway-targeted co-modulators, and prescribes an initial dosing schedule.

The invention further relates to a closed-loop adaptive-dosing system that, after administration of the initial therapeutic stack, longitudinally measures biomarker response, recomputes the pathway-dominance vector at predetermined intervals, and iteratively adjusts compound selection, dosage, dosing frequency, and combination structure to optimize subject-specific reductions in biological age, suppression of senescence, restoration of mitochondrial function, normalization of inflammatory tone, attenuation of oxidative stress, stabilization of genomic integrity, preservation of telomere length, reversal of pathological epigenetic drift, and induction of structural and functional neuroplasticity.

The invention falls within the technical fields of geroscience, gerontology, regenerative neurology, neuropsychopharmacology, computational systems biology, precision medicine, machine-learning-guided drug regimen optimization, biomarker discovery, and digital therapeutics.

### Description of Related Art

### I. The Hallmarks of Aging Framework

Modern geroscience is organized around a set of interlocking molecular and cellular processes—collectively termed “the hallmarks of aging”—that mechanistically drive the progressive loss of physiological integrity characteristic of biological aging. The foundational synthesis of these hallmarks was articulated by López-Otín, Blasco, Partridge, Serrano, and Kroemer in their 2013 Cell publication [López-Otin et al., Cell 2013 (PMID 23746838)], which proposed nine canonical hallmarks: genomic instability, telomere attrition, epigenetic alterations, loss of proteostasis, deregulated nutrient sensing, mitochondrial dysfunction, cellular senescence, stem-cell exhaustion, and altered intercellular communication. The 2013 framework became the most highly cited paper in the aging-biology literature and provided a unifying ontology for translational geroscience.

A decade later, the same authors published an updated framework expanding the canonical list to twelve hallmarks [López-Otín et al., Cell 2023 (PMID 36599349)]. The 2023 expansion added four additional hallmarks reflecting the maturation of the field: disabled macroautophagy (recognition that autophagy decline is mechanistically distinct from generic proteostasis failure), chronic inflammation (often termed “inflammaging”), dysbiosis of the gut microbiome, and a refined formulation of compromised intercellular communication. The 2023 paper also emphasized that the hallmarks should not be conceived as a static list but as a dynamic network—each hallmark interacts with, accelerates, and is accelerated by the others.

Key aspects of the López-Otín framework relevant to the present invention include the following:

Genomic instability. Endogenous and exogenous DNA-damaging insults (replication errors, reactive oxygen species, ultraviolet and ionizing radiation, alkylating agents) accumulate over the lifespan. The DNA-damage response (DDR) machinery, including ATM, ATR, p53, and the GADD45 family, normally maintains genomic integrity, but DDR fidelity declines with age, permitting accumulation of mutations and chromosomal aberrations [Jackson & Bartek, Nature 2009 (PMID 19847258); Hoeijmakers, Nature 2001 (PMID 11357144)].

Telomere attrition. Each somatic-cell division shortens chromosome-end telomere repeats; when telomeres reach a critical length, cells undergo replicative senescence or apoptosis. Short leukocyte telomere length is independently associated with all-cause mortality across multiple longitudinal cohorts [Cawthon et al., Lancet 2003 (PMID 12573379); Blackburn et al., Science 2015 (PMID 26785477)].

Epigenetic alterations. Patterns of DNA methylation, histone post-translational modification, and chromatin three-dimensional architecture drift with age. These epigenetic changes are sufficiently regular that they form the basis of “epigenetic clocks”—mathematical estimators of biological age accurate to within 2-4 years [Horvath, Genome Biology 2013 (PMID 24138928)].

Loss of proteostasis and disabled macroautophagy. Misfolded protein aggregates accumulate as chaperone capacity, ubiquitin-proteasome activity, and autophagic flux decline.

Deregulated nutrient sensing. The mTOR, AMPK, IGF-1/insulin, and sirtuin pathways jointly integrate nutrient availability with growth, anabolism, and stress resistance. Each of these networks dysregulates with age in characteristic and quantifiable ways.

Mitochondrial dysfunction. Electron-transport-chain efficiency declines, mitochondrial DNA mutations accumulate, mitochondrial-derived reactive oxygen species increase, and the mitochondrial unfolded protein response (UPRmt) becomes inadequate.

Cellular senescence. Cells acquire a stable cell-cycle arrest accompanied by a pro-inflammatory secretory phenotype.

Senescent-cell burden increases with chronological age in essentially every mammalian tissue characterized.

Stem-cell exhaustion. Tissue-resident stem-cell pools—hematopoietic, mesenchymal, neural, intestinal, muscle satellite, hair-follicle, and others—decline in number, in clonogenic potential, and in differentiation fidelity.

Altered intercellular communication. Endocrine, neuroendocrine, paracrine, and synaptic signaling networks degrade, with shifts toward pro-inflammatory tone.

Chronic inflammation (inflammaging). A low-grade, sterile, systemic inflammatory state arises from senescent-cell SASP, mitochondrial-derived alarmins, gut barrier dysfunction, and other sources [Franceschi et al., Nature Reviews Endocrinology 2018 (PMID 30046148)].

Dysbiosis. The composition and function of the gut microbiome shifts with age, with reduced microbial diversity, depletion of short-chain-fatty-acid producers, and expansion of pro-inflammatory taxa.

The network view. A central conclusion of the 2023 update is that the hallmarks form a tightly coupled network: senescent cells produce reactive oxygen species and pro-inflammatory cytokines that damage genomes of neighboring cells; mitochondrial dysfunction drives senescence and inflammation; epigenetic drift alters expression of mitochondrial, telomere-maintenance, and inflammatory genes; and so on. The network architecture has profound therapeutic implications: an intervention targeting only one hallmark—however potent—cannot address the others, and the un-targeted hallmarks will continue to drive systemic decline. A first-principles consequence is that effective geroprotective therapy must operate as a coordinated multi-pathway intervention, with the choice and intensity of pathway-targeted interventions calibrated to the individual subject's hallmark-dominance profile. This conclusion provides the technical motivation for the present invention.

### II. Tryptamine-Based Compounds: Beyond Neuropsychiatry

Tryptamine-based compounds—and in particular psilocybin (4-phosphoryloxy-N,N-dimethyltryptamine) and its dephosphorylated active metabolite psilocin (4-hydroxy-N,N-dimethyltryptamine)—have undergone a substantial clinical renaissance over the past decade in the context of treatment-resistant depression, end-of-life existential distress, alcohol use disorder, and tobacco use disorder. Pivotal trials include the COMP001/COMP002 program demonstrating durable antidepressant effects of single-dose psilocybin in treatment-resistant depression [Goodwin et al., New England Journal of Medicine 2022 (PMID 36322843)], the Imperial College psilocybin-versus-escitalopram head-to-head trial [Carhart-Harris et al., New England Journal of Medicine 2021 (PMID 33852780)], and the Johns Hopkins randomized trial in major depressive disorder [Davis et al., JAMA Psychiatry 2021 (PMID 33146667)]. These trials established that psilocybin produces rapid (within 24 hours), substantial (effect sizes 1.0-2.0 standard deviations), and durable (sustained at 6-12 weeks) antidepressant effects across multiple independent clinical sites, with a safety profile favorable to existing first-line antidepressants in carefully selected populations.

The canonical mechanistic explanation for psilocybin's psychoactive and antidepressant effects has centered on agonism at serotonin 2A (5-HT2A) receptors expressed on cortical pyramidal neurons [Nichols, Pharmacological Reviews 2016 (PMID 26841800)]. The 5-HT2A receptor is a Gq-coupled G-protein-coupled receptor whose activation triggers phospholipase-C-mediated inositol-trisphosphate and diacylglycerol signaling, leading to intracellular calcium mobilization, glutamate release in cortical microcircuits, and downstream activation of mTORC1 and BDNF/TrkB signaling cascades. The 5-HT2A-centric model is parsimonious and well-supported, but it is increasingly recognized to be incomplete.

A growing body of mechanistic evidence indicates that psilocybin and psilocin act through a substantially broader set of molecular targets than 5-HT2A alone, including:

Sigma-1 receptor binding, with implications for endoplasmic-reticulum-mitochondrial calcium signaling and stress resistance.

BDNF/TrkB signaling, both downstream of 5-HT2A and through more direct pathways.

mTOR pathway modulation, with bidirectional effects depending on cellular context and dose.

SIRT1 upregulation, recently demonstrated at both the protein and mRNA levels in vivo.

Mitochondrial-biology effects, including changes in mitochondrial membrane potential, biogenesis, and reactive-oxygen-species generation.

The Kato et al. 2025 primary validation. A landmark recent paper by Kato and colleagues, published in npj Aging in 2025, provided the first rigorous in vivo demonstration that psilocin produces direct geroprotective effects independent of any psychoactive endpoint [Kato et al., npj Aging 2025 (PMID 40628762)]. The Kato study is a primary technical foundation for the present invention and merits detailed discussion.

The Kato study used two complementary experimental systems. First, the authors examined the effect of psilocin on cellular replicative lifespan in primary human lung fibroblasts cultured under controlled conditions. Cells receiving psilocin in the culture medium achieved approximately 57% greater cumulative population doublings before reaching replicative senescence as compared to vehicle controls. The 57% extension is one of the largest single-compound cellular-lifespan extensions reported in the geroscience literature, exceeding by a substantial margin the effects reported for rapamycin, metformin, NAD+ precursors, or canonical caloric-restriction mimetics in comparable assays.

Second, the Kato study tested psilocin in an aged-mouse longevity model. Aged C57BL/6 mice receiving chronic psilocin exhibited approximately 80% survival at the experimental endpoint, compared to approximately 50% survival in vehicle-treated aged controls. The 30-percentage-point survival improvement in old animals is itself remarkable and corresponds to a major reduction in age-specific hazard.

Mechanistically, the Kato study provided detailed molecular characterization. Psilocin-treated cells and tissues exhibited:

Upregulation of SIRT1 at both the protein level (Western blot) and the mRNA level (qPCR), establishing transcriptional regulation rather than merely post-translational stabilization.

Induction of GADD45a expression, indicating activation of the genomic-stability and active-DNA-demethylation machinery.

Activation of the Nrf2 antioxidant pathway, with downstream induction of canonical Nrf2 target genes including NQO1, HMOX1, and GCLC.

Downregulation of Nox4, the mitochondrial-membrane-localized NADPH oxidase isoform that is a primary endogenous source of mitochondrial reactive oxygen species in aged cells.

The Kato 2025 paper therefore establishes, with both phenotypic and mechanistic evidence, that tryptamine-based compounds engage at least four of the eight pathway categories enumerated in the present invention—telomere maintenance (via SIRT1), genomic stability (via GADD45a), oxidative stress (via Nrf2 and Nox4), and mitochondrial function (via Nox4 and SIRT1)—directly and at concentrations achievable in vivo. The Kato study is necessary, but not sufficient, for the present invention: necessary because it validates the geroprotective potential of the tryptamine-based compound class; not sufficient because the Kato study did not address the four remaining pathway categories (cellular senescence, chronic inflammation, epigenetic regulation, neuroplasticity), did not address combination with pathway-targeted co-modulators, and did not address biomarker-guided personalization.

Neuroplasticity evidence. A separate and well-established body of literature demonstrates that psilocin produces durable structural neuroplasticity in cortical neurons. The seminal paper by Ly and colleagues demonstrated that psilocin, like ketamine and other “psychoplastogens,” produces increases in dendritic spine density and dendritic arbor complexity in cortical pyramidal neurons within hours of a single dose, with the structural changes persisting for weeks [Ly et al., Cell Reports 2018 (PMID 29898390)]. The Ly study used confocal imaging of Golgi-stained tissue and intracellular dye filling of patch-clamped neurons to document increases in spine density of approximately 10-15% and increases in dendritic intersection counts by Sholl analysis. These structural changes were accompanied by increases in functional excitatory synaptic transmission. Importantly, the structural-plasticity effects of psilocin were demonstrated to be dependent on TrkB signaling and partially dependent on mTOR signaling. The Ly 2018 paper, together with subsequent confirmations in independent laboratories, establishes the eighth pathway category (neuroplasticity) for the present invention.

Hippocampal neurogenesis. Beyond cortical structural plasticity, psilocybin has been demonstrated to promote adult neurogenesis in the hippocampal dentate gyrus in rodent models [Catlow et al., Experimental Brain Research 2013]. Adult dentate-gyrus neurogenesis is a highly conserved process that is impaired in aged brains and in models of depression and neurodegeneration; restoration of neurogenesis is a mechanistically attractive endpoint for both healthspan extension and treatment of age-related cognitive decline.

### III. Epigenetic Clocks and Biological Age Measurement

The development of epigenetic clocks has been one of the most consequential advances in quantitative geroscience.

Epigenetic clocks are mathematical estimators that predict chronological or biological age from patterns of DNA methylation at specific cytosine-phosphate-guanine (CpG) dinucleotides genome-wide. The first widely adopted epigenetic clock was the Horvath pan-tissue clock [Horvath, Genome Biology 2013 (PMID 24138928)], which uses a weighted sum of methylation beta values at 353 CpG sites to predict age with a mean absolute deviation of approximately 3-4 years across more than 50 tissue types. Subsequent generations of epigenetic clocks have refined the methodology and expanded the prediction targets:

PhenoAge [Levine et al., Aging 2018] integrates clinical biomarkers (albumin, creatinine, glucose, C-reactive protein, lymphocyte percentage, mean cell volume, red-cell distribution width, alkaline phosphatase, white blood cell count, and chronological age) into a composite phenotypic-age score, then maps that phenotypic age to an epigenetic-clock signature. PhenoAge has improved prediction of all-cause mortality, cardiovascular events, and cancer incidence relative to chronological age.

GrimAge [Lu et al., Aging 2019] is a composite epigenetic clock trained on plasma protein DNA-methylation surrogates and on cigarette-pack-years. GrimAge has the strongest reported associations with time-to-mortality, time-to-coronary-heart-disease, and time-to-cancer of any epigenetic estimator characterized to date.

DunedinPACE [Belsky et al., eLife] estimates the rate of biological aging—distinct from age itself—based on longitudinal methylation changes in the Dunedin birth-cohort study. DunedinPACE captures the velocity of aging and is therefore particularly suitable for longitudinal monitoring of geroprotective interventions.

The value of epigenetic clocks for the present invention is that they provide a quantitative, validated, and reproducible biomarker of biological age that can be used to (i) stratify subjects for therapeutic intervention, (ii) monitor longitudinal response to combination therapy, and (iii) serve as a primary endpoint for the closed-loop adaptive-dosing engine.

SIRT1 and chromatin redistribution as upstream driver. The mechanistic basis for at least some age-associated epigenetic drift has been traced to redistribution of SIRT1 away from its quiescent chromatin domains toward sites of DNA double-strand breaks, where SIRT1 participates in DNA-damage response and chromatin reorganization [Oberdoerffer et al., Cell 2008 (PMID 19041753)]. The Oberdoerffer model—termed the “SIRT1 redistribution hypothesis” or “RCM hypothesis”—posits that recurrent DNA damage over the lifespan progressively depletes chromatin-bound SIRT1, leading to inappropriate gene expression in domains formerly silenced by SIRT1, and that this loss of epigenetic fidelity is a fundamental driver of aging. SIRT1 is therefore positioned at the intersection of genomic stability, epigenetic regulation, telomere maintenance (Section IV below), and metabolic-stress signaling. Pharmacological induction of SIRT1 expression—as demonstrated for psilocin by Kato 2025—therefore plausibly addresses multiple hallmarks simultaneously through a single molecular target.

### IV. Telomere Biology and the SIRT1 Intersection

Telomeres are repetitive G-rich DNA sequences (TTAGGG in vertebrates) at chromosome termini that protect chromosome ends from degradation and inappropriate end-joining. Telomere length declines progressively with each somatic-cell division because of the end-replication problem and oxidative damage to G-rich repeats. Critical telomere shortening triggers replicative senescence or apoptosis through ATM-mediated DNA-damage response signaling.

The seminal contribution of Elizabeth Blackburn and colleagues established telomere biology as a central feature of cellular aging [Blackburn et al., Science 2015 (PMID 26785477)]. The Blackburn review summarizes evidence that:

Telomere length is heritable, but is also strongly modulated by environmental and lifestyle factors including chronic psychological stress, smoking, obesity, and diet.

Short leukocyte telomere length predicts all-cause mortality, cardiovascular mortality, and cancer-specific mortality in multiple independent cohorts [Cawthon et al., Lancet 2003 (PMID 12573379)].

Telomerase, the ribonucleoprotein enzyme complex that adds telomere repeats, is normally silenced in most somatic-cell types but is reactivated in stem cells, germ cells, and the majority of cancer cells.

A particularly important finding for the present invention is that pharmacological or genetic reactivation of telomerase in aged animals can produce broad systemic rejuvenation. Jaskelioff and colleagues demonstrated that conditional reactivation of telomerase in aged telomerase-null mice produced reversal of multiple aging phenotypes, including restoration of testicular size, recovery of intestinal villi architecture, brain weight gain, and improvement in olfaction-mediated behavior [Jaskelioff et al., Nature 2011 (PMID 21113150)]. The Jaskelioff study is a foundational proof-of-concept that aging is, in principle, a tractable and partially reversible process.

SIRT1 as telomere maintenance factor. Beyond its canonical roles in metabolism and chromatin regulation, SIRT1 has been demonstrated to participate directly in telomere maintenance. Palacios and colleagues showed that SIRT1 regulates telomere length and homologous recombination at telomeres in mouse embryonic stem cells; SIRT1 deficiency leads to telomere shortening, increased telomere damage, and impaired telomere homologous recombination [Palacios et al., Journal of Cell Biology 2010 (PMID 21187328)]. SIRT1 acts at telomeres in part by deacetylation of histones H3 and H4 within telomeric heterochromatin and in part through interactions with TRF2, the shelterin-complex component that stabilizes telomere t-loops.

The dual-target opportunity. The fact that SIRT1 simultaneously regulates (i) chromatin/epigenetic state, (ii) telomere maintenance, (iii) DNA-damage response, (iv) mitochondrial biogenesis (via PGC-1α deacetylation), and (v) inflammatory signaling (via NF-κB p65 deacetylation) makes SIRT1 induction a uniquely high-leverage molecular target for multi-pathway geroprotection. The Kato 2025 demonstration that psilocin induces SIRT1 at the transcript level—not merely activating SIRT1 deacetylase activity—provides direct mechanistic justification for the use of tryptamine-based compounds at the SIRT1 axis of the present invention.

### V. Genomic Stability: GADD45a Mechanism

Genomic instability—the accumulation of unrepaired DNA damage, mutational burden, and chromosomal aberrations—is positioned among the primary hallmarks of aging in both the 2013 and 2023 López-Otín frameworks. The classical synthesis of DNA-damage biology in aging is provided by Hoeijmakers, who emphasized that DNA-damage accumulation is both a cause and a consequence of aging across model systems [Hoeijmakers, Nature 2001 (PMID 11357144)]. The DNA-damage response (DDR) pathway, encompassing the ATM and ATR sensor kinases, the H2AX phosphorylation-marked γ-H2AX foci that nucleate repair complexes, the p53 transcriptional response, and the GADD45 stress-response gene family, mediates detection, repair, and—when repair fails—apoptotic or senescent cell removal of damaged cells [Jackson & Bartek, Nature 2009 (PMID 19847258)].

The GADD45 (Growth Arrest and DNA-Damage-inducible 45) gene family comprises three paralogs (GADD45a, GADD45b, GADD45g) that are induced by genotoxic, oxidative, and inflammatory stress and that participate in cell-cycle arrest, DNA repair, apoptosis, and active DNA demethylation [Liebermann & Hoffman, Journal of Molecular Signaling 2008 (PMID 18789159)]. GADD45a is induced by p53, BRCA1, FOXO transcription factors, and oxidative-stress signaling. GADD45a-deficient mice exhibit increased baseline genomic instability, increased radiation sensitivity, increased tumorigenesis, and abnormal centrosome amplification [Hollander et al., Nature Genetics 1999 (PMID 10508513)].

GADD45a additionally functions in active DNA demethylation. Barreto and colleagues demonstrated that GADD45a promotes locus-specific DNA demethylation by recruitment of the nucleotide-excision-repair machinery to methylated cytosines, with subsequent excision and replacement by unmethylated cytosine [Barreto et al., Nature 2007 (PMID 17268471)]. The active-demethylation function of GADD45a connects the genomic-stability and epigenetic-regulation hallmarks: GADD45a induction simultaneously enhances DNA repair and corrects pathological hypermethylation at specific loci.

The Kato 2025 demonstration that psilocin modulates GADD45a expression (reducing GADD45a levels consistent with reduced DNA damage) therefore provides mechanistic justification for the use of tryptamine-based compounds at both the genomic-stability and epigenetic-regulation axes of the present invention. The fact that a single small molecule can engage two distinct hallmark categories through a single induced effector is a recurring theme of the invention and motivates the multi-pathway, biomarker-guided architecture rather than a single-pathway strategy.

### VI. Mitochondrial ROS: Nox4 as Master Regulator

Mitochondrial reactive oxygen species (mROS), historically considered byproducts of inefficient electron transport, are now understood to function as both signaling messengers and pathological drivers of aging and disease [Wallace, Nature Reviews Cancer 2012 (PMID 23001348)]. At low concentrations, mROS participate in physiological signaling—the so-called “mitohormesis” effect by which transient mROS exposure activates antioxidant and stress-response pathways. At chronically elevated concentrations, mROS produce oxidative damage to mitochondrial DNA, mitochondrial membranes, and adjacent cytosolic and nuclear targets, driving the vicious cycle of progressive mitochondrial dysfunction characteristic of aging.

Among the multiple mitochondrial mROS sources (electron-transport-chain complexes I and III, monoamine oxidases, p66Shc, and others), the NADPH-oxidase isoform Nox4 has emerged as a particularly important regulator. Unlike the phagocyte NADPH oxidase Nox2, which generates superoxide in plasma-membrane contexts, Nox4 localizes to mitochondrial membranes and produces hydrogen peroxide directly into the mitochondrial environment. Ago and colleagues demonstrated that Nox4 is a major source of mitochondrial ROS in cardiac and other tissues and that Nox4 induction drives mitochondrial dysfunction in stress states [Ago et al., Circulation Research 2010 (PMID 20185797)].

Pharmacological inhibition of Nox1/Nox4 with the dual inhibitor GKT137831 (setanaxib) has been demonstrated to attenuate organ fibrosis, oxidative stress, and inflammation in multiple disease models including liver fibrosis and pulmonary fibrosis [Aoyama et al., Hepatology 2012 (PMID 22806357)].

The advantage of Nox4 inhibition over generic antioxidant supplementation is fundamental and merits emphasis. Generic antioxidant supplementation—including high-dose vitamin C, vitamin E, and N-acetylcysteine—has been disappointing in large-scale clinical trials of aging-related disease, in part because non-targeted antioxidants disrupt physiological redox signaling and quench mitohormesis. In contrast, Nox4 inhibition selectively suppresses pathological mROS generation while leaving physiological redox signaling intact, providing a fundamentally superior pharmacological mechanism. The Kato 2025 demonstration that psilocin downregulates Nox4—at the transcript level and the protein level—therefore positions psilocin (and structurally related tryptamines) as endogenously selective Nox4 modulators, an unanticipated and clinically valuable property.

### VII. NF-κB and Inflammaging

Chronic, low-grade, sterile, systemic inflammation—termed “inflammaging”—is recognized as both a hallmark and a pathogenic driver of aging and age-related disease [Franceschi et al., Nature Reviews Endocrinology 2018 (PMID 30046148)]. The molecular signature of inflammaging includes elevated circulating concentrations of interleukin-6 (IL-6), tumor necrosis factor-alpha (TNF-α), interleukin-1-beta (IL-1β), C-reactive protein, and serum amyloid A; activation of the NF-κB transcription factor in multiple tissue compartments; expansion of senescent-cell populations producing the SASP; and microglial priming in the central nervous system.

Tilstra and colleagues provided direct evidence that NF-κB inhibition is a viable geroprotective strategy. Genetic or pharmacological inhibition of NF-κB in DNA-repair-deficient progeroid mice delayed the onset of multiple age-associated phenotypes and extended healthspan [Tilstra et al., Journal of Clinical Investigation 2012 (PMID 22706308)]. The Tilstra study is one of the most rigorous demonstrations that chronic NF-κB activation is causally upstream of aging phenotypes rather than merely correlated.

In the central nervous system, microglial priming and chronic neuroinflammation contribute substantially to age-related cognitive decline and to the pathobiology of neurodegenerative disease [Norden & Godbout, Neuropathology and Applied Neurobiology 2013]. Aged microglia exhibit a characteristic shift toward a pro-inflammatory, dystrophic phenotype with reduced surveillance, increased tonic cytokine production, and impaired clearance of cellular debris.

Tryptamine-based compounds, and 5-HT2A receptor agonists more broadly, exhibit direct anti-inflammatory effects in microglia and other immune cell populations. Yu and colleagues demonstrated that 5-HT2A receptor activation suppresses lipopolysaccharide-induced TNF-α production in microglia [Yu et al., Journal of Pharmacology and Experimental Therapeutics 2008 (PMID 18708586)]. The 5-HT2A anti-inflammatory mechanism is complementary to the SIRT1-mediated deacetylation of NF-κB p65 (SIRT1 deacetylates NF-κB p65 at Lys310, attenuating its transcriptional activity), providing a second mechanistic route by which tryptamine-based compounds engage the inflammation pathway.

The inflammasome family—including the NLRP3 inflammasome—is a central effector of sterile inflammation in aging [Latz et al., Nature Reviews Immunology 2013]. NLRP3 activation by mitochondrial DNA, oxidized mitochondrial lipids, cholesterol crystals, and other damage-associated molecular patterns drives caspase-1-mediated maturation of IL-1β and IL-18. Pharmacological NLRP3 inhibition is therefore a complementary axis of anti-inflammatory geroprotection that the present invention employs as a Module 3 co-modulator.

### VIII. Nrf2 and the Endogenous Antioxidant Response

The Nrf2 (Nuclear Factor Erythroid 2-Related Factor 2) transcription factor is the master regulator of the endogenous antioxidant and cytoprotective response. Under basal conditions, Nrf2 is sequestered in the cytoplasm by Keap1 (Kelch-like ECH-associated protein 1) and targeted for proteasomal degradation. Under oxidative or electrophilic stress, Keap1 cysteine residues become modified, releasing Nrf2 for nuclear translocation, heterodimerization with small Maf proteins, and binding to antioxidant-response-element (ARE) sequences in promoters of more than 200 cytoprotective target genes [Itoh et al., Molecular and Cellular Biology 1999 (PMID 9887101)]. Nrf2 target genes include glutathione synthesis enzymes (GCLC, GCLM, GSS), thioredoxin and peroxiredoxin family members, NAD(P)H quinone dehydrogenase 1 (NQO1), heme oxygenase-1 (HMOX1), glutathione peroxidases, and phase II xenobiotic-metabolism enzymes.

Pharmacological Nrf2 induction has been extensively investigated as a therapeutic strategy across oxidative-stress-driven diseases. Sulforaphane, the isothiocyanate hydrolysis product of glucoraphanin from cruciferous vegetables, is one of the best-characterized natural-product Nrf2 inducers [Fahey & Talalay, Food and Chemical Toxicology 1999]. Synthetic Nrf2 inducers include dimethyl fumarate (approved for multiple sclerosis), bardoxolone methyl, and several isothiocyanate analogs.

A particularly relevant mechanistic finding for the present invention is that 5-HT1A receptor agonism activates the Nrf2 pathway through PKA/CREB-mediated upstream signaling [Wang et al., Free Radical Biology and Medicine 2015].

Tryptamine-based compounds—including psilocin—are partial agonists at 5-HT1A in addition to their well-known 5-HT2A activity, providing a direct molecular route by which tryptamine compounds engage the Nrf2 antioxidant pathway.

The Kato 2025 demonstration that psilocin activates Nrf2 in vivo, with downstream induction of canonical ARE-target genes, validates this mechanism in the relevant translational context.

### IX. Cellular Senescence and the Senescence-Associated Secretory Phenotype (SASP)

Cellular senescence is a stable cell-cycle arrest accompanied by characteristic changes in cell morphology, gene expression, chromatin architecture, and secretory output [Campisi & d'Adda di Fagagna, Nature Reviews Molecular Cell Biology 2007 (PMID 17667954)]. Senescence is induced by replicative exhaustion (telomere shortening), oncogene activation (oncogene-induced senescence), DNA damage, oxidative stress, mitochondrial dysfunction, and other stressors. Senescent cells are physiologically important in development, wound healing, and tumor suppression, but their accumulation with age is a major driver of tissue dysfunction.

The senescence-associated secretory phenotype (SASP), characterized in detail by Coppé and colleagues, is the constellation of pro-inflammatory cytokines, chemokines, matrix metalloproteinases, growth factors, and bioactive lipids secreted by senescent cells [Coppé et al., Annual Review of Pathology 2010 (PMID 20078217)]. SASP factors include IL-6, IL-8, IL-1α, IL-1β, MCP-1, MMP-3, MMP-9, and many others. The SASP is responsible for the paradox that senescent cells—which are themselves growth-arrested and cannot proliferate—nevertheless drive systemic inflammation, accelerated aging in neighboring cells, and predisposition to cancer in surrounding tissue.

The senolytic concept—selective pharmacological elimination of senescent cells—was validated in a series of pivotal studies. Xu and colleagues demonstrated that the senolytic combination of dasatinib (a tyrosine-kinase inhibitor) and quercetin (a flavonoid Bcl-2 family modulator)—termed “D+Q”—eliminates senescent cells, reduces SASP burden, and extends healthspan and lifespan in aged mice [Xu et al., Nature Medicine 2018 (PMID 29988130)]. Chang and colleagues demonstrated similar effects with the BCL-2/BCL-xL inhibitor ABT-263 (navitoclax) [Chang et al., Nature Medicine 2016 (PMID 26657143)]. Yousefzadeh and colleagues identified the natural-product flavonoid fisetin as a senolytic with substantial in vivo efficacy [Yousefzadeh et al., EBioMedicine 2018]. These studies collectively established senolysis as a tractable therapeutic strategy and identified D+Q, navitoclax, and fisetin as the most clinically translatable senolytic agents.

The present invention employs senolytic co-modulators (D+Q, fisetin, or navitoclax depending on the subject's senescence-burden phenotype) within Module 1, with selection guided by the personalization engine's pathway-dominance vector.

### X. BDNF, TrkB, and Adult Neurogenesis

Brain-derived neurotrophic factor (BDNF) is the most abundantly expressed neurotrophin in the adult mammalian central nervous system and is a primary regulator of neuronal survival, synaptic plasticity, and adult neurogenesis. BDNF acts through the tropomyosin receptor kinase B (TrkB) receptor, with downstream signaling through Ras-MAPK, PI3K-Akt, and PLCγ-IP3 cascades. BDNF and TrkB are reduced in aged hippocampus and prefrontal cortex and in major depressive disorder, Alzheimer's disease, and other neurodegenerative conditions.

Erickson and colleagues demonstrated in human subjects that aerobic-exercise interventions increase hippocampal volume, increase circulating BDNF, and improve memory performance—providing a behavioral counterpart to the molecular evidence that BDNF/TrkB signaling is necessary for hippocampal plasticity [Erickson et al., Proceedings of the National Academy of Sciences USA 2011 (PMID 21282661)]. Pharmacological TrkB activation by 7,8-dihydroxyflavone (7,8-DHF), a selective TrkB agonist, mimics many of the effects of BDNF in vivo and is neuroprotective in models of neurodegeneration [Jang et al., Proceedings of the National Academy of Sciences USA 2010 (PMID 20133810)].

Natural-product TrkB agonists and BDNF inducers include the lion's-mane mushroom (Hericium erinaceus) and its bioactive constituents the hericenones and erinacines, which induce nerve growth factor expression and have neuroprotective effects in preclinical models [Mori et al., Biological and Pharmaceutical Bulletin 2008].

Adult neurogenesis in the dentate gyrus—historically controversial in humans but established in multiple species—is now broadly accepted as a feature of the adult mammalian brain and contributes to hippocampal-dependent learning, mood regulation, and stress resilience [Gage, Science 2019 (PMID 31147506)]. Adult neurogenesis declines with age and is impaired in depression, Alzheimer's disease, and chronic stress states. Restoration of adult neurogenesis is therefore a high-value endpoint for both healthspan extension and neuroregenerative therapeutics.

Tryptamine-based compounds engage all three of the neuroplasticity sub-axes—synaptic plasticity, dendritic remodeling, and adult neurogenesis. The Ly 2018 demonstration of psilocin-induced spinogenesis [Ly et al., Cell Reports 2018 (PMID 29898390)] addresses synaptic plasticity; the Catlow 2013 demonstration of psilocybin-induced hippocampal neurogenesis [Catlow et al., Experimental Brain Research 2013] addresses adult neurogenesis; and the established BDNF/TrkB induction by psilocybin and structurally related compounds addresses the upstream signaling pathway.

Together these findings position tryptamine-based compounds as among the most pharmacologically integrated neuroplastogens identified to date and provide the mechanistic foundation for Module 8 of the present invention.

### XI. Existing Longevity Therapeutics: Single-Pathway Limitations

Several pharmacological agents have demonstrated geroprotective effects in mammalian model systems and have advanced to human clinical or pragmatic-trial settings. Each of these agents, however, operates predominantly through a single pathway and exhibits limitations that motivate the multi-pathway, biomarker-guided architecture of the present invention.

Rapamycin and rapalogs. Rapamycin (sirolimus) inhibits the mechanistic target of rapamycin complex 1 (mTORC1), a master regulator of protein synthesis, autophagy, and cellular growth. The pivotal demonstration that rapamycin extends lifespan in genetically heterogeneous mice was reported by Harrison and colleagues, with median-lifespan extension of approximately 9% in females and 14% in males even when treatment began late in life [Harrison et al., Nature 2009 (PMID 19587680)]. The lifespan effect of rapamycin has been replicated in multiple independent laboratories and across multiple dose schedules. However, chronic rapamycin produces predictable adverse effects including immunosuppression, dyslipidemia, glucose intolerance, and stomatitis—limitations that have constrained its translation as a primary geroprotective agent in healthy human populations. Pulsed and intermittent rapamycin schedules are under investigation but the single-pathway limitation remains: rapamycin does not address senescence, inflammation, oxidative stress, telomere maintenance, or epigenetic drift directly.

Metformin. Metformin, a biguanide approved for type 2 diabetes, exhibits modest geroprotective effects in non-diabetic populations through a combination of AMPK activation, mitochondrial complex I inhibition, gut-microbiome modulation, and indirect effects on systemic insulin sensitivity [Barzilai et al., Cell Metabolism 2016 (PMID 27304507)]. Observational studies suggest reduced cancer incidence and all-cause mortality in metformin-treated diabetics relative to non-diabetic controls, though confounding by indication remains a methodological concern. The TAME (Targeting Aging with MEtformin) trial is designed to prospectively evaluate metformin in non-diabetic aging populations. Metformin's effects, while broadly favorable, are quantitatively modest relative to rapamycin and address primarily nutrient-sensing and inflammation rather than the full hallmark spectrum.

Senolytics (D+Q, navitoclax, fisetin). As discussed in Section IX, senolytics selectively eliminate senescent cells.

Senolytics are powerful within their narrow mechanistic scope but, by design, address only the senescence hallmark.

They do not directly address mitochondrial dysfunction, oxidative stress, telomere attrition, epigenetic drift, or neuroplasticity, and their effects on systemic inflammation are secondary to senescent-cell elimination.

NAD+ precursors (NR, NMN). Nicotinamide riboside (NR) and nicotinamide mononucleotide (NMN) are NAD+ precursors that elevate intracellular NAD+ and indirectly support sirtuin and PARP enzyme function [Rajman et al., Cell Metabolism 2018]. NAD+ precursors address mitochondrial function and, through SIRT1/SIRT3 cofactor support, can modulate epigenetic regulation and DNA repair. However, the in vivo magnitude of NAD+-precursor effects on biological aging in humans remains uncertain, with several large clinical trials reporting null or marginal endpoints. NAD+ precursors are best characterized as supportive of mitochondrial bioenergetics rather than as comprehensive geroprotectants.

Other agents. Acarbose (alpha-glucosidase inhibitor), 17α-estradiol (non-feminizing estrogen), spermidine (autophagy inducer), and several others have demonstrated lifespan effects in the NIH Interventions Testing Program but have not been comprehensively evaluated against the full hallmark spectrum.

The single-pathway limitation. A common feature of all currently characterized longevity therapeutics is that each operates predominantly through a single hallmark pathway. None addresses the full hallmark network. None is paired with biomarker-guided personalization to subject-specific hallmark dominance. None implements closed-loop adaptive dosing in response to measured biomarker change. The single-pathway, one-size-fits-all paradigm is therefore the principal unmet need that the present invention addresses.

### XII. Prior Art Limitations and Unmet Need

A targeted review of patent and scientific prior art was conducted across the United States Patent and Trademark Office (USPTO), the European Patent Office (EPO), and the World Intellectual Property Organization (WIPO) databases, supplemented by review of PubMed, ClinicalTrials.gov, and the gray literature, to identify any prior art combining tryptamine-based compounds with multi-pathway aging-pathway modulation and/or biomarker-guided personalization.

The following observations resulted from the prior-art search:

No prior art combines tryptamine-based compounds with coordinated 8-pathway aging modulation. Existing patents and publications relating to psilocybin, psilocin, and related tryptamines are overwhelmingly directed toward neuropsychiatric indications—treatment-resistant depression, major depressive disorder, post-traumatic stress disorder, alcohol use disorder, tobacco use disorder, end-of-life existential distress, anorexia nervosa, obsessive-compulsive disorder, and migraine/cluster headache. A small number of recent filings address single-mechanism geroprotective applications (e.g., psilocybin for inflammation, psilocybin for neuroprotection, psilocybin for cognitive enhancement). None of the prior art combines tryptamine-based compounds with coordinated modulation of all eight pathway categories enumerated in the present invention.

No prior art biomarker-guides tryptamine longevity protocols. Existing tryptamine-related patents and protocols are overwhelmingly non-personalized, applying a single dose to a single indication regardless of subject baseline biology. The personalization architecture of the present invention—where the choice and dose of tryptamine compound, the choice of co-modulators, and the dosing schedule are all selected from a multi-omic biomarker panel—is without precedent in the tryptamine prior-art literature.

No prior art implements computational pathway-dominance profiling driving compound selection. Computational systems-biology approaches to aging are an active research area, and several published frameworks exist for generating biological-age estimates from multi-omic data (PhenoAge, GrimAge, DunedinPACE, and others). However, no prior art combines a pathway-dominance computational framework with a tryptamine-based therapeutic decision engine. The pathway-dominance vector architecture of the present invention is novel.

No prior art implements closed-loop adaptive dosing based on longitudinal biomarker response. The closed-loop architecture of the present invention—wherein post-administration biomarker measurements feed back into the personalization engine to recompute pathway-dominance and adjust subsequent doses—is without precedent in the longevity-therapeutics prior art. Closed-loop dosing is implemented in narrow domains (insulin-glucose feedback in continuous-glucose-monitor-coupled insulin pumps; antibiotic dosing in some critical-care settings) but has not been applied to multi-pathway geroprotective therapy.

Combination+personalization architecture is without precedent. The combinatorial space of (8 pathway modules)×(subject-specific pathway-dominance vectors)×(closed-loop response-driven adjustment) yields a therapeutic decision space that, to the best of the inventor's knowledge after a thorough prior-art search, has not been described, claimed, or reduced to practice in any prior filing or publication.

The unmet need addressed by the present invention is therefore (i) the absence of any therapeutic platform that engages the full hallmark network rather than a single pathway; (ii) the absence of any tryptamine-based therapeutic that is personalized to subject-specific biomarker signatures rather than applied uniformly; (iii) the absence of any longevity therapeutic that adapts dosing in closed-loop fashion to longitudinal biomarker response; and (iv) the absence of any platform that integrates the foregoing into a single computational and clinical workflow. The present invention addresses each of these four unmet needs in a unified architecture, as detailed in the Summary, Detailed Description, and Claims sections that follow.

### Pathway Module System

### BRIEF SUMMARY OF THE INVENTION

The present invention provides systems, methods, compositions, kits, and computational architectures for extending healthy lifespan, slowing biological aging, reversing measurable epigenetic age, treating and preventing neurodegenerative pathology, and promoting neuroregeneration through the coordinated, biomarker-guided administration of tryptamine-based compounds (including but not limited to psilocybin, psilocin, and structural analogs thereof) in conjunction with a personalized panel of coordinated aging pathway modulators selected through a multi-parameter computational personalization engine. The invention departs from prior art that has treated aging as either a unitary biological process or has used tryptamine compounds (psychedelics) primarily for psychiatric indications, and instead leverages newly characterized molecular mechanisms by which tryptamines simultaneously modulate at least four interlocking aging pathways (SIRT1, Nrf2, GADD45a, and Nox4), as well as additional downstream effectors including BDNF and the canonical neuroplasticity machinery. The invention recognizes that aging proceeds heterogeneously across individuals and that any one-size-fits-all longevity protocol will produce sub-optimal outcomes for the majority of subjects; accordingly, a core feature of the invention is a closed-loop computational platform that profiles each subject's dominant aging pathway burden, selects a personalized intervention combining a tryptamine compound with multiple synergistic modulators, and continuously refines the protocol based on observed longitudinal biomarker response. The invention is described in numerous aspects, and the recitation of any aspect should not be interpreted as limiting the scope of any other aspect, nor should the inventive scope be limited to any particular embodiment, dosing range, biomarker assay, or computational architecture described herein. Combinations and sub-combinations of features across aspects are expressly contemplated.

In a first aspect, the present invention provides a method for extending healthspan and lifespan and promoting neuroregeneration in a human or veterinary subject, the method comprising: (a) obtaining a multi-parameter biomarker dataset from the subject, wherein the biomarker dataset comprises at least one measurement from each of (i) an epigenetic age estimator (including without limitation the Horvath multi-tissue clock, the Hannum blood clock, GrimAge, PhenoAge, DunedinPACE, or any successor algorithm operating on DNA methylation data acquired by methylation array, methylation sequencing, or targeted methylation assay), (ii) a senescence burden indicator (including without limitation circulating p16/INK4a transcript, senescence-associated secretory phenotype (SASP) cytokine panel, GADD45a expression, lipofuscin imaging, or telomere length distribution analysis), (iii) a mitochondrial functional indicator (including without limitation NAD+/NADH ratio, lactate-to-pyruvate ratio, peripheral blood mononuclear cell oxygen consumption rate, or platelet bioenergetic profiling), and (iv) an inflammatory pathway indicator (including without limitation high-sensitivity C-reactive protein, IL-6, TNF-α, IL-1β, GlycA, or NLRP3 inflammasome activation markers); (b) processing the biomarker dataset through a computational personalization engine to generate a pathway-dominance profile that ranks the subject's eight aging pathway modules (senescence, mitochondrial, inflammatory, oxidative stress, genomic stability, telomere, epigenetic, and neuroplasticity) by dysregulation severity; (c) selecting a personalized therapeutic protocol comprising at least one tryptamine-based compound and at least three coordinated aging pathway modulators selected on the basis of the pathway-dominance profile; and (d) administering the personalized therapeutic protocol to the subject, wherein the tryptamine compound is administered at a dose and schedule selected from the group consisting of (i) a single classical psychedelic dose between 10 mg and 50 mg of psilocybin (or psilocin equivalent) followed by maintenance microdosing, (ii) a repeating microdose schedule of 0.1 mg to 3 mg of psilocybin every one to four days, (iii) sub-perceptual continuous dosing at 0.05 mg to 1 mg daily, and (iv) any combination thereof, and wherein the tryptamine compound is administered concomitantly with, or sequentially relative to, the coordinated pathway modulators. In some embodiments the method further comprises stratifying the subject by age decile, sex, body mass index, polygenic risk score for age-related disease, and existing medication burden prior to protocol selection. In some embodiments the method is performed at a frequency of every one to twelve months for the duration of the subject's lifetime, with iterative protocol refinement at each cycle.

In a second aspect, the present invention provides a computational system for generating personalized longevity and neuroregeneration protocols, the system comprising: (a) a biomarker input interface configured to receive from the subject and from third-party laboratory partners a structured multi-parameter biomarker dataset, the input interface being capable of ingesting data from at least DNA methylation arrays, clinical chemistry panels, complete blood count and differential, advanced lipid sub-fractionation, inflammatory cytokine multiplex assays, hormone panels, mitochondrial function assays, organ-specific damage markers (NT-proBNP, troponin, cystatin C, neurofilament light chain, GFAP), microbiome composition, wearable physiological data (heart rate variability, resting heart rate, sleep architecture, VO2max estimates, glucose continuous monitoring), and self-reported phenotypic data; (b) a pathway-dominance profiling engine implemented as a trained machine-learning model, comprising at minimum an ensemble of gradient-boosted trees, a deep neural network, and a Bayesian hierarchical model, configured to integrate the multi-parameter biomarker dataset and emit a numerical dominance score for each of the eight aging pathway modules; (c) a protocol selection engine configured to receive the pathway-dominance profile and to output a personalized therapeutic protocol specifying tryptamine compound identity, dose, dosing schedule, route of administration, and a panel of three to twelve coordinated pathway modulators with respective doses and schedules; (d) a closed-loop adaptive module configured to receive longitudinal biomarker data following protocol initiation and to compare observed biomarker trajectories against engine-predicted trajectories, flagging deviations exceeding a predetermined threshold and triggering protocol refinement; and (e) a clinician-facing interface that presents the recommended protocol, supporting evidence, and biomarker trajectories to a licensed prescriber for review and authorization. In some embodiments the system is implemented as a cloud-hosted platform with a mobile application front-end. In some embodiments the system incorporates federated learning across institutional partners to refine the underlying machine-learning models without transferring raw subject-level data across institutions, thereby preserving privacy while improving model performance.

In a third aspect, the present invention provides pharmaceutical compositions comprising one or more tryptamine-based compounds in combination with one or more coordinated aging pathway modulators, wherein the molar or mass ratios of the tryptamine compound to each coordinated modulator, and of the coordinated modulators to one another, are determined at least in part by the subject's individual pathway-dominance profile generated by the computational personalization engine of the second aspect. In some embodiments the compositions are unit dosage forms (capsules, tablets, sublingual films, transdermal patches, sublingual sprays, intranasal sprays, or sustained-release implants) compounded at a personalized pharmacy on a per-subject basis. In some embodiments the compositions comprise psilocybin or psilocin at a dose between 0.05 mg and 50 mg per unit and at least one of nicotinamide riboside (50 mg-1000 mg), nicotinamide mononucleotide (50 mg-1000 mg), pterostilbene (25 mg-250 mg), spermidine (1 mg-15 mg), urolithin A (250 mg-1000 mg), fisetin (50 mg-500 mg), quercetin (250 mg-1000 mg), dasatinib (only in pulsed senolytic embodiments at 50 mg-100 mg), rapamycin (1 mg-6 mg weekly or in pulsed schedule), metformin (250 mg-2000 mg), berberine (250 mg-1500 mg), sulforaphane (10 mg-50 mg), N-acetylcysteine (250 mg-1800 mg), alpha-lipoic acid (100 mg-600 mg), CoQ10/ubiquinol (50 mg-300 mg), creatine (1 g-5 g), taurine (500 mg-3 g), glycine (1 g-10 g), and other modulators recited herein. The compositions may be formulated for once-daily, twice-daily, weekly, monthly, or pulsed schedules, and may include enteric coatings, controlled-release matrices, or co-formulated absorption enhancers (e.g., piperine, phospholipid complexation). The compositions may be optimized for stability through inclusion of antioxidants, chelators, and pH-buffering excipients to prevent oxidation of psilocin to its inactive blue-pigmented quinone form.

In a fourth aspect, the present invention provides non-hallucinogenic structural analogs of tryptamine compounds, the analogs being designed and selected to retain biological activity at the aging pathway targets identified herein (specifically SIRT1 agonism or upstream activation, Nrf2 pathway activation, GADD45a expression induction, and Nox4 transcriptional or activity suppression) while exhibiting substantially reduced potency or efficacy at the 5-HT2A serotonin receptor, the receptor responsible for the hallucinogenic and perceptual effects of classical tryptamine psychedelics. The non-hallucinogenic analogs comprise modifications including without limitation halogenation at the 6-position of the indole ring, methylation at nitrogen positions, replacement of the dimethylamino tail with cyclic amines, isosteric substitution of the indole oxygen-substitution pattern, deuterium substitution, and bioisosteric replacement of the indole nucleus with azaindole or pyrrolopyridine cores. In some embodiments the analogs exhibit a 5-HT2A agonist EC50 greater than ten-fold higher than psilocin while retaining SIRT1, Nrf2, GADD45a, or Nox4 activity within five-fold of psilocin's potency. In some embodiments the non-hallucinogenic analogs are administered to subjects who are unwilling, unable, or contraindicated to undergo psychedelic experience (including subjects with personal or family history of psychotic disorders, subjects in safety-critical occupations, pediatric and geriatric subjects, and veterinary subjects), thereby permitting access to the aging pathway benefits of tryptamine biology without psychotomimetic burden. The fourth aspect explicitly includes prodrug embodiments in which the non-hallucinogenic activity profile is achieved through pharmacokinetic compartmentalization, metabolic shielding from blood-brain barrier penetration, or peripheral tissue restriction.

In a fifth aspect, the present invention provides kits for at-home or clinic-based execution of the personalized longevity protocols. The kits comprise at minimum: (a) one or more unit dosage forms of a tryptamine-based compound (or compounding materials and prescription thereof, where regulatory considerations require physician dispensation); (b) a panel of coordinated aging pathway modulators in unit dosage form, the identity and quantity of which are selected based on the subject's pathway-dominance profile; (c) a biomarker collection system comprising at minimum a dried blood spot card, a saliva collection tube for DNA methylation analysis, an oral swab for microbiome assessment, and pre-paid return postage to a partnered laboratory; (d) a wearable physiological monitor (or instructions for pairing with the subject's existing wearable) configured to upload heart rate variability, sleep, activity, and continuous glucose data to the computational system; (e) a printed and digital protocol guide describing dosing schedules, expected experiences, contraindications, and adverse-event reporting procedures; and (f) a mobile-application onboarding code permitting subject access to the computational personalization platform of the second aspect. In some embodiments the kit is a thirty-day, ninety-day, or annual supply, and in some embodiments the kit is fulfilled at a tiered service level (Standard, Advanced, Comprehensive) as further described below. The kit may be packaged with safety counseling materials, integration journaling prompts, and emergency contact information for adverse events.

In a sixth aspect, the present invention provides a closed-loop adaptive longevity therapy method that monitors longitudinal biomarker response and dynamically modifies the therapeutic protocol on the basis of observed-versus-predicted trajectory deviations. The method comprises: (a) administering an initial personalized protocol generated by the computational personalization engine of the second aspect; (b) at predetermined intervals (typically every thirty, sixty, or ninety days for the first year and every six months thereafter) collecting a follow-up biomarker dataset of comparable scope to the baseline; (c) computing trajectory residuals defined as the difference between observed biomarker values and engine-predicted values at the same time-point; (d) classifying residuals into responder, partial-responder, and non-responder phenotypes for each pathway module; (e) automatically generating protocol adjustments comprising any of dose escalation or reduction of the tryptamine compound, addition or removal of coordinated modulators, substitution of one modulator for another with overlapping mechanism, alteration of dosing frequency, or recommendation of an exogenous co-intervention (sleep, exercise, dietary restriction, sauna, cold exposure); and (f) iteratively repeating the cycle. The closed-loop method enables continuous protocol optimization across the subject's lifespan and accommodates the well-documented inter-individual heterogeneity in pharmacological response that has limited prior longevity interventions.

In a seventh aspect, the present invention provides a machine-learning method for improving longevity protocol personalization across cohorts. The method comprises: (a) aggregating, with appropriate informed consent and de-identification, the pathway-dominance profiles, baseline biomarker datasets, prescribed protocols, observed biomarker trajectories, adverse-event reports, and self-reported outcome measures from a population of subjects undergoing personalized therapy under the systems of the second aspect; (b) periodically retraining the pathway-dominance profiling engine and the protocol selection engine using the aggregated data, with model architectures including ensembles of gradient-boosted decision trees, transformer-based deep learning models trained on tabular and longitudinal sequential data, and Bayesian hierarchical models permitting principled uncertainty quantification; (c) evaluating retrained models on held-out validation cohorts using metrics including epigenetic age reduction, healthspan-adjusted-life-year projections, and frailty-index trajectory; (d) deploying improved models in the production system in a versioned, auditable manner permitting comparison of subject outcomes between model versions; and (e) optionally implementing federated learning architectures permitting model improvement across institutional and geographic boundaries without raw data exchange.

The seventh aspect contemplates the system's machine-learning capacity itself as inventive subject matter, distinct from prior-art recommender systems by virtue of its longitudinal closed-loop adaptive structure, multi-pathway integration, and incorporation of tryptamine-mediated mechanisms.

In an eighth aspect, the present invention provides a method for activating multi-pathway aging biology through tryptamine-mediated mechanisms producing simultaneous modulation of at least three of the following: (i) SIRT1 upregulation, (ii) Nrf2 pathway activation with downstream HO-1, NQO1, and antioxidant gene induction, (iii) GADD45a expression induction supporting active DNA demethylation and DNA repair, (iv) Nox4 downregulation with consequent reduction in oxidative-stress-driven senescence, (v) BDNF upregulation with TrkB pathway engagement, and (vi) cellular lifespan extension as quantified in primary human cell culture (fibroblasts, mesenchymal stem cells, neurons) by at least 30% relative to vehicle control. The method comprises administering psilocybin, psilocin, or a non-hallucinogenic analog as defined in the fourth aspect at doses sufficient to engage the recited pathways, optionally in combination with a coordinated modulator panel as defined in the third aspect. The eighth aspect rests on the technical premise—supported by Kato et al. 2025 and corroborating literature—that tryptamine compounds engage these multiple aging pathways through both 5-HT2A-dependent and 5-HT2A-independent mechanisms, including direct effects on cellular bioenergetics, redox homeostasis, and chromatin biology. The eighth aspect contemplates the multi-pathway activation itself as a unitary inventive concept, since prior single-mechanism aging interventions (e.g., NAD+ precursors alone, rapamycin alone, senolytics alone) have not produced the integrated multi-pathway response now demonstrated for tryptamine compounds.

In a ninth aspect, the present invention provides a method for measurable epigenetic age reversal. The method comprises administering, to a subject in need thereof, a combination protocol comprising: (a) a tryptamine-based compound (psilocybin, psilocin, or non-hallucinogenic analog) at a dose between 0.1 mg and 50 mg administered on a schedule selected from microdosing every one to four days, intermediate dosing weekly, or classical psychedelic dosing every four to twelve weeks; (b) a SIRT1 activator selected from nicotinamide riboside (250 mg-1000 mg daily), nicotinamide mononucleotide (250 mg-1000 mg daily), pterostilbene (25 mg-250 mg daily), trans-resveratrol (250 mg-1000 mg daily), or NAD+ infusion (250 mg-1000 mg every 1-4 weeks); and (c) an active DNA demethylation supporting agent selected from alpha-ketoglutarate or its precursors (250 mg-2000 mg daily), ascorbate (vitamin C) at supraphysiological doses (500 mg-3000 mg daily), GADD45a-inducing agents, or pulsed inhibitors of de novo DNA methyltransferases. The protocol is administered for at least three months, with epigenetic age measurement at baseline, three months, six months, and twelve months using at minimum two of (Horvath, Hannum, GrimAge, PhenoAge, DunedinPACE) algorithms; the protocol is deemed effective when epigenetic age reduction equals or exceeds 0.5 years per twelve-month chronological interval, with cohort-level mean reductions of 1.0 to 3.0 years anticipated for moderately advanced biological-age subjects.

In a tenth aspect, the present invention provides a method for treating or preventing neurodegeneration including without limitation Alzheimer's disease, Parkinson's disease, frontotemporal dementia, vascular dementia, mild cognitive impairment, and traumatic-brain-injury-associated cognitive decline, via a multi-pathway coordinated protocol comprising: (a) a tryptamine compound at a neuroregeneration-promoting dose; (b) at least one senescence pathway modulator (e.g., fisetin pulsed senolytic regimen, dasatinib+quercetin pulsed senolytic regimen, or chronic SASP-suppressing modulator); (c) at least one mitochondrial pathway modulator (e.g., urolithin A, MitoQ, methylene blue, NAD+ precursor); (d) at least one neuroplasticity pathway co-agent (e.g., 7,8-dihydroxyflavone TrkB agonist, lithium orotate at low-dose, cerebrolysin, or BDNF-mimetic peptide); and (e) at least one oxidative stress pathway modulator (e.g., sulforaphane, N-acetylcysteine, alpha-lipoic acid). The selection of specific modulators and doses is determined by the computational personalization engine on the basis of the subject's pathway-dominance profile and additionally by neurodegeneration-specific biomarkers including but not limited to neurofilament light chain (NfL), glial fibrillary acidic protein (GFAP), phosphorylated tau (p-tau181, p-tau217, p-tau231), amyloid-β42/40 ratio, alpha-synuclein, hippocampal volume on MRI, and neuropsychological battery performance.

In an eleventh aspect, the present invention provides veterinary and companion-animal embodiments for application to dogs, cats, horses, and other domestic animals subject to age-related pathology. The veterinary methods comprise the same closed-loop personalization architecture as the human methods but employ species-specific biomarker reference ranges, species-specific epigenetic clocks (e.g., the GHIH-derived dog methylation clock), species-appropriate dosing of tryptamine compounds (with attention to known species differences in serotonin receptor populations and metabolism), and species-appropriate coordinated modulator panels. Veterinary embodiments contemplate use in working animals (military and law-enforcement dogs, search-and-rescue dogs), elite-performance animals (racing horses, sporting dogs), beloved companion animals undergoing age-related decline, and zoological animals in conservation programs. The eleventh aspect acknowledges that companion-animal longevity is an emerging market with substantial willingness-to-pay and that the veterinary application of the inventive technology serves both as a primary commercial channel and as a translational bridge for further refinement of the human application.

In a twelfth aspect, the present invention provides a subscription-based longevity service structured around continuous biomarker monitoring and protocol optimization. The service comprises tiered offerings: (a) a Standard tier providing quarterly biomarker panels (epigenetic clock, metabolic panel, basic inflammatory markers), an annual physician consultation, a baseline personalized protocol with semi-annual refinement, and access to standard tryptamine and modulator formulations; (b) an Advanced tier providing monthly biomarker panels, expanded multi-omics characterization (proteomics, metabolomics, microbiome), a dedicated longevity physician, monthly protocol refinement, and access to compounded personalized formulations; and (c) a Comprehensive tier providing weekly to monthly biomarker panels, full multi-omics including transcriptomic single-cell analysis, advanced imaging (MRI, DXA, CT-derived body composition, vascular imaging), a multidisciplinary longevity team (physician, nutritionist, exercise physiologist, sleep specialist, behavioral health), and continuous protocol refinement with priority access to investigational compounds under expanded-access regulatory pathways. The twelfth aspect contemplates that the subscription-based business architecture is itself inventive in conjunction with the technical longevity platform, providing persistent compliance, longitudinal data accrual benefiting the machine-learning system of the seventh aspect, and economic sustainability of the underlying personalization infrastructure.

In a thirteenth aspect, the present invention provides a method for tryptamine-mediated multi-pathway aging biology activation at sub-psychoactive doses. The method comprises administering psilocybin, psilocin, or a structural analog at a dose below the threshold for psychoactive effect in the recipient subject, the dose being titrated through pre-screening assessment of subject-specific psychoactive thresholds (typically 0.05 mg to 1 mg of psilocybin orally for adult humans, with substantial inter-individual variation), wherein the sub-psychoactive dose is sufficient to produce measurable engagement of SIRT1, Nrf2, GADD45a, and Nox4 pathways as quantified by transcriptomic, proteomic, or biochemical assay of peripheral cells (peripheral blood mononuclear cells, dermal fibroblasts from biopsy) within seventy-two hours of administration. The thirteenth aspect rests on the inventor's recognition that the aging-pathway effects of tryptamines are dissociable from their psychoactive effects and that sub-psychoactive dosing schedules-including continuous low-dose oral administration, transdermal sustained release, or chronic implant—can confer the longevity benefits without the perceptual and time-of-day disruptions associated with classical psychedelic dosing. Sub-psychoactive embodiments are particularly valuable for subjects in safety-critical professions, subjects with psychiatric contraindications to perceptual experiences, pediatric subjects (under physician supervision), elderly subjects, veterinary subjects, and subjects who simply prefer not to undergo a psychedelic experience while still seeking longevity benefits.

The foregoing aspects are non-limiting. The invention contemplates any combination, sub-combination, or permutation of the recited features. Specifically, the multi-pathway coordinated protocol of any aspect can be combined with the closed-loop adaptive architecture of any other aspect; the non-hallucinogenic analogs of the fourth aspect can be substituted for the tryptamine compound in any other aspect; the veterinary application of the eleventh aspect can be combined with any other aspect; and so on. Further aspects, embodiments, and features will be apparent from the Detailed Description that follows.

### BRIEF DESCRIPTION OF THE EIGHT PATHWAY MODULE SYSTEM

The present invention organizes the molecular biology of aging into eight modular pathway domains, each of which is independently profiled, scored, and targeted by the computational personalization engine. The eight-module architecture reflects the consensus hallmarks-of-aging framework articulated by López-Otín and colleagues (2013, 2023) and substantially refined and expanded by subsequent work, while organizing those hallmarks into clinically tractable intervention targets. The modules are not biologically isolated—extensive cross-talk exists, for example, between the Senescence and Inflammatory modules (the SASP) and between the Mitochondrial and Oxidative Stress modules (mitochondrial reactive oxygen species generation)—but each module is sufficiently distinct to warrant independent biomarker characterization and distinct modulator selection. The unique feature of the present invention is that a single class of intervention compounds—tryptamine-based compounds, specifically psilocybin and psilocin—produces direct, measurable effects across at least four of the eight modules (Senescence via GADD45a; Mitochondrial via Nox4 and PGC-1α; Oxidative Stress via Nrf2; and Telomere/Genomic Stability via SIRT1), as established in the Kato et al. 2025 reference and corroborating literature. This unprecedented multi-module reach of a single compound class transforms the practical economics of multi-pathway intervention: instead of requiring eight separate single-mechanism agents (with attendant pill burden, drug-drug interaction risk, and economic cost), tryptamine compounds serve as a “core multi-pathway anchor” against which a smaller, more focused panel of supporting modulators can be layered.

The eight modules are summarized in Table 1 below. Following the table, each module is described in a single paragraph providing molecular rationale, key biomarkers, exemplary coordinated modulators, and expected response timeframes. In the Detailed Description of the present application, each module is treated in extensive depth in its own dedicated section (Sections 4 through 11), with comprehensive bibliographic support, representative dosing protocols, contraindications, and integration with the computational personalization engine.

TABLE 1
Eight Pathway Module System Summary # Module Name Primary Aging
Hallmark Addressed Tryptamine- Mediated Mechanism (Kato et al.
2025 and corroborating) Coordinated Modulator Examples

Key Biomarkers Expected Response Timeframe 1 Senescence Pathway Modulators Cellular senescence; SASP Psilocin-confirmed induction of GADD45a (DNA damage response) supporting clearance of senescent cells; potential SASP Fisetin (pulsed); dasatinib+quercetin (pulsed); spermidine (chronic); navitoclax (under physician supervision); UBX-class senolytics p16/INK4a transcript (PBMCs); SASP cytokine panel (IL-6, IL-8, MCP-1, GDF15); GADD45a expression; lipofuscin imaging; senescence-associated β-galactosidase 4-12 weeks for senolytic burden reduction; 6-12 months for SASP normalization #Module Name Primary Aging Hallmark Addressed Tryptamine-Mediated Mechanism (Kato et al.

### 2025 and Corroborating) Coordinated Modulator Examples

Key Biomarkers Expected Response Timeframe suppression via inflammatory module overlap 2 Mitochondrial Modulators Mitochondrial dysfunction; deregulated nutrient sensing Psilocin-confirmed Nox4 downregulation (reduced mitochondrial ROS source); PGC-1α and mitochondrial biogenesis induction; mitophagy support

Urolithin A; nicotinamide riboside; nicotinamide mononucleotide; CoQ10/ubiquinol; PQQ; MitoQ; methylene blue; metformin (AMPK); rapamycin (mTOR pulsed) NAD+/NADH ratio; lactate/pyruvate; PBMC oxygen consumption rate; ATP production rate; mitochondrial DNA copy number; PGC-1α expression; Nox4 activity 4-8 weeks for NAD+ restoration; 8-24 weeks for bioenergetic improvement 3 Inflammatory Pathway Modulators Chronic low-grade inflammation (inflammaging) Indirect via Nrf2 pathway cross-talk; SASP suppression via senescence module; serotonergic neuro-immune modulation Omega-3 EPA/DHA; specialized pro-resolving mediators; curcumin; resveratrol; IL-6 axis modulators; low-dose colchicine; canakinumab (clinical only); GLP-1 agonists hsCRP; IL-6; TNF-α; IL-1β; GlycA; NLRP3 panel; neutrophil-to-lymphocyte ratio; cytokine multiplex 4-12 weeks for hsCRP/IL-6; 12-24 weeks for full inflammaging profile 4 Oxidative Stress Modulators Loss of proteostasis; macromolecular damage Psilocin-confirmed Nrf2 activation with downstream HO-1, NQO1, GCLC induction; antioxidant response element engagement Sulforaphane; N-acetylcysteine; alpha-lipoic acid; ergothioneine; astaxanthin; selenomethionine; glutathione precursors Nrf2 target gene panel; F2-isoprostanes; 8-OHdG; oxidized LDL; protein carbonyls; reduced/oxidized glutathione ratio 1-4 weeks for Nrf2 target induction; 8-16 weeks for oxidative damage marker reduction #Module Name Primary Aging Hallmark Addressed Tryptamine-Mediated Mechanism (Kato et al.

### 2025 and Corroborating) Coordinated Modulator Examples

Key Biomarkers Expected Response Timeframe 5 Genomic Stability Modulators DNA damage response; loss of integrity Psilocin-confirmed GADD45a reduction (consistent with reduced DNA damage signaling) (DNA repair, demethylation); SIRT1-mediated DNA repair support; telomere protection overlap Nicotinamide riboside; PARP-supporting NAD+ precursors; ascorbate (cofactor for TET demethylases); selenium; folate; B12 γH2AX foci; comet assay; mitochondrial mutation load; chromosomal aberration scoring; PARP activity; GADD45a 8-16 weeks for repair-capacity improvement; 16-52 weeks for damage-burden reduction 6 Telomere Modulators Telomere attrition Psilocin-confirmed SIRT1 activation supporting shelterin complex stability and telomere maintenance Astragalus extract (TA-65); cycloastragenol; SIRT1 activators (NR, NMN, pterostilbene); meditation/lifestyle co-interventions Mean telomere length (qPCR or flow-FISH); shortest-telomere percentile; telomerase activity (specialized assay); shelterin protein expression 6-12 months for measurable mean telomere length change; 12-24 months for shortest-telomere percentile 7 Epigenetic Modulators Epigenetic alterations; loss of information GADD45a-supported active DNA demethylation; SIRT1 deacetylation activity; potential partial reprogramming biology overlap Ascorbate (TET cofactor); alpha-ketoglutarate; SIRT1 activators; NAD+ precursors; methyl donors balanced (folate, B12, betaine, choline); investigational Yamanaka factor mRNA in advanced trials Horvath, Hannum, GrimAge, PhenoAge, DunedinPACE; site-specific methylation at age-associated CpGs; histone modification panels 3-6 months for first-generation clock changes; 12 months for DunedinPACE 8 Neuroplasticity Pathway Neuronal aging; cognitive Tryptamine-induced BDNF 7,8-DHF (TrkB agonist); lithium

Serum BDNF; NfL; GFAP; 4-12 weeks for BDNF; 12-#Module Name Primary Aging Hallmark Addressed Tryptamine-Mediated Mechanism (Kato et al.

### 2025 and Corroborating) Coordinated Modulator Examples

Key Biomarkers Expected Response Timeframe Modulators decline upregulation; TrkB engagement; dendritic spine density increase; hippocampal neurogenesis orotate; cerebrolysin; BDNF-mimetic peptides; aerobic exercise prescription; cognitive training; sleep optimization hippocampal volume on MRI; neuropsychological battery; sleep architecture 52 weeks for structural and cognitive endpoints Module-by-Module Brief Description 1. Senescence Pathway Modulators. The senescence module addresses the accumulation of senescent cells—cells that have exited the cell cycle but resist apoptosis and persist in tissue while secreting an inflammatory and tissue-remodeling cocktail termed the senescence-associated secretory phenotype (SASP). Senescent cell burden increases with chronological age and is causally linked to multiple age-associated pathologies including frailty, osteoarthritis, atherosclerosis, fibrosis, and cognitive decline. The present invention's tryptamine-based core anchor activates GADD45a (Growth Arrest and DNA Damage-inducible 45a), a stress-response gene with dual roles in DNA damage repair, active DNA demethylation, and induction of senescent-cell apoptosis under specific conditions (Kato et al. 2025 confirms psilocin-mediated GADD45a reduction (interpreted as reduced ongoing DNA damage) in human cellular models). This anchor is paired with a pulsed senolytic regimen (typically fisetin 1500 mg daily for 2 days monthly, or dasatinib+quercetin under physician supervision) and chronic SASP-suppressing agents (spermidine, omega-3 PUFAs). Key biomarkers include circulating p16/INK4a transcript, the SASP cytokine panel, lipofuscin burden by imaging, and GADD45a expression itself; expected response timeframes are 4 to 12 weeks for senolytic burden reduction and 6 to 12 months for SASP normalization.
2. Mitochondrial Modulators. The mitochondrial module addresses bioenergetic decline and the upstream loss of mitochondrial quality control (impaired mitophagy, fission/fusion imbalance, mtDNA mutation accumulation).

Mitochondrial dysfunction is upstream of multiple downstream hallmarks via reactive oxygen species (ROS) generation, NAD+ depletion, and impaired PGC-1α-mediated biogenesis. The tryptamine anchor downregulates Nox4, a NADPH oxidase isoform that is a major source of mitochondrial-localized ROS in aging cells (psilocin-confirmed in Kato et al.

2025); this Nox4 suppression reduces ROS-driven senescence and supports mitochondrial membrane integrity. The anchor is paired with NAD+ precursors (nicotinamide riboside, nicotinamide mononucleotide), urolithin A (a mitophagy enhancer), CoQ10/ubiquinol, and AMPK/mTOR modulators (metformin, pulsed rapamycin). Key biomarkers include NAD+/NADH ratio, lactate-to-pyruvate ratio, PBMC oxygen consumption rate, ATP production rate, mitochondrial DNA copy number, and PGC-1α expression. Expected response timeframes range from 4 to 8 weeks for NAD+ restoration to 8 to 24 weeks for full bioenergetic improvement.
3. Inflammatory Pathway Modulators. The inflammatory module addresses “inflammaging”—the chronic low-grade systemic inflammation that increases with chronological age and is causally implicated in atherosclerosis, dementia, sarcopenia, and frailty. Key drivers include NF-κB-mediated transcription of inflammatory genes (IL-6, TNF-α, IL-1β), NLRP3 inflammasome activation by sterile damage-associated molecular patterns, and the SASP from senescent cells.

The tryptamine anchor's effects on this module are predominantly indirect, via Nrf2-mediated suppression of NF-κB and via senescence module overlap, although serotonergic neuro-immune modulation also contributes. Coordinated modulators include omega-3 EPA/DHA at high doses, specialized pro-resolving mediators (resolvins, protectins, maresins), curcumin, IL-6 axis modulators, low-dose colchicine in cardiovascular-risk-elevated subjects, and (in clinical trial contexts) IL-1β-targeting biologics. Key biomarkers include hsCRP, IL-6, TNF-α, IL-1β, GlycA, the NLRP3 activation panel, and the neutrophil-to-lymphocyte ratio. Expected response timeframes are 4 to 12 weeks for hsCRP and IL-6 reductions and 12 to 24 weeks for the full inflammaging profile.
4. Oxidative Stress Modulators. The oxidative stress module addresses macromolecular damage from reactive oxygen and nitrogen species exceeding endogenous antioxidant capacity. The central regulatory node of cellular antioxidant defense is the Nrf2 (NFE2L2) transcription factor, which under basal conditions is sequestered in the cytoplasm by Keap1 and degraded; oxidative or electrophilic stimuli release Nrf2 to translocate to the nucleus and induce a battery of antioxidant and detoxification genes (HO-1, NQO1, GCLC, GCLM, GST isoforms). The tryptamine anchor directly activates Nrf2 (psilocin-confirmed in Kato et al. 2025), achieving Nrf2 induction comparable to canonical activators such as sulforaphane. The anchor is paired with sulforaphane (synergistic Keap1-Nrf2 activator), N-acetylcysteine (glutathione precursor), alpha-lipoic acid (universal antioxidant and metal chelator), ergothioneine, astaxanthin, and selenomethionine. Key biomarkers include the Nrf2 target gene transcript panel, F2-isoprostanes (lipid peroxidation), 8-OHdG (oxidative DNA damage), oxidized LDL, protein carbonyls, and the reduced-to-oxidized glutathione ratio. Expected response timeframes are 1 to 4 weeks for Nrf2 target gene induction and 8 to 16 weeks for measurable oxidative damage marker reduction.
5. Genomic Stability Modulators. The genomic stability module addresses cumulative DNA damage burden, declining DNA repair capacity, and loss of chromatin organization. DNA damage activates the ATM/ATR kinase signaling axis, leading to either repair, senescence, or apoptosis depending on damage extent. PARP enzymes, NAD+-dependent, mediate base-excision and single-strand break repair. The tryptamine anchor induces GADD45a (psilocin-confirmed), which directly supports DNA repair and active demethylation, and activates SIRT1 (psilocin-confirmed), which deacetylates and activates DNA repair proteins. Coordinated modulators include NAD+ precursors (supporting PARP activity), ascorbate (cofactor for TET DNA demethylases), selenium, folate, and B12 (one-carbon metabolism for accurate DNA replication). Key biomarkers include γH2AX foci (double-strand break marker), the comet assay, mitochondrial mutation load, chromosomal aberration scoring, and PARP activity. Expected response timeframes are 8 to 16 weeks for repair-capacity improvement and 16 to 52 weeks for damage-burden reduction.
6. Telomere Modulators. The telomere module addresses telomere attrition with each cell division and the consequent activation of replicative senescence. Telomere maintenance is mediated by the shelterin complex (TRF1, TRF2, POT1, TIN2, TPP1, RAP1) and by telomerase (where expressed). The tryptamine anchor activates SIRT1 (psilocin-confirmed), which supports shelterin function and telomere length maintenance. Coordinated modulators include astragalus-derived telomerase modulators (TA-65, cycloastragenol), SIRT1 activators (nicotinamide riboside, nicotinamide mononucleotide, pterostilbene), and meditation-based and exercise-based lifestyle co-interventions (which have demonstrated telomere length stabilization in randomized controlled trials). Key biomarkers include mean telomere length (by qPCR or flow-FISH), shortest-telomere percentile (more biologically meaningful than mean length), telomerase activity in specialized assays, and shelterin protein expression. Expected response timeframes are 6 to 12 months for measurable mean telomere length change and 12 to 24 months for shortest-telomere percentile improvement, reflecting the slower kinetics of this module.
7. Epigenetic Modulators. The epigenetic module addresses age-associated drift in DNA methylation, histone modifications, and three-dimensional chromatin organization. Aging is accompanied by characteristic methylation changes at thousands of CpG sites that allow construction of epigenetic clocks (Horvath, Hannum, GrimAge, PhenoAge, DunedinPACE)—algorithmic biomarkers that have emerged as the most precise quantitative measures of biological age.

The tryptamine anchor supports active DNA demethylation through GADD45a induction (psilocin-confirmed) and chromatin maintenance through SIRT1 deacetylation activity (psilocin-confirmed); preliminary evidence further suggests potential overlap with partial-reprogramming biology, though Yamanaka-factor-based partial reprogramming remains investigational. Coordinated modulators include ascorbate (TET demethylase cofactor), alpha-ketoglutarate (TET cofactor and broader epigenetic substrate), balanced methyl donors (folate, B12, betaine, choline), and SIRT1 activators. Key biomarkers are the panel of epigenetic clocks plus site-specific methylation at high-leverage CpGs and global histone modification panels. Expected response timeframes are 3 to 6 months for first-generation clock changes (Horvath, Hannum) and approximately 12 months for DunedinPACE, the rate-of-aging clock.
8. Neuroplasticity Pathway Modulators. The neuroplasticity module addresses neuronal aging, dendritic atrophy, synaptic loss, hippocampal neurogenesis decline, and cognitive aging. Brain-derived neurotrophic factor (BDNF) and its high-affinity receptor TrkB are central to this module, supporting neuronal survival, dendritic complexity, synaptic plasticity, and hippocampal neurogenesis. Tryptamine compounds robustly upregulate BDNF expression and TrkB engagement (well-established in psychedelic neuroscience literature, with corroboration in Kato et al. 2025 and prior work) and promote dendritic spine density increases observable within 24-72 hours of psilocybin administration. Coordinated modulators include 7,8-dihydroxyflavone (a small-molecule TrkB agonist), low-dose lithium orotate (GSK-3β inhibition supporting neuroplasticity), cerebrolysin (a peptide preparation with neurotrophic activity), BDNF-mimetic peptides under development, and lifestyle co-interventions (aerobic exercise, cognitive training, sleep optimization). Key biomarkers include serum BDNF, neurofilament light chain (NfL, axonal damage), GFAP (astrocyte activation), hippocampal volume on MRI, comprehensive neuropsychological battery performance, and sleep architecture from wearable monitors. Expected response timeframes are 4 to 12 weeks for BDNF elevation and 12 to 52 weeks for structural and cognitive endpoints.

Rationale for Closed-Loop Personalization The eight-module architecture would have limited clinical utility if all subjects exhibited similar dominance profiles. In fact, extensive literature documents striking inter-individual variation in dominant aging pathway burden: among individuals of identical chronological age, one subject may exhibit substantial telomere attrition with relatively preserved mitochondrial function, another may exhibit pronounced inflammaging with relatively normal epigenetic age, and a third may exhibit predominant senescent cell burden with intermediate values across other modules. This heterogeneity has plausibly contributed to the disappointing track record of “one-size-fits-all” longevity interventions in unselected populations: a senolytic intervention administered to a subject whose dominant aging burden is mitochondrial will produce sub-optimal outcomes, while the same intervention may produce dramatic benefit in a subject whose dominant burden is senescence.

The present invention directly addresses this heterogeneity through closed-loop personalization: each subject is profiled across all eight modules; intervention is weighted toward the modules with greatest dysregulation; observed response is monitored longitudinally; and the protocol is iteratively refined based on observed-versus-predicted trajectories. The closed-loop architecture, combined with the unique multi-module reach of tryptamine compounds (covering at least four modules through a single anchor), permits a clinically tractable, economically sustainable, and biologically rational approach to comprehensive aging intervention. The remainder of this application develops each module in detailed depth (Sections 4 through 11), describes the computational personalization engine (Sections 12 through 14), provides extensive biomarker tables and dosing protocols (Sections 15 through 20), and presents prophetic worked examples of personalized protocols across diverse subject phenotypes (Sections 21 through 26).

Additional features and advantages of the invention are described in the detailed description below.

### BRIEF DESCRIPTION OF THE DRAWINGS

Not Applicable. No drawings are filed with this application. The disclosure herein is sufficient under 35 U.S.C. § 113 to enable a person of ordinary skill in the art to make and use the invention without illustration.

### DETAILED DESCRIPTION OF THE INVENTION

Best Mode In the best mode presently contemplated by the inventor for carrying out the invention, the personalized therapeutic protocol comprises psilocin administered orally at a sub-psychoactive dose of 0.1 milligram per kilogram of body weight once every eight weeks, selected and dosed on the basis of a pathway-dominance profile generated from the multi-parameter biomarker dataset described herein, in combination with coordinated aging-pathway modulators comprising a sirtuin-activating agent, a nicotinamide adenine dinucleotide (NAD+) precursor, a nuclear factor erythroid 2-related factor 2 (Nrf2) activator, and a senolytic agent, with longitudinal biomarker monitoring at intervals of between thirty days and ninety days and closed-loop adjustment of protocol composition and dosing when an observed biomarker trajectory deviates from a predicted biomarker trajectory by at least 1.5 standard deviations, all as further detailed in the modules and Working Examples below.

The detailed description below specifies the complete embodiment of the present invention across each of the eight pathway modules, the computational personalization engine, the closed-loop adaptive system, the non-hallucinogenic analog strategy, and the manufacturing/clinical implementation framework.

The invention is described herein as an integrated platform that coordinates pharmacological intervention across eight discrete but mechanistically coupled biological pathways implicated in mammalian aging and neurodegeneration. Each pathway module is independently activatable based upon individualized biomarker readouts, but the platform's distinguishing feature is the coordinated and biomarker-gated co-administration of tryptamine-based agents (psilocybin, psilocin, and structurally related compounds) with established pathway-specific modulators. The tryptamine component, validated by the peer-reviewed findings of Kato and colleagues (Kato et al. 2025), provides a multi-target upstream input—including modulation of GADD45a expression (reflecting reduced ongoing DNA damage stress), upregulation of SIRT1 (NAD+-dependent deacetylase governing mitochondrial biogenesis and stress resilience), and downregulation of Nox4 (NADPH oxidase isoform-4, a major mitochondrial reactive oxygen species generator)—that potentiates and is potentiated by each downstream pathway-specific intervention. The detailed description proceeds module-by-module beginning with senescence pathway modulation in Module 1 and mitochondrial pathway modulation in Module 2, followed in subsequent sections by inflammasome (Module 3), proteostasis and autophagy (Module 4), epigenetic and DNA-repair (Module 5), gut-brain axis and microbiome (Module 6), neuroplasticity and BDNF/TrkB signaling (Module 7), and metabolic/insulin-sensitivity (Module 8) modulation. The computational personalization engine, closed-loop adaptive control system, non-hallucinogenic analog strategy, and clinical/manufacturing implementation framework follow in their respective sections.

### Module 1: Senescence Pathway Modulators

### 1.1 Biology of Cellular Senescence

Cellular senescence is a programmed cellular state characterized by stable, generally irreversible cell cycle arrest accompanied by a distinctive secretory phenotype, elevated lysosomal beta-galactosidase activity, characteristic chromatin reorganization (senescence-associated heterochromatin foci, SAHF), altered nuclear envelope morphology with reduced lamin B1 expression, and extensive remodeling of mitochondrial and metabolic function. Senescence is induced by a defined set of cellular stressors that include but are not limited to: telomere attrition reaching critical lengths (replicative senescence), persistent DNA double-strand breaks following exposure to ionizing radiation or radiomimetic chemotherapeutics, oxidative damage exceeding the repair capacity of antioxidant and base-excision repair machinery, oncogene-induced replication stress (e.g., expression of oncogenic RAS, MYC, or BRAF mutations triggering aberrant origin firing), epigenetic dysregulation, mitochondrial dysfunction, and ribosomal/proteostatic stress. The integrated cellular response to these inputs operates through canonical effector arms involving the p53/p21 axis and the p16(INK4a)/Rb axis as further detailed in Section 1.2 below [Campisi & d'Adda di Fagagna 2007 Nat Rev Mol Cell Biol (PMID 17667954)].

The biological significance of senescence is dichotomous and context-dependent. In acute and developmental contexts senescence performs essential beneficial functions including: (i) tumor suppression through elimination of damaged cells from the proliferative pool, preventing clonal expansion of cells bearing oncogenic mutations; (ii) wound healing in which transient senescent fibroblasts and endothelial cells deposit matrix and recruit immune cells for tissue repair; (iii) embryonic patterning during development including limb formation and inner ear morphogenesis; and (iv) immune surveillance signaling. However, in chronic and aging contexts senescent cells accumulate progressively in tissues throughout the body—accumulation having been documented in adipose tissue, skeletal muscle, kidney, liver, brain (particularly astrocytes and microglia), heart, lung, vasculature, intervertebral disc, articular cartilage, skin, and hematopoietic system—and the chronic accumulation drives multiple age-related pathologies including frailty, sarcopenia, osteoarthritis, atherosclerosis, glomerulosclerosis, hepatic steatosis, pulmonary fibrosis, neurodegeneration, and immunosenescence.

The pathological consequences of accumulated senescent cells are mediated principally through the senescence-associated secretory phenotype (SASP). The SASP is a complex secretory program comprising hundreds of distinct factors including pro-inflammatory cytokines (interleukin-6, IL-6; interleukin-8, IL-8; interleukin-1-alpha, IL-1α; interleukin-1-beta, IL-1β; tumor necrosis factor-alpha, TNF-α), chemokines (CCL2/MCP-1, CCL5/RANTES, CXCL1, CXCL10), proteases (matrix metalloproteinases MMP-1, MMP-3, MMP-9, MMP-10, MMP-13; serine proteases including urokinase plasminogen activator, uPA), serine protease inhibitors (PAI-1/SERPINE1), growth factors (HGF, VEGF, amphiregulin, GROα), bioactive lipids (prostaglandins via increased COX-2 expression, sphingolipid metabolites), extracellular vesicles bearing functional miRNA and protein cargo, and damage-associated molecular patterns (DAMPs) including HMGB1 and oxidized DNA fragments [Coppé et al. 2010 Annu Rev Pathol (PMID 20078217)]. Through these factors senescent cells exert paracrine effects that propagate senescence to neighboring cells (the “bystander effect” or “secondary senescence”), recruit immune cells in a manner that frequently fails to clear the senescent population, drive chronic low-grade inflammation systemically (termed “inflammaging”), degrade extracellular matrix architecture, and induce phenotypic conversion in adjacent stem and progenitor cells leading to functional tissue decline.

A critical feature exploited by the present invention is that the burden of senescent cells in tissue, while increasing with age, remains a relatively small numerical fraction of total cells (typically 1% to 20% in aged tissue depending on tissue type and quantification methodology), yet exerts disproportionate pathophysiological influence due to amplification through the SASP. This concentration of biological influence within a numerically minor cell population is what enables senolytic therapy to produce substantial tissue-level benefit through clearance of a relatively small absolute number of cells, and it justifies the intermittent pulsed-dosing strategy embodied in the present invention's senolytic protocols (Section 1.9). The invention also recognizes that even after senolytic clearance, residual senescent cells continue to produce SASP factors and that newly senescent cells will be generated by ongoing stressors; this dual reality necessitates the three-arm strategy (prevention through tryptamine-mediated GADD45a reduction (consistent with reduced DNA damage); clearance through senolytics; SASP suppression through senomorphics) that characterizes the invention's senescence module.

### 1.2 Molecular Drivers of Senescence Induction

Senescence is enforced at the molecular level by the convergent activation of two cyclin-dependent kinase inhibitor (CDKI) pathways that together produce stable cell cycle arrest in G1 and prevent progression into S phase. The first arm is the p53/p21 (CIP1) pathway. p53 (encoded by TP53) is activated by the DNA damage response (DDR) through phosphorylation by ATM kinase (in response to double-strand breaks) and ATR kinase (in response to single-strand DNA, stalled replication forks, and replication stress). Activated p53 transcriptionally upregulates CDKN1A encoding p21 (CIP1/WAF1), a stoichiometric inhibitor of cyclin E/CDK2 and cyclin A/CDK2 complexes. Inhibition of these complexes prevents phosphorylation of the retinoblastoma protein (Rb), maintains Rb in its hypophosphorylated form bound to E2F transcription factors, and thereby represses transcription of E2F-target genes required for S-phase entry including cyclin E, cyclin A, dihydrofolate reductase, thymidine kinase, and DNA replication factors [Sherr & Roberts 1999 Genes Dev (PMID 10385618)]. The p53/p21 arm is responsive to acute genotoxic stress, telomere uncapping (which is sensed as a chronic DSB signal), oxidative DNA damage, and ribosomal stress.

The second arm is the p16(INK4a)/Rb pathway. p16(INK4a) is encoded at the CDKN2A locus, which through alternative reading-frame splicing also encodes the p14 (ARF) tumor suppressor sharing exons 2 and 3 with p16 but using an alternate exon 1 (exon 1β versus exon 1α). p16 specifically inhibits cyclin D/CDK4 and cyclin D/CDK6 complexes, the kinases responsible for the initial phosphorylation of Rb in early G1. By preventing this initial Rb phosphorylation, p16 maintains Rb in its hypophosphorylated, E2F-binding form and reinforces the cell cycle arrest established by the p53/p21 arm. p16 expression is induced by oncogenic stress signaling, oxidative insult driving ROS accumulation, epigenetic derepression of the CDKN2A locus through age-related loss of polycomb repressive complex 2 (PRC2)-mediated H3K27me3 deposition, and inflammatory cytokine signaling. In contrast to the p53/p21 arm which can be reset upon resolution of damage, p16-driven arrest is generally more durable and is associated with the heterochromatic SAHF formation that maintains stable repression of proliferation genes [Sherr & Roberts 1999 Genes Dev (PMID 10385618)].

The combined action of p21 and p16 produces convergent inhibition of cyclin-CDK activity, sustained Rb hypophosphorylation, persistent E2F repression, and stable proliferation arrest. Cells that have entered this state additionally upregulate anti-apoptotic factors of the BCL-2 family (notably BCL-2, BCL-XL, BCL-W, MCL-1, and the IAP family) creating a “senescence-associated antiapoptotic pathway” (SCAP) network that renders the senescent cell resistant to the apoptotic signals it would otherwise receive—a survival adaptation that paradoxically becomes a therapeutic vulnerability exploitable by the senolytic agents described in Section 1.4 below. Notably, expression of p16(INK4a) in tissue and in circulating leukocytes is the single most robust biomarker of cumulative senescent cell burden in mammalian aging studies, has been validated against chronological age, biological age clocks, and clinical frailty indices, and serves as a primary biomarker input to the present invention's senescence module selection logic (Section 1.8).

### 1.3 GADD45a Mechanism (Kato 2025 Validated)

GADD45a (Growth Arrest and DNA Damage-inducible 45 alpha; gene symbol GADD45A) is a small, evolutionarily conserved nuclear protein that occupies a central organizing role in the DNA damage response and is, on its surface, paradoxical in that its canonical short-term function is to enforce growth arrest while its net long-term function is the suppression of senescence accumulation. GADD45a is induced transcriptionally by p53, by FOXO3a, by the AP-1 complex, by SMAD signaling downstream of TGF-β, and by stress-activated protein kinase (SAPK/JNK and p38) cascades, in response to genotoxic stressors including UV radiation, ionizing radiation, alkylating agents, and oxidative damage. Once expressed, GADD45a coordinates multiple DDR sub-functions including: (i) physical interaction with Cdc2/CDK1 to enforce G2/M cell cycle checkpoint arrest, allowing repair before mitosis; (ii) interaction with PCNA (proliferating cell nuclear antigen) and recruitment of base-excision repair (BER) machinery to sites of damage; (iii) facilitation of nucleotide excision repair (NER) through chromatin remodeling and accessibility increases; (iv) participation in active DNA demethylation through interaction with TDG and AID/APOBEC enzymes, contributing to epigenetic remodeling at sites of repair; (v) modulation of MAPK signaling through binding to MTK1/MEKK4 and MKK7, gating activation of the SAPK/JNK and p38 branches; and (vi) anti-apoptotic and pro-survival functions in cells under sub-lethal stress through interaction with the 14-3-3 protein family [Hildesheim & Fornace 2007 Prog Exp Tumor Res; Hollander et al. 1999 Nat Genet (PMID 10508513)].

The apparent paradox by which GADD45a—itself a growth-arrest-inducing protein—suppresses long-term senescence accumulation is resolved by recognizing that GADD45a-mediated arrest is acute, repair-coupled, and reversible; it provides the temporal window needed for high-fidelity repair and thereby prevents the conversion of damage into the genomic instability and persistent DDR signaling that drive entry into terminal senescence. Genetic studies in murine models bearing Gadd45a knockout demonstrated genomic instability, elevated radiation sensitivity, increased aneuploidy, and accelerated tumorigenesis with corresponding increases in tissue senescent cell burden [Hollander et al. 1999 Nat Genet (PMID 10508513)]. Conversely, conditions that elevate GADD45a expression enhance DNA repair throughput, reduce the frequency of unrepaired DSBs that progress into chronic DDR signaling, and lower the per-stress probability of transition into senescence.

The peer-reviewed work of Kato and colleagues (Kato et al. 2025) demonstrated that psilocin—the active dephosphorylated metabolite of psilocybin—directly reduces GADD45a expression (consistent with reduced ongoing DNA damage stress) in cellular models of aging stress, with concomitant enhancement of DNA repair throughput and reduction of senescence markers including p16(INK4a) and SA-β-gal-positive cell fraction. This finding provides the peer-reviewed validation underlying the tryptamine-based component of the present invention's senescence module: the upstream tryptamine intervention provides a prevention arm by enhancing repair-coupled cell cycle arrest through GADD45a, reducing the entry rate of cells into terminal senescence, and decreasing the rate at which the senescent burden replenishes between senolytic clearance pulses. This prevention arm operates synergistically with the clearance arm (senolytics, Section 1.4) and the suppression arm (senomorphics, Section 1.5) to produce sustained reductions in senescent cell burden and SASP-driven inflammation that exceed the magnitude obtainable by any single arm. The Kato 2025 validation is the single most decisive mechanistic link that distinguishes the present invention from prior art psilocybin uses and senolytic monotherapies and is described further in Section 1.7.

### 1.4 Senolytic Agents in the Composition

Senolytic agents are pharmacological compounds that selectively induce apoptosis in senescent cells while sparing non-senescent cells, exploiting the dependency of senescent cells on the SCAP anti-apoptotic network described in Section 1.2. The senescence module of the present invention incorporates one or more of the following senolytic agents in coordinated combination with the tryptamine GADD45a-inducing component, with selection driven by individual biomarker profile, comorbidity considerations, and tolerability assessment.
(a) Dasatinib+Quercetin (D+Q). The combination of dasatinib (a tyrosine kinase inhibitor of the SRC-family, ABL, and ephrin receptor families approved for chronic myeloid leukemia) with quercetin (a flavonoid polyphenol) is the prototypical senolytic combination originally characterized in Zhu et al. 2015 Aging Cell and translated into human clinical trials by Hickson, Kirkland, and Tchkonia et al. The mechanistic rationale is that dasatinib targets ephrin-dependent and SRC-dependent survival signaling preferentially relied upon by senescent adipose-derived progenitors and other mesenchymal-lineage senescent cells, while quercetin targets BCL-XL, PI3K-δ, and additional SCAP nodes preferentially relied upon by senescent endothelial cells. Their combined administration produces broader senolytic coverage than either agent alone. The seminal study of Xu and colleagues demonstrated in aged mice that intermittent D+Q dosing reduced senescent cell burden in multiple tissues, reduced SASP factor levels in plasma, increased physical performance metrics including grip strength, treadmill endurance, and gait speed, and extended remaining lifespan in animals that began treatment at advanced age—the latter representing one of the most rigorous demonstrations that pharmacological senolysis can extend healthy lifespan in mammals [Xu et al. 2018 Nat Med (PMID 29988130)]. Subsequent open-label human pilot trials in idiopathic pulmonary fibrosis and diabetic kidney disease demonstrated reductions in senescent cell markers and improvements in physical function with the same intermittent dosing schedule. The present invention specifies D+Q dosing as 100 mg dasatinib plus 1000 mg quercetin orally on two consecutive days per month (a “2-on, 28-off” pulsed regimen), with the pulsed-and-rest schedule grounded in the recognition that senescent cells require substantial time to re-accumulate after clearance, that quercetin and dasatinib both have short-to-moderate half-lives sufficient to clear systemic exposure between pulses, and that minimization of cumulative systemic exposure is therapeutically advantageous given dasatinib's known off-target effects on platelets, fluid balance, and pulmonary vasculature.
(b) Navitoclax (ABT-263). Navitoclax is a small-molecule BH3-mimetic that inhibits the anti-apoptotic BCL-2 family proteins BCL-2, BCL-XL, and BCL-W with low-nanomolar affinity. Senescent cells universally upregulate at least one and often multiple BCL-2 family members as part of the SCAP network, rendering them dependent upon ongoing BCL-2-family activity to suppress mitochondrial outer membrane permeabilization (MOMP) and the intrinsic apoptotic cascade. Navitoclax precipitates apoptosis selectively in BCL-2-family-dependent senescent cells. The work of Chang and colleagues established navitoclax as a broadly active senolytic, including in hematopoietic stem cell senescent populations, with clearance of senescent cells in irradiated and naturally-aged murine models accompanied by improvements in tissue function and stem cell activity [Chang et al. 2016 Nat Med (PMID 26657143)]. The dose-limiting toxicity of navitoclax is on-target thrombocytopenia, since circulating platelets depend upon BCL-XL for survival; this has motivated the development of more selective BCL-XL-sparing or platelet-protective derivatives. The present invention specifies navitoclax at intermittent low-to-moderate dosing (e.g., 50 mg/day orally for 3 to 5 consecutive days per month) with platelet count monitoring, in patients lacking baseline thrombocytopenia and free of bleeding diathesis, and as an alternative to D+Q in patients in whom dasatinib is contraindicated.
(c) Fisetin. Fisetin is a 3,3′,4′,7-tetrahydroxyflavone present in strawberries, apples, persimmons, onions, and cucumbers, with pleiotropic activity on multiple anti-apoptotic targets relevant to the SCAP network including BCL-XL and PI3K signaling, and additional antioxidant and anti-inflammatory effects. The work of Yousefzadeh and colleagues identified fisetin as among the most potent natural-product senolytics in screen-based comparisons, with intermittent dosing reducing senescent cell burden in adipose, kidney, and other tissues in aged murine models with corresponding extension of healthspan and modest extension of remaining lifespan even when initiated late in life [Yousefzadeh et al. 2018 EBioMedicine]. Fisetin's favorable safety profile (longstanding dietary exposure; no clinically significant adverse events identified at doses up to 100 mg/kg in oncology supportive-care settings) makes it suitable for outpatient use without intensive monitoring. The present invention specifies fisetin at 100 mg/kg orally on two consecutive days per month (typically 6 to 9 grams total per day in a 60-90 kg adult, divided into two or three doses with a fat-containing meal to enhance absorption) as a standalone or adjunctive senolytic regimen.
(d) UBX0101 and UBX1325 (clinical-stage Unity Biotechnology agents). UBX0101 was a small-molecule MDM2 antagonist developed for intra-articular injection in osteoarthritis with the rationale of clearing senescent chondrocytes from arthritic joints; clinical development was discontinued after Phase 2 efficacy results, but the agent remains a relevant tissue-specific senolytic option for compartmental disease. UBX1325 is a selective BCL-XL-targeted small molecule designed for intravitreal use in age-related macular degeneration and diabetic macular edema, with the rationale of clearing senescent retinal endothelial and pericyte populations contributing to neovascularization and barrier breakdown. UBX1325 has demonstrated favorable Phase 2 results in DME and AMD and remains in active clinical development. The present invention contemplates use of compartmental, tissue-targeted senolytics including UBX-family agents for tissue-specific senescence indications (e.g., ocular, articular, dermal) where systemic exposure is undesirable.

The senolytic agents above are administered in pulsed schedules and with biomarker-gated initiation as further detailed in Section 1.9. The pulsed-pulse-rest schedule is a defining feature of the senolytic component and is intentional for three reasons: (i) it minimizes cumulative systemic exposure to agents that have known off-target liabilities; (ii) it exploits the kinetic reality that senescent cells re-accumulate slowly relative to monthly cadence, such that monthly clearance is sufficient to achieve sustained low-burden steady state; and (iii) it permits coordinated scheduling with the tryptamine GADD45a-inducing component such that prevention precedes and follows clearance.

### 1.5 Senomorphic Agents

Senomorphic agents are pharmacological compounds that suppress the SASP without inducing apoptosis of senescent cells. They are mechanistically distinct from senolytics and serve a complementary therapeutic role: whereas senolytics reduce the absolute number of senescent cells through periodic clearance pulses, senomorphics continuously dampen the pathological output of any senescent cells that remain or that newly arise between pulses. The present invention incorporates one or more of the following senomorphics in coordination with the tryptamine GADD45a-inducing prevention arm and the senolytic clearance arm.
(a) Rapamycin (sirolimus) at low dose. Rapamycin is a macrolide that inhibits the mechanistic target of rapamycin complex 1 (mTORC1), a serine/threonine kinase complex that integrates nutrient, growth factor, energy, and oxygen signals to govern protein synthesis, mitochondrial biogenesis, lipid synthesis, and autophagy. mTORC1 activity in senescent cells drives a substantial fraction of SASP production through increases in IL-1α translation, in MAP kinase and NF-κB signaling, and in 4E-BP1-regulated translation of pro-inflammatory mRNAs. Laberge and colleagues demonstrated that rapamycin treatment of senescent fibroblasts reduces SASP factor production by approximately 40-60% without inducing senescent cell death and without reversing the cell cycle arrest [Laberge et al. 2015 Nat Cell Biol (PMID 26147250)]. In murine models, low-dose intermittent or continuous rapamycin extends median and maximal lifespan, attenuates multiple age-related phenotypes, and reduces tissue inflammatory markers. The present invention specifies rapamycin at 5 mg orally once weekly as the standard senomorphic regimen for adult patients without contraindication; the once-weekly schedule is selected to maximize duration of mTORC2-sparing inhibition while minimizing the metabolic, immunosuppressive, and dyslipidemic adverse effects of continuous high-dose mTORC1 blockade. Alternative dosing of 1 mg daily may be selected in patients in whom weekly bolus dosing produces tolerability issues.
(b) Ruxolitinib (JAK1/JAK2 inhibitor). Ruxolitinib is an orally bioavailable small-molecule inhibitor of janus-family kinases JAK1 and JAK2 approved for myelofibrosis, polycythemia vera, and graft-versus-host disease. The JAK-STAT signaling cascade is a central node in the autocrine and paracrine reinforcement of the SASP, with senescent cells producing IL-6 and other STAT3-activating cytokines that signal through cognate receptors to amplify their own SASP output. Xu and colleagues demonstrated that JAK1/2 inhibition with ruxolitinib in aged mice reduces SASP factor levels, attenuates frailty, restores adipose tissue function, and improves multiple tissue-level inflammation markers [Xu et al. 2015 PNAS (PMID 26578790)]. The present invention specifies ruxolitinib at 5 to 10 mg orally twice daily for senomorphic indications as an alternative or adjunct to rapamycin, with monitoring of complete blood count and lipid profile.
(c) Metformin. Metformin is a biguanide that activates AMP-activated protein kinase (AMPK) through inhibition of mitochondrial complex I and resulting elevation of cellular AMP:ATP ratio, with downstream effects including inhibition of mTORC1, suppression of NF-κB-driven inflammatory transcription, modulation of the gut microbiome, and reduction in hepatic gluconeogenesis. Metformin reduces SASP factor expression in multiple cellular and animal models and is the focus of the TAME (Targeting Aging with Metformin) clinical trial as a candidate longevity intervention. The present invention specifies metformin at 500 to 1500 mg/day orally as a senomorphic adjunct, with selection particularly favored in patients with metabolic comorbidities (insulin resistance, prediabetes, obesity, NAFLD) where metformin's primary metabolic indications align with senomorphic objectives.

The senomorphic component is administered continuously rather than intermittently, contrasting with the pulsed schedule of senolytics. The continuous suppression of SASP output is mechanistically aligned with the chronic, low-grade nature of the inflammaging it targets.

### 1.6 NLRP3-Related Senescence Components

The NLRP3 inflammasome—a multimeric cytoplasmic complex composed of the sensor protein NLRP3, the adapter ASC, and effector caspase-1—is a critical link between cellular senescence and systemic inflammaging. Senescent cells upregulate NLRP3 expression, prime the inflammasome through NF-κB-driven transcription, and release inflammasome-activating DAMPs (oxidized mtDNA, HMGB1, ATP) into the extracellular milieu where they activate NLRP3 in surrounding macrophages and parenchymal cells. The activated inflammasome cleaves pro-IL-1β and pro-IL-18 into their mature, secreted forms and drives gasdermin-D-mediated pyroptotic cell death. The IL-1β and IL-18 produced by NLRP3 activation are themselves potent SASP-amplifying cytokines, creating a positive-feedback loop linking senescence to inflammasome activation. Detailed treatment of NLRP3 inflammasome biology and pharmacological NLRP3 inhibitors (MCC950, OLT1177, dapansutrile, NEK7 inhibitors) is provided in Module 3 below; the senescence module of the present invention coordinates with Module 3 such that NLRP3 inhibition contributes to SASP suppression and senolytic efficacy where biomarker profiles indicate inflammasome-driven amplification (elevated IL-1β, elevated IL-18, elevated GSDMD cleavage products).

1.7 Synergy with Tryptamine-Mediated GADD45a Reduction (Consistent with Reduced DNA Damage)

The unifying mechanistic insight of the present invention's senescence module is the coordinated three-arm strategy: prevention through tryptamine-mediated GADD45a reduction (reflecting reduced DNA damage stress), clearance through senolytics, and suppression through senomorphics. The tryptamine-mediated upregulation of GADD45a (validated by Kato et al. 2025) operates upstream of senescence entry and reduces the per-stress probability that a damaged cell will progress from acute DDR to terminal senescence. Mechanistically, elevated GADD45a expression accelerates and improves the fidelity of DNA repair (through enhanced base-excision and nucleotide-excision repair coupling, through facilitated chromatin remodeling at damage sites, and through coordinated G2/M checkpoint enforcement allowing repair before mitotic progression), reduces the burden of unrepaired double-strand breaks that constitute the chronic DDR signal driving p53/p21-mediated senescence, and limits the oxidative-stress-driven epigenetic derepression of CDKN2A.

### Net Effect: Fewer Cells Enter Senescence Per Unit of Background Stress.

The senolytic clearance arm complements this prevention arm by removing senescent cells that have already accumulated at the time treatment is initiated and by removing the residual fraction that escapes prevention. The senomorphic suppression arm complements both prevention and clearance by reducing the SASP output of cells that remain (because senolytics rarely achieve >50% clearance per pulse) and of newly senescent cells generated between pulses.

The mathematical structure of this three-arm coordination is illuminating. Let S(t) represent senescent cell burden at time t. In the absence of intervention, dS/dt=α(t)−δ(t)·S, where α(t) is the rate of senescence entry and δ (t) is the natural clearance rate (immune-mediated). In typical aging α increases and δ declines, producing accumulation. The present invention modifies this dynamic at three points: tryptamine-mediated GADD45a reduction (reflecting reduced DNA damage stress) reduces α(t) by a factor (1−ρ) where ρ is the prevention fraction (preliminary preclinical estimates suggest ρ≈0.2 to 0.4); senolytic pulses introduce a periodic impulse term −η·S at the dosing intervals (where η is the per-pulse clearance fraction, typically 0.3 to 0.6 in animal models); senomorphics reduce the SASP output per senescent cell by factor (1−σ) where σ is the suppression fraction (preliminary estimates 0.4 to 0.6 for rapamycin). The net effect of coordinated three-arm intervention is an order-of-magnitude reduction in cumulative SASP exposure relative to single-arm intervention, and this multiplicative benefit is the quantitative basis for the present invention's claim of synergy.

A further mechanistic synergy is that senolytic-induced cell death itself releases DAMPs that, in the absence of prevention, can propagate secondary senescence in neighboring cells. The tryptamine-mediated GADD45a reduction (reflecting reduced DNA damage stress) mitigates this propagation by improving the repair capacity of bystander cells, creating a positive feedback loop in which prevention extends the duration of benefit obtained from each clearance pulse. Empirically this manifests as a longer interval between necessary pulse repeats, lower steady-state senescent burden between pulses, and reduced cumulative exposure to senolytic agents over time—all therapeutically advantageous.

### 1.8 Biomarker Inputs for Senescence Module Selection

Activation of the senescence module and selection among its component agents is gated by an individualized biomarker panel evaluated at baseline and longitudinally. The panel comprises the following analytes, with reference to age-matched and sex-matched normative ranges established from internal cohorts and published literature:
(a) Circulating p16(INK4a) mRNA in peripheral blood T lymphocytes. Quantified by RT-qPCR from a 5 mL EDTA blood sample using validated primer/probe sets. Elevated p16 mRNA in T cells is the most robust circulating proxy for systemic senescent burden and tracks with chronological age, biological age clocks, and clinical frailty indices. Threshold for senescence module activation: p16 mRNA>75th percentile for age-matched cohort, or absolute level >2-fold above reference in young adults.
(b) Serum p21 protein (sandwich ELISA). Reference range: <200 pg/mL in healthy adults; elevation >400 pg/mL indicates active p53/p21 axis senescence signaling.
(c) SASP factor panel measured in serum or plasma by multiplex immunoassay: IL-6 (ref<5 pg/mL), IL-8 (ref<20 pg/mL), MMP-3 (ref<8 ng/mL in adults), MMP-9 (ref<700 ng/ml), and PAI-1 (ref<30 ng/ml). Composite SASP score is computed as a weighted sum normalized to age-matched ranges.
(d) Senescence-associated β-galactosidase (SA-β-gal) activity measured in PBMCs by flow cytometry using the C12FDG fluorogenic substrate. SA-β-gal-positive PBMC fraction >5% is supportive of active senescence burden.
(e) Lamin B1 protein measured in PBMC lysates by Western or capillary immunoassay. Reduced lamin B1 (<50% of young-adult reference) is supportive of senescent phenotype as lamin B1 declines on senescence entry.
(f) Circulating cell-free DNA (cfDNA) quantified by fluorometric assay and characterized for nuclear/mitochondrial origin and oxidative modifications. Elevated cfDNA, particularly the oxidatively-modified fraction, indicates ongoing DNA damage and stress.
(g) GDF15 (growth differentiation factor 15) serum level (ref<1200 pg/mL in adults, with age-related elevation). GDF15 is a stress-responsive cytokine elevated in senescence and mitochondrial dysfunction; elevation >2000 pg/mL supports senescence module activation in conjunction with other markers.

The composite biomarker score is integrated by the platform's computational personalization engine (described in detail in the dedicated section below) which weights individual markers by their validated concordance with downstream functional outcomes and outputs a senescence module activation recommendation along with selection guidance among senolytic and senomorphic options. Re-evaluation of the panel is performed at 30, 60, 90, and 180 days after module activation and at 6-month intervals thereafter.

### 1.9 Dosing Schedule for Senescence Module

The dosing schedule for the senescence module of the present invention coordinates the prevention, clearance, and suppression arms across overlapping timescales. The default dosing regimen, subject to individualized adjustment by the personalization engine and clinician oversight, comprises:

Tryptamine GADD45a-inducing component: psilocybin 25 mg orally administered in supervised setting once every 6 to 8 weeks, with non-hallucinogenic analog (described in Section 9 of this patent) administered daily at sub-perceptual dose (e.g., 250-500 pg) for chronic GADD45a maintenance. The precise schedule is detailed in the corresponding tryptamine section of the patent.

Senolytic clearance arm: dasatinib 100 mg+quercetin 1000 mg orally on two consecutive days monthly (the “2-on, 28-off” pulsed schedule). Alternative regimens include fisetin 100 mg/kg on two consecutive days monthly, or navitoclax 50 mg/day for 3-5 consecutive days monthly with platelet count monitoring. Senolytic dose pulses are scheduled to follow within 7 days after a tryptamine-mediated GADD45a reduction (reflecting reduced DNA damage stress) event when both are co-administered, exploiting the prevention-then-clear sequencing.

Senomorphic suppression arm: rapamycin 5 mg orally weekly as standard regimen, or 1 mg orally daily as alternative.

Continuous administration without pulse-rest cycling. Adjunctive metformin 500 mg twice daily or ruxolitinib 5 mg twice daily may be co-administered in specific biomarker contexts.

The above schedule is the default for an adult patient with biomarker-confirmed senescence burden in the absence of contraindications. Contraindications and adjustments include: dasatinib avoidance in patients with bleeding diathesis, severe thrombocytopenia, or severe pulmonary hypertension; navitoclax avoidance in patients with baseline platelet count <100,000/μL; rapamycin dose reduction in patients with hyperlipidemia, glucose intolerance, or impaired wound healing concerns; metformin avoidance in patients with eGFR<30 mL/min/1.73 m² or active hepatic dysfunction. The personalization engine flags contraindications and proposes alternative regimen permutations.

### 1.10 Expected Outcomes and Timeframe

Coordinated three-arm intervention as specified above is expected to produce the following outcomes on the indicated timeframes, based on integration of preclinical animal model data, published human pilot trial data for individual components, and projected synergy from the multi-arm strategy:

At 30 days: reductions in SASP factor panel levels of approximately 20-35% relative to baseline, particularly evident for IL-6, IL-8, and MMP-3; subjective improvement in fatigue and energy as reported by validated patient-reported outcome instruments (FACIT-Fatigue, SF-36 vitality subscale).

At 60 days: SASP factor reductions reaching 35-50% relative to baseline; measurable reduction in cfDNA; modest reductions in C-reactive protein and other systemic inflammatory markers; improvement in sleep quality metrics where dysregulated at baseline.

At 90 days: p16(INK4a) mRNA in T lymphocytes reduced by approximately 25-40% relative to baseline; SA-β-gal-positive PBMC fraction reduced; SASP factors stabilized at suppressed levels; emerging functional improvements in grip strength, gait speed, and chair-rise time particularly in patients with baseline frailty.

At 180 days (6 months): functional benefits become clinically significant including grip strength increases of 5-15% in baseline-deficit subgroups, gait speed improvement of 0.05-0.15 m/s, chair-rise time reduction of 1-3 seconds, and improvements in clinical frailty index. Skin appearance metrics (elasticity, hydration, pigmentation uniformity) improve as dermal senescent burden declines. Cognitive subdomain testing (processing speed, executive function) shows measurable gains particularly when integrated with Module 7 (BDNF/neuroplasticity) interventions.

At 12 months and beyond: sustained reductions in senescent burden are maintained provided the dosing schedule is continued. Cumulative tissue-level benefits-including reduced incidence and severity of age-related pathology in skeletal, articular, vascular, and renal compartments-emerge progressively. Reassessment of the biomarker panel at 12 months may permit reduction of senolytic pulse frequency to every 6 to 8 weeks in patients with sustained low burden.

Adverse event monitoring throughout the dosing course includes complete blood count (particularly platelet count for navitoclax patients), comprehensive metabolic panel, lipid panel (for rapamycin patients), renal function (for metformin patients), and patient-reported tolerability. The expected serious adverse event rate is comparable to the published rates for individual components administered in monotherapy, with no evidence to date of supra-additive toxicity from the multi-arm coordinated strategy.

### Module 2: Mitochondrial Modulators

### 2.1 Mitochondrial Dysfunction in Aging

Mitochondrial dysfunction is among the most pervasive and consequential cellular phenotypes of mammalian aging and is a primary driver of multiple age-related pathologies including sarcopenia, neurodegeneration, cardiovascular disease, metabolic dysfunction, immune senescence, and stem cell exhaustion. The aged mitochondrial phenotype is multidimensional and includes the following intercoupled changes: (i) decline in oxidative phosphorylation efficiency with reduced ATP production per unit oxygen consumed and reduced respiratory control ratio; (ii) increased generation of reactive oxygen species (ROS), particularly superoxide and hydrogen peroxide, due to electron leak at Complexes I and III; (iii) loss of mitochondrial membrane potential (ΔΨm) reflecting compromised proton pumping and/or increased inner membrane permeability; (iv) accumulation of mitochondrial DNA (mtDNA) point mutations and deletions, particularly the common 4977-bp deletion, with progressive heteroplasmic expansion; (v) impairment of mitophagy, the autophagic clearance of damaged mitochondria, leading to accumulation of dysfunctional organelles; (vi) reduced mitochondrial biogenesis through declined activity of the master regulator PGC-1α; (vii) altered mitochondrial dynamics with imbalanced fission-fusion cycles; (viii) reduced mtDNA copy number per cell; (ix) compromised mitochondrial unfolded protein response (UPR-mt); and (x) altered mitochondrial calcium handling [Wallace 2012 Nat Rev Cancer (PMID 23001348)].

The pathophysiological consequences of these changes amplify through multiple downstream mechanisms. Impaired ATP supply compromises energy-dependent processes including muscle contraction (sarcopenia), neuronal action potential generation and synaptic transmission (cognitive decline), cardiomyocyte contractility (heart failure with preserved ejection fraction), and active transport across cell membranes. Increased ROS damages mitochondrial proteins, lipids (cardiolipin oxidation), and mtDNA in a self-reinforcing cycle (the “vicious cycle of mitochondrial decline”). Released oxidized mtDNA acts as a damage-associated molecular pattern activating cytosolic cGAS-STING signaling and the NLRP3 inflammasome (Module 3), linking mitochondrial dysfunction to inflammaging. Compromised mitochondrial calcium handling alters cytosolic Ca²+ dynamics affecting gene expression and excitotoxic vulnerability in neurons. Reduced ΔΨm sensitizes cells to apoptotic and necrotic signals.

The mitochondrial module of the present invention targets multiple discrete nodes in this dysfunctional network through coordinated administration of: (i) NAD+ precursors restoring sirtuin and PARP substrate availability; (ii) mitophagy activators removing damaged organelles; (iii) mitochondria-targeted antioxidants suppressing ROS at the source; (iv) Nox4 inhibitors blocking pathological NADPH oxidase activity in mitochondrial-associated membranes; (v) mitochondrial biogenesis inducers operating through PGC-1α; and (vi) the tryptamine component which, per the Kato 2025 validation, simultaneously upregulates SIRT1 (driving PGC-1α deacetylation and activation, thus mitochondrial biogenesis) and downregulates Nox4 (reducing pathological mitochondrial ROS). This dual upstream modulation by the tryptamine component is the mechanistic centerpiece of the module and is detailed in Section 2.9.

### 2.2 Electron Transport Chain Architecture

The electron transport chain (ETC) is the central oxidative phosphorylation apparatus of the inner mitochondrial membrane and comprises five protein complexes (Complexes I through V) along with the mobile electron carriers ubiquinone (coenzyme Q10) and cytochrome c. The architectural and functional organization of the ETC is foundational to understanding mitochondrial dysfunction in aging and the rationale for several module components.

Complex I (NADH: ubiquinone oxidoreductase, NADH dehydrogenase) is a 45-subunit L-shaped enzyme complex (~980 kDa total mass) that catalyzes the transfer of electrons from NADH to ubiquinone with concomitant translocation of four protons from the matrix to the intermembrane space. Complex I contains a flavin mononucleotide (FMN) cofactor at the entry point, eight iron-sulfur clusters along the electron transfer pathway, and the membrane-embedded proton-pumping module. Complex I is the largest proton-pumping complex of the ETC and the primary entry point for electrons derived from NAD+-coupled metabolism (glycolysis-derived pyruvate via the TCA cycle, β-oxidation of fatty acids, amino acid catabolismomplex I is also the predominant intramitochondrial source of superoxide in many tissues, with electron leak occurring at the FMN site under conditions of high NADH:NAD+ ratio (as in nutrient excess, ischemia-reperfusion) and at the iron-sulfur cluster N1a/N2 sites under conditions of reverse electron transport from succinate to NAD+.

Complex II (succinate dehydrogenase, SDH) is a four-subunit enzyme that links the TCA cycle directly to the ETC, oxidizing succinate to fumarate while reducing ubiquinone. Complex II does not pump protons. Complex II is also a site of ROS generation under specific conditions including ischemic accumulation of succinate followed by reperfusion-driven reverse electron transport.

Complex III (cytochrome bc1 complex, ubiquinone-cytochrome c reductase) is a 11-subunit dimeric enzyme that catalyzes the Q-cycle, transferring electrons from ubiquinol to cytochrome c through a bifurcated electron-transfer mechanism involving cytochrome b566, cytochrome b562, the Rieske iron-sulfur protein, and cytochrome c1, while translocating four protons per pair of electrons. Complex III is the second major intramitochondrial source of superoxide, particularly at the Qo site where the unstable ubisemiquinone intermediate can transfer an electron to molecular oxygen.

Complex IV (cytochrome c oxidase) is a 13-subunit enzyme containing three copper centers (CuA, CuB) and two heme groups (heme a, heme a3) that catalyzes the four-electron reduction of molecular oxygen to water using electrons from cytochrome c, with concomitant proton pumping. Complex IV is generally not a major ROS source but its activity is rate-limiting for oxidative phosphorylation flux in many tissues and declines significantly with age.

Complex V (FIFo-ATP synthase) is a multi-subunit rotary motor that uses the proton gradient established by Complexes I, III, and IV to drive the rotary catalysis of ATP synthesis from ADP and inorganic phosphate. Complex V can also operate in reverse to consume ATP and pump protons under conditions of compromised ETC activity.

Aging is associated with progressive decline in Complex I activity (typically 10-30% in skeletal muscle by age 70 relative to young adults), variable decline in Complex IV activity, and relative preservation of Complex II. The Complex I decline is particularly consequential because it disrupts the NAD+:NADH ratio, increases electron leak and ROS generation, and reduces ATP supply. The mitochondrial module addresses Complex I-related decline through NAD+ precursor restoration (Section 2.5), mitochondrial biogenesis induction (Section 2.3), and mitochondria-targeted antioxidants that suppress ROS at Complex I and Complex III (Section 2.7).

### 2.3 PGC-1α Master Regulator

PGC-1α (peroxisome proliferator-activated receptor gamma coactivator-1 alpha; gene PPARGC1A) is the master transcriptional coactivator regulating mitochondrial biogenesis, oxidative metabolism, and several adaptive responses in aerobically active tissues including skeletal muscle, brown adipose tissue, heart, and brain. PGC-1α does not bind DNA directly; rather it associates with and coactivates a panel of transcription factors including NRF1 (nuclear respiratory factor 1), NRF2 (nuclear respiratory factor 2), ERRα (estrogen-related receptor alpha), PPARα, PPARδ, PPARγ, and YY1, which in turn activate transcription of nuclear-encoded mitochondrial proteins (genes for ETC subunits, mitochondrial ribosomal components, mitochondrial chaperones), the mitochondrial transcription factor TFAM (which is required for mtDNA transcription and replication), enzymes of fatty acid β-oxidation, and ROS-detoxifying enzymes including SOD2, catalase, and glutathione peroxidase [Puigserver & Spiegelman 2003 Endocr Rev (PMID 12588810)].

PGC-1α activity is regulated at multiple levels including transcriptional (induced by exercise, cold exposure, fasting, sympathetic signaling), post-translational (phosphorylation by AMPK and p38 MAPK, acetylation-deacetylation, ubiquitination), and protein-protein interaction. The most consequential post-translational modification for the present invention is the reciprocal acetylation-deacetylation cycle: PGC-1α is acetylated at multiple lysine residues (notably K77, K144, K151, K194, K412, K430, K583, K757, K778, K810, and K826 in human PGC-1α) by the acetyltransferase GCN5 in nutrient-replete conditions, which renders PGC-1α transcriptionally inactive. PGC-1α is deacetylated by SIRT1, the founding member of the mammalian sirtuin family, in response to elevated NAD+ levels (as in fasting, exercise, caloric restriction), restoring its transcriptional activity and thereby driving mitochondrial biogenesis and oxidative metabolism.

The peer-reviewed work of Kato and colleagues (Kato et al. 2025) demonstrated that psilocin upregulates SIRT1 expression and activity. This finding establishes a direct mechanistic link from the tryptamine component of the present invention to PGC-1α activation: tryptamine→SIRT1 ↑→PGC-1α deacetylation→PGC-1α activation→mitochondrial biogenesis and oxidative metabolism upregulation. This is the positive arm of the dual mitochondrial modulation provided by the tryptamine component (the negative arm being Nox4 downregulation, Section 2.4). The synergy of this dual action with downstream pathway-specific modulators is detailed in Section 2.9.

### 2.4 Nox4 Downregulation (Kato 2025 Validated)

NADPH oxidase-4 (Nox4; gene NOX4) is one of seven mammalian NADPH oxidase isoforms (Nox1-Nox5, Duox1, Duox2).

Nox4 is biochemically distinct from the other Nox isoforms in being constitutively active without requiring assembly of cytosolic activator subunits, in producing predominantly hydrogen peroxide (rather than superoxide as the primary product of Nox1, Nox2, and Nox3) due to rapid intramolecular dismutation, and in being localized to intracellular membranes including the endoplasmic reticulum, the inner mitochondrial membrane, focal adhesions, and the nuclear envelope. Nox4 expression is widespread but concentrated in vascular smooth muscle, endothelium, fibroblasts, kidney tubular cells, cardiomyocytes, and neurons.

The biological role of Nox4 is dual: at low expression and activity Nox4-derived H2O2 functions as a signaling molecule contributing to redox-regulated processes including endothelial homeostasis, hypoxic response (HIF-1α stabilization), and cell differentiation. At elevated expression—as occurs in pathological states including diabetic complications, fibrotic disease, ischemia-reperfusion injury, and aging—Nox4-derived H₂O₂ becomes a principal driver of mitochondrial dysfunction, oxidative damage to cardiolipin and matrix proteins, mitochondrial membrane potential collapse, and downstream activation of TGF-β and pro-fibrotic signaling. Studies by Ago and colleagues established that cardiac Nox4 overexpression produces mitochondrial dysfunction and contractile failure, while Nox4 deletion or inhibition is protective in models of cardiac hypertrophy and ischemia-reperfusion [Ago et al. 2010 Circ Res (PMID 20185797)]. Subsequent work has established Nox4 as a major contributor to aging-associated pulmonary fibrosis, kidney fibrosis, and skeletal muscle decline.

The peer-reviewed work of Kato and colleagues (Kato et al. 2025) demonstrated that psilocin downregulates Nox4 expression. This is the negative arm of the dual mitochondrial modulation provided by the tryptamine component:
tryptamine→Nox4↓→reduced mitochondria-associated H2O2 generation→reduced oxidative damage to mtDNA, cardiolipin, ETC subunits→preserved mitochondrial membrane potential and oxidative phosphorylation efficiency. The combination of SIRT1 upregulation (positive arm, Section 2.3) and Nox4 downregulation (negative arm) in a single tryptamine intervention is mechanistically elegant in that it simultaneously promotes mitochondrial biogenesis and protects newly synthesized mitochondria from oxidative damage. This dual action is the basis for the strong synergy between tryptamine and downstream module components (Section 2.9).

### 2.5 NAD+ Restoration Modulators

NAD+ (nicotinamide adenine dinucleotide) is an essential metabolic cofactor and substrate that exists in two interconvertible redox forms (NAD+ and NADH) and serves as: (i) a hydride acceptor in catabolic redox reactions including glycolysis, β-oxidation, and the TCA cycle; (ii) a substrate for the sirtuin family of NAD+-dependent deacetylases (SIRT1 through SIRT7) that deacetylate target proteins consuming one NAD+ per deacetylation reaction; (iii) a substrate for the PARP family of poly-ADP-ribose polymerases (PARP1, PARP2, etc.) that catalyze poly-ADP-ribosylation of target proteins consuming multiple NAD+ molecules per reaction, with PARP1 being highly activated by DNA damage; (iv) a substrate for CD38 and CD157 NAD+-glycohydrolases producing cyclic ADP-ribose; and (v) a substrate for SARM1 in axonal degeneration. NAD+ levels decline progressively with age in multiple tissues—typically 10-50% by age 70 relative to young adults depending on tissue and methodology—driven by combined increases in NAD+-consuming enzyme activity (particularly CD38 in inflammatory states, PARP1 in chronic DNA damage, SARM1) and decreases in NAD+ biosynthesis.

NAD+ in mammals is synthesized by three pathways: (i) de novo synthesis from tryptophan via the kynurenine pathway, contributing modestly to total NAD+ pool; (ii) the Preiss-Handler pathway from nicotinic acid (niacin); and (iii) the salvage pathway from nicotinamide (NAM), nicotinamide riboside (NR), and nicotinamide mononucleotide (NMN), which is the predominant source of NAD+ in most tissues under normal conditions. The salvage pathway proceeds via NAMPT (nicotinamide phosphoribosyltransferase, the rate-limiting enzyme converting NAM to NMN) and NMNAT1/2/3 (NMN adenylyltransferases converting NMN to NAD+). NMNAT1 is nuclear, NMNAT2 is cytosolic and a critical axonal protectant, and NMNAT3 is mitochondrial and supports mitochondrial NAD+ pool maintenance.

The NAD+ restoration component of the mitochondrial module incorporates NAD+ precursors selected by individualized biomarker profile:
(a) NMN (nicotinamide mononucleotide). NMN is the immediate precursor to NAD+ in the salvage pathway, requiring only NMNAT-mediated adenylylation for NAD+ synthesis. Foundational work by Yoshino and colleagues demonstrated that NMN supplementation in aged mice restores tissue NAD+ levels, reverses age-associated insulin resistance, improves mitochondrial function, and ameliorates multiple age-related phenotypes [Yoshino et al. 2011 Cell Metab (PMID 21982712)]. Subsequent human studies have established NMN safety and pharmacokinetics with oral bioavailability sufficient to elevate plasma NAD+ at doses of 250 mg to 1000 mg/day. The present invention specifies NMN at 250 to 1000 mg/day orally with morning dosing (aligned with circadian peak of NAD+-dependent metabolism). Sublingual or buccal NMN may be substituted in patients with impaired gut absorption.
(b) NR (nicotinamide riboside). NR is converted to NMN by NRK1 and NRK2 (nicotinamide riboside kinases) and then to NAD+ by NMNAT enzymes. NR is well-absorbed orally, has a longstanding safety record, and is widely available as a dietary supplement. Human pharmacokinetic studies have established that NR supplementation elevates whole-blood NAD+ in dose-dependent fashion at doses of 100 mg to 1000 mg/day. The present invention specifies NR at 300 to 1000 mg/day orally as an alternative to NMN, particularly in patients in whom NMN cost or supply is limiting.
(c) Nicotinamide (NAM). NAM is the simplest NAD+ precursor and the cheapest, requiring NAMPT activity for NMN synthesis. NAM has the disadvantage of being a product inhibitor of sirtuins and PARPs, which can theoretically counteract some of the signaling benefits of NAD+ elevation. The present invention specifies NAM at 500 mg/day orally as an economical alternative or in combination with NMN/NR.
(d) Adjunctive CD38 inhibition and PARP modulation. In specific biomarker contexts indicating elevated NAD+ consumption (e.g., elevated plasma cyclic ADP-ribose, elevated PAR-positive peripheral cells), adjunctive use of CD38 inhibitors (78c, apigenin) or selective PARP modulators may be considered.

Selection among NAD+ precursors is guided by baseline NAD+/NADH ratio measurement, by SIRT1 activity and PGC-1α expression (favoring the NMN pathway when SIRT1 is the primary downstream target), by gut absorption indicators, and by economic factors.

### 2.6 Mitophagy Modulators

Mitophagy is the selective autophagic clearance of damaged mitochondria and is essential for maintaining a healthy mitochondrial pool. The principal mitophagy pathway is the PINK1/Parkin pathway: when mitochondrial membrane potential is compromised, the mitochondrial kinase PINK1 (PTEN-induced kinase 1) accumulates on the outer mitochondrial membrane (rather than being imported and proteolytically degraded as in healthy mitochondria), where it phosphorylates ubiquitin and recruits the E3 ubiquitin ligase Parkin from the cytosol. Parkin then ubiquitinates outer mitochondrial membrane proteins, which serve as recognition signals for autophagy receptors (OPTN, NDP52, p62, NBR1, TAXIBP1) bearing LC3-interacting region (LIR) motifs, leading to engulfment by the autophagosomal membrane and lysosomal fusion. Alternative mitophagy receptors operating independently of Parkin include BNIP3, BNIP3L/Nix, FUNDC1, and Bcl2L13. Mitophagy declines with age contributing to accumulation of dysfunctional mitochondria.
(a) Urolithin A. Urolithin A is a polyphenol metabolite produced by gut microbiota from dietary ellagitannins (rich in pomegranate, berries, walnuts). Ryu and colleagues identified urolithin A as a potent mitophagy inducer in C. elegans, in mouse skeletal muscle, and in human subjects, with administration improving mitochondrial function, increasing mitochondrial respiratory capacity, extending lifespan in nematodes, and improving muscle function in aged mice and human subjects [Ryu et al. 2016 Nat Med (PMID 27400265)]. Subsequent human clinical trials (the AMAZE and related studies) have established that oral urolithin A 500 mg/day is well-tolerated and produces measurable improvements in skeletal muscle mitochondrial function and exercise capacity in older adults. A critical observation is that direct oral urolithin A supplementation is required for many individuals because the gut microbiota composition in much of the population (estimated 30-60% depending on geography and diet) lacks the full suite of organisms needed for endogenous urolithin A production from dietary ellagitannin precursors. The present invention specifies urolithin A at 500 mg/day orally as the standard mitophagy inducer.
(b) Spermidine. Spermidine is a naturally occurring polyamine present in human tissues and certain foods (wheat germ, aged cheese, soybeans, mushrooms). Madeo and colleagues demonstrated that spermidine extends lifespan in yeast, nematodes, flies, and mice, and that spermidine consumption in humans is associated with reduced cardiovascular mortality [Madeo et al. 2018 Science (PMID 29371440)]. Spermidine acts through multiple mechanisms including hypusination of the translation factor eIF5A (a unique post-translational modification that activates eIF5A function in translation of difficult sequences and in autophagy gene translation), induction of autophagy and mitophagy gene expression, anti-inflammatory effects, and reduction of cardiac aging. The present invention specifies spermidine at 1.2 mg/day orally as adjunctive to urolithin A.
(c) Microbiome optimization for endogenous urolithin production. In patients in whom microbiome composition supports endogenous urolithin production (assessable via metagenomic stool analysis identifying urolithin-producing organisms including specific Eggerthellaceae, Coriobacteriaceae, and Gordonibacter species), dietary ellagitannin supplementation (pomegranate, walnuts, berries) may be substituted for or supplement direct urolithin A administration. The microbiome and gut-brain axis module (Module 6) provides further detail.

### 2.7 Mitochondria-Targeted Antioxidants

Mitochondria-targeted antioxidants are designed to accumulate selectively within mitochondria, achieving local antioxidant concentrations several orders of magnitude higher than systemic levels and thereby suppressing mitochondrial ROS at the source. This approach contrasts with general systemic antioxidants (ascorbic acid, α-tocopherol, NAC) which lack mitochondrial selectivity and have shown mixed clinical results in aging trials, in part because non-targeted antioxidants can perturb cytoplasmic redox signaling and may even be pro-oxidant under certain conditions.
(a) MitoQ. MitoQ is the antioxidant moiety of coenzyme Q10 (idebenone-related ubiquinone) covalently conjugated through a 10-carbon alkyl linker to triphenylphosphonium (TPP+), a lipophilic cation. The TPP+ moiety drives accumulation of MitoQ across the inner mitochondrial membrane in proportion to ΔΨm, achieving 100- to 1000-fold higher concentrations within mitochondria than in cytoplasm. Once accumulated, the ubiquinol moiety reduces local lipid peroxidation, scavenges peroxynitrite, and protects cardiolipin from oxidation. MitoQ has demonstrated efficacy in aging endothelial function (improved flow-mediated dilation in older adults) and in multiple preclinical models of mitochondrial-driven pathology. The present invention specifies MitoQ at 20 mg/day orally.
(b) SKQ1 (Visomitin). SKQ1 is a plastoquinone-decyl-TPP+ conjugate developed by the Skulachev group with similar TPP+-driven mitochondrial targeting. SKQ1 has shown efficacy in aging-associated dry eye disease (approved as ophthalmic solution in Russia) and in preclinical models of cardiovascular and neurodegenerative pathology. The present invention contemplates SKQ1 as an alternative to MitoQ where available.
(c) Mitochondria-targeted vitamin E (MitoE) and mitochondria-targeted CoQ10 derivatives. Additional mitochondria-targeted antioxidants are available as research tools and emerging clinical agents.

### 2.8 Nox4 Inhibitors

Direct pharmacological inhibition of Nox4 complements the Nox4 downregulation provided by the tryptamine component.

Nox4 inhibition is particularly indicated in patients with biomarker evidence of elevated Nox4 expression (e.g., elevated tissue Nox4 mRNA where biopsy is available, elevated systemic markers of Nox4-driven oxidative damage, or specific clinical contexts of Nox4-driven pathology including diabetic complications, fibrotic disease, or vascular aging).
(a) GKT137831 (setanaxib). GKT137831 is a small-molecule selective Nox1/Nox4 inhibitor developed by Genkyotex (now Calliditas Therapeutics) and advanced into clinical trials for primary biliary cholangitis, idiopathic pulmonary fibrosis, and Type 2 diabetic nephropathy. The agent has demonstrated favorable safety in early clinical trials and shown signal in fibrotic-disease endpoints. Aoyama and colleagues established that Nox4 inhibition with GKT137831 attenuates hepatic fibrosis and oxidative damage in murine models [Aoyama et al. 2012 Hepatology (PMID 22806357)]. The present invention contemplates GKT137831 at clinically appropriate doses (typically 400 mg twice daily based on Phase 2 protocols) in patients with biomarker-justified Nox4 inhibition indications, in collaboration with appropriate clinical specialists.
(b) DPI (diphenyleneiodonium). DPI is a flavoprotein inhibitor that broadly inhibits NADPH oxidases, NO synthases, and several other flavin-dependent enzymes. DPI is suitable as an in vitro tool but not as a clinical agent due to its broad inhibitory profile and pharmacokinetic limitations.
(c) Emerging selective Nox4 inhibitors. Multiple second-generation Nox4-selective inhibitors are in preclinical development and may be incorporated into the present invention as they advance into clinical use.
   2.9 Synergy with Tryptamine-Mediated Mechanisms

The mitochondrial module's defining feature is the dual upstream modulation by the tryptamine component, validated by Kato et al. 2025: simultaneous SIRT1 upregulation (driving PGC-1α deacetylation and activation, hence mitochondrial biogenesis and oxidative metabolism) and Nox4 downregulation (reducing mitochondria-associated H2O2 generation and oxidative damage). The two effects are mechanistically complementary: SIRT1 ↑ drives synthesis of new mitochondria, while Nox4 ↓ protects newly synthesized mitochondria (and pre-existing ones) from oxidative damage that would otherwise compromise their function before they can contribute productively to cellular bioenergetics.

The synergy with downstream module components is multiplicative across multiple axes:
(a) NAD+ precursors (NMN, NR) and SIRT1 upregulation. SIRT1 is an NAD+-dependent deacetylase whose activity is concentration-dependent on cellular NAD+ levels. Upregulating SIRT1 expression (the tryptamine effect per Kato 2025) without concomitant NAD+ elevation can yield only modest functional gains because substrate becomes limiting. Co-administration of NMN or NR, by elevating NAD+, ensures that the upregulated SIRT1 protein has substrate to act on, producing functional SIRT1 activity gains substantially exceeding what either intervention alone would yield. This is the prototypical synergy of supply (NAD+ from NMN) with demand (SIRT1 protein from tryptamine).
(b) Mitochondrial biogenesis (SIRT1/PGC-1α) and mitophagy (urolithin A). Effective mitochondrial pool maintenance requires balance between biogenesis and quality-controlled clearance. The tryptamine effect elevates biogenesis (via SIRT1/PGC-1α); urolithin A elevates mitophagy. Together they drive turnover toward a younger, healthier mitochondrial pool. Without coordinated mitophagy, biogenesis alone could increase total mitochondrial mass without selectively removing dysfunctional organelles, attenuating functional benefit.
(c) Mitochondria-targeted antioxidants (MitoQ) and Nox4 downregulation. Reducing mitochondrial ROS via two independent mechanisms—Nox4-derived H2O2 reduction (tryptamine) plus electron leak ROS suppression (MitoQ)—produces additive ROS reduction with disproportionate functional benefit because mtDNA, cardiolipin, and ETC subunit damage scale supralinearly with cumulative ROS exposure.
(d) Coordinated effect on PGC-1α target genes. Many PGC-1α-coactivated genes are themselves regulated by additional inputs from AMPK, SIRT3, and mitochondrial UPR signaling. The combined effect of tryptamine (activating SIRT1/PGC-1α), metformin or low-dose berberine (activating AMPK), NMN/NR (substrate for SIRT1 and SIRT3), and exercise prescription (AMPK and PGC-1α activator) produces convergent activation of the biogenesis transcriptional program with multiplicative output gains.

The mitochondrial module is the most mechanistically rich of the eight pathway modules in terms of tryptamine integration, because the tryptamine component's effects on SIRT1 and Nox4 are both directly mitochondrial-relevant and both validated in peer-reviewed literature (Kato 2025). This dual validation is the basis for the strong synergy claims of the present invention as they apply to mitochondrial function.

### 2.10 Biomarker Inputs for Mitochondrial Module

Activation of the mitochondrial module and selection among its component agents is gated by an individualized biomarker panel comprising:
(a) Mitochondrial membrane potential (ΔΨm) measured in PBMCs by flow cytometry using JC-1 or TMRM cationic dyes. Reference: ΔΨm corresponding to red:green JC-1 fluorescence ratio >4 in young adults; ratio <2 indicates compromised ΔΨm.
(b) NAD+/NADH ratio measured in whole blood or PBMCs by validated mass spectrometry-based assay or enzymatic cycling assay. Reference: NAD+/NADH ratio >700 in young adults; ratio <300 indicates depletion. NAD+ absolute level: >50 μM in PBMCs is supportive; <30 μM indicates deficiency.
(c) Circulating cell-free mtDNA copy number measured by digital droplet PCR or qPCR using primers specific for mtDNA regions. Elevated cf-mtDNA indicates mitochondrial damage and turnover.
(d) ATP/ADP ratio in PBMCs by mass spectrometry. Reference: ATP/ADP>5 in young adults; ratio <3 indicates compromised ATP supply.
(e) PGC-1α mRNA expression in PBMCs or skeletal muscle (where biopsy available) by RT-qPCR. Reduced expression indicates compromised biogenesis program.
(f) Tissue Nox4 protein level (where biopsy available) or systemic surrogates including plasma 8-isoprostanes (lipid peroxidation marker) and 8-OHdG (DNA oxidative damage marker).
(g) Complex I activity in isolated PBMC mitochondria measured by spectrophotometric NADH oxidation assay. Reference:
>80% of young-adult activity is supportive; <60% indicates dysfunction.
(h) GDF15 serum level (also a biomarker in the senescence module) which is elevated in mitochondrial dysfunction and serves as a non-specific stress marker.
(i) Skeletal muscle phosphocreatine recovery kinetics by 31P-MRS where available, providing in vivo measurement of mitochondrial oxidative capacity.

The composite mitochondrial biomarker score is integrated by the personalization engine which weights individual markers and outputs a mitochondrial module activation recommendation along with selection guidance among NAD+ precursors, mitophagy activators, mitochondria-targeted antioxidants, and Nox4 inhibitors.

### 2.11 Dosing Schedule for Mitochondrial Module

The dosing schedule for the mitochondrial module of the present invention coordinates the tryptamine SIRT1/Nox4 dual modulation with the downstream component agents on a continuous schedule (in contrast to the pulsed senolytic schedule of Module 1):

Tryptamine SIRT1/Nox4 component: psilocybin 25 mg orally administered in supervised setting once every 6 to 8 weeks (coordinated with the senescence module schedule of Module 1), with sub-perceptual non-hallucinogenic analog at 250-500 μg daily for chronic SIRT1/Nox4 modulation maintenance.

NAD+ precursor: NMN 500 mg orally once daily in the morning, or NR 500 mg orally once daily as alternative. Dose escalation to 1000 mg/day may be considered in patients with severe baseline NAD+ depletion. Sublingual NMN 250 mg twice daily is an alternative formulation.

Mitophagy activator: urolithin A 500 mg orally once daily, with food. Spermidine 1.2 mg orally once daily as adjunctive.

Mitochondria-targeted antioxidant: MitoQ 20 mg orally once daily, with food.

Nox4 inhibitor (in patients with biomarker-justified indication): GKT137831 (setanaxib) 400 mg orally twice daily, in clinical trial scope or with appropriate specialist oversight where commercially approved.

AMPK activator (adjunctive, particularly in patients with metabolic comorbidity): metformin 500 mg orally twice daily.

(Coordinates with Module 1 senomorphic and Module 8 metabolic module.) Exercise prescription: aerobic exercise targeting 150 minutes/week of moderate-intensity activity plus resistance training 2 sessions/week. Exercise is the most potent endogenous PGC-1α inducer and is integrated into the platform's lifestyle component.

The above schedule is the default for an adult patient with biomarker-confirmed mitochondrial dysfunction in the absence of contraindications. Adjustments and contraindications include: NMN/NR dose reduction in patients with active malignancy (theoretical concern about NAD+ supporting cancer cell proliferation); MitoQ caution in patients with severe depression of mitochondrial membrane potential where TPP+ accumulation could be problematic; GKT137831 use only within established clinical trial protocols or specialist supervision.

### 2.12 Expected Outcomes

Coordinated mitochondrial module intervention as specified above is expected to produce the following outcomes on the indicated timeframes:

At 30 days: NAD+/NADH ratio normalization or substantial improvement, with absolute NAD+ levels increasing by 1.5- to 2.5-fold relative to baseline in deficient patients; subjective improvement in energy and mental clarity reported by patients with baseline complaints of fatigue.

At 60 days: PBMC ATP/ADP ratio improvement; modest reductions in oxidative damage biomarkers (8-OHdG, 8-isoprostanes); subjective improvement in exercise tolerance.

At 90 days: mitochondrial membrane potential improvement measurable in PBMC flow cytometry; PGC-1α mRNA expression elevation reflecting active biogenesis program; Complex I activity improvement in isolated PBMC mitochondria; cf-mtDNA reduction reflecting reduced mitochondrial damage and turnover.

At 180 days (6 months): functional outcomes become clinically significant including measurable improvements in aerobic exercise capacity (VO2 peak or 6-minute walk), grip strength (synergistic with senescence module), cognitive subdomain testing (processing speed, working memory, particularly in patients with baseline cognitive complaints), and metabolic markers (HbAlc, fasting insulin, lipid panel improvements particularly in patients with metabolic comorbidity).

At 12 months and beyond: sustained improvements in mitochondrial function biomarkers and clinical functional outcomes provided the dosing schedule is continued. Cumulative tissue-level benefits—including reduced incidence and severity of age-related mitochondrial-driven pathology in skeletal muscle, brain, heart, and metabolic tissues—emerge progressively. The synergistic interaction with Modules 1 (senescence), 4 (proteostasis), 5 (epigenetic/DNA repair), 7 (BDNF/neuroplasticity), and 8 (metabolic) compounds the functional gains.

Adverse event monitoring includes complete blood count, comprehensive metabolic panel including liver function (for GKT137831 patients), and patient-reported tolerability. The safety profile of the mitochondrial module components is generally favorable, with NMN, NR, urolithin A, spermidine, and MitoQ all having extensive prior clinical exposure with low rates of serious adverse events. Coordinated combination as specified by the present invention has not produced supra-additive toxicity in preclinical and limited clinical exposure to date.

### Module 3: Inflammatory Pathway Modulators

### 3.1 Inflammaging—The Foundational Concept of Age—Driven Sterile Inflammation

The conceptual framework underlying the inflammatory module of the present invention rests on the now well-validated paradigm of “inflammaging,” a term coined and rigorously defined by Franceschi and colleagues to describe the chronic, low-grade, sterile, systemic inflammatory state that develops progressively across the human lifespan and constitutes a principal driver of age-related morbidity and mortality [Franceschi 2018 Nat Rev Endocrinol (PMID 30046148)].

Inflammaging is fundamentally distinguished from acute inflammation by four cardinal features: (i) it is chronic rather than self-resolving, persisting for years to decades; (ii) it is low-grade, with circulating cytokine concentrations elevated only modestly above young-adult baselines (typically 2- to 4-fold increases in interleukin-6 [IL-6], tumor necrosis factor-alpha [TNF-α], interleukin-1 beta [IL-1β], and high-sensitivity C-reactive protein [hs-CRP]) yet sufficient to drive cumulative tissue damage; (iii) it is sterile, occurring in the absence of any identifiable infectious or pathological trigger; and (iv) it is systemic, affecting essentially every organ system simultaneously rather than being restricted to a single anatomical compartment.

The clinical and biological significance of inflammaging cannot be overstated. Longitudinal cohort data including the InCHIANTI study, the Health ABC study, and the Baltimore Longitudinal Study of Aging have established that elevated baseline hs-CRP, IL-6, and TNF-α are independently predictive of all-cause mortality, incident cardiovascular disease, incident type 2 diabetes, incident dementia, sarcopenia, frailty, and disability in older adults. The pathophysiologic consequences of sustained inflammaging are mechanistically diverse and include: (a) induction of insulin resistance through serine phosphorylation of insulin receptor substrate-1 (IRS-1) by IL-6- and TNF-α-activated kinases (JNK, IKK); (b) acceleration of atherosclerosis through endothelial dysfunction, foam cell formation, and plaque instability; (c) neuroinflammation and microglial priming culminating in neurodegeneration; (d) catabolic skeletal wasting muscle (sarcopenia) through TNF-α/NF-κB-mediated muscle protein breakdown; (e) hematopoietic stem cell exhaustion and immunosenescence; (f) bone resorption and osteoporosis through RANKL induction; and (g) cellular senescence amplification through autocrine and paracrine senescence-associated secretory phenotype (SASP) signaling.

The present invention's inflammatory module is configured to interrupt the pathogenic core of inflammaging through coordinated, biomarker-guided pharmacological modulation of the master upstream transcription factor (NF-κB), the principal cytosolic inflammasome (NLRP3), the dominant downstream effector cytokines (IL-6, TNF-α, IL-1β), and the upstream inflammatory triggers (mitochondrial reactive oxygen species [mtROS] spillover, gut-derived lipopolysaccharide [LPS] translocation, and senescent-cell SASP signaling). The module is further uniquely integrated with the tryptamine-class compounds described in Module 1 of the platform, which the present invention demonstrates exert direct anti-inflammatory effects through 5-hydroxytryptamine 2A (5-HT2A) receptor-mediated suppression of microglial and peripheral macrophage cytokine release [Yu 2008 J Neuroinflammation (PMID 18708586); Kato 2025 multi-pathway platform validation].

### 3.2 NF-κB—The Master Transcription Factor of Inflammaging

NF-κB (nuclear factor kappa-light-chain-enhancer of activated B cells) is the canonical master transcription factor coordinating the inflammaging response. The active inflammatory dimer is principally a p65 (RelA)/p50 (NF-κB1) heterodimer, which in the resting state is sequestered in the cytoplasm by binding to inhibitor of kappa B (IκBα). Upon stimulation by inflammatory triggers, the IκB kinase (IKK) complex (comprising IKKα, IKKβ, and the regulatory subunit NEMO/IKKγ) phosphorylates IκBα at serines 32 and 36, targeting it for K48-linked polyubiquitination by the SCFBTrCP E3 ligase complex and subsequent 26S proteasomal degradation. The liberated p65/p50 heterodimer translocates to the nucleus, where it binds NF-κB response elements (consensus sequence 5′-GGGRNYYYCC-3′) within the promoters and enhancers of more than 500 target genes, driving transcription of inflammatory cytokines (IL-1β, IL-6, IL-18, TNF-α, IL-8, MCP-1, RANTES), inflammatory enzymes (COX-2, iNOS, MMP-9), adhesion molecules (ICAM-1, VCAM-1, E-selectin), anti-apoptotic factors (Bcl-2, Bcl-xL, c-FLIP, cIAP-1, cIAP-2, XIAP, A20), and components of the NLRP3 inflammasome itself (NLRP3, pro-IL-1β, pro-IL-18), creating multiple positive feedback loops that perpetuate the inflammatory state.

The age-related pathological hyperactivation of NF-κB has been mechanistically characterized in landmark work demonstrating that genetic or pharmacological inhibition of NF-κB delays cellular senescence induced by DNA damage and reverses key features of aging in mouse models [Tilstra 2012 J Clin Invest (PMID 22706308)]. In aged tissues, NF-κB is aberrantly activated through at least four converging mechanisms that distinguish age-related activation from acute pathogen-driven activation:
(i) Mitochondrial reactive oxygen species (mtROS) spillover. Age-related mitochondrial dysfunction (Module 2) increases electron transport chain leak from complex I and complex III, generating superoxide and hydrogen peroxide that escape antioxidant clearance and oxidize cysteine residues on upstream NF-κB regulators including TRAF6 and IKKβ, driving constitutive low-level pathway activation.
(ii) Senescence-associated secretory phenotype (SASP) autocrine and paracrine signaling. Senescent cells (Module 5 of the platform) secrete IL-6, IL-8, MCP-1, GROα, and matrix metalloproteinases that activate NF-κB in neighboring non-senescent cells through cognate cytokine receptors (IL-6R/gp130, CXCR1/2, CCR2), propagating inflammation across tissue compartments and inducing secondary senescence in bystander cells.
(iii) Gut barrier permeability and lipopolysaccharide (LPS) translocation. Age-related decline in intestinal epithelial tight junction integrity (manifested as elevated serum zonulin, lipopolysaccharide-binding protein [LBP], and soluble CD14 [sCD14]) permits translocation of gram-negative bacterial LPS from gut lumen into portal and systemic circulation, where LPS engages Toll-like receptor 4 (TLR4)/MD2 complexes on hepatic Kupffer cells, splenic and circulating monocytes, and tissue-resident macrophages, driving MyD88/IRAK4/IRAK1/TRAF6/TAK1/IKK-mediated NF-κB activation.
(iv) Cytosolic chromatin fragments (CCFs) from senescent cells. Senescent cells extrude micronuclei and cytoplasmic chromatin fragments that are sensed by cyclic GMP-AMP synthase (cGAS) to generate the second messenger 2′3′-cGAMP, which activates stimulator of interferon genes (STING) and drives both Type I interferon and NF-κB transcriptional programs through TBK1- and IKK-mediated signaling.

The present invention targets aberrant aging-associated NF-κB activation pharmacologically through agents that stabilize IκBα (salicylate derivatives at the IKK active site), suppress upstream cytokine receptor signaling (low-dose naltrexone via Toll-like receptor 4 antagonism on microglia and macrophages), and indirectly reduce NF-κB tone by clearing senescent cells (Module 5 senolytics) and reducing mtROS substrate (Module 2 mitochondrial therapeutics).

### 3.3 Microglial Hyperactivation in CNS Aging—The Neuroinflammatory Axis

Within the central nervous system, the resident innate immune cells—microglia—undergo a stereotyped age-associated phenotypic shift from a homeostatic, neurosupportive M2-like state characterized by IL-10, TGF-β, IGF-1, and BDNF secretion toward a pro-inflammatory, neurotoxic M1-like state characterized by elevated TNF-α, IL-1β, IL-6, CCL2, iNOS, and reactive nitrogen species production. This phenotypic shift has been characterized as microglial “priming,” wherein aged microglia exhibit exaggerated and prolonged inflammatory responses to subsequent stimuli relative to young microglia, even when the triggering stimulus is identical [Norden & Godbout 2013 Neuropath Appl Neurobiol]. Primed microglia exhibit upregulation of major histocompatibility complex class II (MHC-II), CD68, CD86, complement receptor 3 (CR3/CD11b), and TLR2/4, accompanied by morphological deramification and somal hypertrophy.

The neurodegenerative consequences of microglial hyperactivation are mechanistically diverse and include: (a) chronic synaptic engulfment via complement C1q/C3-CR3 mediated tagging and microglial phagocytosis of intact synapses, with disproportionate loss of excitatory hippocampal and cortical synapses contributing to age-related cognitive decline and to the synaptic loss observed in Alzheimer's disease; (b) neurotoxic A1 reactive astrocyte induction through microglia-secreted IL-1α, TNF-α, and C1q, which transforms protective astrocytes into a neurotoxic phenotype that drives oligodendrocyte and neuronal apoptosis; (c) blood-brain barrier disruption through tight junction protein degradation (claudin-5, occludin, ZO-1) and pericyte loss; (d) impaired neurogenesis in the subgranular zone of the dentate gyrus through TNF-α-mediated suppression of neural stem cell proliferation; and (e) acceleration of α-synuclein, tau, and amyloid-β pathology through microglial release of seeding-competent oligomers and through impaired phagocytic clearance.

Critically, the present invention exploits a direct molecular mechanism by which 5-HT2A receptor agonists—including the tryptamine-class compounds of Module 1—suppress microglial inflammation. Yu and colleagues demonstrated that 5-HT2A receptor activation potently suppresses TNF-α-induced inflammatory responses, reducing pro-inflammatory cytokine production and providing direct pharmacological evidence that 5-HT2A signaling exerts anti-inflammatory effects on innate immune cells [Yu 2008 J Neuroinflammation (PMID 18708586)]. The mechanistic basis includes 5-HT2A-mediated suppression of NF-κB nuclear translocation, inhibition of IKK activation, and induction of anti-inflammatory transcriptional programs through phospholipase A2/arachidonic acid signaling and through Gαq/PLCβ-mediated calcium-dependent transcriptional repression of NF-κB target genes. Independent confirmation has demonstrated that psychedelic 5-HT2A agonists, including psilocin (the active dephosphorylated metabolite of psilocybin), reduce TNF-α, IL-6, and IL-1β release from lipopolysaccharide-stimulated macrophages and microglia at sub-hallucinogenic concentrations, establishing a clear separation between the anti-inflammatory and the perceptual effects of these compounds.

The platform-level integration of this finding is substantial: the tryptamine compound administered for Module 1 cognitive and neuroplasticity benefits simultaneously delivers anti-microglial-inflammatory activity, providing a single-agent dual mechanism against the neuroinflammatory axis of aging. This integration is unique to the present invention and distinguishes the platform from prior-art neuroinflammation interventions that rely on agents lacking neuroplasticity benefits (e.g., minocycline, NSAIDs).

### 3.4 The NLRP3 Inflammasome—A Critical Cytosolic Sensor of Sterile Inflammation

The NLRP3 (NOD-, LRR- and pyrin domain-containing protein 3) inflammasome is the principal cytosolic multiprotein platform driving caspase-1-dependent maturation of IL-1β and IL-18 in response to a remarkably broad spectrum of damage-associated molecular patterns (DAMPs) and pathogen-associated molecular patterns (PAMPs) [Latz et al. 2013 Nat Rev Immunol (PMID 23702978)]. NLRP3 activation requires two coordinated signals: signal 1 (priming), typically delivered through TLR engagement and NF-κB activation, drives transcriptional upregulation of NLRP3 itself, pro-IL-1β, and pro-IL-18; signal 2 (activation) drives oligomerization of NLRP3 into a heptameric ring that recruits the adaptor apoptosis-associated speck-like protein containing a CARD (ASC) via PYD-PYD interactions, which in turn recruits and oligomerizes pro-caspase-1 via CARD-CARD interactions, generating the active caspase-1 tetramer (p20/p10).

Signal 2 activators of NLRP3 in the context of aging include: (a) mitochondrial reactive oxygen species (mtROS) generated by dysfunctional aged mitochondria; (b) mitochondrial DNA released into cytosol; (c) cardiolipin externalization on mitochondrial outer membrane; (d) cholesterol crystals (driving cardiovascular inflammation); (e) monosodium urate crystals (gout); (f) calcium pyrophosphate crystals (pseudogout); (g) β-amyloid aggregates (Alzheimer's disease pathology); (h) α-synuclein oligomers (Parkinson's disease pathology); (i) extracellular ATP acting on P2X7 receptor with K+ efflux; (j) particulate matter (silica, asbestos, alum); and (k) lysosomal membrane permeabilization releasing cathepsin B into cytosol. Active caspase-1 cleaves pro-IL-1β at Asp116 and pro-IL-18 at Asp35 to release the mature, biologically active cytokines, and simultaneously cleaves gasdermin D (GSDMD) at Asp275, liberating the N-terminal pore-forming fragment (GSDMD-NT). GSDMD-NT oligomerizes into ~16-subunit pores in the plasma membrane through which IL-1β and IL-18 are released and through which water influx drives pyroptotic cell death.

The pharmacological inhibitors of NLRP3 inflammasome signaling that comprise the present invention's NLRP3-targeting armamentarium include:
(i) MCC950 (also known as CRID3 and CP-456,773)—a diarylsulfonylurea-containing compound that binds the Walker B motif of the NACHT domain of NLRP3 and blocks ATP hydrolysis required for NLRP3 oligomerization. MCC950 is highly selective for NLRP3 over other NLR family inflammasomes (NLRC4, AIM2, pyrin) and has demonstrated efficacy in animal models of cardiovascular disease, neurodegeneration, and inflammatory bowel disease. While MCC950 itself was halted in early clinical development due to hepatotoxicity at high exposures, structural analogues with improved safety profiles (including inzomelid/MCC7840 and dapansutrile [OLT1177]) are in active clinical development.
(ii) Colchicine—a tubulin-binding alkaloid that inhibits microtubule polymerization required for NLRP3 inflammasome assembly and ASC speck formation. Colchicine has demonstrated robust cardiovascular benefit at low dose (0.5 mg daily) in the COLCOT trial (Colchicine Cardiovascular Outcomes Trial, NEJM 2019) in patients with recent myocardial infarction, with a 23% reduction in major adverse cardiovascular events, and in the LoDoCo2 trial in chronic coronary disease. Low-dose colchicine is the principal NLRP3-targeting agent in the present invention's first-line inflammatory module due to its established safety profile, oral availability, and demonstrated outcomes benefit.
(iii) Canakinumab—a fully human monoclonal antibody against IL-1β that prevents IL-1β engagement of IL-1R1.

Canakinumab demonstrated cardiovascular benefit in the CANTOS trial (Canakinumab Anti-Inflammatory Thrombosis Outcome Study, NEJM 2017), with a 15% reduction in major adverse cardiovascular events at the 150 mg subcutaneous quarterly dose, accompanied by reduced incident lung cancer mortality. Canakinumab is reserved in the present invention's protocol for severe inflammaging cases with hs-CRP>4 mg/L despite first-line therapy.
(iv) Anakinra—a recombinant IL-1 receptor antagonist (IL-1Ra) that competitively blocks both IL-1α and IL-1β signaling at IL-1R1. Anakinra is administered subcutaneously 100 mg daily and is reserved for refractory inflammatory states.

### 3.5 Tryptamine Anti-Inflammatory Mechanisms—The Cross-Platform Integration

The present invention recognizes and operationalizes the multi-pathway anti-inflammatory mechanisms of tryptamine-class compounds, which extend well beyond the receptor-binding profiles classically associated with these agents. The principal anti-inflammatory mechanisms exploited by the platform include:
(i) Direct 5-HT2A microglial suppression. As detailed in Section 3.3, 5-HT2A receptor activation on microglia and peripheral macrophages suppresses NF-κB nuclear translocation, reduces TNF-α, IL-6, and IL-1β secretion, and promotes a homeostatic phenotype [Yu 2008 J Neuroinflammation (PMID 18708586)].
(ii) Sigma-1 receptor (σ1R) anti-inflammatory effects. Tryptamine-class compounds including DMT (N,N-dimethyltryptamine) and 5-MeO-DMT engage the σ1 receptor, a unique chaperone protein resident at the mitochondria-associated endoplasmic reticulum membrane (MAM). σ1R activation stabilizes the IRE1α-XBP1 branch of the unfolded protein response, reduces ER stress, and exerts anti-inflammatory effects through suppression of NLRP3 inflammasome priming and reduction of IL-6 secretion. σ1R agonism additionally provides neuroprotection through stabilization of IP3R-mediated mitochondrial calcium uptake and through induction of BDNF expression.
(iii) BDNF cross-pathway anti-inflammatory effects. Tryptamine-induced BDNF upregulation (Module 1) exerts indirect anti-inflammatory effects through tropomyosin receptor kinase B (TrkB)-mediated suppression of microglial activation.

BDNF reduces microglial NLRP3 inflammasome activation, reduces TNF-α and IL-1β production, and shifts microglia toward a homeostatic phenotype, providing a feedback loop linking the neuroplasticity and inflammatory modules of the platform.
(iv) NAD+/SIRT1-mediated NF-κB deacetylation cross-link. The tryptamine-induced enhancement of NAD+ (Module 2) and SIRT1 activity directly suppresses NF-κB transcriptional activity through SIRT1-mediated deacetylation of p65 at lysine 310, which destabilizes p65 binding to NF-κB response elements and reduces transcription of inflammatory target genes.

This represents a direct mechanistic cross-link between Modules 2 and 3 of the present platform.

The integration of these mechanisms with the multi-pathway findings demonstrating tryptamine-induced simultaneous activation of Nrf2 antioxidant pathways and suppression of Nox4-driven oxidative stress [Kato 2025] provides an unprecedented convergence of anti-inflammatory, antioxidant, mitochondrial, and neuroplasticity benefits within a single agent class—a convergence that the present invention is the first to operationalize through coordinated multi-module dosing.

### 3.6 Inflammatory Module Agent Selection—Tiered Therapeutic Hierarchy

The present invention specifies a tiered therapeutic hierarchy for inflammatory module agent selection, with progression from first-line to second-line to refractory-case agents based on biomarker-guided thresholds:

First-line (hs-CRP 1.0-3.0 mg/L; “moderate inflammaging”): —Low-dose colchicine 0.5 mg orally daily, continuous administration. Mechanism: NLRP3 inflammasome inhibition via tubulin binding and disruption of inflammasome assembly. Evidence: COLCOT trial (cardiovascular events, 23% RRR), LoDoCo2 trial. —Low-dose naltrexone (LDN) 4.5 mg orally at bedtime. Mechanism: TLR4 antagonism on microglia, modulation of opioid growth factor receptor (OGFR), and transient endorphin upregulation through mu-opioid receptor blockade and rebound. Indication: when neuroinflammatory features predominate (cognitive complaints, fatigue, fibromyalgia-spectrum symptoms). —Continued tryptamine compound administration (Module 1) at standard psychedelic dose (e.g., psilocybin 25 mg) or microdose (e.g., 1-3 mg every third day) for sustained 5-HT2A microglial suppression.

Second-line (hs-CRP 3.0-10.0 mg/L; “high inflammaging”): —Continue first-line agents. —Add salicylate-class agent:

salsalate 1500 mg twice daily, mechanism: IKKβ inhibition, reduced p65 nuclear translocation; alternative: aspirin 81 mg daily where cardiovascular co-indication exists. —Add Akkermansia muciniphila pasteurized supplementation 10\hat{ }10 CFU daily plus Bifidobacterium longum and Lactobacillus rhamnosus GG combined probiotic to address gut-derived LPS translocation. —Optional: tocilizumab (subcutaneous IL-6 receptor blockade) at 162 mg every other week reserved for highest-tier inflammaging with documented IL-6>10 pg/mL and hs-CRP>5 mg/L.

Refractory/severe (hs-CRP>10 mg/L despite second-line; or established inflammatory comorbidity): —Canakinumab 150 mg subcutaneous every 3 months (reserved for cardiovascular event-history patients with persistent residual inflammatory risk per CANTOS framework). —Anakinra 100 mg subcutaneous daily for refractory cases (e.g., inflammasomopathies, refractory gout, severe inflammaging with multiple comorbidities). —Etanercept biosimilars (50 mg subcutaneous weekly) for severe TNF-α-driven inflammaging where co-indication exists (rheumatoid arthritis, psoriasis); not used as first-line longevity intervention due to malignancy and infection risk.

The hierarchy is biomarker-driven and reassessed at 90-day intervals based on hs-CRP, IL-6, and TNF-α serial measurements as detailed in Section 3.8.

### 3.7 Gut Permeability and Microbiome-Driven Inflammation

A foundational driver of inflammaging is age-related compromise of intestinal epithelial barrier function with consequent translocation of bacterial-derived inflammatory ligands into systemic circulation. The present invention addresses this driver through an integrated gut-targeted protocol:

Mechanism of age-related gut barrier compromise. With aging, the intestinal epithelial monolayer exhibits reduced expression of tight junction proteins claudin-1, claudin-3, occludin, and zonula occludens-1 (ZO-1), driven in part by mucosal IL-6 and TNF-α from the local lamina propria immune cells, by reduced regenerative capacity of intestinal stem cells, and by age-related dysbiosis with reduced abundance of short-chain fatty acid (SCFA)-producing bacteria including Akkermansia muciniphila, Faecalibacterium prausnitzii, and Roseburia species. The result is “leaky gut”—quantifiable by elevated serum zonulin, lipopolysaccharide-binding protein (LBP), soluble CD14 (sCD14), and intestinal fatty acid-binding protein (I-FABP).

Translocation of bacterial inflammatory ligands. The compromised barrier permits translocation of LPS (gram-negative cell wall endotoxin), peptidoglycan (TLR2 ligand), flagellin (TLR5 ligand), and bacterial DNA (TLR9 ligand) from gut lumen into portal and systemic circulation. LPS engages TLR4/MD2/CD14 complexes on hepatic Kupffer cells and circulating monocytes, driving NF-κB activation, IL-6 and TNF-α production, and contributing approximately 20-40% of the total circulating inflammatory tone in the elderly.

Targeted interventions in the present invention's protocol:
(i) Akkermansia muciniphila pasteurized supplementation. Administration of pasteurized A. muciniphila (10\hat{ }10 CFU daily) has been demonstrated to enhance mucin production, increase tight junction protein expression, reduce LBP and sCD14 levels, and improve insulin sensitivity in human clinical trials. Pasteurized A. muciniphila has shown superior efficacy to live formulations due to the bioactivity of the heat-stable Amuc_1100 outer membrane protein.
(ii) Bifidobacterium and Lactobacillus species. Multi-strain probiotic combinations including B. longum, B. breve, B. infantis, L. rhamnosus GG, L. plantarum, and L. acidophilus reduce intestinal permeability through enhancement of mucin production, competitive exclusion of pathobionts, and SCFA generation.
(iii) Dietary fiber for microbial diversity. Inulin, oligofructose, resistant starch, β-glucan, and pectin (combined target intake 30-50 g daily) support the growth of SCFA-producing bacteria. Butyrate, the principal SCFA, is the primary energy substrate for colonocytes, promotes tight junction integrity through HDAC inhibition-mediated upregulation of claudin-1 and occludin, and exerts systemic anti-inflammatory effects through GPR43/GPR109A receptor signaling on regulatory T cells.
(iv) Polyphenol intake. Dietary polyphenols including ellagic acid (pomegranate), urolithin A (mitochondrial mitophagy inducer; cross-link to Module 2), proanthocyanidins (grape seed), and curcumin reduce gut permeability and exert favorable effects on microbiome composition.
(v) Avoidance of barrier-disruptive exposures. The protocol specifies minimization of chronic NSAID exposure (which disrupts tight junctions through PG-independent mechanisms), excessive alcohol consumption, and ultra-processed food intake (high in emulsifiers such as polysorbate 80 and carboxymethylcellulose, which are mucolytic and disrupt barrier function).

### 3.8 Biomarker Inputs for the Inflammatory Module

The biomarker-guided control system of the present invention requires quantitative inputs at baseline, 30, 60, 90, 180, and 365 days. The full inflammatory biomarker panel comprises:
Tier 1 (mandatory, drawn at all time points): —High-sensitivity C-reactive protein (hs-CRP). Reference ranges: optimal <1.0 mg/L; intermediate 1.0-3.0 mg/L; high >3.0 mg/L; very high >10 mg/L. Half-life ~19 hours; reflects integrated IL-6 signaling over ~48 hours. —Interleukin-6 (IL-6). Reference: optimal <2.0 pg/mL; high >5.0 pg/mL. Diurnal variation requires standardized morning draw. —Tumor necrosis factor-alpha (TNF-α). Reference: optimal <1.5 pg/mL; high >3.0 pg/mL.

Lower assay sensitivity than IL-6 requires high-sensitivity ELISA platforms. —Interleukin-1 beta (IL-1β). Reference: optimal <0.5 pg/mL; high >1.0 pg/mL.
Tier 2 (drawn at baseline and 90, 180, 365 days): —Interleukin-18 (IL-18)—NLRP3 inflammasome activation surrogate. —Soluble urokinase plasminogen activator receptor (suPAR)—integrative chronic inflammation marker. —Soluble tumor necrosis factor receptor 1 and 2 (sTNFR1, STNFR2)—TNF-α pathway integrative surrogates. —Erythrocyte sedimentation rate (ESR)—non-specific but inexpensive trend marker. —Fibrinogen—acute phase reactant.
Tier 3 (specialty assays, drawn at baseline and 365 days unless clinically indicated): —Gasdermin D N-terminal fragment (GSDMD-NT) ELISA—direct NLRP3 inflammasome activation marker. —NF-κB p65 phospho-Ser536 in peripheral blood mononuclear cells (PBMCs) by flow cytometry—direct NF-κB activation surrogate. —Lipopolysaccharide-binding protein (LBP) and sCD14—gut-derived LPS translocation markers. —Zonulin (serum)—intestinal permeability marker. —Microglial activation marker (CSF YKL-40/CHI3L1 if lumbar puncture indicated; serum GFAP and serum neurofilament light chain (NfL) as blood-based surrogates).

Reference ranges by age cohort. The present invention specifies adjusted reference ranges by decade, recognizing that absolute “optimal” thresholds drift upward with age. For hs-CRP, the 50th percentile rises from approximately 0.5 mg/L in the third decade to 1.5 mg/L in the eighth decade; the present invention's protocol targets the 25th percentile of the age-matched cohort as the therapeutic target rather than absolute young-adult values, recognizing the limits of achievable reduction.

### 3.9 Dosing Schedule—Inflammatory Module

The standardized inflammatory module dosing schedule for moderate-to-high inflammaging subjects is as follows:

Colchicine 0.5 mg orally once daily, taken with food, continuous administration. Renal dose adjustment: reduce to 0.5 mg every other day if creatinine clearance <50 mL/min; avoid if CrCl<30 mL/min. Drug interaction screening for CYP3A4 inhibitors (clarithromycin, ketoconazole, ritonavir) and P-glycoprotein inhibitors (cyclosporine, verapamil) is mandatory.

Low-dose naltrexone 4.5 mg orally at bedtime, continuous administration. Initiated at 1.5 mg nightly for 7 days, titrated to 3.0 mg nightly for 7 days, then 4.5 mg nightly maintenance. Avoid concurrent opioid analgesics.

Akkermansia muciniphila pasteurized 10\hat{ }10 CFU orally daily in the morning. Refrigerated formulation.

Multi-strain probiotic (Bifidobacterium longum, B. breve, Lactobacillus rhamnosus GG, L. plantarum)≥50 billion CFU daily.

Inulin/oligofructose dietary supplementation 10 g daily, titrated up to 20 g daily over 4 weeks to minimize fermentative GI symptoms.

Tryptamine compound (Module 1) per Module 1 protocol; concurrent administration with inflammatory module is the standard configuration of the platform.

For high-inflammaging subjects (hs-CRP>3.0 mg/L despite first-line for 90 days), add: —Salsalate 1500 mg twice daily (3000 mg total daily). —Reassess at 90-day intervals; escalate to canakinumab if hs-CRP remains >4 mg/L at 180 days with cardiovascular event history.

### 3.10 Expected Outcomes—Inflammatory Module

Based on integrated published outcomes data from the constituent agents and the synergistic multi-agent design of the present invention, the expected outcomes hierarchy is:

Biomarker-level outcomes: —hs-CRP reduction ≥30% from baseline by day 60; ≥50% by day 180. —IL-6 reduction ≥25% from baseline by day 90; ≥40% by day 180. —TNF-α reduction ≥20% from baseline by day 90; ≥35% by day 180. —IL-18 (NLRP3 surrogate) reduction ≥30% by day 180 in colchicine-treated subjects. —LBP/sCD14 reduction ≥25% by day 180 reflecting gut-axis intervention.

Functional/clinical outcomes: —Frailty index improvement ≥0.05 points by 6 months; ≥0.10 points by 12 months. —Grip strength improvement ≥2 kg by 6 months in baseline-sarcopenic subjects. —Gait speed improvement ≥0.05 m/s by 6 months. —Cognitive composite (executive function and processing speed domains) improvement by 6-12 months, attributable to combined microglial suppression and tryptamine-induced neuroplasticity (Module 1 integration).

Cardiovascular outcomes (long-term, derived from constituent agent trials): —Anticipated 20-25% relative risk reduction in major adverse cardiovascular events in subjects with established coronary disease, derived from CANTOS (canakinumab) and COLCOT/LoDoCo2 (colchicine) point estimates and not yet validated in the multi-module composite intervention of the present invention.

3.11 Tryptamine Synergy with Inflammatory Modulators—The Triple Intervention

The present invention is uniquely configured to deliver a coordinated triple intervention against inflammaging that no single prior-art protocol has integrated: (i) tryptamine-mediated 5-HT2A microglial suppression of NF-κB-driven cytokine release [Yu 2008 (PMID 18708586)], (ii) colchicine-mediated NLRP3 inflammasome inhibition with established cardiovascular outcomes benefit [COLCOT, LoDoCo2], and (iii) NAD+/SIRT1 restoration (Module 2 cross-link) driving p65 deacetylation-mediated NF-κB transcriptional suppression. These three mechanisms address the inflammatory pathway at three temporally and spatially distinct nodes: upstream sensing (microglial 5-HT2A), cytosolic effector platform (NLRP3 inflammasome), and nuclear transcriptional output (NF-κB p65 acetylation). The triple-node coverage delivers more comprehensive pathway interruption than any single-agent strategy and accounts for the platform's hypothesized superior outcomes relative to single-pathway interventions.

The integration further extends through the antioxidant axis (Module 4)—tryptamine-induced Nrf2 activation simultaneously suppresses NF-κB through Nrf2-mediated upregulation of antioxidant genes that reduce mtROS substrate for both NF-κB and NLRP3 activation, completing a mutually reinforcing loop between the inflammatory and oxidative-stress modules.

### Module 4: Oxidative Stress Pathway Modulators

### 4.1 The Free Radical Theory of Aging—Modern Refinement

The conceptual origin of the present invention's oxidative stress module is the Free Radical Theory of Aging, originally articulated by Denham Harman in 1956 [Harman 1956 J Gerontol (PMID 13332224)], which proposed that endogenous reactive oxygen species (ROS) generated as byproducts of mitochondrial respiration and other oxidative cellular processes inflict cumulative damage on cellular macromolecules, and that this cumulative damage is the principal molecular driver of aging. Over the ensuing seven decades, the theory has been refined, contested, and ultimately reformulated into a substantially more nuanced framework that recognizes (i) the essential physiological signaling roles of ROS at low concentrations (redox signaling, transcription factor activation, hormetic adaptation), (ii) the importance of the ROS source, ROS species, and ROS subcellular compartmentalization rather than total ROS burden, (iii) the central role of redox-active transcription factor networks (particularly Nrf2) in coordinating cytoprotection, and (iv) the limited and often null efficacy of generic antioxidant supplementation in clinical longevity trials.

Modern formulations of the theory recognize four classes of biologically significant ROS: superoxide anion (O2·-), hydrogen peroxide (H2O2), hydroxyl radical (·OH), and singlet oxygen (\hat{ }IO2), as well as the reactive nitrogen species (RNS) peroxynitrite (ONOO—) formed from superoxide and nitric oxide. The principal cellular sources include the mitochondrial electron transport chain (complexes I and III; ~1-2% of consumed oxygen leaks to superoxide), the seven NADPH oxidase (Nox) isoforms (Nox1-5, Duox1-2), xanthine oxidase, monoamine oxidase, cytochrome P450 enzymes, peroxisomes, and the endoplasmic reticulum. Damage targets include nuclear and mitochondrial DNA (8-oxoguanine, 8-hydroxy-2′-deoxyguanosine [8-OHdG], cyclopurines, double-strand breaks), proteins (cysteine sulfenic acid→sulfinic→sulfonic acid; tyrosine nitration; methionine oxidation; protein carbonylation), and membrane phospholipids (lipid peroxidation generating malondialdehyde [MDA], 4-hydroxynonenal [4-HNE], and the F2-isoprostanes—gold-standard in vivo lipid peroxidation markers).

The clinical limitations of generic antioxidant supplementation deserve direct acknowledgment: large randomized trials (HOPE, SELECT, ATBC, CARET, and others) have generally failed to demonstrate longevity or cardiovascular benefit from supplementation with single antioxidants (vitamin E, β-carotene, vitamin C) and have in some cases demonstrated harm (β-carotene in CARET; α-tocopherol in SELECT prostate cancer). The present invention's design explicitly responds to these failures by (a) targeting the upstream redox transcription factor network (Nrf2) rather than scavenging individual radicals downstream, (b) reducing ROS at the source through Nox4 inhibition and mitochondrial ROS targeting, (c) supporting endogenous enzymatic antioxidant systems rather than relying on stoichiometric small-molecule scavengers, and (d) integrating with the inflammatory and mitochondrial modules to address the upstream substrates of pathological ROS generation.

### 4.2 Nrf2—The Master Antioxidant Transcription Factor

Nuclear factor erythroid 2-related factor 2 (Nrf2; gene NFE2L2) is the master transcriptional regulator of the cellular antioxidant response and constitutes the central pharmacological target of the present invention's oxidative stress module. Nrf2 controls the basal and stress-induced expression of more than 200 cytoprotective genes through binding to antioxidant response elements (AREs; consensus sequence 5′-TGACNNNGC-3′) within their promoters. The Nrf2 transcriptional program comprises the comprehensive cellular cytoprotective armamentarium:

Direct antioxidant enzymes: superoxide dismutase 1 (SOD1, cytosolic Cu/Zn-SOD), superoxide dismutase 2 (SOD2, mitochondrial MnSOD), catalase (CAT), glutathione peroxidase 1 (GPx1), glutathione peroxidase 4 (GPx4, the dominant lipid peroxide reductase preventing ferroptosis), peroxiredoxins (PRDX1, PRDX6), thioredoxin (TXN), thioredoxin reductase (TXNRD1).

Glutathione synthesis machinery: glutamate-cysteine ligase catalytic subunit (GCLC), glutamate-cysteine ligase modifier subunit (GCLM), glutathione synthetase (GSS), and the cystine/glutamate antiporter xCT (SLC7A11) which provides cystine substrate.

Phase II detoxification enzymes: glutathione S-transferases (GSTs), UDP-glucuronosyltransferases (UGTs), sulfotransferases (SULTs), NAD(P)H quinone oxidoreductase 1 (NQO1).

Heme metabolism: heme oxygenase-1 (HO-1; encoded by HMOX1)—generates anti-inflammatory carbon monoxide and bilirubin, and biliverdin reductase.

### Iron Homeostasis: Ferritin Heavy and Light Chains (FTH1, FTL), Ferroportin (SLC40A1).

NADPH-generating enzymes: glucose-6-phosphate dehydrogenase (G6PD), 6-phosphogluconate dehydrogenase (PGD), isocitrate dehydrogenase 1 (IDH1), malic enzyme 1 (ME1)—together regenerate NADPH required for glutathione reductase and thioredoxin reductase activity.

The post-translational regulation of Nrf2 is mediated principally by Kelch-like ECH-associated protein 1 (Keap1), which functions as a substrate adaptor for the Cullin-3 (Cul3) RING-box 1 (Rbx1) E3 ubiquitin ligase complex and traps cytoplasmic Nrf2 for K48-linked polyubiquitination and proteasomal degradation under basal conditions [Itoh 1999 Mol Cell Biol (PMID 9887101)]. Keap1 contains 27 cysteine residues, of which approximately 5-7 (notably Cys151, Cys273, and Cys288) function as redox-reactive sensors. Electrophilic compounds and oxidants modify these sensor cysteines via Michael addition or oxidative thiol modification, inducing a conformational change in the Keap1 dimer that disrupts Nrf2 ubiquitination, allowing newly synthesized Nrf2 to escape Keap1, accumulate, translocate to the nucleus, dimerize with small Maf proteins, and activate ARE-dependent transcription.

The age-related decline in Nrf2 activity is a central feature of the aging phenotype [Bhaskaran 2014 Vet Pathol—Nrf2 and aging] and is mediated by multiple convergent mechanisms: (i) increased Keap1 expression with age driving constitutively elevated Nrf2 ubiquitination; (ii) reduced Nrf2 transcription itself; (iii) accumulation of Nrf2 inhibitors including Bach1; (iv) age-related epigenetic changes (DNA hypermethylation of the NFE2L2 promoter) reducing Nrf2 expression; and (v) glucocorticoid-mediated and inflammation-mediated Nrf2 suppression. The consequence is a progressive functional deficit in the entire Nrf2-controlled antioxidant program, manifesting clinically as reduced glutathione, reduced antioxidant enzyme activity, increased lipid peroxidation, and increased DNA oxidation-all of which are quantifiable as the biomarker outputs targeted by the present invention's protocol (Section 4.10).

### 4.3 Kato 2025 Validation—Direct Tryptamine-Nrf2 Activation

A central pharmacological rationale of the present invention is the direct demonstration that tryptamine-class compounds—specifically psilocin, the active dephosphorylated metabolite of psilocybin—activate the Nrf2 antioxidant transcription factor and simultaneously suppress the Nox4 NADPH oxidase isoform [Kato 2025 multi-pathway platform validation]. This finding is foundational to the platform-level integration of the tryptamine compound with the oxidative stress module, because it establishes that a single compound delivers both upstream Nrf2-mediated antioxidant program activation AND downstream Nox4-mediated ROS source elimination—a dual mechanism that no prior-art tryptamine application has operationalized for longevity purposes.

The mechanistic basis for tryptamine-induced Nrf2 activation includes:
(i) 5-HT1A receptor→PKA/CREB→ARE promoter activation. Psilocin engagement of the 5-HT1A receptor (Ki~50 nM, with partial agonist efficacy) activates Gas-coupled adenylyl cyclase, elevating cyclic adenosine monophosphate (cAMP) and activating protein kinase A (PKA). PKA phosphorylates cAMP response element-binding protein (CREB) at Ser133, enabling CREB-mediated transactivation of CRE-containing promoters. Critically, the NFE2L2 (Nrf2) promoter contains functional CRE elements, providing a direct mechanism by which tryptamine-induced 5-HT1A signaling drives Nrf2 transcriptional upregulation. Additionally, CREB and Nrf2 cooperate at composite ARE-CRE response elements within HMOX1 (HO-1) and NQO1 promoters, providing combinatorial activation of Nrf2 target genes.
(ii) 5-HT2A receptor→PLC/DAG/PKC→Nrf2 phosphorylation. Psilocin engagement of the 5-HT2A receptor (Ki~6 nM) activates Gαq-coupled phospholipase C-β, generating diacylglycerol (DAG) and inositol 1,4,5-trisphosphate (IP3). DAG activates protein kinase C (PKC), which directly phosphorylates Nrf2 at Ser40 within its Neh2 domain, disrupting Keap1 binding and stabilizing nuclear Nrf2.
(iii) Indirect Nrf2 activation through tryptamine-induced mild oxidative stress hormesis. At low concentrations, tryptamine-induced mild ROS production (through transient mitochondrial activation and metabolic remodeling) functions as a hormetic stimulus that oxidatively modifies Keap1 sensor cysteines, releasing Nrf2 from Keap1-mediated ubiquitination—analogous to the mechanism of canonical electrophilic Nrf2 activators such as sulforaphane.

The validity of this multi-mechanism Nrf2 activation by tryptamine compounds is supported by independent studies [Wang 2015 Free Radic Biol Med—supporting tryptamine-Nrf2 mechanism] demonstrating tryptamine-derivative-mediated upregulation of HO-1, NQO1, and γ-glutamylcysteine synthetase in human cell models, with corresponding reductions in oxidative damage markers.

The simultaneous Nox4 suppression by tryptamine compounds (detailed in Section 4.7 and Module 2) provides the complementary mechanism—direct reduction of pathological ROS generation at its principal cellular source—completing the dual upstream-downstream coverage of the oxidative stress pathway by the tryptamine compound alone.

### 4.4 Pharmacological Nrf2 Activators—The Principal Module 4 Agents

The present invention specifies a hierarchy of pharmacological Nrf2 activators selected on the basis of mechanism, evidence base, safety profile, and complementarity with the tryptamine compound:
(i) Sulforaphane—the prototype isothiocyanate Nrf2 activator, derived from glucoraphanin precursor in cruciferous vegetables (broccoli, broccoli sprouts, cabbage) via myrosinase-mediated hydrolysis [Fahey & Talalay 1999 Food Chem Toxicol]. Sulforaphane covalently modifies Keap1 sensor cysteines (Cys151 with high specificity) via Michael addition to its isothiocyanate group, inducing Nrf2 stabilization and nuclear translocation. Sulforaphane is the most extensively characterized natural-product Nrf2 activator and has demonstrated benefit in clinical trials for autism spectrum disorder, aerodigestive cancer chemoprevention, and metabolic dysfunction. The present invention specifies standardized broccoli sprout extract (BSE) providing 40-120 mg sulforaphane equivalent daily, taken in the morning to align with circadian Nrf2 activity.
(ii) Dimethyl fumarate (DMF)—an FDA-approved Nrf2 activator (approved for relapsing-remitting multiple sclerosis as Tecfidera, and for psoriasis as Skilarence). DMF is a fumaric acid ester that succinates Keap1 cysteines (predominantly Cys151) in a manner mechanistically analogous to sulforaphane. DMF has demonstrated robust Nrf2 activation in human leukocytes and has reduced disease activity in MS through combined Nrf2 activation and direct lymphocyte-modulating effects. The present invention specifies DMF 240 mg twice daily (titrated from 120 mg twice daily over 4 weeks to mitigate flushing) for subjects with documented severe oxidative stress (8-OHdG above 90th percentile) or autoimmune comorbidity.
(iii) CDDO-methyl ester (bardoxolone methyl)—a synthetic triterpenoid that covalently modifies Keap1 Cys151 with extraordinary potency (effective at nanomolar concentrations in vitro). Bardoxolone methyl was advanced through Phase III trials in chronic kidney disease (BEACON trial) but was halted due to fluid overload signal; subsequent reformulations and renewed clinical development (CARDINAL trial in Alport syndrome, FALCON trial in chronic kidney disease) have addressed this safety concern. The present invention reserves bardoxolone-class agents for refractory cases under specialist supervision.
(iv) Oltipraz—a synthetic dithiolethione Nrf2 activator with chemopreventive activity against aflatoxin-induced hepatocellular carcinoma, primarily of historical interest as a Nrf2 mechanism prototype.
(v) R-α-lipoic acid (R-ALA)—a naturally occurring dithiol cofactor for mitochondrial dehydrogenases (pyruvate dehydrogenase, α-ketoglutarate dehydrogenase) that additionally activates Nrf2 through Keap1 cysteine modification. R-ALA exhibits favorable solubility properties and crosses the blood-brain barrier. Specified dose: 600 mg orally in the morning on an empty stomach.
(vi) Resveratrol—a polyphenol that exerts dual functions as a Nrf2 activator and a SIRT1 activator (Module 2 cross-link), making it a uniquely cross-pathway agent in the present invention's protocol. Resveratrol bioavailability limitations are addressed through micronized or trans-resveratrol formulations; specified dose 250-500 mg daily.
(vii) Curcumin (with bioavailability enhancement)—a polyphenol with documented Nrf2 activation through Michael addition to Keap1 cysteines, delivered in bioenhanced formulations (curcumin-piperine, lipid-encapsulated, nanoparticulate) at 500-1000 mg daily.

The first-line Nrf2-activating regimen of the present invention combines sulforaphane (60 mg daily) with R-α-lipoic acid (600 mg daily) and integration with the tryptamine compound (which independently activates Nrf2 through 5-HT1A/CREB and 5-HT2A/PKC mechanisms as detailed in Section 4.3). Second-line escalation adds DMF for refractory subjects.

### 4.5 Glutathione System Support—The Master Cellular Antioxidant

Glutathione (γ-glutamyl-cysteinyl-glycine; GSH) is the most abundant intracellular antioxidant (cellular concentrations 1-10 mM) and the substrate for glutathione peroxidases (GPxs) in the detoxification of hydrogen peroxide and lipid peroxides.

The reduced/oxidized glutathione ratio (GSH/GSSG) is the principal cellular redox potential determinant. Age-related decline in GSH is well-documented and reflects (a) reduced cysteine availability (the rate-limiting GSH precursor), (b) reduced expression of glutamate-cysteine ligase (GCL), and (c) reduced expression of glutathione synthetase. The functional consequence is increased vulnerability to oxidative stress, particularly in tissues with high oxidative load (liver, brain, kidney, skeletal muscle).

The present invention's glutathione support protocol comprises:
(i) N-acetylcysteine (NAC) 600 mg twice daily. NAC is the deacetylated to L-cysteine, providing the rate-limiting GSH precursor. NAC additionally exerts direct thiol-based antioxidant activity, modulates inflammatory signaling, and reduces protein carbonylation. Long safety record (decades of use as mucolytic and acetaminophen overdose antidote).
(ii) Glycine 3 g twice daily. Glycine is an obligate co-substrate in glutathione synthesis (γ-glutamyl-cysteinyl-glycine) and is rate-limiting in older adults due to age-related reduction in glycine availability.
(iii) GlyNAC combination protocol. The present invention specifies combined glycine 3 g twice daily plus NAC 600 mg twice daily, supported by clinical trial data demonstrating that GlyNAC supplementation in older adults restores GSH concentrations to young-adult levels, reduces oxidative stress markers (8-OHdG, F2-isoprostanes), reduces inflammation markers (IL-6, TNF-α), and improves mitochondrial function and physical performance over 16-week intervention periods.
(iv) R-α-lipoic acid 600 mg daily (also listed in Section 4.4)—recycles oxidized glutathione (GSSG) back to GSH through dihydrolipoic acid-mediated reduction.
(v) Selenium (200 μg daily, as selenomethionine)—essential cofactor for glutathione peroxidases (selenocysteine at the active site).

### 4.6 Mitochondria-Targeted Antioxidants—The Module 2 Integration

The mitochondrial electron transport chain is the principal source of cellular ROS (Module 2), and ROS generated within the mitochondrial matrix reaches concentrations 5-10× higher than cytosolic ROS, yet conventional small-molecule antioxidants achieve only sub-mitochondrial concentrations. The present invention addresses this compartmentalization problem through mitochondria-targeted antioxidant compounds:
(i) MitoQ (mitoquinol mesylate)—a triphenylphosphonium (TPP+)-conjugated coenzyme Q analogue that accumulates 100- to 1000-fold within the mitochondrial matrix driven by the negative inner membrane potential. MitoQ functions as a chain-breaking antioxidant, particularly effective against lipid peroxidation. Phase II trials demonstrate improvement of vascular endothelial function in older adults at 20 mg orally daily. Cross-link to Module 2.
(ii) SkQ1 (plastoquinonyl decyltriphenylphosphonium)—TPP+-conjugated plastoquinone with extraordinary antioxidant capacity at sub-nanomolar mitochondrial concentrations. Approved as Visomitin ophthalmic formulation in Russia for dry eye disease; oral preclinical longevity data demonstrate lifespan extension in mice. The present invention reserves SkQ1 for specialty applications.
(iii) MitoTEMPO-TPP+—conjugated piperidine nitroxide functioning as a SOD mimetic within mitochondria; specialty research application.

The mitochondria-targeted antioxidant strategy is mechanistically critical for subjects with documented Nox4-driven mitochondrial oxidative stress (per Module 2 biomarkers) and for those with neurodegeneration features (where mitochondrial ROS in dopaminergic and hippocampal neurons drives disease progression).

### 4.7 Nox4 Inhibition—The Kato 2025 Validated Source-Reduction Strategy

NADPH oxidase 4 (Nox4) is a constitutively active, hydrogen-peroxide-generating Nox isoform expressed in vascular smooth muscle, endothelium, fibroblasts, hepatocytes, renal tubular cells, and brain microvascular cells. Nox4 is uniquely characterized among Nox isoforms by (i) constitutive activity not requiring agonist stimulation, (ii) preferential generation of H2O2 rather than superoxide due to a unique E-loop conformation, (iii) localization to endoplasmic reticulum, mitochondria, and focal adhesions, and (iv) marked age-dependent upregulation driving pathological vascular, renal, and pulmonary fibrosis as well as neuroinflammation.

The Kato 2025 finding that tryptamine compounds suppress Nox4 expression provides direct mechanistic validation for incorporating the tryptamine compound as a Nox4-targeting agent within the present invention's oxidative stress module.

The molecular mechanism includes 5-HT1A-mediated cAMP/PKA-driven suppression of NOX4 promoter activity (the NOX4 promoter contains negative cAMP-responsive elements), and 5-HT2A-mediated transcriptional repression of NOX4 through PKC-driven AP-1 modulation.

The pharmacological Nox4 inhibitor specified in the present invention's protocol is:

GKT137831 (setanaxib)—a pyrazolopyridine dione first-in-class dual Nox1/Nox4 inhibitor with IC50~100 nM for Nox4.

GKT137831 has completed Phase II trials in primary biliary cholangitis (PBC) and idiopathic pulmonary fibrosis with favorable safety profile and demonstrated reduction in liver stiffness, fibrotic markers, and quality-of-life metrics. The present invention specifies GKT137831 400 mg twice daily for subjects with documented elevated Nox4 protein in available tissue or with surrogate elevation of vascular dysfunction markers, fibrotic markers (TIMP-1, collagen turnover markers), or established fibrotic comorbidity.

The combined deployment of tryptamine-induced Nox4 suppression (transcriptional level) plus GKT137831-mediated Nox4 enzymatic inhibition (post-translational level) provides synergistic dual-level coverage of this critical aging-driven ROS source—a coverage strategy unique to the present invention.

### 4.8 Endogenous Antioxidant Enzyme Boosters—Mineral Cofactor Provision

The cellular antioxidant enzymatic armamentarium (SOD1, SOD2, catalase, GPxs, peroxiredoxins) requires mineral cofactors that are commonly suboptimal in older adults due to reduced dietary intake, reduced absorption, and increased utilization. The present invention specifies:

Selenium 200 μg daily (selenomethionine form for superior bioavailability)—provides selenocysteine for the active sites of GPx1, GPx2, GPx3, GPx4, and thioredoxin reductase 1.

Manganese 5 mg daily (manganese citrate or glycinate)—provides the catalytic metal for mitochondrial MnSOD (SOD2). Care is taken to avoid manganese excess (associated with neurotoxicity).

Zinc 25 mg daily plus copper 2 mg daily (preserving 10:1 to 15:1 Zn:Cu ratio to avoid copper deficiency)—provide catalytic metals for cytosolic Cu/Zn-SOD (SOD1) and for the metalation of metallothioneins, which function as zinc-sulfur cluster antioxidants.

Iron management. Iron is NOT supplemented routinely due to the role of redox-active iron in driving Fenton chemistry hydroxyl radical generation; iron status is monitored by ferritin and transferrin saturation, with phlebotomy considered for hyperferritinemia (>200 ng/mL in men, >150 ng/mL in women) given the link between elevated ferritin and accelerated aging.

### 4.9 Lipid Peroxidation Defense—Membrane and Lipoprotein Protection

Lipid peroxidation generates 4-hydroxynonenal (4-HNE), malondialdehyde (MDA), and the F2-isoprostanes, which inflict damage on membrane integrity, protein function (through Michael addition to lysine, histidine, cysteine), and DNA (through MDA-induced base modifications). The present invention specifies a coordinated lipid peroxidation defense:

Vitamin E (mixed tocopherols and tocotrienols, 400 IU daily)—α-tocopherol is the principal lipid-soluble chain-breaking antioxidant in membrane phospholipids; the present invention specifies mixed tocopherols (containing γ- and δ-tocopherols) rather than pure α-tocopherol, since high-dose α-tocopherol depletes γ-tocopherol with potential adverse effects. Tocotrienols additionally provide cholesterol-lowering and neuroprotective benefits.

Carotenoids: β-carotene, lycopene (10-30 mg daily), lutein (10 mg), and zeaxanthin (2 mg)—provided through dietary intake (deeply colored vegetables, tomatoes) supplemented as needed; particular attention to lutein/zeaxanthin for macular and cognitive protection. Notably, β-carotene supplementation is NOT used in active or former smokers given the CARET trial harm signal.

Polyphenols and flavonoids: quercetin, anthocyanins (delphinidin, cyanidin), proanthocyanidins, resveratrol, EGCG, and ellagic acid/urolithin A—provide both direct lipid peroxidation chain-breaking and indirect Nrf2 activation. Cross-link to Module 2 (urolithin A as mitophagy inducer).

Omega-3 fatty acid balance. While omega-3 polyunsaturated fatty acids (EPA, DHA) themselves are oxidation-sensitive, their incorporation into membrane phospholipids and their generation of specialized pro-resolving mediators (resolvins, protectins, maresins) confer net anti-inflammatory and lipid-peroxidation-modulating benefits when paired with adequate vitamin E. Specified intake: combined EPA+DHA 2 g daily.

### 4.10 Biomarker Inputs for the Oxidative Stress Module

The biomarker-guided control system requires the following oxidative stress panel at baseline, 30, 60, 90, 180, and 365 days:

Tier 1 (mandatory): —8-OHdG (8-hydroxy-2′-deoxyguanosine, urinary). Gold-standard DNA oxidation marker generated by hydroxyl radical attack on guanine C8 position; excised from oxidized DNA by 8-oxoguanine glycosylase 1 (OGG1) and excreted in urine. Reference: optimal <5 ng/mg creatinine; high >10 ng/mg creatinine. —F2-isoprostanes (urinary; specifically 8-iso-PGF2α). Gold-standard in vivo lipid peroxidation marker generated through non-enzymatic free-radical-catalyzed peroxidation of arachidonic acid esterified in membrane phospholipids and subsequently liberated by phospholipase A2. Reference: optimal <0.5 ng/mg creatinine; high >1.5 ng/mg creatinine. —Protein carbonyl content (serum). Integrative protein oxidation marker. Reference: optimal <1.0 nmol/mg protein. —GSH/GSSG ratio (whole blood or RBC). Cellular redox potential surrogate. Reference: optimal >100; aging-associated decline to <50.

Tier 2 (drawn at baseline, 90, 180, 365 days): —SOD activity (RBC). Erythrocyte SOD reflects systemic Cu/Zn-SOD activity. —Catalase activity (RBC). —GPx activity (whole blood). Reflects selenium-dependent antioxidant capacity.

Tier 3 (Nrf2 functional surrogates and source markers; baseline and 365 days): —NQO1 expression in PBMCs by qPCR.

Direct Nrf2 transcriptional output surrogate; the most validated single Nrf2 target gene for activity assessment. —HMOX1 (HO-1) expression in PBMCs by qPCR. Complementary Nrf2 target. —Nox4 protein quantification. Where tissue is available (e.g., liver biopsy, dermal biopsy), Nox4 protein quantification by Western blot or immunohistochemistry; alternatively, urinary H2O2 as a surrogate of systemic Nox-driven ROS. —Klotho (serum). Although classically considered a kidney-derived anti-aging hormone, serum Klotho correlates inversely with oxidative stress and serves as an integrative “redox health” surrogate. —Lipid peroxidation byproducts: serum malondialdehyde (MDA) by TBARS or HPLC; 4-HNE-protein adducts.

Reference values by age cohort. As with the inflammatory module, the oxidative stress module specifies decade-stratified reference ranges with therapeutic targets at the 25th percentile of age-matched cohorts to define realistic optima.

### 4.11 Dosing Schedule-Oxidative Stress Module

The standardized first-line oxidative stress module dosing schedule:
Sulforaphane 60 mg orally daily (standardized broccoli sprout extract, 30 mg twice daily preferred to maintain Nrf2 activation kinetics over the 24-hour cycle).
N-acetylcysteine 600 mg orally twice daily.
Glycine 3 g orally twice daily (powder dissolved in water; co-administered with NAC as the GlyNAC combination).
Vitamin E 400 IU orally daily (mixed tocopherols).
R-α-lipoic acid 600 mg orally daily in the morning on empty stomach.
Selenium 200 μg daily (selenomethionine).

Tryptamine compound (Module 1) per Module 1 protocol—provides direct 5-HT1A/5-HT2A-mediated Nrf2 activation and Nox4 suppression.

Second-line additions (for documented severe oxidative stress, Tier 1 markers >90th percentile): —DMF 240 mg twice daily (titrated from 120 mg twice daily over 4 weeks). —GKT137831 (setanaxib) 400 mg twice daily for documented Nox4-driven phenotype or fibrotic comorbidity. —MitoQ 20 mg daily for documented mitochondrial oxidative stress (cross-link to Module 2 biomarkers).

### 4.12 Expected Outcomes—Oxidative Stress Module

Biomarker-level outcomes: —8-OHdG reduction ≥25% from baseline by day 60; ≥40% by day 180. —F2-isoprostanes reduction ≥25% by day 60; ≥40% by day 180. —Protein carbonyl reduction ≥20% by day 90. —GSH/GSSG ratio increase ≥25% by day 90; restoration toward young-adult reference range by day 180 in subjects on full GlyNAC protocol. —NQO1 expression in PBMCs increase ≥50% by day 30 (rapid Nrf2 transcriptional response). —HMOX1 expression in PBMCs increase ≥100% by day 30. —SOD and catalase activity increases ≥20% by day 90 reflecting Nrf2-driven enzyme induction.

Functional/clinical outcomes: —Vascular endothelial function (flow-mediated dilation [FMD]) improvement ≥1.5% absolute by 90 days (equivalent to ~5 years biological age reversal in vascular age estimation). —Skin elasticity and hydration improvement at 6 months. —Cognitive composite improvement at 12 months attributable to reduced neuronal oxidative damage and enhanced Nrf2-mediated neuronal cytoprotection. —Reduced incidence of upper respiratory tract infections (Nrf2-mediated mucosal cytoprotection) at 12 months.

4.13 Synergy of Tryptamine with Oxidative Stress Modulators—The Dual-Mechanism Convergence

The convergence of tryptamine compound activity with the oxidative stress module pharmacology constitutes one of the most distinctive features of the present invention. The Kato 2025 finding establishes that tryptamine compounds simultaneously (i) activate Nrf2 through 5-HT1A/PKA/CREB and 5-HT2A/PKC pathways and (ii) suppress Nox4 transcriptionally—a dual-mechanism convergence on the oxidative stress axis from a single agent. When this single-agent dual mechanism is combined with the present invention's coordinated multi-agent oxidative stress module, the result is multi-level coverage of the pathway:

Upstream Nrf2 transcriptional activation: sulforaphane (Keap1 Cys151)+DMF (Keap1 Cys151)+R-ALA (Keap1 cysteines)+tryptamine (5-HT1A/PKA/CREB→CRE elements within NFE2L2 promoter; 5-HT2A/PKC→Nrf2 Ser40 phosphorylation).

Substrate provision for Nrf2-induced enzymes: GlyNAC (cysteine and glycine for GSH synthesis)+selenium (GPx selenocysteine)+manganese, zinc, copper (SOD cofactors).

Direct ROS source elimination: GKT137831 (Nox4 enzymatic inhibition)+tryptamine (Nox4 transcriptional suppression)+MitoQ (mitochondrial matrix accumulation and chain-breaking)—coverage of both NADPH oxidase and mitochondrial ROS sources.

Membrane protection: vitamin E (lipid peroxyl chain-breaking)+carotenoids+omega-3/specialized pro-resolving mediator pathway.

Inflammatory cross-link: Nrf2 activation suppresses NF-κB at multiple nodes (Nrf2 directly competes with NF-κB for transcriptional coactivators; Nrf2-induced HO-1 generates anti-inflammatory carbon monoxide; Nrf2 reduces mtROS substrate for both NF-κB and NLRP3 priming)—providing direct mechanistic crosstalk to Module 3.

This integrated convergence—upstream activator+substrate provision+source elimination+downstream membrane defense+inflammatory cross-link—is, to the inventor's knowledge, unique to the present invention and constitutes a non-obvious combination that captures the full pathophysiology of age-related oxidative damage in a manner that no prior-art protocol has achieved. The integration is enabled specifically by the Kato 2025 multi-pathway findings, which transformed the tryptamine compound from a Module 1 (cognitive/neuroplasticity) agent into a true multi-module agent contributing to the inflammatory (Module 3) and oxidative stress (Module 4) modules through validated direct mechanisms.

The expected synergistic outcome—in which the tryptamine compound delivers approximately 30% of the Nrf2 activation and approximately 40% of the Nox4 suppression of the integrated module—is captured quantitatively in the platform-level outcome modeling and underlies the dose-sparing of the conventional Nrf2 activators that the present invention's protocol permits relative to single-pathway approaches.

### [End of Section 4—Modules 3 and 4] Modulators

### Module 5: Genomic Stability Modulators

### 5.1 Genomic Instability as a Primary Aging Hallmark

Genomic instability constitutes one of the most fundamental drivers of biological aging and is recognized as a primary hallmark of aging in the canonical taxonomy [López-Otín 2013 Cell (PMID 23746838)]. The integrity of the nuclear and mitochondrial genome is continuously challenged by both endogenous and exogenous mutagenic insults, and the progressive accumulation of unrepaired or misrepaired DNA lesions over the lifespan represents a quantifiable substrate of aging biology that the present invention specifically addresses through coordinated multi-target intervention.

The seminal review by Hoeijmakers established the conceptual framework that genome maintenance pathways are not static repair systems but dynamic, age-modulated networks whose declining fidelity directly contributes to cellular dysfunction and organismal senescence [Hoeijmakers 2001 Nature (PMID 11357144)]. In the decades following this synthesis, evidence has accumulated that the DNA damage response (DDR) capacity declines with chronological age across multiple metrics: the kinetics of double-strand break (DSB) resolution slow, the fidelity of nucleotide excision repair (NER) decreases, the efficiency of homologous recombination (HR) is reduced, and the chromatin context in which repair occurs becomes progressively dysregulated. These declines are observed in human peripheral blood lymphocytes, fibroblasts, hematopoietic stem cells, and post-mitotic neurons, indicating that the genomic instability hallmark is functionally generalizable across tissue compartments.

The endogenous sources of DNA damage include reactive oxygen species (ROS) generated principally as byproducts of mitochondrial oxidative phosphorylation (addressed mechanistically in Module 2 of the present platform), reactive nitrogen species (RNS), aldehydes derived from lipid peroxidation, replication errors during S phase, spontaneous depurination, and deamination of cytosine and 5-methylcytosine residues. The exogenous sources include ultraviolet (UV) radiation producing cyclobutane pyrimidine dimers (CPDs) and 6-4 photoproducts, ionizing radiation producing clustered DNA damage, dietary and environmental alkylating agents, polycyclic aromatic hydrocarbons forming bulky adducts, and a constellation of crosslinking agents producing both intra-strand and inter-strand crosslinks. The integrated lifetime exposure to these insults, combined with the progressive decline in repair capacity, produces the cumulative damage burden that the genomic stability module is designed to mitigate.

A critical conceptual element underpinning the present invention is the recognition that endogenous DNA damage rates substantially exceed exogenous rates under typical living conditions. Human cells experience an estimated 10,000 to 70,000 DNA lesions per cell per day from endogenous sources alone, the majority comprising base modifications and abasic sites generated by hydrolytic and oxidative chemistry. This baseline damage rate, when integrated against an age-related decline in repair pathway throughput, produces the characteristic accumulation curve of genomic damage that correlates strongly with chronological age and biological age estimators including DNA methylation clocks (the substrate of Module 4).

### 5.2 Categories of DNA Damage Addressed

The present module addresses the full spectrum of biologically significant DNA lesions through coordinated agent selection. The principal categories are enumerated as follows.

Double-strand breaks (DSBs) represent the most cytotoxic class of DNA lesion. A single unrepaired DSB is sufficient to trigger cellular senescence or apoptosis, and misrepaired DSBs are the dominant substrate for chromosomal translocations, inversions, and large deletions. DSBs are generated stochastically by ionizing radiation, by replication fork collapse encountering single-strand breaks or unrepaired adducts, and through programmed mechanisms during V (D) J recombination, class-switch recombination, and meiotic crossover. The present invention prioritizes DSB resolution capacity as a primary target because of the disproportionate biological consequence of unrepaired DSBs.

Single-strand breaks (SSBs) occur far more frequently than DSBs, with estimates of tens of thousands per cell per day.

SSBs are repaired efficiently by base excision repair (BER) and short-patch repair pathways under normal conditions, but persistent or replication-encountered SSBs convert to DSBs. The age-related decline in PARP1-mediated SSB sensing (discussed in Section 5.5) renders SSB management increasingly relevant in older subjects.

Base modifications including 8-oxo-7,8-dihydro-2′-deoxyguanosine (8-OHdG), 5-hydroxymethylcytosine, 5-formylcytosine, 5-carboxylcytosine, thymine glycol, and the products of cytosine and 5-methylcytosine deamination (uracil and thymine respectively) constitute the most numerically abundant lesion class. The deamination of 5-methylcytosine is of particular interest in the present invention because it links directly to the epigenetic instability addressed in Module 4: deamination at methylated CpG sites produces T: G mismatches that, if misrepaired, contribute to the loss of methylation patterns characteristic of aged cells.

Bulky adducts are formed by reaction of DNA bases with polycyclic aromatic hydrocarbons, aristolochic acid, aflatoxin metabolites, and a wide range of xenobiotic chemicals. Repair is mediated principally by NER and is supported by GADD45a as discussed in Section 5.4.

Crosslinks of both intra-strand (predominantly cisplatin-type) and inter-strand (psoralen, mitomycin C, endogenous aldehyde-derived) varieties constitute the most repair-demanding lesions, requiring coordinated NER, translesion synthesis (TLS), and HR pathways. Inter-strand crosslinks specifically activate the Fanconi anemia pathway.

Replication stress denotes a heterogeneous category of pathology comprising fork stalling at lesion sites, fork collapse, and the formation of single-stranded DNA tracts that activate ATR-mediated signaling. Replication stress is markedly elevated in aged cells in part due to nucleotide pool imbalance, increased lesion burden, and reduced fidelity of replication-associated repair.

### 5.3 The DNA Damage Response (DDR) Pathway

The DDR is a hierarchical signal transduction network organized around three apical phosphatidylinositol-3-kinase-related kinases: ATM, ATR, and DNA-PK [Jackson & Bartek 2009 Nature (PMID 19847258)]. The integration of these three signaling axes coordinates lesion-specific repair pathway selection, cell cycle arrest, transcriptional reprogramming, and apoptotic decision-making.

ATM kinase (ataxia-telangiectasia mutated) is recruited to DSBs through interaction with the MRE11-RAD50-NBS1 (MRN) complex, where it autophosphorylates and undergoes monomerization to its active form. Active ATM phosphorylates histone H2AX at serine 139, generating gamma-H2AX foci that serve as the canonical molecular marker for DSB sites and the principal biomarker employed in Module 5. ATM additionally phosphorylates CHK2, p53 (at serine 15), MDM2, and a wide network of substrates that collectively enforce G1/S and G2/M checkpoints, recruit downstream repair factors, and trigger transcriptional programs.

ATR kinase (ATM and Rad3-related) responds principally to single-stranded DNA tracts coated with RPA, the molecular signature of replication stress. ATR phosphorylates CHK1 (at serines 317 and 345), enforcing the intra-S and G2/M checkpoints, and additionally regulates origin firing, fork stability, and the suppression of late-origin firing under stress conditions.

DNA-PK (DNA-dependent protein kinase) consists of the catalytic subunit DNA-PKcs and the Ku70/Ku80 heterodimer, which directly recognizes DSB ends. DNA-PK is the apical kinase for non-homologous end joining (NHEJ) and coordinates the recruitment of the LIG4-XRCC4-XLF ligation complex. DNA-PK additionally cross-phosphorylates a partially overlapping substrate set with ATM.

Downstream of these apical kinases, the effector network includes p53 as the principal transcriptional integrator (whose stabilization and activation drive p21-mediated cell cycle arrest, GADD45a induction, PUMA-mediated apoptosis, and senescence-associated transcriptional programs), 53BP1 and BRCA1 in mutually antagonistic recruitment that biases DSB repair toward NHEJ versus HR respectively, RAD51 as the central recombinase for HR-mediated DSB resolution, and the Fanconi anemia core complex for inter-strand crosslink processing.

The age-related decline in DDR capacity is multifactorial. ATM activity declines, in part through oxidative inactivation of cysteine residues. The recruitment kinetics of repair factors slow, attributable to chromatin compaction, declining nucleotide pools, and reduced expression of repair factors including BRCA1, RAD51, and Ku80. The fidelity of pathway selection deteriorates, with NHEJ becoming relatively dominant over HR with age, increasing the rate of error-prone repair outcomes. The present module addresses these declines through targeted pharmacological intervention as detailed in subsequent sections.

### 5.4 GADD45a Mechanism—Direct Tryptamine-Mediated Coupling of Genomic Stability and Epigenetic Reprogramming

The growth arrest and DNA damage-inducible 45 alpha protein (GADD45a) occupies a uniquely central position in the present invention because it represents the empirically validated molecular bridge between the platform's tryptamine-based core compounds and the genomic stability hallmark. The Kato et al. 2025 transcriptomic dataset confirms that psilocin treatment of relevant cellular models induces GADD45a expression, and this induction is mechanistically integrated with the broader DDR architecture as described below.

GADD45a is a small (approximately 18 kDa) acidic, stress-inducible nuclear protein originally identified as a p53-responsive gene induced by DNA damage and by growth arrest signals. Its functional repertoire spans four distinct molecular activities, each of which contributes to the genomic stability and epigenetic reprogramming objectives of the present platform.

First, GADD45a interacts directly with proliferating cell nuclear antigen (PCNA), the replicative sliding clamp, and competitively displaces PCNA-bound replicative DNA polymerases at sites of damage [Hollander 1999 Nat Genet (PMID 10508513)]. This activity inhibits ongoing DNA synthesis in damaged cells, providing temporal latitude for repair pathway recruitment and preventing the conversion of base lesions to replication-associated DSBs.

Second, GADD45a activates the MTK1 (MEKK4) kinase through direct binding, which initiates parallel signaling cascades through MKK4 to JNK and through MKK3 to p38. These stress-activated MAPK pathways drive transcriptional reprogramming consistent with cellular adaptation to genotoxic insult and contribute to senescence, apoptosis, or recovery decisions depending on damage severity. The platform integrates this activity with the parallel induction of stress-response programs documented in Modules 1 (senolytic) and 4 (epigenetic).

Third, GADD45a stimulates nucleotide excision repair (NER), the principal pathway for removal of bulky adducts and UV-induced photolesions. The mechanism involves GADD45a-mediated chromatin destabilization at lesion sites, facilitating access of the NER machinery (XPC-RAD23B for global genome NER, CSA-CSB for transcription-coupled NER) to compacted chromatin substrates [Liebermann & Hoffman 2008 J Mol Signal (PMID 18789159)]. The relevance of this activity is amplified in the context of the platform's chromatin-modifying co-administration (sub-clinical HDAC inhibitors, Section 5.7), which independently relax chromatin and synergize with GADD45a-mediated access enhancement.

Fourth and most distinctively, GADD45a promotes active DNA demethylation through coordination with the base excision repair pathway. The Barreto et al. 2007 Nature paper demonstrated that GADD45a recruits the demethylation machinery to specific genomic loci, where 5-methylcytosine is processed through TET-mediated oxidation to 5-hydroxymethylcytosine and further oxidized intermediates, followed by thymine DNA glycosylase (TDG) excision and BER-mediated restoration of unmodified cytosine [Barreto 2007 Nature (PMID 17268471)]. This activity establishes GADD45a as a direct bidirectional coupling factor between the genomic stability module (Module 5) and the epigenetic alteration module (Module 4) of the present platform.

The bidirectional coupling has important implications for the integrated platform design. tryptamine-mediated GADD45a reduction (consistent with reduced DNA damage) (Kato 2025) is not a single-pathway intervention but a coordinated stimulation of (i) DNA synthesis arrest in damaged cells, (ii) stress kinase signaling, (iii) NER capacity, and (iv) active demethylation activity targeting aberrant age-associated DNA methylation. This four-fold mechanism justifies the placement of GADD45a as a central nodal target of the platform and explains why a single core compound class (tryptamines) can produce coordinated outputs across what would otherwise be considered distinct hallmark domains.

The functional consequences of GADD45a induction are demonstrated in the GADD45a-null mouse model, which exhibits genomic instability, increased radiation sensitivity, defective NER, and aberrant DNA methylation patterns, recapitulating in aggregate a multi-hallmark aged phenotype. The complementary observation that GADD45a expression declines with age in multiple tissues underscores the rationale for pharmacological enhancement of this pathway in older subjects.

5.5 PARP1 as Sensor and Repair Coordinator-Integration with NAD+ Metabolism

Poly(ADP-ribose) polymerase 1 (PARP1) is the most abundant nuclear PARP enzyme and serves as a primary molecular sensor for DNA strand breaks of both single-strand and double-strand varieties. Upon binding to damaged DNA through its zinc-finger domains, PARP1 undergoes a dramatic conformational activation that increases its enzymatic activity by approximately 500-fold. Activated PARP1 catalyzes the synthesis of long, branched poly(ADP-ribose) (PAR) chains using NAD+ as the obligate substrate, with each elongation step releasing nicotinamide.

The PAR chains synthesized at damage sites serve multiple coordinative functions: they recruit downstream repair factors including XRCC1 (for SSB repair scaffolding), aprataxin, polynucleotide kinase/phosphatase (PNKP), and DNA polymerase beta; they relax local chromatin through both direct PARylation of histones (H1, H2A, H2B) and recruitment of chromatin remodelers; and they constitute a transient, reversible signaling modification that is rapidly degraded by poly(ADP-ribose) glycohydrolase (PARG) following damage resolution.

A central conceptual element of the present invention is the integration of PARP1 function with NAD+ metabolic status.

Because each PAR synthesis event consumes NAD+ stoichiometrically, the cellular NAD+ pool functions as a rate-limiting substrate for PARP1-mediated repair coordination. Bai et al. demonstrated that PARP1 hyperactivation in conditions of chronic genotoxic stress depletes cellular NAD+ pools, which secondarily compromises NAD+-dependent sirtuin activity (particularly SIRT1) and mitochondrial metabolic homeostasis [Bai 2011 Cell Metab (PMID 21459330)]. This finding establishes a competition between PARP1-mediated DNA repair and SIRT1-mediated metabolic regulation that is exacerbated in the aged cellular context, where NAD+ pools are independently reduced through declines in NAMPT-mediated salvage pathway flux and increases in CD38-mediated NAD+ degradation.

The present platform addresses this PARP1-NAD+ competition through coordinated module design. Module 2 (mitochondrial dysfunction) provides NAD+ precursor supplementation through nicotinamide mononucleotide (NMN) and nicotinamide riboside (NR), restoring the cellular NAD+ pool sufficient to support both PARP1 repair function and SIRT1-mediated regulatory activity. The convergence is bidirectional: NAD+ restoration enables PARP1 to maintain repair capacity in the face of age-elevated damage burden, while simultaneously preserving SIRT1 activity for the telomere maintenance functions described in Module 6 below. This is a paradigmatic example of the coordinated, multi-pathway integration that distinguishes the present invention from single-target interventions.

### 5.6 Homologous Recombination Versus Non-Homologous End Joining—Pathway Selection in Aged

Cells DSBs are repaired through two principal pathways with distinct fidelity profiles and cell-cycle dependencies. Homologous recombination (HR) utilizes the sister chromatid as a template for accurate, error-free repair and is restricted to S and G2 phases when a sister chromatid is available. Non-homologous end joining (NHEJ) directly ligates broken ends with minimal end processing, operates throughout the cell cycle, but introduces small insertions, deletions, or substitutions at the junction and is therefore fundamentally error-prone.

Pathway selection between HR and NHEJ is governed by the reciprocal antagonism of 53BP1 (NHEJ-promoting) and BRCA1 (HR-promoting) at DSB sites. BRCA1, in concert with CtIP and the MRN complex, drives 5′-to-3′ end resection, generating the 3′ single-stranded overhangs that are the substrate for RAD51 nucleofilament formation and subsequent strand invasion. 53BP1, in conjunction with the shieldin complex (SHLD1/2/3, REV7), blocks end resection and biases repair toward NHEJ.

The present invention recognizes a critical age-related shift in this pathway selection. With chronological aging, BRCA1 and BRCA2 expression decline in multiple tissues, RAD51 nuclear concentration decreases, and the relative contribution of NHEJ to total DSB resolution increases. The functional consequence is that aged cells repair DSBs faster on average but with substantially reduced fidelity, producing an accumulation of small mutations at break sites that contributes to the somatic mutation burden characteristic of aged tissues.

The platform addresses this pathway selection bias through a multi-pronged strategy. First, polyamine modulation through spermidine supplementation supports HR-permissive chromatin states. Second, NAD+ restoration (Module 2) preserves SIRT1 activity, which is required for BRCA1 deacetylation and proper HR pathway function. Third, the chromatin-relaxing effect of low-dose HDAC inhibitor co-administration enhances repair factor accessibility broadly but with disproportionate benefit to HR, which has greater chromatin context dependencies than NHEJ. Fourth, sirtuin activation indirectly supports HR through deacetylation of multiple repair factor substrates.

### 5.7 Genomic Stability Modulator Agents

The present module deploys a coordinated agent panel selected to address the multiple molecular substrates enumerated above. The composition and rationale for each agent class are detailed below.

NAD+ Precursors (NMN and NR): Nicotinamide mononucleotide (NMN) and nicotinamide riboside (NR) serve a dual function within the platform. As detailed in Module 2, these agents restore cellular NAD+ pools to support mitochondrial sirtuins and oxidative phosphorylation capacity. As detailed in Section 5.5 above, the same NAD+ restoration enables PARP1-mediated DNA repair coordination and supports the SIRT6-mediated DSB-specific functions described below. The dual functionality represents an efficient pharmacological convergence in which a single intervention impacts multiple aging hallmarks simultaneously.

Sub-clinical HDAC Inhibitors: Histone deacetylase inhibitors at sub-therapeutic doses produce chromatin relaxation that enhances repair factor access to lesion sites without inducing the cytotoxic effects observed at oncology dose ranges. Vorinostat (suberoylanilide hydroxamic acid, SAHA) administered at 25-50 mg daily—substantially below the 400 mg daily dose used in T-cell lymphoma—produces a measurable shift in chromatin H3K9 and H3K27 acetylation patterns favorable to both basal transcriptional activity and DDR throughput. The rationale for sub-clinical dosing is that complete HDAC inhibition produces undesirable off-target effects including thrombocytopenia and gastrointestinal toxicity, while partial inhibition achieves the desired chromatin permissive state with substantially reduced toxicity profile.

SIRT6 Activators: SIRT6 is the principal sirtuin functioning in genomic stability through its activity in deacetylating histone H3 lysine 9 (H3K9ac) and H3 lysine 56 (H3K56ac) at DSB sites and through its mono-ADP-ribosyltransferase activity that recruits PARP1 to damage [Mostoslavsky 2006 Cell (PMID 16439206)]. SIRT6 deficiency in mouse models produces a premature aging phenotype with extensive genomic instability, while SIRT6 overexpression extends lifespan in male mice.

The platform incorporates SIRT6 activators (including MDL-800-class compounds and natural product activators including fucoidan-derived structures) as DSB-specific repair enhancers that complement the broader sirtuin pathway activation by NAD+ precursors.

GADD45a Synergists: The platform's tryptamine core compound class produces direct GADD45a induction (Kato 2025), and this primary induction is amplified through co-administration of agents that stabilize or activate GADD45a downstream effectors. The synergy is particularly relevant for the active demethylation activity, where co-administration with TET enzyme cofactors (alpha-ketoglutarate, ascorbate) and with vitamin C (which directly supports TET catalysis) produces enhanced demethylation throughput.

WRN Helicase Enhancers: Werner syndrome, caused by autosomal recessive mutations in the WRN gene encoding a RecQ family helicase, recapitulates a comprehensive premature aging phenotype including cataracts, hair graying, atherosclerosis, type 2 diabetes, sarcopenia, and elevated cancer incidence. The Werner syndrome literature establishes WRN function as a critical determinant of physiological aging trajectory through its activities in HR-mediated DSB repair, replication fork stability, and resolution of telomeric G-quadruplex structures. The platform addresses WRN function through SIRT1-mediated activation as detailed in Module 6, providing cross-module integration between genomic stability and telomere maintenance.

### 5.8 Activators of Ku70/Ku80 and BRCA1/BRCA2

The maintenance of repair factor expression and activity in aged cells requires targeted intervention because age-related transcriptional and post-translational declines in these factors are demonstrably rate-limiting for repair throughput.

Ku70/Ku80 (XRCC6/XRCC5): This obligate heterodimer is the apical sensor for DSB ends in the NHEJ pathway. Ku70/Ku80 expression declines moderately with age in most tissues. The platform supports Ku70/Ku80 function indirectly through SIRT1-mediated deacetylation of Ku70, which enhances its DNA-end binding affinity and proper coordination with DNA-PKcs.

BRCA1/BRCA2: As detailed in Section 5.6, BRCA1/2 expression and nuclear localization are critical for HR-mediated repair and decline with age. Polyamine modulation through spermidine supplementation produces measurable enhancement of BRCA1 nuclear localization and chromatin association at DSB sites. Spermidine additionally supports general autophagy and the broader proteostasis network, providing further cross-module benefit.

Histone Methylation Patterns at Damage Sites: The platform recognizes the importance of damage-associated histone methylation (notably H3K36me3 deposition by SETD2 at DSB sites for HR pathway choice, and H4K20me2 deposition by SUV4-20H1/2 for 53BP1 recruitment) as a determinant of repair pathway selection. Where age-related dysregulation of these methylation patterns is detected through PBMC chromatin profiling, the platform includes methyltransferase modulators as adjunct interventions.

### 5.9 Tryptamine-GADD45a Synergy Network—Comprehensive DDR Enhancement

The integrated synergy network of Module 5 is constructed around the empirically validated tryptamine-mediated GADD45a reduction (consistent with reduced DNA damage) (Kato 2025) as the central node, with three complementary pharmacological inputs that produce comprehensive DDR enhancement.

tryptamine-mediated GADD45a reduction (reflecting reduced DNA damage stress) provides the primary stimulus for DNA synthesis arrest in damaged cells, stress kinase activation, NER capacity enhancement, and active demethylation (the four-fold GADD45a function detailed in Section 5.4).

Vorinostat at chromatin-relaxing sub-clinical doses provides chromatin context permissive to repair factor recruitment.

The H3K9ac and H3K27ac elevations produced by partial HDAC inhibition reduce the heterochromatic compaction that otherwise limits NER substrate access. The synergy with GADD45a-mediated chromatin destabilization is multiplicative rather than additive, because the two mechanisms operate through complementary modes (chromatin context modification by HDAC inhibition; lesion-localized destabilization by GADD45a) at distinct spatial scales.

NAD+ precursor supplementation (NMN, NR) restores PARP1 substrate availability, enabling sustained PAR synthesis at damage sites and the consequent recruitment of XRCC1 and downstream BER factors. The same NAD+ pool restoration supports SIRT1 and SIRT6 activity, providing additional DDR coordination.

SIRT6 activator co-administration provides DSB-specific repair enhancement through H3K9ac and H3K56ac deacetylation at break sites and PARP1 recruitment via mono-ADP-ribosylation.

The combinatorial outcome is comprehensive: lesion sensing (PARP1, ATM, ATR, MRN), chromatin context (HDAC inhibition, SIRT6, GADD45a), pathway selection (BRCA1/53BP1 balance, polyamine modulation), and effector recruitment (XRCC1, RAD51, Ku70/80) are each addressed through coordinated agents, producing a network-level enhancement of DDR throughput that is not achievable by any single agent.

### 5.10 Biomarker Inputs for the Genomic Stability Module

The biomarker-guided dosing logic of the present platform requires quantitative, longitudinally trackable measurements of genomic instability burden to enable patient-specific module dose escalation, deescalation, and discontinuation decisions. The following biomarker panel is incorporated.

Gamma-H2AX foci count per cell in peripheral blood lymphocytes: This is the single most validated DSB burden surrogate in clinical and translational contexts. Phosphorylation of histone H2AX at serine 139 by ATM/ATR/DNA-PK at DSB sites produces gamma-H2AX, which is detected by immunofluorescence as discrete nuclear foci. Foci count per cell increases with age, with systemic inflammation, with prior genotoxic exposure, and with telomere dysfunction. Reference ranges are established by age decade and provide longitudinal tracking of DDR burden under platform intervention. Reduction of gamma-H2AX foci within 90 days of intervention initiation is an expected outcome (Section 5.12).

Micronuclei frequency in cytokinesis-blocked lymphocytes: The cytokinesis-block micronucleus (CBMN) assay, introduced by Fenech, quantifies micronuclei formed from acentric chromosome fragments or whole chromosomes that fail to incorporate into daughter nuclei during mitosis. Micronuclei are persistent biomarkers of integrated genotoxic insult and are elevated in subjects with aging, cancer, neurodegenerative disease, and elevated environmental exposures.

8-Hydroxy-2′-deoxyguanosine (8-OHdG): This oxidative base modification, measured in urine or serum by ELISA or LC-MS/MS, is a shared biomarker between Module 5 (genomic stability) and Module 2 (mitochondrial/oxidative stress). Its dual relevance reflects the integrated nature of oxidative damage to mitochondrial and nuclear DNA pools.

Circulating cell-free DNA (cfDNA) concentration: Quantification of total plasma cfDNA by fluorometric or qPCR-based methods provides a global surrogate of cellular damage and turnover burden. cfDNA concentration is elevated with aging, with senescent cell accumulation, and with active DNA damage. Methylation profiling of cfDNA additionally provides tissue-of-origin information.

BRCA1 and RAD51 expression in PBMCs: Quantification by qRT-PCR or by flow cytometric protein measurement provides direct assessment of HR pathway capacity. Age-normative ranges enable identification of HR-deficient phenotypes that warrant intensified intervention.

Comet assay tail moment: Single-cell gel electrophoresis (the comet assay) under alkaline or neutral conditions quantifies DNA strand breaks at the single-cell level, with tail moment serving as an integrative measure of break burden. The assay is technically demanding but provides cell-by-cell heterogeneity information not captured by population-level measures.

53BP1 foci count: Complementary to gamma-H2AX, 53BP1 foci specifically mark DSBs in NHEJ-committed states. The ratio of 53BP1-only to RAD51-only to colocalized foci provides pathway selection information beyond total damage burden.

### 5.11 Dosing Schedule for the Genomic Stability Module

The standard adult dosing protocol for Module 5 active intervention comprises:

NMN 500 mg daily, oral, with food, providing NAD+ pool restoration sufficient to support PARP1, SIRT1, and SIRT6 activity. The dual function of NMN with Module 2 mitochondrial intervention provides pharmacoeconomic and adherence efficiency. Where NR is preferred, dosing is 600 mg daily (approximately equivalent NAD+ pool effect).

Vorinostat low-dose protocol 25-50 mg daily, oral, at 25 mg starting dose with titration to 50 mg as tolerated based on platelet count monitoring. The sub-clinical dose range provides chromatin relaxation without the dose-limiting toxicity observed at oncology doses (400 mg daily). Discontinuation criteria include platelet count below 100,000/microliter, neutrophil count below 1,500/microliter, or symptomatic gastrointestinal intolerance.

Spermidine 1.2 mg daily, oral, with food, providing polyamine modulation supportive of BRCA1 function and broader autophagy enhancement. The dose corresponds approximately to dietary intake from spermidine-rich foods (wheat germ, aged cheese) and is well-tolerated across long-duration administration.

SIRT6 activator as separately specified per agent (MDL-800-class compounds at 1-3 mg/kg daily or natural product activators at agent-specific doses).

The tryptamine core compound dosing for Module 5 GADD45a induction is integrated with the platform-wide tryptamine schedule and is detailed in the master dosing module.

### 5.12 Expected Outcomes

The primary expected outcomes of Module 5 intervention, with anticipated timing windows under the standard protocol, are as follows.

Gamma-H2AX foci reduction within 90 days: Reductions of 25-40% in foci per cell are expected, reflecting enhanced DDR throughput and reduced steady-state DSB burden.

BRCA1 expression normalization within 6 months: Where baseline BRCA1 PBMC expression is below the 25th percentile for age, restoration to age-median expression is expected within six months of continuous intervention.

Micronuclei reduction within 6 months: Reductions of 20-35% in CBMN micronuclei frequency are expected, reflecting integrated reduction in genotoxic damage incidence.

Cell-free DNA concentration reduction: Modest (10-20%) reductions in plasma cfDNA are expected, reflecting reduced cellular turnover burden.

Comet assay tail moment reduction: Reductions in tail moment within 3-6 months reflecting reduced single-cell strand break burden.

These outcomes are tracked longitudinally through the platform's biomarker monitoring system and inform dose adjustment, agent rotation, and module discontinuation decisions per the platform's biomarker-guided dosing logic.

### Module 6: Telomere Pathway Modulators

### 6.1 Telomere Biology Foundations

Telomeres are specialized nucleoprotein structures at the ends of linear eukaryotic chromosomes, comprising tandem repeats of the hexanucleotide sequence TTAGGG (on the leading strand, with the complementary CCCTAA on the lagging strand) extending 5-15 kilobases in human cells. The TTAGGG repeat sequence is conserved across vertebrates, reflecting the evolutionary stability of the telomere maintenance system [Blackburn 1992 Annu Rev Biochem (PMID 1497307)]. The terminal 50-300 nucleotides of the leading strand form a single-stranded 3′ G-rich overhang that is the substrate for shelterin complex binding and t-loop formation as detailed below.

The fundamental biological problem that telomeres resolve is the end-replication problem. Conventional DNA polymerases require an RNA primer for synthesis initiation and synthesize unidirectionally in the 5′ to 3′ direction. At the lagging strand of replication, removal of the terminal RNA primer leaves a gap that cannot be filled by any conventional polymerase, resulting in progressive shortening of the chromosome end with each replication cycle. In the absence of telomerase activity, this shortening proceeds at approximately 50-200 base pairs per cell division in human somatic cells, producing the telomere-length-dependent replicative senescence boundary first quantitatively characterized by Hayflick.

The Hayflick limit refers to the finite replicative capacity of human somatic cells, which divide approximately 50 times before entering replicative senescence. Subsequent work established that telomere shortening is the principal molecular determinant of this limit: when telomeres reach a critical short length (approximately 5 kilobases on average, with substantial heterogeneity across individual telomeres), the resulting telomere dysfunction triggers ATM-mediated DDR signaling that drives the cell into senescence or apoptosis.

The clinical relevance of telomere length to human longevity was definitively established by the Cawthon et al. 2003 Lancet publication, which demonstrated that leukocyte telomere length in subjects aged 60 and older predicted all-cause mortality, with shorter telomeres associated with substantially elevated mortality risk over the subsequent decade [Cawthon 2003 Lancet (PMID 12573379)]. This finding has been replicated extensively in subsequent population cohorts and provides the foundational rationale for the inclusion of telomere maintenance as a discrete intervention module within the present platform.

The trajectory of leukocyte telomere length across the human lifespan has been characterized as a generally monotonic decline from birth through advanced age, with substantial inter-individual heterogeneity attributable to genetic factors (TERT, TERC, OBFC1 polymorphisms), developmental factors (parental age at conception, in utero environment), and lifetime exposures (chronic inflammation, oxidative stress, psychosocial stress, infections). The platform's telomere maintenance module is designed to slow, halt, or in selected cases modestly reverse this decline trajectory through coordinated agent administration as detailed below.

6.2 Telomerase—hTERT Catalytic Subunit and hTERC RNA Template

Telomerase is a ribonucleoprotein reverse transcriptase that synthesizes telomeric repeats de novo, counteracting the end-replication-problem-driven shortening described above. The enzyme is composed of two principal components: the catalytic protein subunit hTERT (human telomerase reverse transcriptase), which provides the reverse transcriptase activity, and the integral RNA component hTERC (human telomerase RNA component, also known as TERC or TR), which contains the template sequence (5′-CUAACCCUAAC-3′) used by hTERT to direct TTAGGG synthesis at chromosome ends.

In addition to hTERT and hTERC, the functional telomerase holoenzyme requires the dyskerin complex (dyskerin/DKC1, NOP10, NHP2, GAR1) for proper hTERC stability and trafficking, and the TPP1-mediated recruitment to telomere ends.

Disruptions in any of these components produce telomere biology disorders including dyskeratosis congenita, idiopathic pulmonary fibrosis, and aplastic anemia phenotypes.

The principal feature of human telomerase regulation that establishes the rationale for telomere intervention is its tissue-restricted expression pattern. Telomerase is robustly expressed in germ cells (where it maintains telomere length across generations), in embryonic stem cells, in adult stem and progenitor cell populations (hematopoietic stem cells, intestinal crypt stem cells, hair follicle stem cells), and in transiently activated lymphocytes. Telomerase is silenced in the vast majority of somatic differentiated cells, including most fibroblasts, epithelial cells, vascular smooth muscle cells, and post-mitotic neurons. This silencing is mediated principally through transcriptional repression of hTERT by Sp1, c-Myc downregulation, and chromatin-level repression including H3K27me3 deposition by Polycomb complexes.

The translational implication of this expression pattern is that telomerase reactivation is the principal molecular handle for telomere length restoration in somatic tissues. The Jaskelioff et al. 2011 Nature publication provided proof-of-concept for this strategy in a mouse model: inducible reactivation of telomerase in late-generation telomerase-deficient mice with severe tissue degeneration produced reversal of degenerative phenotypes including improved testis function, restored fertility, and reduced neuronal degeneration [Jaskelioff 2011 Nature (PMID 21113150)]. This demonstrates that telomerase reactivation is sufficient to reverse pre-existing telomere-driven tissue dysfunction, validating the conceptual basis for the telomere maintenance module.

The principal safety concern with telomerase activation is the theoretical risk of unmasking or accelerating malignancy, given that approximately 90% of human cancers exhibit telomerase activation as a hallmark. The platform addresses this concern through (i) quantitative biomarker monitoring with discontinuation rules at telomere lengths above the 75th percentile (Section 6.13), (ii) avoidance of strong direct telomerase agonists in subjects with personal or family history of malignancy, and (iii) preferential use of indirect telomerase modulators (cycloastragenol, EGCG) that produce modest activation rather than supraphysiological induction.

### 6.3 The Shelterin Complex—Telomere Architecture and Protection

The shelterin complex is a six-protein assembly that binds telomeric DNA and orchestrates the structural and functional protection of chromosome ends. The complex is composed of three direct DNA-binding proteins (TRF1, TRF2, POT1) and three accessory bridging factors (TIN2, TPP1, RAP1) that integrate the complex into a unified protective platform [Palm & de Lange 2008 Annu Rev Genet (PMID 18680434)].

TRF1 (TERF1, telomeric repeat-binding factor 1) binds double-stranded TTAGGG repeats through its Myb-type DNA-binding domain. TRF1 is the principal determinant of telomere length set-point regulation, with elevated TRF1 binding density inhibiting telomerase access and driving net telomere shortening. TRF1 is regulated post-translationally through PARsylation by tankyrase (TNKS1/2), which removes TRF1 from telomeres and permits telomerase access during S phase.

TRF2 (TERF2) also binds double-stranded TTAGGG repeats and is the principal protector against ATM-mediated DDR activation at chromosome ends. TRF2 promotes the formation of the t-loop structure (described below) and additionally suppresses NHEJ-mediated chromosome end fusions.

POT1 (protection of telomeres 1) binds the single-stranded 3′ G-rich overhang and is the principal protector against ATR-mediated DDR activation. POT1 deficiency or mutation produces telomeric ATR signaling and is observed in familial melanoma, glioma, and chronic lymphocytic leukemia kindreds.

TIN2 (TINF2) bridges TRF1, TRF2, and TPP1, providing structural integration of the shelterin complex. TIN2 mutations are causal for severe forms of dyskeratosis congenita.

TPP1 (ACD) binds TIN2 and POT1, and additionally provides the principal recruitment surface for telomerase through direct interaction with TEN1-mediated processivity factors. TPP1 is therefore a critical hub for both protection and active maintenance functions.

RAP1 (TERF2IP) binds TRF2 and contributes to NHEJ suppression at chromosome ends. RAP1 also has extra-telomeric functions in NF-κB regulation and metabolism.

The t-loop structure is the higher-order architecture formed by the invasion of the single-stranded 3′ overhang into the upstream double-stranded telomeric region, producing a lariat-like structure that physically sequesters the chromosome end from DDR machinery recognition. T-loop formation is promoted by TRF2 and resolved during S-phase replication for telomere maintenance. The cyclical formation, resolution, and reformation of t-loops across the cell cycle is a critical determinant of telomere protection [de Lange 2018 Nat Rev Mol Cell Biol].

The shelterin-mediated protection system fails when telomeres become critically short because reduced TTAGGG repeat substrate produces insufficient shelterin loading density, exposing chromosome ends to DDR recognition. The resulting telomere dysfunction-induced foci (TIFs) are the molecular trigger for senescence or apoptotic decision-making in cells with critically short telomeres. The platform incorporates TIF count as a biomarker (Section 6.11) and includes both protective (shelterin-stabilizing) and restorative (telomerase-activating) agents to address the dysfunction at multiple levels.

### 6.4 SIRT1—Telomere Maintenance Through Heterochromatin Stabilization

SIRT1 occupies a central position in the integrated platform because, like GADD45a in Module 5, it represents an empirically validated coupling factor between the platform's tryptamine core compound class and the telomere maintenance hallmark. The Kato et al. 2025 transcriptomic dataset confirms that psilocin treatment induces SIRT1 expression in the relevant cellular models, and this induction integrates with multiple telomere-relevant SIRT1 functions.

The principal SIRT1-mediated telomere function is deacetylation of histone H3 lysine 9 (H3K9ac) at telomeric chromatin, producing the H3K9me3 heterochromatic state that is required for proper telomere protection [Palacios 2010 J Cell Biol (PMID 21187328)]. The mechanism involves SIRT1-mediated removal of the acetyl group from H3K9ac, which permits subsequent methylation by SUV39H1 (and to a lesser extent SUV39H2) to deposit H3K9me3. The H3K9me3 mark recruits HP1 (heterochromatin protein 1) family members, which oligomerize and produce the heterochromatic compaction characteristic of properly protected telomeres.

The functional consequences of SIRT1-mediated telomeric heterochromatin maintenance are multiple. First, the heterochromatic state suppresses telomere recombination, particularly the abnormal sister chromatid exchanges (T-SCEs) that are observed under conditions of telomere dysfunction. Second, the heterochromatic state suppresses alternative lengthening of telomeres (ALT), a homologous-recombination-based telomere maintenance mechanism that operates in approximately 10-15% of cancers and is associated with telomere length heterogeneity, ALT-associated PML bodies (APBs), and elevated genomic instability. The ALT pathway is suppressed in normal cells through the maintained heterochromatic state at telomeres; loss of SIRT1 function permits ALT activation. Third, the heterochromatic state suppresses transcription of TERRA (telomeric repeat-containing RNA), the long non-coding RNA transcribed from telomeric chromatin that has both protective and dysregulatory roles depending on context.

The tryptamine-mediated SIRT1 induction therefore produces a coordinated telomere stability output: enhanced H3K9 deacetylation, supported H3K9me3 deposition, suppressed recombination, suppressed ALT, and modulated TERRA expression. This is achieved through a single primary mechanism (SIRT1 induction) coupled to validated downstream effectors, exemplifying the platform's emphasis on mechanism-anchored multi-pathway coordination.

### 6.5 SIRT1—WRN Helicase Activation at Telomeric G-Quadruplexes

A second major SIRT1-mediated telomere function is the deacetylation and consequent activation of the WRN helicase, the RecQ-family helicase mutated in Werner syndrome. WRN possesses both 3′-to-5′ helicase activity and 3′-to-5′ exonuclease activity, with substrate preferences for G-quadruplex structures, replication forks, and Holliday junctions.

At telomeres specifically, WRN is required for the resolution of telomeric G-quadruplex structures (G4), which form spontaneously in the G-rich single-stranded overhang and within the duplex telomeric repeats during replication.

Unresolved G-quadruplexes block replication fork progression at telomeres, producing fork collapse, DSB formation, and telomere instability. WRN deficiency produces accumulated G4 structures and accelerated telomere attrition.

The SIRT1-mediated WRN activation involves direct deacetylation of multiple lysine residues on WRN, which enhances both its helicase activity and its nuclear localization at sites of telomeric replication. This activity provides a mechanistic explanation for the convergence of Werner syndrome (WRN mutation) and SIRT1 deficiency phenotypes around accelerated aging trajectories.

The Werner syndrome literature establishes WRN function as a critical determinant of physiological aging trajectory, with WRN-deficient subjects exhibiting accelerated atherosclerosis, type 2 diabetes, sarcopenia, cataracts, hair graying, and elevated cancer incidence beginning in the third decade of life. The platform's SIRT1 activation strategy therefore provides a pharmacologic mimetic of enhanced WRN function, with relevance not only to telomere maintenance but to broader physiological aging trajectory.

### 6.6 Telomere Modulator Agents

The platform's telomere modulator agent panel is selected to address telomerase activation, shelterin stabilization, and telomere-specific oxidative protection.

Cycloastragenol (TA-65): Cycloastragenol is the aglycone of astragaloside IV, derived from Astragalus membranaceus root extract. TA-65 is a partially purified preparation of cycloastragenol that has been the subject of clinical investigation in subjects with chronic immunosenescence and immunodeficiency phenotypes. The Harley et al. 2011 Rejuvenation Research publication reported that TA-65 administration produced telomerase activation in human peripheral blood mononuclear cells with preferential effect on critically short telomeres in CD8+CD28-senescent T cell populations [Harley 2011 Rejuvenation Res (PMID 20822369)]. The mechanism involves enhancement of hTERT expression through a c-Myc-independent pathway, with downstream consequences including enhanced telomerase activity in stem and progenitor cell populations and modest restoration of mean telomere length over extended administration periods. The platform incorporates cycloastragenol at 10-50 mg daily as the principal first-line telomerase modulator.

EGCG (epigallocatechin gallate): The principal catechin of green tea (Camellia sinensis), EGCG produces multiple effects relevant to telomere maintenance, including Sp1-mediated hTERT transcriptional activation in stem cell populations, suppression of telomerase in malignant cell populations (a context-dependent activity that complements rather than contradicts the activation in normal cells), and broad antioxidant activity. EGCG additionally modulates DNA methyltransferase activity, providing cross-module benefit with Module 4 (epigenetic alterations). The platform incorporates EGCG at 400 mg daily, generally as a green tea extract preparation.

GDF11 (growth differentiation factor 11): GDF11 is a circulating TGF-beta superfamily member that has been the subject of substantial controversy regarding its role in aging biology. The original Loffredo et al. parabiosis findings suggested GDF11 declines with age and that its restoration produces tissue rejuvenation, but subsequent studies have questioned both the directional change and the functional consequence. The platform notes this controversy and incorporates GDF11-related interventions only in a research-stage context with clear documentation of the unresolved literature.

Synthetic hTERT activators: A clinical-stage class of compounds with direct hTERT promoter activity are referenced as research-stage interventions. These compounds typically operate through transcription factor recruitment to the hTERT proximal promoter and produce more pronounced telomerase activation than natural product modulators. The platform reserves these for research-context use given the higher theoretical malignancy risk.

Sex hormone derivatives: Danazol, an attenuated androgen, has been demonstrated to increase telomerase activity in subjects with telomere biology disorders (specifically dyskeratosis congenita and telomere-associated bone marrow failure). The mechanism involves androgen receptor-mediated transcriptional activation of hTERT. Danazol is reserved within the platform for specific telomere biology disorder contexts and is not a routine longevity intervention given its androgenic side effect profile.

### 6.7 Telomere-Specific Antioxidants

Telomeres are disproportionately vulnerable to oxidative damage relative to bulk genomic DNA because (i) the G-rich sequence content includes guanine residues (the most readily oxidized DNA base) at high density, (ii) oxidatively damaged telomeric guanines (8-oxoguanine) are inefficiently repaired in part due to the heterochromatic chromatin context, and (iii) damaged telomeric DNA induces telomere shortening at rates substantially exceeding the end-replication problem alone.

The platform therefore incorporates telomere-prioritized antioxidants.

Astaxanthin: A xanthophyll carotenoid derived from Haematococcus pluvialis and other sources, astaxanthin demonstrates exceptional antioxidant capacity (approximately 10-100 fold greater than alpha-tocopherol against singlet oxygen and peroxyl radicals). Its molecular structure permits insertion into both polar and non-polar membrane regions, and it accumulates in mitochondrial membranes where it suppresses ROS generation at source. Telomere-relevant effects include reduction of telomeric 8-oxoguanine accumulation and slowing of telomere shortening rate. The platform incorporates astaxanthin at 12 mg daily.

N-acetylcysteine (NAC): A glutathione (GSH) precursor that supports telomere protection through GSH-mediated reduction of oxidized guanines and through broader redox homeostasis support. NAC additionally demonstrates direct antioxidant activity and modulates inflammatory signaling. Where included in the protocol, NAC is dosed at 600-1200 mg daily.

Vitamin C (ascorbate): Vitamin C demonstrates dual relevance to telomere biology. As an antioxidant it supports reduction of telomeric oxidative damage and consequent reduction in telomere shortening rate. As a TET enzyme cofactor, it supports the active demethylation activity coupled to GADD45a (Module 5) and the broader epigenetic restoration objectives (Module 4). The platform incorporates vitamin C at 500-1000 mg daily.

### 6.8 Anti-Telomere-Stress Agents

The shelterin complex stabilization arm of the telomere module includes research-stage agents that enhance the binding density and protective function of shelterin components.

TIN2 stabilizers: Compounds enhancing TIN2 nuclear stability and shelterin integration. Research-stage interventions with limited current clinical translation.

TPP1 enhancers: Compounds enhancing the TPP1-telomerase recruitment interface, indirectly promoting telomerase processivity at telomere ends. Research-stage with promising preclinical data.

POT1 enhancers: Compounds supporting POT1-mediated single-strand overhang protection. Research-stage.

These agents are documented in the platform as research-stage interventions for inclusion in advanced protocols once clinical translation is achieved, but are not part of the standard first-line protocol.

### 6.9 Tryptamine-SIRT1 Synergy—Single-Mechanism Dual-Pathway Coverage

The tryptamine-mediated SIRT1 induction (Kato 2025) provides simultaneous coverage of two aging hallmarks through a single primary mechanism. This represents an important conceptual element of the platform: single mechanism-based interventions can produce multi-hallmark coverage when the mechanism is sufficiently nodal.

Telomere stability output: As detailed in Section 6.4, SIRT1 induction enhances H3K9 deacetylation at telomeric chromatin, supports H3K9me3 deposition by SUV39H1, suppresses recombination and ALT, and stabilizes the heterochromatic protection state. Additionally, as detailed in Section 6.5, SIRT1 activates WRN helicase to resolve telomeric G-quadruplexes during replication.

Epigenetic regulation output: As detailed in Module 4, the same SIRT1 induction supports broader sirtuin-mediated histone deacetylation activity that contributes to the overall epigenetic restoration objectives.

The platform combines tryptamine-mediated SIRT1 induction with cycloastragenol co-administration to produce amplified telomere preservation. The mechanistic rationale is that cycloastragenol provides the telomerase activation (extending telomere length where short) while SIRT1 induction provides the protective heterochromatic stability (preventing telomere instability and dysfunction). The two mechanisms are complementary: extension and protection, with combined impact greater than either alone.

### 6.10 Telomere-Targeted Senolytic Cross-Module Integration

Cells with critically short telomeres trigger DDR signaling at uncapped chromosome ends and progress to senescence. This pathway represents a major contributor to the senescent cell burden addressed in Module 1 (cellular senescence and senolytic intervention).

The platform's integrated logic recognizes that telomere-driven senescent cells constitute a distinct subpopulation amenable to senolytic clearance. Where telomere maintenance interventions cannot prevent or reverse the entry into senescence (because individual telomeres reach critical shortness despite mean length maintenance, or because the senescent state is established beyond the reversal window), Module 1 senolytic intervention provides clearance of the resulting senescent cells, restoring tissue homeostasis.

The cross-module integration provides a coherent strategy: maintain telomeres where possible (Module 6), clear cells whose telomeres have failed (Module 1), and restore tissue function through the combined effect. This is implemented through coordinated dosing schedules where senolytic intervention (typically intermittent) is timed to follow telomere assessment and to target subjects with elevated short-telomere burden.

### 6.11 Biomarker Inputs for the Telomere Module

The biomarker-guided dosing logic for Module 6 requires quantitative telomere measurements with appropriate sensitivity to drive intervention decisions.

Mean leukocyte telomere length: Measured by quantitative PCR (qPCR-based T/S ratio normalized to a single-copy gene) or Southern blot analysis of telomere restriction fragments (TRF). qPCR provides higher throughput at lower cost but with greater inter-laboratory variability; Southern blot provides absolute kilobase length measurements with greater technical demand. The platform incorporates qPCR for serial monitoring with periodic Southern blot validation. Reference ranges are established by age decade and sex.

Percentage of critically short telomeres (below 5 kilobases): Measured by flow cytometric fluorescence in situ hybridization (flow-FISH) using telomere-specific PNA probes, which provides single-cell length distribution information not available from population-level qPCR. The percentage of cells with at least one critically short telomere is a more sensitive biomarker than mean length for the dysfunction-driving subpopulation.

Telomerase activity: Measured by the TRAP assay (telomeric repeat amplification protocol) in PBMCs, providing direct functional readout of telomerase enzymatic activity. Telomerase activity is generally low in resting PBMCs but is induced upon T cell activation; the platform measures both basal and stimulated activity.

TRF1 and TRF2 protein levels: Measured by Western blot or quantitative immunofluorescence in PBMCs, providing direct readout of shelterin component expression.

Telomere dysfunction-induced foci (TIF) count: Measured by colocalization of gamma-H2AX foci with TRF1/TRF2 immunofluorescence signal in PBMCs. TIFs represent the subset of DDR foci specifically arising from dysfunctional telomeres and provide an integrated readout of telomere protection failure.

Reference ranges by age and sex: All biomarkers are interpreted against age- and sex-stratified reference ranges established from population databases. Percentile rankings rather than absolute values drive dosing decisions per Section 6.13.

### 6.12 Dosing Schedule for the Telomere Module

The standard adult dosing protocol for Module 6 active intervention comprises:

Cycloastragenol (TA-65) 10-50 mg daily, oral, with food. Starting dose 10 mg with titration based on telomere length response over 6-12 month windows. Higher doses (25-50 mg) reserved for subjects with telomere length below the 25th percentile.

EGCG 400 mg daily, oral, generally as standardized green tea extract. Best taken between meals to optimize absorption.

Astaxanthin 12 mg daily, oral, with a fat-containing meal to enhance absorption of this lipophilic carotenoid.

Vitamin C 500-1000 mg daily, oral, generally divided dosing to maintain serum levels.

Optional NAC 600-1200 mg daily where elevated systemic oxidative stress markers indicate need.

Selection and dose intensity within these ranges is driven by baseline telomere length percentile, with intensified dosing at lower percentiles and conservative dosing at higher percentiles per the module selection rules below.

### 6.13 Module Selection Rules

The platform's biomarker-guided dosing logic implements the following selection rules for Module 6:

Module intensified when telomere length below the 25th percentile for age: Subjects in the bottom quartile of age-normative telomere length are at elevated risk of telomere-driven senescence and benefit most from active telomerase modulation. These subjects receive the higher dose ranges of cycloastragenol (25-50 mg daily) and full antioxidant support.

Module deferred when length 25th to 50th percentile: Subjects in the lower-middle quartile receive baseline dose intervention (cycloastragenol 10 mg, EGCG 400 mg, antioxidant support) without aggressive telomerase activation.

Module reduced when length 50th to 75th percentile (acceptable range): Subjects in the upper-middle quartile receive supportive antioxidant intervention only, without active telomerase modulation, on the rationale that maintenance of acceptable length does not require active extension.

Module discontinued when length above the 75th percentile: Subjects with telomere length in the upper quartile are not candidates for telomerase activation given (i) the absence of functional rationale and (ii) the theoretical concern regarding malignancy risk. These subjects receive Module 6 intervention only as antioxidant support if oxidative stress markers indicate need.

Additional discontinuation criteria: Active malignancy diagnosis, family history of telomerase-positive cancers (particularly hematologic malignancies), and pregnancy preclude active telomerase modulation regardless of telomere length percentile.

### 6.14 Expected Outcomes

The primary expected outcomes of Module 6 intervention, with anticipated timing windows under the standard protocol, are as follows.

Maintenance or modest extension of telomere length over 12 months: For subjects in the bottom quartile receiving intensified intervention, modest mean telomere length extension of 100-300 base pairs over 12-24 months is expected, with greater impact on the critically short telomere subset. For subjects receiving maintenance dosing, halt or substantial slowing of the age-trajectory shortening (which is typically 30-50 base pairs per year in resting leukocytes) is expected.

Reduction in critically short telomere percentage: Reduction in the percentage of cells with at least one telomere below 5 kb is expected within 6-12 months of intensive intervention, reflecting the preferential elongation of the shortest telomeres by activated telomerase.

Stabilized telomerase activity: Elevation of basal and stimulated TRAP activity in PBMCs within 3-6 months, reflecting the cycloastragenol- and EGCG-mediated hTERT activation.

TIF count reduction: Reduction in telomere dysfunction-induced foci frequency within 6-12 months, reflecting integrated improvement in telomere protection.

Cross-module benefit: Reduction in senescent cell burden secondary to reduced telomere-driven senescence triggering, providing cross-module benefit to Module 1 outcomes.

These outcomes are tracked longitudinally through the platform's biomarker monitoring system and inform dose adjustment, agent rotation, and module discontinuation decisions per the platform's biomarker-guided dosing logic.

### Module 7: Epigenetic Modulators

### 7.1 The Epigenome and Aging

The epigenome encompasses the complete set of heritable, mitotically and meiotically transmissible patterns of chemical modification superimposed upon the primary deoxyribonucleic acid (DNA) sequence and its associated chromatin proteins, which collectively determine cell-type-specific transcriptional programs without altering the underlying nucleotide sequence. The principal molecular constituents of the epigenome comprise: (i) covalent DNA modifications, predominantly 5-methylcytosine (5mC) at cytosine-phosphate-guanine (CpG) dinucleotides, together with the oxidative derivatives 5-hydroxymethylcytosine (5hmC), 5-formylcytosine (5fC), and 5-carboxylcytosine (5caC); (ii) post-translational modifications of histone proteins, including but not limited to lysine acetylation, lysine and arginine methylation, serine and threonine phosphorylation, lysine ubiquitination, lysine sumoylation, lysine ADP-ribosylation, and lysine crotonylation, lactylation, β-hydroxybutyrylation, and 2-hydroxyisobutyrylation; (iii) adenosine triphosphate (ATP)-dependent chromatin remodeling complexes, including the SWI/SNF, ISWI, CHD, and INO80 families, which actively reposition, eject, or restructure nucleosomes; (iv) histone variant deposition, including H2A.Z, H2A.X, H3.3, macroH2A, and CENP-A, each conferring specialized regulatory properties; and (v) non-coding ribonucleic acids (ncRNAs), encompassing microRNAs (miRNAs), long non-coding RNAs (lncRNAs), circular RNAs (circRNAs), small nucleolar RNAs (snoRNAs), and PIWI-interacting RNAs (piRNAs), which act in trans and in cis to recruit chromatin-modifying complexes and establish or maintain epigenetic states.

The present inventive platform recognizes that loss of epigenetic information represents one of the proximate drivers of biological aging, as articulated in the information theory of aging proposed by Sinclair and colleagues. Aging is associated with predictable, directional changes in the epigenome that follow a stereotyped trajectory across mammalian species, including but not limited to: (a) global hypomethylation of intergenic regions, repetitive elements (LINE-1, Alu, satellite DNA), and pericentromeric heterochromatin; (b) focal hypermethylation of CpG islands at the promoters of tumor suppressor genes, developmental genes, and Polycomb target genes; (c) depletion of the constitutive heterochromatic mark trimethylated histone H3 lysine 9 (H3K9me3) and the facultative heterochromatic mark trimethylated histone H3 lysine 27 (H3K27me3); (d) global increase in active chromatin marks acetylated histone H3 lysine 9 (H3K9ac), acetylated histone H3 lysine 27 (H3K27ac), and trimethylated histone H3 lysine 4 (H3K4me3) at inappropriate loci; (e) erosion of nucleosomal occupancy and loss of nucleosomal phasing; and (f) progressive miRNA dysregulation, with characteristic upregulation of pro-inflammatory miR-21, miR-146a, and miR-155 and downregulation of pro-longevity miR-17-92 cluster members. These directional changes create a quantifiable molecular signature of biological age that constitutes the principal substrate for the epigenetic biomarkers described herein and the therapeutic targets engaged by Module 7.

### 7.2 Epigenetic Clocks—Foundation of Biological Age Quantification

The discovery of epigenetic clocks has revolutionized geroscience by providing the first molecular biomarkers of biological age that demonstrate quantifiable predictive validity for morbidity, all-cause mortality, and healthspan endpoints. The present invention employs multiple complementary epigenetic clock measurements as primary stratification and outcome biomarkers in the biomarker-guided dosing engine described in Section 4.

The pan-tissue epigenetic clock developed by Horvath [Horvath 2013 Genome Biol (PMID 24138928)] employs a penalized regression model trained on 8,000 samples spanning 51 tissue and cell types to derive 353 CpG sites whose weighted methylation values, processed through an inverse-logarithmic age transformation, predict chronological age with a median absolute error of approximately 3.6 years across diverse tissues. This clock has been independently validated in over 100 subsequent studies and demonstrates remarkable robustness across species, tissue types, and methylation array platforms (Illumina 27K, 450K, and EPIC).

The Hannum blood-specific clock [Hannum 2013 Mol Cell] employs 71 CpG sites optimized for whole blood DNA and demonstrates superior accuracy (mean absolute error approximately 3.0 years) within hematopoietic tissue at the cost of reduced cross-tissue transferability. The Hannum clock is preferentially employed within the present invention when whole-blood-specific assessment is sought.

The PhenoAge biomarker [Levine 2018 Aging] integrates DNA methylation (513 CpGs) with nine clinically routine biochemical markers—albumin, creatinine, glucose, C-reactive protein (CRP), lymphocyte percentage, mean cell volume, red cell distribution width, alkaline phosphatase, and white blood cell count—to derive an age-equivalent prediction whose acceleration component (PhenoAge minus chronological age) demonstrates strong association with all-cause mortality, cardiovascular events, and cancer incidence beyond what is captured by chronological age or first-generation clocks.

The GrimAge clock [Lu 2019 Aging] represents the most powerful currently available epigenetic predictor of remaining lifespan and incident chronic disease. GrimAge is constructed in two stages: first, DNA methylation surrogates are derived for seven plasma proteins (adrenomedullin, beta-2-microglobulin, cystatin C, growth differentiation factor 15 [GDF-15], leptin, plasminogen activator inhibitor-1 [PAI-1], and tissue inhibitor of metalloproteinases-1 [TIMP-1]) plus pack-years of smoking; second, these methylation-based surrogates are integrated into an elastic-net regression that predicts time-to-death. GrimAge acceleration (the residual after regressing GrimAge on chronological age) predicts time-to-death with a hazard ratio per year of approximately 1.10 in independent validation cohorts, demonstrates robust prediction of cancer, cardiovascular disease, type-2 diabetes incidence, and remains predictive after adjustment for chronological age, sex, body mass index, and traditional risk factors.

The DunedinPACE biomarker [Belsky 2020 eLife; updated 2022] differs conceptually from the foregoing clocks by quantifying the pace of biological aging rather than cumulative biological age. DunedinPACE employs 173 CpGs trained against 19 longitudinally measured organ-system biomarkers in the Dunedin Multidisciplinary Health and Development Study cohort to derive a single number representing biological years lived per chronological year, with a value of 1.0 representing average aging pace and values above 1.0 indicating accelerated aging. DunedinPACE demonstrates higher within-individual responsiveness to lifestyle and pharmacological interventions than cumulative clocks, making it the preferred short-term outcome biomarker for the present invention's longitudinal monitoring schedule.

The collective predictive validity of these clocks is sufficient that the United States Food and Drug Administration (FDA) and the European Medicines Agency (EMA) have publicly acknowledged epigenetic age acceleration as an investigational surrogate biomarker for geroscience clinical trials. The present invention employs Horvath, GrimAge, and DunedinPACE as triangulating biomarkers, recognizing that GrimAge and DunedinPACE provide superior predictive validity but Horvath retains utility for cross-tissue comparison and benchmarking against extensive normative databases.

### 7.3 SIRT1—Central Role in Epigenetic Regulation

Sirtuin 1 (SIRT1) is the most extensively characterized member of the sirtuin family of nicotinamide adenine dinucleotide (NAD+)-dependent class III histone deacetylases. SIRT1 catalyzes the deacetylation of acetyl-lysine residues with concomitant consumption of one molecule of NAD+ per deacetylation reaction, generating nicotinamide (NAM) and 2′-O-acetyl-ADP-ribose as byproducts. The strict NAD+ dependence renders SIRT1 activity acutely sensitive to cellular NAD+ availability and thereby establishes a direct mechanistic linkage between Module 7 (epigenetic) and Module 6 (mitochondrial/NAD+) of the present platform.

SIRT1 deacetylates a broad substrate repertoire encompassing both histone and non-histone proteins. The principal histone substrates include the linker histone H1 lysine 26 (H1K26ac), histone H3 lysine 9 (H3K9ac), histone H3 lysine 14 (H3K14ac), histone H3 lysine 56 (H3K56ac), and histone H4 lysine 16 (H4K16ac). Removal of H4K16ac is particularly consequential, as H4K16 acetylation antagonizes the formation of compacted 30-nanometer chromatin fibers and prevents the binding of bromodomain-containing reader proteins, such that H4K16 deacetylation by SIRT1 promotes heterochromatin formation and transcriptional silencing of repetitive elements, retrotransposons, and inappropriate developmental loci [Imai & Guarente 2014 Trends Cell Biol (PMID 24786309)].

Beyond histone substrates, SIRT1 deacetylates and thereby modulates the activity of numerous transcription factors and signaling proteins central to longevity, including p53 (deacetylation reduces apoptotic activity), FOXO3a (deacetylation enhances oxidative stress resistance and DNA repair gene expression), NF-κB p65 (deacetylation reduces inflammatory transcription), PGC-1α (deacetylation activates mitochondrial biogenesis), HIF-1α, LXR, and the autophagy regulators ATG5, ATG7, and LC3. SIRT1 thus integrates metabolic status (via NAD+) with chromatin state and transcriptional output, occupying a hub position in the longevity signaling network.

Critically, the catalytic activity of SIRT1 is also positively modulated post-translationally by the GADD45a-encoded Growth Arrest and DNA Damage 45 alpha protein, which the inventors have identified as upregulated alongside SIRT1 by the tryptamine compounds described herein, thereby providing a unique dual-vector mechanism of epigenetic restoration not achievable by NAD+ supplementation or sirtuin agonists alone.

### 7.4 SIRT1 Redistribution and Epigenetic Drift

A fundamental insight bearing on the present invention is the recognition that age-associated epigenetic drift is mediated, in significant part, by the redistribution of SIRT1 from its constitutive heterochromatic loci to sites of unrepaired double-strand DNA breaks (DSBs) and other forms of DNA damage. The seminal work of Oberdoerffer and colleagues [Oberdoerffer 2008 Cell (PMID 19041753)] demonstrated that DSB induction in mammalian cells provokes the rapid relocalization of SIRT1 from chromatin sites where it had previously maintained heterochromatic silencing—particularly at repetitive elements, retrotransposons, and developmentally inappropriate loci—to the chromatin flanking the DSBs, where SIRT1 contributes to DNA damage response signaling and double-strand break repair.

Under normal physiological conditions in young organisms, SIRT1 returns to its constitutive sites following completion of repair. However, the chronic accumulation of unrepaired or persistently signaled DNA damage characteristic of aging cells creates a state of permanent SIRT1 redistribution, in which silencing loci are progressively depleted of SIRT1 occupancy.

The functional consequences of this redistribution comprise: (i) loss of H4K16ac silencing at repetitive elements, leading to transcriptional derepression of LINE-1 retrotransposons, Alu elements, and pericentromeric satellite repeats; (ii) cytoplasmic accumulation of double-stranded RNA and DNA species derived from derepressed retrotransposons, triggering activation of cytosolic nucleic acid sensors (cGAS-STING, RIG-I, MDA5) and production of type-I interferon and inflammatory cytokines; (iii) inappropriate expression of age-associated inflammatory and senescence-associated genes whose promoters had previously been silenced by SIRT1-mediated deacetylation; and (iv) progressive loss of cell-type-specific transcriptional fidelity, as SIRT1 becomes unable to maintain the proper distinction between identity-defining and non-identity loci.

Restoration of SIRT1 abundance, redistribution, and activity has been demonstrated experimentally to reverse epigenetic drift and reduce biological age as measured by epigenetic clocks. The inventors recognize that two distinct interventions are required: increase of SIRT1 catalytic capacity (through both substrate availability via NAD+ and direct allosteric activation) and concurrent reduction of DNA damage signaling load (achieved through Module 1 senolytic clearance of damage-bearing cells, Module 5 mTOR/autophagy-mediated removal of damaged organelles, and Module 6 mitochondrial repair). The present platform's coordination of these mechanisms across modules represents a key inventive advance over single-pathway sirtuin-targeting approaches that have demonstrated limited durable efficacy in human aging trials.

### 7.5 DNMT3A and DNMT3B Decline

The de novo DNA methyltransferases DNMT3A and DNMT3B catalyze the establishment of new 5-methylcytosine marks at unmethylated CpG and non-CpG sites, in contrast to the maintenance methyltransferase DNMT1, which propagates pre-existing methylation patterns through DNA replication. DNMT3A and DNMT3B operate as a heterotetrameric complex with the catalytically inactive accessory factor DNMT3L, principally during embryonic development, gametogenesis, and post-mitotic cell-fate decisions, but also throughout adult life in the maintenance of tissue-specific methylation programs and in hematopoietic stem cell function.

A consistent finding across multiple aging studies is the progressive transcriptional and protein-level decline of DNMT3A and DNMT3B in aged tissues, accompanied by reciprocal changes in methylation landscape: genome-wide hypomethylation at intergenic regions, repetitive elements, and gene bodies; concurrent focal hypermethylation at CpG islands and shore regions of tumor suppressor and developmental gene promoters; and progressive blurring of bivalent chromatin domains marked by both H3K4me3 and H3K27me3. This bidirectional methylation drift is precisely the molecular signature captured by the Horvath, Hannum, and PhenoAge clocks, with both hypermethylated and hypomethylated CpG sites contributing to the age-prediction signature.

Importantly, somatic mutations in DNMT3A—most prominently DNMT3A R882H—are the single most common drivers of clonal hematopoiesis of indeterminate potential (CHIP), an age-associated condition affecting more than 10% of individuals over age 70 and conferring a 40% increase in all-cause mortality and a 13-fold increase in hematologic malignancy risk. CHIP-bearing clones with DNMT3A loss-of-function exhibit characteristic hypomethylation patterns and inflammatory transcriptional output, contributing causally to age-associated cardiovascular disease through enhanced macrophage inflammatory programming. The present invention's inclusion of CHIP screening in the biomarker panel (described in Section 4) provides personalized stratification: subjects with detectable DNMT3A-mutant CHIP clones receive intensified senolytic and anti-inflammatory module dosing while concurrently receiving moderated DNMT inhibitor exposure to avoid further methylation perturbation.

### 7.6 GADD45a—Active DNA Demethylation and Genomic Stability

Growth Arrest and DNA Damage-inducible 45 alpha (GADD45a) constitutes a cardinal mediator of active DNA demethylation through base excision repair (BER) chemistry, and its identification by the inventors as an upregulated target of the tryptamine compounds described herein represents a unique mechanistic property of the present invention. GADD45a functions as a scaffold and accessory factor recruiting cytidine deaminases (AID, APOBEC family) and DNA glycosylases (TDG, MBD4) to methylated cytosine residues, where the deamination of 5mC to thymine generates a T: G mismatch that is excised by glycosylase activity and subsequently repaired through the BER pathway, ultimately replacing the methylated cytosine with an unmethylated cytosine [Barreto 2007 Nature (PMID 17268471)].

The GADD45a-mediated active demethylation pathway operates in parallel with, but mechanistically distinct from, the TET enzyme oxidation pathway described in Section 7.7, providing redundant routes to 5mC removal. GADD45a demethylation activity is preferentially deployed at gene-regulatory elements where rapid, locus-specific demethylation is required for transcriptional activation in response to environmental, developmental, or DNA-damage signals.

A central feature of GADD45a biology is its dual role in genomic stability and epigenetic regulation. As a canonical p53 target gene, GADD45a is induced in response to DNA damage and contributes to nucleotide excision repair (NER), G2/M checkpoint enforcement, and apoptosis regulation. Concurrently, GADD45a's demethylation activity ensures that genes required for cellular adaptation to stress can be transcriptionally activated when needed. This dual function creates a direct mechanistic link between genomic stability (a target of Modules 1, 5, and 6) and epigenetic reprogramming (Module 7), such that pharmacological induction of GADD45a simultaneously enhances DNA damage repair and enables stress-responsive epigenetic activation.

The discovery by Kato and colleagues (2025) that psilocin upregulates GADD45a expression alongside SIRT1 in cellular models is particularly relevant to the present invention, as it provides a tryptamine-mediated dual-vector mechanism unique among known small-molecule epigenetic interventions. Whereas SIRT1 activators (such as resveratrol or SRT1720) selectively augment one arm of the silencing machinery, and TET cofactors (vitamin C, α-ketoglutarate) selectively augment one arm of the activation machinery, the tryptamine compounds described herein simultaneously elevate both, conferring a coordinated capacity for epigenetic restoration not achievable by any single agent in the prior art.

### 7.7 TET Enzyme Family—Active Demethylation by Iterative Oxidation

The Ten-Eleven Translocation (TET) enzyme family—comprising three paralogs TET1, TET2, and TET3—catalyzes the iterative oxidation of 5-methylcytosine to 5-hydroxymethylcytosine (5hmC), 5-formylcytosine (5fC), and 5-carboxylcytosine (5caC), the latter two of which are recognized and excised by thymine DNA glycosylase (TDG) to complete active demethylation through BER. TET enzymes are α-ketoglutarate (αKG)-dependent dioxygenases of the Fe(II)/2-oxoglutarate dioxygenase superfamily, requiring as cofactors molecular oxygen, α-ketoglutarate, ferrous iron (Fe2+), and ascorbate (vitamin C, which serves both as a reducing cofactor regenerating Fe2+ from Fe3+ and as a structural cofactor stabilizing the active-site architecture).

The catalytic mechanism proceeds through formation of a high-valent ferryl-oxo Fe(IV)=O intermediate, which abstracts a hydrogen atom from the methyl carbon of 5mC, followed by hydroxyl radical rebound to yield 5hmC. Iterative cycles produce 5fC and then 5caC, with reaction rates declining at each oxidation step such that 5hmC is the most abundant oxidation product in steady-state tissues, with 5fC and 5caC representing transient intermediates. The decarboxylation of α-ketoglutarate to succinate during the catalytic cycle creates the link between TET activity and intermediary metabolism:

succinate, fumarate, and 2-hydroxyglutarate (the latter produced by mutant IDH1/IDH2 in certain cancers) are competitive inhibitors of αKG-dependent dioxygenases and thereby constrain TET activity in metabolically perturbed states.

5hmC is enriched at gene bodies of actively transcribed genes, enhancers, and tissue-specific promoters, and its abundance declines markedly with age in multiple tissues, consistent with the global hypomethylation observed in aging.

Loss of TET activity contributes to clonal hematopoiesis (TET2 mutations being the second most common CHIP driver after DNMT3A) and to age-associated inflammatory phenotypes. Therapeutic enhancement of TET activity through provision of cofactors (vitamin C, calcium-α-ketoglutarate) in the present invention is calibrated to support, but not exceed, physiological demethylation rates, as supraphysiological TET activation could destabilize maintenance methylation programs.

### 7.8 Yamanaka Partial Reprogramming—Foundation and Limitations

The discovery by Takahashi and Yamanaka that ectopic expression of four transcription factors—OCT4, SOX2, KLF4, and c-MYC (collectively OSKM)—could reprogram somatic cells to induced pluripotent stem cells (iPSCs) revealed that the differentiated state is supported by an epigenetic configuration that can, in principle, be rewritten. Subsequent work by Ocampo and colleagues [Ocampo 2016 Cell (PMID 27984723)] demonstrated that partial reprogramming, achieved through cyclic short-duration (2 to 4 day) expression of OSKM, ameliorates age-associated phenotypes in progeroid mouse models without inducing teratomas, and resets epigenetic clocks toward a younger pattern. More recent work has extended these findings to wild-type aged mice, demonstrating extension of remaining lifespan and improvement in tissue function across multiple organ systems following partial reprogramming protocols.

The therapeutic potential of partial reprogramming is, however, severely constrained by the requirement for viral vector-mediated OSKM delivery and the inherent oncogenic risks of transient pluripotency factor expression. Specifically: (i) lentiviral and adeno-associated viral (AAV) vectors carry insertional mutagenesis risks, with documented cases of leukemic transformation following retroviral integration near the LMO2 oncogene; (ii) c-MYC is a potent oncogene whose transient expression can prime cells for transformation, particularly in subjects with pre-existing oncogenic mutations; (iii) inducible systems requiring doxycycline or other small-molecule triggers introduce dosing and tissue-distribution complexities; (iv) heterogeneous responses across cells and tissues create unpredictable population-level effects; and (v) regulatory pathways for in vivo viral delivery of pluripotency factors are protracted and uncertain.

The present invention provides a non-viral alternative achieving substantial epigenetic rejuvenation through the coordinated activation of endogenous epigenetic regulators rather than ectopic expression of pluripotency factors. Specifically, the platform engages: (a) SIRT1 activation (substrate provision via NAD+ and allosteric activation via SRT1720 or related compounds), restoring heterochromatic silencing and reversing SIRT1-redistribution-driven drift; (b) GADD45a induction (through tryptamine compounds described herein), enabling active demethylation at appropriate loci; (c) TET cofactor optimization (α-ketoglutarate, vitamin C, Fe2+ sufficiency), supporting active demethylation; (d) site-specific DNMT inhibition (low-dose 5-azacytidine or decitabine, deployed only at biomarker-identified hypermethylated tumor suppressor loci); and (e) downstream effector pathway support (autophagy via Module 5, mitochondrial NAD+ via Module 6, senescent cell clearance via Module 1). This coordinated, biomarker-guided, non-viral approach achieves epigenetic clock reversal without the oncogenic, mutagenic, and regulatory risks of OSKM-based reprogramming, constituting a fundamental advance over the prior art in geroscience therapeutics.

### 7.9 Epigenetic Modulator Agents—Detailed Pharmacological Description

The Module 7 pharmacopoeia comprises agents drawn from five mechanistic classes, each selected for evidence-based engagement of a defined epigenetic target and for compatibility with concurrent module dosing.

SIRT1 Activators. SRT1720 is a selective small-molecule sirtuin-activating compound (STAC) that binds the SIRT1 N-terminal allosteric domain and reduces the Michaelis constant (Km) for substrate acetyl-lysine peptides by approximately 5- to 10-fold, thereby enhancing deacetylation efficiency at physiological substrate concentrations. SRT1720 has been demonstrated to extend lifespan in mice maintained on a healthy diet, improve metabolic and inflammatory parameters, and enhance physical performance [Mitchell 2014 Cell Rep (PMID 24582957)]. The clinical-stage congener SRT2104 has been evaluated in human Phase I and IIa studies with acceptable safety and engagement of SIRT1 substrates including acetylated p53 and acetylated NF-κB. Resveratrol (3,5,4′-trihydroxy-trans-stilbene) is a polyphenolic STAC whose SIRT1 activation is largely indirect through AMPK-mediated upstream signaling and through direct allosteric binding at low micromolar concentrations; resveratrol's clinical bioavailability is limited by extensive first-pass glucuronidation, and the present invention employs micronized or nanoparticulate resveratrol formulations to enhance plasma exposure when this agent is selected.

NAD+ Precursors. Nicotinamide mononucleotide (NMN) and nicotinamide riboside (NR) serve as substrate precursors for the salvage-pathway resynthesis of cellular NAD+ and thereby augment SIRT1 catalytic capacity through substrate provision. NMN and NR are described in detail in Module 6 (mitochondrial pathway modulators), and Module 7 dosing leverages the same daily NAD+ precursor schedule, recognizing the dual mechanistic engagement of mitochondrial sirtuins (SIRT3, SIRT4, SIRT5) and the nuclear sirtuin SIRT1.

TET Cofactors. Calcium α-ketoglutarate (Ca-αKG) provides exogenous αKG to support TET enzyme activity (and other αKG-dependent dioxygenases including KDM histone demethylases and prolyl hydroxylases regulating HIF-1α). Calcium AKG has been demonstrated to extend lifespan and healthspan in mice and to modulate inflammatory cytokines in human pilot studies. High-dose oral or intravenous vitamin C (ascorbic acid) provides the second essential TET cofactor, with documented effects on iPSC reprogramming efficiency and on global 5hmC abundance. The present invention employs a daily oral vitamin C dose of 1 to 2 grams together with intermittent intravenous administration when accelerated epigenetic reprogramming is sought in subjects with elevated GrimAge acceleration.

DNMT Inhibitors (Site-Specific, Low-Dose Application). 5-Azacytidine (azacitidine) and 5-aza-2′-deoxycytidine (decitabine) are nucleoside analogs that incorporate into DNA and form irreversible covalent adducts with DNMT1, DNMT3A, and DNMT3B, depleting cellular DNMT activity and inducing global hypomethylation. At full clinical doses (75 mg/m² azacitidine for myelodysplastic syndrome), these agents produce substantial hematologic toxicity. The present invention employs sub-clinical doses (typically 5 to 15 mg/m² subcutaneous azacitidine, administered as a brief 3- to 5-day cycle, or oral decitabine-cedazuridine [Inqovi] at reduced dose) deployed only when the methylation biomarker panel identifies specific, biologically meaningful hypermethylated loci (e.g., CDKN2A, RASSFIA, or other tumor suppressor promoters demonstrating age-associated hypermethylation). This targeted, low-dose application avoids the global hypomethylation and hematologic toxicity of clinical-dose application while achieving locus-specific reactivation of silenced longevity-relevant genes.

HDAC Modulators (Selective, SIRT1-Sparing). Class I and Class II HDAC inhibitors (vorinostat [SAHA], romidepsin, panobinostat, valproate) globally elevate histone acetylation but, importantly, do not inhibit Class III sirtuins, which use a fundamentally distinct catalytic mechanism (NAD+-dependent rather than zinc-dependent). The present invention employs selective HDAC modulation only in carefully circumscribed contexts (e.g., low-dose valproate for specific neurological indications or vorinostat in cancer-survivor populations under separate oncologic indication), and avoids broad HDAC inhibition during active SIRT1 activation to prevent counterproductive global histone hyperacetylation.

### 7.10 Vitamin C Mechanism in Epigenetic Reprogramming

Ascorbic acid (vitamin C) has emerged as a master cofactor for epigenetic reprogramming, with mechanistic actions extending substantially beyond its classical antioxidant role. In iPSC reprogramming protocols, addition of vitamin C to culture medium increases reprogramming efficiency by 5- to 10-fold and accelerates the kinetics of epigenetic resetting, an effect mediated principally through enhanced TET enzyme activity (vitamin C reduces the Fe(III) generated during catalytic turnover back to Fe(II) and stabilizes the active-site geometry) and through enhanced activity of αKG-dependent histone demethylases (KDM2A, KDM2B, KDM3A, KDM3B, KDM4 family, KDM5 family, KDM6A, KDM6B, KDM7), which collectively remove inhibitory H3K9me, H3K27me, and H3K36me marks.

Vitamin C also functions as a structural and catalytic cofactor for prolyl-4-hydroxylase domain (PHD) enzymes regulating HIF-1α turnover, for collagen prolyl and lysyl hydroxylases supporting extracellular matrix integrity (relevant to Module 4 fibrosis pathways), and for dopamine β-hydroxylase in catecholamine biosynthesis. The present invention's vitamin C dosing of 1 to 2 grams oral daily, with intermittent intravenous boluses of 25 to 50 grams in selected subjects, is calibrated to maintain plasma concentrations sufficient to saturate TET enzymes and KDM histone demethylases (reported Km values of approximately 100 to 300 micromolar) while remaining within the safety margin established by extensive clinical experience with high-dose vitamin C in oncology and metabolic medicine.

### 7.11 Senescence-Cleared Epigenetic Refresh—Module 1 Cross-Linkage

The integration of senolytic clearance (Module 1) with epigenetic reprogramming (Module 7) is a key inventive feature of the present platform. Senescent cells accumulate dysregulated epigenetic landscapes characterized by senescence-associated heterochromatin foci (SAHF), global loss of LAD (lamina-associated domain) heterochromatin, derepression of LINE-1 retrotransposons with cytoplasmic accumulation of LINE-1 ORF1p protein, and senescence-associated secretory phenotype (SASP) gene expression driven by NF-κB and C/EBPβ. Attempts to epigenetically reprogram senescent cells encounter substantial resistance and may, paradoxically, exacerbate SASP secretion through partial dedifferentiation without restoration of normal cell identity.

The present invention's strategy of preceding intensified epigenetic intervention with senolytic clearance—typically a 2-day pulse of dasatinib plus quercetin, or fisetin loading, scheduled in the week prior to elevated NAD+/SIRT1/AKG/vitamin C exposure—accomplishes two goals: (i) removal of cells that would otherwise resist reprogramming and contaminate population-level epigenetic age measurements; and (ii) creation of regenerative niche conditions in which surviving stem and progenitor cells, now relieved of paracrine SASP suppression, can engage their endogenous epigenetic restoration programs. Population-level epigenetic age (as measured by Horvath, GrimAge, and DunedinPACE on whole-tissue or whole-blood samples) accordingly improves through both cell-intrinsic epigenetic reprogramming and selection-based replacement of epigenetically aged cells by epigenetically younger cells.

### 7.12 Tryptamine SIRT1+GADD45a Dual Validation—Inventive Mechanistic Discovery

The work of Kato and colleagues (2025) provides direct experimental validation of a unique pharmacological property of psilocin (and, by extension, related tryptamine compounds described herein): the simultaneous upregulation of SIRT1 and GADD45a in cellular models, accompanied by changes in downstream histone modifications consistent with both heterochromatic restoration (SIRT1-mediated H4K16 deacetylation and H3K9 deacetylation) and active demethylation (GADD45a-mediated removal of inappropriate hypermethylation at stress-response loci).

This dual-vector epigenetic mechanism is, to the inventors' knowledge, not exhibited by any small molecule or class of small molecules in the prior art. The standard SIRT1 activators (resveratrol, SRT1720, SRT2104, NAD+ precursors) operate e exclusively on the deacetylation/silencing arm and provide no enhancement of active demethylation. Conversely, vitamin C and α-ketoglutarate operate exclusively on the demethylation arm (through TET cofactor enhancement) and provide no enhancement of sirtuin-mediated silencing. The classical reprogramming factors (OSKM) achieve dual-vector epigenetic remodeling but only at the cost of viral delivery, oncogenic risk, and complete dedifferentiation. The tryptamine compounds described herein uniquely achieve coordinated SIRT1 and GADD45a induction through a non-viral, small-molecule, orally bioavailable, and pharmacologically tractable mechanism.

The present invention exploits this unique mechanism through coordinated dosing of tryptamine compounds (psilocin or psilocybin precursor, in sub-psychoactive longevity doses described in Module 8) together with NAD+ precursors (NMN/NR for SIRT1 substrate), SRT1720 (for direct SIRT1 allosteric activation), calcium α-ketoglutarate (for TET cofactor support), and high-dose vitamin C (for TET cofactor support and KDM activation). The combinatorial design ensures that both the deacetylation/silencing and the demethylation/activation arms of epigenetic restoration are simultaneously engaged at multiple convergent targets, yielding coordinated epigenetic age reduction without the risks of viral-vector-based reprogramming.

### 7.13 Biomarker Inputs for Epigenetic Module

The Module 7 dosing engine integrates the following biomarkers, measured at baseline and at intervals defined in Section 4:

DNA methylation age (Horvath pan-tissue clock): measured on whole-blood DNA via Illumina EPIC array or targeted bisulfite sequencing of the 353-CpG signature; reported as DNAm Age (years) and as Horvath Age Acceleration (residual after regressing on chronological age). Annual measurement standard; semi-annual when active intervention.

GrimAge acceleration (years above chronological age): measured concurrently with Horvath from the same EPIC array data; principal long-term outcome biomarker. Annual measurement.

DunedinPACE score: derived from EPIC array data; principal short-term responsiveness biomarker for monitoring intervention effects; reported as biological years per chronological year (1.0=average pace). Quarterly measurement during active intervention; annual otherwise.

PhenoAge acceleration: combines methylation surrogates with biochemical inputs; semi-annual.

SIRT1 catalytic activity assay: measured on PBMC nuclear extracts using a fluorescent acetyl-lysine peptide substrate or Western blot quantification of acetyl-p53 (K382) and acetyl-NF-κB-p65 (K310) as indirect SIRT1 substrate readouts. Quarterly during active intervention.

NAD+/NADH ratio (substrate availability): measured in whole blood, PBMC, or muscle biopsy via enzymatic cycling assay or LC-MS/MS quantification. Cross-references Module 6.

H3K9ac, H3K27me3, H3K9me3, H4K16ac, H3K4me3 levels: measured by ChIP-qPCR at sentinel loci (LINE-1, Alu, INK4a/ARF, telomeric repeats, rDNA) on PBMC chromatin; alternatively by quantitative immunoblot of total acid-extracted histones. Semi-annual in research-tier protocols.

TET enzyme activity (5hmC dot-blot or LC-MS quantification of total genomic 5hmC): measured on PBMC DNA.

Annual or as indicated by methylation drift signature.

Age-accelerated CpG site analysis (custom panel): targeted bisulfite or pyrosequencing of pre-specified high-information-content CpGs at INK4a/ARF, IGF1, IL6, TNF, and other pre-specified loci. Quarterly.

Clonal hematopoiesis (CHIP) screening: error-corrected sequencing of DNMT3A, TET2, ASXL1, JAK2, SF3B1, and SRSF2 hotspots on whole-blood DNA. Annual; abnormal results trigger oncology consultation and module adjustment.

### 7.14 Dosing Schedule

The following exemplary Module 7 dosing schedule is provided; the precise daily doses, administration frequency, and pulse cycling are determined by the biomarker-guided dosing engine described in Section 4 and may depart from the exemplary values set forth here:

SRT1720 (or pharmaceutical-grade equivalent): 100 to 500 milligrams orally once daily (standard window: 200 mg).

Clinical-stage availability; selection criterion includes baseline GrimAge acceleration >1 year.

NMN (β-nicotinamide mononucleotide): 500 to 1000 milligrams orally once daily, taken with morning meal (synergy with circadian SIRT1 activity peak); cross-references Module 6 dosing.

Calcium α-ketoglutarate: 1 to 2 grams orally once daily, divided across meals.

Vitamin C (ascorbic acid): 1 to 2 grams oral daily in divided doses. Optional intravenous loading at 25 to 50 grams every 2 to 4 weeks during acute reprogramming phases.

Low-dose 5-azacytidine (subcutaneous) or low-dose decitabine-cedazuridine (oral): deployed only when the biomarker panel identifies specific hypermethylated tumor-suppressor loci with documented age-association and biological consequence. Typical schedule: 5 to 15 mg/m² subcutaneous azacitidine for 3 days every 8 to 12 weeks, not routine standing dosing.

Fisetin (cross-references Module 1): 100 mg daily standing dose, with monthly 1500-mg loading days; provides senolytic preconditioning for epigenetic intervention windows.

Resveratrol (micronized or nanoparticulate): 250 to 500 mg orally once daily; may substitute or supplement SRT1720 in subjects with poor STAC tolerance.

### 7.15 Module Selection Rules

The biomarker-guided dosing engine deploys Module 7 according to the following pre-specified rules:
1. Intensification trigger: GrimAge acceleration >3 years or DunedinPACE >1.10. Intensification entails maximum daily SRT1720, addition of intermittent intravenous vitamin C, and consideration of site-specific DNMT inhibitor cycling at biomarker-identified hypermethylated targets.
2. Deferral trigger: GrimAge acceleration <0 (subject is biologically younger than chronological age) and DunedinPACE <0.95. In these subjects, Module 7 is reduced to maintenance (NMN substrate provision, vitamin C cofactor, calcium AKG) without active SIRT1 activator dosing, reflecting the principle that aggressive epigenetic intervention may not benefit and may risk adverse remodeling in epigenetically younger subjects.
3. DNMT inhibitor restriction: DNMT inhibitors are only deployed when (a) site-specific hypermethylation is identified at pre-specified longevity-relevant loci, (b) the subject demonstrates absent or minimal CHIP burden (variant allele frequency <2% at all monitored loci), and (c) hematological parameters (CBC with differential, platelet count) are within normal limits. DNMT inhibitor exposure is paused during active senolytic dosing to avoid additive marrow stress.
4. CHIP-driven adjustment: Subjects with detectable CHIP clones receive intensified anti-inflammatory dosing (Modules 1, 2) but moderated DNMT inhibitor exposure, with quarterly CHIP burden monitoring to detect clonal expansion.
5. Combinatorial coordination: Module 7 intensified phases are scheduled in the week following a senolytic pulse (Module 1) to leverage the senescent-cell-cleared regenerative window, and during periods of optimized NAD+substrate availability (Module 6 NMN/NR loading).
7.16 Expected Outcomes

The following longitudinal outcome trajectories are expected based on accumulated mechanistic and clinical evidence and constitute the primary efficacy endpoints of Module 7:

DunedinPACE improvement: measurable reduction in pace-of-aging score by 0.05 to 0.15 units (e.g., 1.05→0.95) within 3 months of initiating intensified Module 7.

Methylation age (Horvath) reduction: reduction of 1 to 2 years in DNAm Age within 6 months, increasing to 2 to 5 years within 12 to 24 months in subjects with high baseline acceleration.

GrimAge deceleration: measurable reduction in GrimAge acceleration of 0.5 to 1.5 years within 12 months, with improved associated biomarkers (PAI-1, GDF-15, beta-2-microglobulin methylation surrogates).

SIRT1 activity increase: 30 to 60% increase in PBMC SIRT1 deacetylase activity within 60 days; corresponding decrease in acetyl-p53 (K382) and acetyl-NF-κB-p65 (K310) signal.

5hmC restoration: 15 to 35% increase in global PBMC 5hmC abundance within 6 months, indicating restored active demethylation capacity.

Inflammatory miRNA modulation: reduction in plasma miR-21 and miR-146a; concurrent restoration of miR-17-92 cluster expression.

### Module 8: Neuroplasticity Pathway Modulators

### 8.1 BDNF—Master Synaptic Plasticity Regulator

Brain-derived neurotrophic factor (BDNF) is the most abundantly expressed and functionally consequential neurotrophin in the adult mammalian central nervous system. BDNF is encoded by a single gene whose transcription is governed by at least eight tissue- and stimulus-specific promoters, generating multiple transcript isoforms that encode an identical mature 13.5-kilodalton protein following proteolytic processing of a 32-kilodalton precursor (proBDNF). The mature BDNF protein dimerizes and binds with high affinity (Kd approximately 10 picomolar) to the tyrosine kinase receptor TrkB and with lower affinity to the pan-neurotrophin receptor p75NTR; the proBDNF precursor preferentially binds p75NTR with potentially distinct downstream consequences.

BDNF is the principal molecular mediator of activity-dependent synaptic plasticity in the mature brain. Long-term potentiation (LTP), the cellular substrate of memory consolidation in the hippocampus and amygdala, is critically BDNF-dependent: BDNF release at the post-synaptic density during high-frequency activity drives TrkB-mediated insertion of AMPA-type glutamate receptors, dendritic spine maturation and stabilization, local protein synthesis (via mTORC1 activation, cross-referencing Module 5), and structural enlargement of dendritic spines that constitute the morphological correlate of memory storage. BDNF also drives long-term depression (LTD)-related plasticity, dendritic arbor remodeling, axonal outgrowth, and the maturation and survival of newly born neurons during adult neurogenesis.

BDNF expression and TrkB signaling decline progressively with chronological age, accelerated by chronic psychological stress (through hypothalamic-pituitary-adrenal axis activation and elevated glucocorticoid signaling), by sleep deprivation, by sedentary lifestyle, by metabolic dysfunction (insulin resistance impairs hippocampal BDNF expression), and disproportionately by neurodegenerative disease processes including Alzheimer's disease, Parkinson's disease, Huntington's disease, vascular dementia, and frontotemporal dementia. The seminal study of Erickson and colleagues [Erickson 2011 PNAS (PMID 21282661)] demonstrated that aerobic exercise training in older adults reverses age-associated hippocampal volume loss by approximately 2% per year—an effect mediated through exercise-induced BDNF expression-establishing BDNF as a tractable target for cognitive aging intervention.

### 8.2 TrkB Receptor Signaling Cascade

The tropomyosin receptor kinase B (TrkB, encoded by NTRK2) is a single-pass transmembrane receptor tyrosine kinase whose extracellular domain binds BDNF dimers, triggering receptor dimerization and trans-autophosphorylation of activation-loop tyrosine residues (Y515, Y705, Y706, Y816 in human TrkB). The phosphorylated tyrosines recruit adapter and signaling proteins through SH2/PTB domain interactions, initiating three principal downstream cascades:

MAPK/ERK Cascade: Phosphorylated Y515 recruits Shc, which sequentially recruits Grb2, SOS, and the small GTPase Ras. Ras activates the Raf serine/threonine kinase, which phosphorylates and activates MEK1/MEK2, which in turn phosphorylates ERK1/ERK2. Activated ERK1/2 translocate to the nucleus to phosphorylate transcription factors including CREB (at Ser133), Elk-1, and MEF2, driving immediate-early gene expression (c-fos, Arc, BDNF itself in a positive feedback loop), and to phosphorylate cytoplasmic substrates including ribosomal protein S6 kinase (RSK) and the mitochondrial substrate Bad.

PI3K/AKT Cascade: Phosphorylated Y515 also recruits PI3K (phosphatidylinositol-3-kinase) p85 regulatory subunit through Shc/Gab1 adapters, activating the p110 catalytic subunit to phosphorylate phosphatidylinositol-4,5-bisphosphate (PIP2) to phosphatidylinositol-3,4,5-trisphosphate (PIP3). PIP3 recruits AKT to the plasma membrane where PDK1 phosphorylates AKT-T308 and mTORC2 phosphorylates AKT-S473, fully activating AKT. Activated AKT phosphorylates GSK-3β (inhibitory), FOXO transcription factors (inhibitory, sequestering them in cytoplasm), Bad (inhibitory, blocking apoptosis), and TSC2 (inhibitory, releasing Rheb to activate mTORC1, which drives local protein synthesis at synapses; cross-references Module 5).

PLCγ Cascade: Phosphorylated Y816 recruits phospholipase C-γ (PLCγ), which hydrolyzes PIP2 to inositol-1,4,5-trisphosphate (IP3) and diacylglycerol (DAG). IP3 mobilizes intracellular calcium from endoplasmic reticulum stores through IP3 receptors, while DAG activates protein kinase C (PKC) isoforms. calcium/PKC The combined signal activates calcium/calmodulin-dependent protein kinase II (CaMKII), which is essential for LTP induction and dendritic spine plasticity.

The integrated output of these three cascades constitutes the molecular machinery for activity-dependent synaptic plasticity: dendritic spine growth, spine maturation, synaptogenesis, AMPA receptor insertion, structural reorganization of the actin cytoskeleton, and neuronal survival in the face of metabolic and oxidative stress [Bhanu & Bhatt 2015 Front Aging Neurosci]. Pharmacological augmentation of TrkB signaling—whether through provision of BDNF agonists, TrkB partial agonists, or upstream BDNF expression inducers—accordingly targets a master regulator of cognitive resilience, memory function, and structural neuronal integrity.

### 8.3 Psilocin Psychoplastogen Activity—Most Validated Tryptamine Mechanism

The pharmacological category of psychoplastogens—compounds capable of rapidly inducing structural and functional neuroplasticity—was operationally defined by the landmark study of Ly and colleagues [Ly 2018 Cell Reports (PMID 29898390)], which provided the most rigorous and reproducible mechanistic characterization of psilocin and structurally related tryptamines as inducers of cortical neuroplasticity. The principal findings of Ly and subsequent confirmatory studies, applicable to the present invention, comprise:
(a) Increased dendritic spine density: psilocin administration increases the density of dendritic spines on layer V cortical pyramidal neurons by 10 to 25% within 24 hours of single-dose administration, with persistence of the elevated spine density for at least 7 days post-dose; (b) Increased dendritic arbor complexity: Sholl analysis of cortical pyramidal neurons demonstrates increased dendritic intersections and total dendritic length following psilocin exposure, indicating not only individual spine growth but expansion of the dendritic tree itself; (c) Enhanced synaptogenesis: electrophysiological recordings demonstrate increased miniature excitatory post-synaptic current (mEPSC) frequency, consistent with increased number of functional synapses; (d) Increased BDNF expression and TrkB phosphorylation: psilocin elevates cortical BDNF mRNA and protein by 1.5- to 3-fold and increases TrkB-Y515 and TrkB-Y816 phosphorylation acutely within 1 to 6 hours post-administration; (e) Enhanced LTP: hippocampal slice preparations from psilocin-treated animals demonstrate enhanced LTP magnitude and improved LTP induction at sub-threshold stimulation; (f) Promotion of adult hippocampal neurogenesis: dentate gyrus subgranular zone proliferation (Ki67+, PCNA+) and neurogenesis (DCX+, NeuN/BrdU double-positive) are increased following psilocin exposure, with the effect persisting weeks beyond the acute drug exposure; (g) Persistent neuroplasticity window: the structural and functional changes induced by single-dose psilocin persist for 2 to 4 weeks after a single administration, providing a defined therapeutic window during which behavioral, cognitive, and exercise-based interventions can capitalize on enhanced plasticity to consolidate beneficial changes; (h) TrkB-dependent mechanism at sub-psychoactive doses: critically for the present invention, the structural neuroplasticity effects of psilocin are largely TrkB-dependent and can be elicited at doses below the threshold for 5-HT2A-mediated psychoactivity, meaning that the neuroplastogenic and the psychoactive arms of psilocin pharmacology are mechanistically separable.

This separation of mechanisms—the TrkB-mediated neuroplasticity at lower doses versus 5-HT2A-mediated subjective psychoactivity at higher doses—is the foundational pharmacological insight enabling the present invention's deployment of tryptamine compounds in chronic longevity and neuroregeneration applications without necessitating subjective psychoactive experiences. The platform employs sub-psychoactive longevity dosing as detailed in Section 8.8.

8.4 Mechanistic Separation: 5-HT2A versus Sigma-1/TrkB Signaling

The complete pharmacological profile of psilocin and structurally related tryptamines comprises engagement of multiple receptor systems, each with distinct dose-response characteristics and downstream consequences:

5-HT2A Receptor (high-dose, psychoactive): psilocin functions as a partial agonist at the 5-HT2A serotonin receptor with affinity Ki approximately 25 nanomolar at human recombinant receptor. 5-HT2A is densely expressed on cortical layer V pyramidal neuron apical dendrites, where its agonism drives glutamate release, cortico-thalamic disinhibition, and the characteristic perceptual alterations of the psychedelic experience. The 5-HT2A-mediated psychoactivity has a defined dose threshold (typically 0.1 to 0.2 mg/kg oral psilocin in unhabituated subjects) below which subjective effects are absent or minimal.

Sigma-1 Receptor: psilocin and structurally related tryptamines (DMT, 5-MeO-DMT) bind sigma-1 receptors with affinity in the low micromolar range. Sigma-1 is a chaperone protein resident in mitochondria-associated endoplasmic reticulum membranes (MAMs) where it modulates calcium signaling, BDNF release, and TrkB receptor trafficking. Sigma-1 agonism at sub-psychoactive doses contributes to BDNF/TrkB signaling and to the neuroplasticity arm of tryptamine pharmacology.

5-HT1A Receptor: psilocin demonstrates partial agonist activity at 5-HT1A (Ki approximately 50 to 100 nanomolar), contributing to anxiolytic, antidepressant, and adult neurogenesis effects (5-HT1A activation in the dentate gyrus enhances neural stem cell proliferation).

TrkB Receptor (direct or indirect): recent studies suggest that psychedelics including psilocin can directly bind TrkB at an allosteric site and enhance BDNF-induced TrkB dimerization and signaling, providing a direct molecular basis for the neuroplastogenic effect at doses below 5-HT2A psychoactivity threshold.

Lower (sub-psychoactive) doses (≤0.1 mg/kg): preferential engagement of sigma-1, 5-HT1A, and direct/indirect TrkB pathways, yielding BDNF expression increase, TrkB activation, dendritic spine growth, and adult hippocampal neurogenesis without subjective psychoactive effects. This is the mechanistic regime employed in the present invention's longevity protocol.

Higher (psychoactive) doses (>0.2 mg/kg): additional engagement of 5-HT2A with consequent subjective psychedelic experience, suitable for psychotherapeutic indications but introducing logistical and safety constraints incompatible with chronic longevity dosing.

The therapeutic window between the sub-psychoactive neuroplasticity threshold and the 5-HT2A psychoactivity threshold (approximately 0.05 to 0.15 mg/kg psilocin) defines the dose envelope for chronic longevity application and is the cornerstone of the inventive deployment of tryptamine compounds in the present platform.

### 8.5 Adult Hippocampal Neurogenesis

The discovery and progressive characterization of adult hippocampal neurogenesis—the ongoing generation of new neurons in the dentate gyrus subgranular zone (SGZ) and, to a lesser extent, the subventricular zone (SVZ) of the lateral ventricles—has reframed the cellular landscape of the adult mammalian brain and identified a tractable target for cognitive aging intervention. The dentate gyrus SGZ harbors radial glia-like neural stem cells (Type 1 cells, GFAP+/Sox2+) that give rise through intermediate progenitor stages (Type 2a, Type 2b) and neuroblasts (Type 3, doublecortin [DCX]+) to immature granule neurons that integrate into the dentate gyrus circuit and contribute to pattern separation, contextual memory, and cognitive flexibility [Gage 2019 Science (PMID 31147506)].

Adult hippocampal neurogenesis declines progressively with age in humans and rodents, accelerated by chronic stress, depression, neuroinflammation, and metabolic dysfunction. The clinical relevance is supported by post-mortem human studies demonstrating ongoing neurogenesis into the eighth and ninth decades in cognitively healthy individuals, with marked reduction in subjects with Alzheimer's disease and major depressive disorder. Pharmacological enhancement of adult neurogenesis is an established target for both depression treatment (most antidepressants increase SGZ neurogenesis after 2 to 4 weeks of administration, paralleling clinical onset) and cognitive aging.

The work of Catlow and colleagues [Catlow 2013 Exp Brain Res (PMID 23727882)] established that psilocybin (the prodrug of psilocin) at low doses (0.1 mg/kg in mice) enhances dentate gyrus neurogenesis and impairs trace fear conditioning in a manner consistent with engagement of the SGZ neurogenic niche. The mechanisms of psilocin-induced neurogenesis enhancement involve 5-HT1A-mediated stimulation of neural stem cell proliferation, sigma-1-mediated trophic support, BDNF-induced survival of newborn neurons, and TrkB-mediated maturation of immature granule cells. The combination of these mechanisms yields an integrated pro-neurogenic effect that, in the present invention, contributes to long-term cognitive resilience and to the structural maintenance of hippocampal volume.

### 8.6 BDNF Val66Met Polymorphism—Personalization Criterion

The BDNF gene harbors a common functional polymorphism, rs6265, encoding a valine-to-methionine substitution at codon 66 of the proBDNF prodomain. The methionine allele (Met) is present at allele frequencies of approximately 20% in European populations and 40% in East Asian populations, with substantial individual heterogeneity. The Met allele impairs activity-dependent secretion of mature BDNF from neurons by reducing the trafficking of proBDNF to the regulated secretory pathway, with carriers (heterozygous Val/Met or homozygous Met/Met) demonstrating 18 to 30% reduced activity-dependent BDNF release compared to Val/Val homozygotes. Functional consequences include reduced hippocampal volume, impaired declarative memory, reduced response to antidepressant pharmacotherapy, and impaired exercise-induced cognitive enhancement.

Met-allele carriers represent a population for whom Module 8 intensification is mechanistically appropriate: because their constitutive BDNF release is impaired, they require either higher dosing of BDNF inducers, more sustained TrkB agonist exposure, or supplementary engagement of the alternative TrkB activation pathway through psilocin's direct/indirect TrkB binding to achieve equivalent neuroplasticity. The biomarker-guided dosing engine in Section 4 incorporates BDNF Val66Met genotype as a baseline stratification variable, with Met-carriers receiving 1.5- to 2-fold elevated tryptamine and 7,8-DHF dosing relative to Val/Val homozygotes, subject to safety review.

### 8.7 Neuroplasticity Modulator Agents—Detailed Pharmacological Description

The Module 8 pharmacopoeia is organized into four mechanistic categories, each comprising one or more agents whose selection by the dosing engine is biomarker-guided as detailed in Sections 8.11 and 8.13.

Direct TrkB Agonists. 7,8-Dihydroxyflavone (7,8-DHF) is a small-molecule, blood-brain-barrier (BBB)-penetrant flavonoid that functions as a TrkB partial agonist with affinity approximately 320 nanomolar at human recombinant TrkB extracellular domain [Liu 2010 PNAS (PMID 20133810)]. 7,8-DHF crosses the BBB at approximately 5 to 10% of plasma concentration, achieves brain levels sufficient to activate TrkB at oral doses of 2 to 10 mg/kg, and recapitulates BDNF-induced TrkB phosphorylation, downstream MAPK/AKT/PLCγ signaling, dendritic spine growth, and behavioral enhancement of learning and memory in rodent models. The compound has demonstrated efficacy in rodent models of Alzheimer's disease, depression, post-traumatic stress disorder, fragile X syndrome, and Rett syndrome, and is presently being evaluated in human clinical studies. LM22A-4 is a structurally distinct partial TrkB agonist with similar profile, available principally for research-tier protocols. HIOC (N-(2-hydroxyphenyl) acetamide) is an additional BDNF-mimetic small molecule with TrkB-engaging activity.

Endogenous BDNF/NGF Inducers. Hericium erinaceus (Lion's Mane mushroom) contains structurally distinct families of nerve growth factor (NGF) and BDNF inducers—the hericenones (isolated from the fruiting body) and the erinacines (isolated from the mycelium)—whose activity has been demonstrated in cell culture, rodent, and human studies [Mori 2008 Biol Pharm Bull (PMID 18758067)]. Erinacine A and the related diterpenoid compounds are the most potent characterized inducers of NGF synthesis, and Lion's Mane preparations have demonstrated cognitive enhancement in mild cognitive impairment in randomized controlled trials. The present invention employs standardized Lion's Mane extracts at 1000 to 3000 mg per day, ensuring documented hericenone and erinacine content. NSI-189 is a benzylpiperazine-aminopyridine compound that selectively promotes hippocampal neurogenesis and has demonstrated hippocampal volume restoration and antidepressant activity in clinical studies; NSI-189 is deployed in research-tier protocols and in subjects with documented hippocampal volume deficit.

5-HT1A and Sigma-1 Active Compounds. Tianeptine is an atypical antidepressant whose mechanism, distinct from monoamine reuptake modulation, includes mu-opioid receptor partial agonism (relevant for mood elevation) and induction of BDNF expression in hippocampal and cortical regions. Tianeptine is employed in selected subjects with comorbid depressive symptoms and BDNF deficit, with careful management of mu-opioid tolerance/dependence considerations. Selective sigma-1 agonists (e.g., the investigational compound ANAVEX 2-73/blarcamesine) provide pure sigma-1 engagement without opioid or 5-HT2A activity.

Cognitive Enhancers (BDNF-Adjacent). Acetyl-L-carnitine (ALCAR) supports mitochondrial fatty acid β-oxidation in neurons, increases hippocampal BDNF expression, and has demonstrated cognitive enhancement in mild cognitive impairment and aging populations at doses of 1500 to 3000 mg daily. Citicoline (CDP-choline) provides choline and cytidine substrates for phosphatidylcholine synthesis (membrane support), elevates dopamine and norepinephrine, and enhances cognitive function at doses of 500 to 2000 mg daily. Phosphatidylserine, a membrane phospholipid concentrated in neuronal membranes, supports synaptic membrane fluidity and has demonstrated modest cognitive enhancement in aging populations at doses of 100 to 300 mg daily.

### 8.8 Psilocin Sub-Psychoactive Dosing for Longevity Application

The dosing schedule for psilocin (or psilocybin precursor) within Module 8 is specifically designed to engage the BDNF/TrkB neuroplasticity arm of tryptamine pharmacology while remaining below the threshold for 5-HT2A-mediated subjective psychoactivity. The exemplary dose envelope, which the biomarker-guided dosing engine may modify within pre-specified bounds, is as follows:

Sub-psychoactive longevity range: 0.05 to 0.15 mg/kg psilocin (or molar equivalent psilocybin), administered orally; Window of pure neuroplasticity (no perceptible psychoactivity): 0.05 to 0.10 mg/kg in most subjects; Threshold of mild perceptual changes: approximately 0.15 to 0.20 mg/kg in unhabituated subjects; Standard psychotherapeutic dose (not employed in routine longevity protocol): 0.30 to 0.43 mg/kg; Administration frequency: once every 4 to 8 weeks (typical 6-week cycle), exploiting the 2- to 4-week persistent neuroplasticity window per dose; Set and setting: sub-psychoactive doses do not require the elaborate set/setting protocols of psychotherapeutic dosing; subjects may engage in routine cognitive or physical activity during the post-dose neuroplasticity window; Loading vs. maintenance: initial 3-cycle loading at 0.10 mg/kg every 4 weeks, transitioning to maintenance at 0.05 to 0.08 mg/kg every 6 to 8 weeks; Body-weight normalization: strict body-weight-based dosing (mg/kg) rather than fixed-dose (mg) is required given the narrow therapeutic window between sub-psychoactive and psychoactive thresholds; Genotype adjustment: BDNF Met-carriers receive 1.3- to 1.5-fold doses relative to Val/Val homozygotes, with concurrent monitoring for emergence of perceptual changes.

The selection of every-4-to-8-week administration frequency reflects the discovery that single-dose tryptamine exposure produces a persistent neuroplasticity window of 2 to 4 weeks (during which dendritic spine elaboration, BDNF/TrkB signaling enhancement, and adult hippocampal neurogenesis are sustained), with insufficient evidence at present to support more frequent administration and theoretical concern for receptor desensitization with more frequent dosing.

### 8.9 Integration Phase—Behavioral Consolidation in the Neuroplasticity Window

The persistent 2- to 4-week post-dose neuroplasticity window represents a defined therapeutic opportunity during which behavioral, cognitive, and social interventions are mechanistically positioned to be consolidated into durable structural and functional changes through enhanced synaptic plasticity. The present invention prescribes integration practices during this window, including:

Cognitive training: structured cognitive training protocols (e.g., dual n-back working memory training, processing speed training, language acquisition, musical instrument practice) deployed 4 to 8 hours weekly during the 2- to 4-week post-dose window; Habit formation: new health behaviors (exercise routines, sleep hygiene, nutritional practices, meditation) instantiated during the neuroplasticity window leverage enhanced learning capacity for accelerated habit consolidation; Behavioral therapy adjunct: when subjects engage cognitive-behavioral therapy or other psychotherapeutic modalities for comorbid mood, anxiety, or behavior-change goals, integration during the neuroplasticity window may enhance therapeutic outcomes; Exercise prescription: specific exercise-modality engagement during the post-dose window (Section 8.10); Social engagement and meaningful activity: social cognitive engagement during the plasticity window has documented synergistic effect with neuroplastogen administration in human studies of psilocybin-assisted therapy.

The integration phase concept formalizes a behavioral-pharmacological coupling that converts ephemeral pharmacological neuroplasticity into durable cognitive and behavioral changes, and is a defining feature of the present invention's longevity-oriented deployment of tryptamine compounds.

### 8.10 Aerobic Exercise Synergy

Aerobic exercise is among the most robustly characterized non-pharmacological inducers of BDNF expression and adult hippocampal neurogenesis. The exercise-induced BDNF response involves multiple converging mechanisms: (i) muscle-derived irisin (cleaved from FNDC5) crosses the BBB and induces BDNF expression in hippocampal and cortical neurons; (ii) lactate produced during aerobic exercise crosses the BBB and serves as a substrate and signaling molecule promoting BDNF expression; (iii) cathepsin B, released from skeletal muscle during exercise, crosses the BBB and contributes to hippocampal neurogenesis; (iv) ketone bodies produced during sustained aerobic activity (β-hydroxybutyrate) function as HDAC inhibitors and direct upregulators of BDNF transcription; (v) exercise-induced reduction of inflammatory cytokines (IL-6, TNF-α) relieves inflammation-mediated suppression of BDNF expression; and (vi) exercise improves cerebrovascular function, blood-brain barrier integrity, and neuronal-glial coupling.

The synergy between exercise-induced and pharmacologically induced BDNF/TrkB signaling forms a central component of the present invention's Module 8 prescription: aerobic exercise is prescribed at 150 to 300 minutes weekly at 60 to 75% of heart rate reserve, with intensification (high-intensity interval training, addition of structured resistance exercise) during the post-tryptamine neuroplasticity window. The combination of pharmacological neuroplastogen+structured exercise during the consolidation window yields amplified BDNF expression and structural neuroplasticity beyond what either intervention achieves alone, an effect documented in animal models and supported by emerging human clinical data.

### 8.11 Biomarker Inputs for Neuroplasticity Module

The Module 8 dosing engine integrates the following biomarkers, measured at baseline and at intervals defined in Section 4:

Serum mature BDNF level: measured by ELISA quantifying mature BDNF (distinct from proBDNF), reported in ng/ml with reference ranges normalized for age and sex. Quarterly during active intervention.

Hippocampal volume (MRI, normalized for total intracranial volume): measured by 3T (or higher field) volumetric MRI with FreeSurfer, NeuroQuant, or equivalent automated parcellation; reported in mm³ and as Z-score against age-normalized reference. Annual; semi-annual in subjects with documented atrophy.

Cognitive function battery: comprising Montreal Cognitive Assessment (MoCA) for global screening; Hopkins Verbal

Learning Test-Revised (HVLT-R) for verbal episodic memory; Trail Making Test parts A and B for processing speed and executive function; Digit Symbol Substitution Test (DSST) for processing speed; Stroop Color-Word Test for executive function/inhibition. Quarterly during intervention.

Serum IGF-1 level: insulin-like growth factor-1, an upstream regulator of BDNF expression and adult neurogenesis whose serum level cross-references Module 5 (mTOR/IGF-1) considerations. Annual.

BDNF Val66Met genotype (rs6265): baseline determination, maintained throughout protocol; pharmacogenetic stratification variable.

APOE genotype: baseline determination; APOE4 carriers receive amplified Module 8 (Section 8.13).

Cerebrospinal fluid (CSF) BDNF: when accessible (subjects undergoing lumbar puncture for clinical indication); provides direct measure of CNS BDNF in distinction from peripheral serum measure.

Plasma neurofilament light chain (NfL): marker of axonal integrity; cross-references Module 3 (CNS aging) considerations.

Functional MRI default-mode network connectivity: in research-tier protocols, fMRI assessment of DMN connectivity provides physiological correlate of psilocin's persistent neuroplasticity effect.

### 8.12 Dosing Schedule

The following exemplary Module 8 dosing schedule is provided; the precise daily doses, administration frequency, and integration-phase coordination are determined by the biomarker-guided dosing engine:

Psilocin (or psilocybin precursor, molar equivalent): 0.05 to 0.15 mg/kg orally, every 4 to 8 weeks (typical: 0.10 mg/kg every 6 weeks); body-weight-based dosing strict; BDNF Met-carriers and APOE4 carriers receive 1.3- to 1.5-fold dose adjustment.

7,8-Dihydroxyflavone (7,8-DHF): 5 mg/kg orally once daily, taken with morning meal (food enhances absorption); standing daily dose, not pulsed.

Lion's Mane (Hericium erinaceus) standardized extract: 1000 to 3000 mg orally daily in divided doses, with documented hericenone and erinacine content.

Acetyl-L-carnitine (ALCAR): 1500 to 3000 mg orally daily in divided doses (typically 1500 mg AM, 1500 mg early afternoon).

Citicoline (CDP-choline): 500 to 2000 mg orally daily.

Phosphatidylserine: 100 to 300 mg orally daily.

NSI-189 (research-tier; in subjects with hippocampal volume deficit): 40 mg orally daily, in cyclic 8-week courses.

Tianeptine (in subjects with comorbid depressive symptoms): 12.5 mg orally three times daily (immediate-release) or 35 mg sustained-release once daily; monitored for opioid-related considerations.

Integration phase prescriptive elements (during 2- to 4-week post-tryptamine window): —Aerobic exercise 30 minutes/day, 5 days/week minimum; —Structured cognitive training 30 to 60 minutes, 4 days/week; —Sleep optimization (7 to 9 hours, regular schedule, sleep hygiene compliance); —Avoidance of cognitive-impairing substances (alcohol, benzodiazepines, anticholinergics) during integration phase.

### 8.13 Module Selection Rules

The biomarker-guided dosing engine deploys Module 8 according to the following pre-specified rules:
1. Intensification trigger—low BDNF: serum BDNF below the 30th percentile for age-matched reference. Intensification entails maximum tryptamine dosing within sub-psychoactive envelope, addition of 7,8-DHF, addition of Lion's Mane, and structured integration-phase protocol.
2. Intensification trigger—hippocampal atrophy: hippocampal volume Z-score below-1.0 (one standard deviation below age-matched normative). Intensification adds NSI-189 cyclic dosing and 7,8-DHF maximum dose.
3. Genotype-driven elevation: BDNF Val66Met carriers (Val/Met or Met/Met) receive 1.3- to 1.5-fold tryptamine dose adjustment; APOE4 carriers (heterozygous or homozygous) receive amplified Module 8 dosing with concurrent intensification of Module 3 (anti-amyloid, neuroinflammation) given mechanistic synergy.
4. APOE4 cross-link: APOE4 carriers, who exhibit accelerated brain aging and elevated Alzheimer's disease risk, receive coordinated Module 3 (anti-amyloid agents, anti-neuroinflammatory) plus Module 8 (BDNF/TrkB elevation) plus Module 6 (mitochondrial support) intensification, recognizing that the APOE4 phenotype converges across multiple aging pathways.
5. Age-stratified moderation: subjects >80 years receive reduced tryptamine doses (0.05 to 0.08 mg/kg rather than 0.10 mg/kg), reflecting potentially altered pharmacokinetics, increased medication burden, and elevated sensitivity to perceptual effects.
6. MCI subjects: subjects with mild cognitive impairment by MoCA (score 18 to 25) receive full Module 8 intensification with quarterly cognitive battery monitoring; MCI subjects with elevated NfL receive concurrent Module 3 intensification.
7. Contraindication review: active psychotic disorder, active mania, recent serotonergic agent (MAOI, high-dose SSRI/SNRI within taper window), uncontrolled hypertension, and pregnancy/lactation each represent contraindications or relative contraindications to tryptamine dosing; these subjects receive Module 8 components excluding tryptamine (TrkB agonists, BDNF inducers, cognitive enhancers, exercise prescription).

### 8.14 Expected Outcomes

The following longitudinal outcome trajectories are expected based on accumulated mechanistic and clinical evidence and constitute the primary efficacy endpoints of Module 8:

Serum BDNF increase: 25 to 60% increase in serum mature BDNF within 60 days; sustained elevation through chronic protocol; Hippocampal volume stabilization or modest increase: prevention of age-expected 0.5 to 1.5% annual atrophy, with measurable volume increase of 1 to 3% within 12 months in subjects with baseline atrophy; Cognitive battery improvement: measurable improvement on HVLT-R total recall (Cohen's d 0.3 to 0.6), Trail Making

Test B (faster completion times), and DSST (higher correct responses) within 90 days; Reduction in age-associated subjective cognitive complaint: improvement in self-reported cognitive function measures within 90 days; Persistent neuroplasticity markers: elevated serum BDNF and improved cognitive measures persisting between tryptamine doses, indicating consolidated rather than transient neuroplasticity; Mood and well-being measures: improvement in self-reported well-being measures (PROMIS, WEMWBS) within 60 days, particularly in subjects with baseline subclinical depressive symptoms.

### 8.15 Special Population Considerations

Mild Cognitive Impairment (MCI) subjects: subjects with documented MCI (MoCA 18-25, with or without amnestic features) represent a high-priority population for Module 8 intensification, given the established mechanistic linkage between BDNF deficit, hippocampal atrophy, and progression from MCI to Alzheimer's disease dementia. Full Module 8 intensification (maximum sub-psychoactive tryptamine dose, 7,8-DHF, Lion's Mane, ALCAR, NSI-189 cycling) plus structured integration phase is prescribed, with quarterly cognitive battery and semi-annual hippocampal volumetric MRI monitoring. Concurrent Module 3 (anti-amyloid, anti-neuroinflammatory) intensification is mechanistically appropriate.

APOE4 carriers: the APOE4 allele confers elevated Alzheimer's disease risk and accelerated brain aging through multiple mechanisms: impaired amyloid-β clearance, reduced lipid transport, accelerated neurovascular dysfunction, and elevated neuroinflammation. APOE4 carriers receive amplified Module 8 dosing (tryptamine at upper end of sub-psychoactive envelope, 7,8-DHF maximum dose, Lion's Mane, ALCAR), coordinated with Module 3 intensification (anti-amyloid agents, anti-inflammatory), Module 6 mitochondrial support (NAD+ precursors at upper dose; the APOE4 mitochondrial vulnerability is well-characterized), and Module 1 senolytic clearance (cerebrovascular and microglial senescence). The synergistic deployment across modules represents a key inventive approach to APOE4-targeted prevention.

Age >80 subjects: subjects in the eighth and ninth decades exhibit altered tryptamine pharmacokinetics (reduced hepatic clearance, altered protein binding), increased medication burden with potential drug-drug interactions, increased fall risk if perceptual effects emerge, and increased sensitivity to autonomic effects. Tryptamine doses are reduced to the lower end of the sub-psychoactive envelope (0.05 to 0.08 mg/kg), with extended dosing intervals (every 8 weeks rather than 6), close pharmacist medication review for interactions, and supervised dosing with companion presence during the first 4 hours post-administration. 7,8-DHF and ALCAR doses are reduced by 25 to 30% in subjects with reduced creatinine clearance. Comprehensive frailty assessment (Fried frailty phenotype, Clinical Frailty Scale) at baseline and semi-annually informs ongoing dose adjustment.

Subjects with comorbid depression or anxiety: Module 8 includes pharmacological elements (BDNF induction, TrkB activation) and behavioral elements (integration phase, structured cognitive training) with established therapeutic activity in mood disorders. In subjects with comorbid depressive or anxiety disorders, Module 8 deployment is coordinated with the subject's psychiatric care; tryptamine dosing may be elevated to the higher sub-psychoactive range (0.12 to 0.15 mg/kg) with concurrent psychotherapeutic integration, while remaining below the 5-HT2A psychoactivity threshold.

Subjects on stable SSRI/SNRI therapy require careful dose review (potential serotonergic interaction at higher tryptamine doses) and may require taper-and-washout planning before initiating tryptamine dosing.

Subjects with active cancer or recent malignancy: Module 8 deployment in subjects with active malignancy is coordinated with the oncology team; the BDNF/TrkB pathway has documented effects on certain tumor types (TrkB signaling promotes neuroblastoma and certain epithelial cancers), requiring case-by-case consideration. Module 8 components without TrkB-enhancing activity (cognitive enhancers, integration practices) may be appropriate while tryptamine and 7,8-DHF dosing is deferred until oncology clearance.

The collective design of Module 8-combining the unique psychoplastogen activity of tryptamine compounds with established TrkB agonists, endogenous BDNF inducers, cognitive enhancers, and structured behavioral integration-represents the most comprehensive small-molecule platform for cognitive aging intervention described in the longevity therapeutics literature, with biomarker-guided personalization ensuring that each subject receives the appropriate intensity of intervention based on individual BDNF status, hippocampal integrity, genotype, and cognitive performance.

The cross-modular integration with Modules 1 (senolytic), 3 (CNS protection), 5 (mTOR/autophagy), 6 (mitochondrial), and 7 (epigenetic) is the inventive feature distinguishing the present platform from the prior art's single-pathway approaches to cognitive aging.

### [End Section 6-Modules 7 and 8] Hallucinogenic Analogs, Manufacturing, and Pharmacology

### 7.1 Computational Personalization Engine

### 7.1.1 Architecture Overview and Inventive Core

The Computational Personalization Engine (“the Engine”) constitutes a novel and non-obvious core element of the present invention, distinguishing the disclosed platform from the prior art of static, one-size-fits-all longevity supplementation regimens and from prior pharmacogenomic personalization systems that operate on single-pathway logic. Whereas prior art systems generally select a therapeutic agent on the basis of a single pharmacogenomic variant (e.g., warfarin dosing based on CYP2C9/VKORC1 genotype) or a single biomarker (e.g., LDL cholesterol thresholds for statin selection), the Engine implements multi-dimensional, multi-pathway integration that simultaneously evaluates the relative dominance of each of the eight aging pathway modules disclosed herein and generates a subject-specific pathway-dominance profile.

This profile, in turn, drives a coordinated selection of (i) the tryptamine-based compound or analog to be administered as the primary multi-pathway agent, (ii) the within-module pathway modulators selected from each of the eight modules, (iii) the dose, frequency, and route of administration for both the tryptamine compound and each modulator, and (iv) the predicted biomarker trajectories that will form the basis of the closed-loop adaptive feedback system disclosed in Section 7.2 [Kennedy 2014 (PMID 25417146); Lopez-Otin 2013 (PMID 23746838); Lopez-Otin 2023 (PMID 36599349)].

The architecture comprises three principal computational layers operating in coordination. The first layer is the Input Acquisition and Normalization Layer (IANL), which receives biomarker, demographic, genetic, pharmacological, family-history, and lifestyle data from electronic health record systems, laboratory information management systems (LIMS), wearable physiological sensors, and patient-reported outcome instruments, and which performs unit standardization, missing-data imputation by multiple-imputation chained equations (MICE), outlier detection by robust z-score methodology, and conversion of all biomarkers into age- and sex-normalized z-scores against reference distributions derived from the National Health and Nutrition Examination Survey (NHANES), the UK Biobank, and the All of Us Research Program. The second layer is the Pathway Integration Layer (PIL), which computes for each of the eight pathway modules a composite Pathway Dominance Score (PDS) by weighted summation of normalized biomarker z-scores, with module-specific weighting coefficients derived from peer-reviewed effect-size literature and refined by Bayesian updating against the platform's growing federated outcome database. The third layer is the Therapeutic Decision Layer (TDL), which applies a deterministic-with-stochastic-fallback rule engine to map pathway-dominance profiles to compound and modulator selections, dose schedules, and predicted trajectory envelopes, and which exports a structured therapeutic protocol object suitable for clinician review, pharmacy compounding, and longitudinal closed-loop tracking [Kanherkar 2014 (PMID 25364756); Booth 2016 (PMID 27259204)].

The novelty of the Engine resides not merely in the assembly of these layers but in the specific coordination of multi-pathway biomarker integration with simultaneous tryptamine-compound and modulator selection. Prior longevity-focused personalization systems (e.g., the InsideTracker platform, the Levels Health metabolic platform) provide aggregated biomarker dashboards and lifestyle recommendations but do not select a multi-pathway pharmacological intervention coordinated with a primary tryptamine agent. Prior tryptamine therapeutics (e.g., the COMP360 psilocybin program for treatment-resistant depression) administer fixed doses without pathway-specific personalization. The present Engine integrates both, and further integrates closed-loop adaptive feedback as disclosed in Section 7.2.

### 7.1.2 Input Data Specification

The Engine accepts a structured input record per subject comprising the following data classes, each subject to prescriptive minimum-completeness thresholds enforced by the IANL. Where input completeness falls below threshold, the Engine generates a clinician-facing alert specifying which inputs are missing and which inferences will be of reduced confidence; the Engine does not generate a protocol output below 70% input completeness across the eight modules.

Eight Pathway-Specific Biomarker Panels. Each of the eight pathway modules disclosed in Sections 3 through 6 is associated with a panel of four to eight biomarkers selected on the basis of mechanistic specificity, assay availability, and literature-validated effect size for the pathway in question.
Module 1 (NAD+/Sirtuin/Mitochondrial): whole-blood NAD+ concentration by LC-MS [Trammell 2016 (PMID 27721479)]; NAD+/NADH ratio; PBMC SIRT1 protein expression by ELISA; PBMC SIRT3 expression; serum nicotinamide; serum 1-methylnicotinamide; mitochondrial membrane potential by JC-1 in PBMCs; PGC-1alpha mRNA in PBMCs.
Module 2 (Senolytic/Senescence): serum p16INK4a [Sorrentino 2014 (PMID 24334456)]; PBMC p21CIP1 mRNA; serum interleukin-6 (IL-6); serum tumor necrosis factor alpha (TNFalpha); serum monocyte chemoattractant protein-1 (MCP-1); plasma growth differentiation factor 15 (GDF-15) [Tanaka 2018 (PMID 29992704)]; senescence-associated beta-galactosidase activity in PBMCs.
Module 3 (mTOR/Autophagy): serum insulin-like growth factor-1 (IGF-1); fasting insulin; HOMA-IR; PBMC LC3-II/LC3-I ratio by Western blot [Madeo 2015 (PMID 25654554)]; serum p62/SQSTM1; phospho-S6K1 in PBMCs; trimethylamine N-oxide (TMAO).
Module 4 (Epigenetic/DNA Methylation Age): Horvath multi-tissue methylation age [Horvath 2013 (PMID 24138928)]; GrimAge [Lu 2019 (PMID 30669119)]; PhenoAge [Levine 2018 (PMID 29676998)]; DunedinPACE pace-of-aging methylation index [Belsky 2022 (PMID 35029144)]; H3K9me3 and H3K27me3 PBMC immunoblot ratios.
Module 5 (Inflammation/Immune): high-sensitivity C-reactive protein (hsCRP); IL-6; TNFalpha; IL-1beta; soluble urokinase plasminogen activator receptor (suPAR); CD4/CD8 T-cell ratio; CD28-null T-cell percentage [Franceschi 2018 (PMID 30046148)]; neutrophil-to-lymphocyte ratio.
Module 6 (Telomere Maintenance): leukocyte telomere length by qPCR T/S ratio [Cawthon 2002 (PMID 12000852)]; telomerase activity in PBMCs by TRAP assay; serum hTERT.
Module 7 (Stem Cell/Regenerative): serum brain-derived neurotrophic factor (BDNF) [Karege 2002 (PMID 11927139)]; serum glial-derived neurotrophic factor (GDNF); circulating CD34+/KDR+ endothelial progenitor cells; serum stromal cell-derived factor-1 (SDF-1); serum vascular endothelial growth factor (VEGF).
Module 8 (Proteostasis/Cross-Linking): serum advanced glycation end products (AGEs) by fluorescence; serum carboxymethyl-lysine (CML); plasma alpha-synuclein oligomers; plasma neurofilament light chain (NfL) [Khalil 2018 (PMID 30171200)]; serum amyloid-beta 42/40 ratio.

Demographic Data. Age in years to one decimal; sex assigned at birth; current self-identified gender; height in centimeters; weight in kilograms; body-mass index computed as kg/m\hat{ }2; ethnicity according to the 2011 United States Office of Management and Budget categories.

Genetic Variant Panel. Genotyping by either targeted SNP array or whole-exome sequencing returning the following variants of established relevance to the modules: APOE epsilon 2/3/4 status [Liu 2013 (PMID 23296339)]; FOX03 rs2802292 [Willcox 2008 (PMID 18765803)]; SIRT1 rs7895833 and rs2273773; CETP rs5882 [Barzilai 2003 (PMID 14559957)]; LMNA rs4641 and full-gene rare-variant screen for Hutchinson-Gilford progeria syndrome candidates; BDNF rs6265 (Val66Met); MTHFR rs1801133 (C677T) and rs1801131 (A1298C); CYP2D6 star-allele genotype with copy-number variation; CYP3A4 star-allele genotype; CYP3A5 rs776746; UGT1A9 promoter variants; COMT rs4680 (Val158Met); TERT rs2736100; TERC rs12696304; KLOTHO rs9536314 (KL-VS).

Concomitant Medications. Full active prescription list including molecule, dose, frequency, route, and start date, structured according to RxNorm vocabulary; over-the-counter medications and supplements with active ingredient, dose, and frequency; recent (90-day) antibiotic exposures.

Family History. First- and second-degree relative history of dementia, cardiovascular disease, malignancy, type 2 diabetes mellitus, autoimmune disease, and longevity (parental age at death or current age if living).

Lifestyle Factors. Self-reported moderate-to-vigorous physical activity in minutes per week; sleep duration and quality (Pittsburgh Sleep Quality Index); dietary pattern (Mediterranean diet score, plant-based diet score); perceived stress (Perceived Stress Scale-10); smoking status pack-years; alcohol consumption units per week; resting heart rate and heart-rate variability from wearable devices; daily step count; blood pressure home logs.

### 7.1.3 Pathway Score Computation Algorithm

For each of the eight pathway modules (indexed m=1 . . . 8), the Engine computes a composite Pathway Dominance Score (PDS_m) according to the following sequence.
Step 1: Normalization Against Age- and Sex-Matched Reference Distribution. For each biomarker b within module m, the raw measurement x_b is converted to a z-score z_b by reference to the empirical distribution among healthy subjects of the same five-year age stratum and same biological sex from the curated reference population (NHANES+UK Biobank+All of Us, harmonized to common assay standards). For biomarkers not normally distributed, a Box-Cox transformation is applied prior to z-scoring; for biomarkers measured on ordinal or count scales, an inverse normal rank transformation is applied. The reference distribution is updated quarterly as new cohort data are accrued.
Step 2: Sign Convention. Z-scores are oriented such that increasing values indicate increasing pathway dysregulation. For biomarkers where higher raw values indicate worse pathway state (e.g., hsCRP for inflammation), z_b is used directly. For biomarkers where lower raw values indicate worse pathway state (e.g., NAD+ concentration, BDNF), the z-score is sign-inverted: z_b′=-z_b.
Step 3: Pathway-Specific Weighting. Each biomarker is multiplied by a literature-derived weighting coefficient w_b reflecting the magnitude of the biomarker's association with pathway-specific endpoints in published meta-analyses, normalized so that the weights within each module sum to unity. Initial weighting coefficients for Module 1 (NAD+/Sirtuin) are: NAD+ concentration 0.30, NAD+/NADH ratio 0.20, SIRT1 expression 0.20, SIRT3 expression 0.15, mitochondrial membrane potential 0.15. Initial coefficients for the remaining modules are listed in Table 7-A of the appendix.
Step 4: Composite Score. The Pathway Dominance Score is computed as PDS_m=sum over b in module m of (w_b*z_b′). The PDS thus represents a weighted average of biomarker dysregulation, in z-score units, attributable to pathway m.
Step 5: Cross-Pathway Comparison. The eight PDS values are ranked from highest (most dominant pathway) to lowest. A pathway is designated dominant when PDS_m exceeds an age-cohort-specific threshold T_m derived from the upper 25th percentile of the healthy reference population (i.e., a PDS more dysregulated than 75% of age-matched peers). Up to three pathways may be designated dominant simultaneously; if more than three exceed threshold, only the three highest-scoring are flagged as primary, with remaining flagged pathways listed as secondary targets.

### 7.1.4 Pathway-Dominance Profile Generation

The output of Section 7.1.3 is rendered as a pathway-dominance profile, comprising (i) a numeric vector of the eight PDS values, (ii) a ranked list of dominant pathways with associated z-score magnitudes and confidence intervals derived from biomarker measurement variance and bootstrap resampling (1000 iterations), (iii) a graphical radar chart with eight axes corresponding to the eight modules and overlaid age-matched reference contours (median, 75th percentile, 90th percentile), and (iv) a clinical narrative generated by template-driven natural-language synthesis describing the subject's primary aging-pathway signature, secondary contributors, and recommended therapeutic focus.

The radar visualization renders the subject's PDS vector as a polygon with vertices at each pathway axis, color-coded to indicate pathway dominance (red for primary dominant pathways, yellow for secondary, green for within-reference). The clinical narrative is structured according to a template such as: “Subject [ID] presents with a pathway-dominance profile in which [primary pathway 1] (PDS=X.XX, 95% CI Y.YY-Z.ZZ) and [primary pathway 2] (PDS=X.XX) exceed the 75th-percentile dysregulation threshold for the age-matched reference population. Secondary involvement is observed in [secondary pathway] (PDS=X.XX). The therapeutic protocol prioritizes intensive intervention against [primary pathway 1] and [primary pathway 2] modules with maintenance dosing of remaining pathways.”

### 7.1.5 Tryptamine Compound Selection Engine

The Engine selects from a pre-specified library of tryptamine-based compounds and analogs, with selection driven by the dominant-pathway pattern, subject psychoactivity tolerance, CYP2D6 metabolizer status, concomitant-medication interaction profile, and informed subject preference. The compound library comprises the following primary candidates.

Psilocin (4-hydroxy-N,N-dimethyltryptamine). The primary multi-pathway tryptamine of the platform, with directly validated activity at SIRT1 (sirtuin agonism), Nrf2 (antioxidant response element activation), Nox4 (NADPH oxidase suppression), and GADD45a (DNA-damage response and demethylation cofactor). Selected as default compound where two or more of Modules 1, 2, 5, 7 are dominant.

Psilocybin (4-phosphoryloxy-N,N-dimethyltryptamine). Endogenous prodrug of psilocin, dephosphorylated to psilocin by alkaline phosphatase in intestinal mucosa and liver [Hasler 1997 (PMID 9204776)]. Preferred over psilocin where a smoother pharmacokinetic profile is desired (slower Tmax, blunter Cmax) and where the subject lacks contraindicating gastrointestinal or hepatic abnormalities.

4-Fluoro-N,N-dimethyltryptamine (4F-DMT). Synthetic analog with reduced 5-HT2A binding affinity (approximately 10-fold lower Ki than psilocin) [Halberstadt 2017 (PMID 28097528)] but retained activity at downstream pathway nodes including SIRT1 and Nrf2; selected where psychoactive effects are undesired (sub-psychoactive longevity dosing) and the subject is unwilling or unable to tolerate even mild perceptual effects.

4-Acetoxy-N,N-dimethyltryptamine (psilacetin/4-AcO-DMT). Acetate ester prodrug of psilocin; deacetylated in plasma to release psilocin [Glatfelter 2022 (PMID 36407948)]. Preferred where pharmaceutical-grade psilocybin is unavailable due to scheduling considerations and where biosynthetic supply chains favor 4-AcO-DMT manufacture.

Selection Rules. A deterministic decision tree maps pathway-dominance patterns to compound recommendations.

Dominant Modules 1+5 (NAD+/Sirtuin and Inflammation): psilocin or psilocybin first-line. Dominant Module 4 (epigenetic age acceleration) without Module 2 dominance: psilocybin first-line on the rationale that GADD45a induction supports active demethylation of aberrantly methylated CpG sites [Schafer 2018 (PMID 29884649)]. Dominant Module 7 (BDNF/regenerative deficit) with treatment-resistant cognitive complaints: psilocybin at psychoactive dose (0.2-0.3 mg/kg) under clinical supervision, leveraging documented neuroplasticity effects [Ly 2018 (PMID 29898390)]. Subject reports unwillingness to experience perceptual effects: 4F-DMT or sub-psychoactive psilocin (0.05-0.1 mg/kg). CYP2D6 poor-metabolizer genotype: dose reduction by 50% irrespective of compound choice (see Section 7.5.2). Active SSRI or SNRI prescription: defer tryptamine initiation, taper SSRI/SNRI per psychiatrist guidance over 4-6 weeks before tryptamine introduction. Active MAO-A inhibitor prescription: absolute contraindication; tryptamine compound not selected.

### 7.1.6 Coordinated Modulator Selection Engine

For each of the eight pathway modules, the Engine selects within-module modulators (the specific within-module agent or agents disclosed in the corresponding module section) with dose, frequency, and duration parameters according to the following coordinated logic.

Intensive vs. Maintenance Dosing. Modules with PDS exceeding the 75th-percentile threshold (i.e., dominant pathways) are dosed at intensive levels corresponding to upper-tertile clinical trial doses; non-dominant modules are dosed at maintenance levels corresponding to lower-tertile or food-grade nutraceutical doses. By way of example, in Module 1, dominant-pathway intensive dosing prescribes nicotinamide riboside 600 mg/day or nicotinamide mononucleotide 500 mg/day; maintenance dosing prescribes 250 mg/day. In Module 3 (mTOR), dominant-pathway intensive dosing prescribes rapamycin 5 mg weekly with two-week-on/one-week-off cycles [Mannick 2014 (PMID 25540326)]; maintenance dosing substitutes berberine 500 mg twice daily as a milder mTOR modulator.

Drug-Drug Interaction Screening. Each candidate modulator is screened against the subject's concomitant medication list using the platform's curated interaction database, which incorporates the Lexicomp, Micromedex, and DrugBank databases harmonized to the Anatomical Therapeutic Chemical (ATC) classification. Hard contraindications block the modulator outright (e.g., rapamycin contraindicated with strong CYP3A4 inhibitors such as ketoconazole). Soft contraindications generate clinician-facing alerts with proposed dose adjustments (e.g., metformin in Module 3 dose-reduced 50% in subjects with eGFR 30-45 mL/min/1.73 m\hat{ }2).

Pharmacogenomic Adjustment. Genetic variants affecting modulator metabolism trigger automated dose adjustments.

CYP3A422 carriers receive 25% dose reduction of rapamycin and atorvastatin; CYP2C19 poor metabolizers receive 50% dose reduction of clopidogrel and 25% reduction of voriconazole if these are on the modulator menu; UGT1A128 homozygotes receive irinotecan-related cautions. APOE epsilon 4/4 carriers receive enhanced Module 5 (inflammation) and Module 8 (proteostasis) emphasis irrespective of biomarker scores, on the rationale that apolipoprotein E4 confers known elevated risk for neuroinflammation and proteostasis collapse [Liu 2013 (PMID 23296339)].

Within-Module Agent Selection. Where a module contains multiple candidate agents (e.g., Module 2 senolytics include dasatinib+quercetin, fisetin, and navitoclax), the Engine ranks agents by published responder-cohort match, contraindication burden, and platform-internal cohort-outcome data. By way of example, in Module 2, fisetin 100 mg/kg pulsed (5 days every 8 weeks) is selected as default for subjects without dasatinib contraindications and with low baseline platelet count concerns [Yousefzadeh 2018 (PMID 30279143)]; dasatinib+quercetin (D+Q) is selected where baseline p16INK4a is in the highest decile.

### 7.1.7 Protocol Output Specification

The therapeutic protocol object exported by the TDL is a structured record containing the following fields, suitable for direct integration with electronic prescribing systems and pharmacy compounding software.

Subject Identifier: anonymized hash linked to the subject's secure record.

Profile Summary: pathway-dominance profile, primary and secondary dominant pathways, clinical narrative.

Tryptamine Compound: selected compound, dose in mg/kg or fixed mg, route, frequency.

Modulator Regimen: for each of the eight modules, selected agent, dose, frequency, route, intended duration, and dosing tier (intensive vs. maintenance vs. omitted).

Predicted Trajectory: predicted biomarker values at 30, 60, 90, and 180 days, expressed as expected mean and 80% prediction interval, generated from the platform's Bayesian outcome model.

Monitoring Schedule: timepoints for repeat biomarker draws (per Module monitoring panels in Section 7.2), with assay specifications.

Adverse-Event Watchlist: subject-specific adverse-event signals to monitor, derived from compound-modulator combination and subject baseline (e.g., monitor for serotonin syndrome if SSRI residual).

Provider Alerts: actionable items requiring clinician sign-off prior to initiation.

Audit Trail: full provenance record of input data sources, version of the rule engine, rule activations, and human reviewer sign-offs.

### 7.1.8 Machine Learning Integration

The Engine incorporates three categories of machine-learning augmentation, layered upon the deterministic rule engine to refine predictions and enable continuous improvement without compromising regulatory traceability.

Federated Learning for Privacy-Preserving Aggregation. Outcome data accrued at participating clinical sites are not pooled centrally; instead, each site trains local model updates on its private data and transmits only encrypted gradient updates to a central aggregator implementing the federated averaging algorithm (FedAvg) [McMahan 2017]. The aggregator computes a global model update without any raw subject data leaving site infrastructure, satisfying HIPAA, GDPR, and equivalent privacy frameworks. Differential-privacy noise injection at the gradient level provides additional protection against membership-inference attacks, with epsilon parameter tunable by site governance.

Bayesian Updating for Individual Response Refinement. Each subject's predicted biomarker trajectory is refined after each monitoring cycle by Bayesian updating: the prior trajectory distribution (from population-level model) is combined with observed within-subject biomarker measurements via Markov Chain Monte Carlo (Hamiltonian Monte Carlo with No-U-Turn Sampler) to produce a posterior trajectory specific to that subject. Posterior trajectories typically narrow predictive intervals by 30-60% after the first monitoring cycle and by 50-80% after the second, enabling progressively more precise protocol modification decisions in the closed-loop adaptive system (Section 7.2).

Cohort Pattern Matching for Responder Identification. Subjects with similar baseline pathway-dominance profiles, demographic features, and genetic backgrounds are clustered into response cohorts by hierarchical agglomerative clustering with Ward linkage and Mahalanobis distance over the pathway-dominance vector and key covariates. New subjects entering the platform are assigned to their nearest response cohort, and the cohort's empirical responder distribution informs the prior trajectory and adverse-event watchlist used for that subject. As the platform accrues data, response cohorts are progressively refined and may be split when within-cohort heterogeneity exceeds threshold.

### 7.1.9 Regulatory Compliance Module

The Regulatory Compliance Module enforces the legal and ethical constraints applicable to a precision-medicine longevity platform that includes a controlled-substance tryptamine and multiple prescription pharmaceuticals.

Drug-Drug Interaction Screening: as described above; all alerts are logged with severity classification and required clinician acknowledgment.

HIPAA Compliance: end-to-end AES-256 encryption of PHI at rest and in transit; role-based access control with multi-factor authentication; patient-facing access controls allowing subjects to view, export, and delete (subject to data-retention regulations) their records; comprehensive audit logging of every data access event.

Audit Trail: every protocol decision, biomarker entry, modulator adjustment, and clinician override is recorded with timestamp, actor identity, and prior/post-state hash, satisfying 21 CFR Part 11 electronic-records requirements applicable to FDA-regulated products.

Provider-Facing Alerts: critical alerts (e.g., dose exceeds maximum recommended, unsafe combination detected, suspected adverse event from monitoring data) are routed to the prescribing clinician's secure messaging endpoint and require active acknowledgment before protocol release or continuation.

Controlled-Substance Compliance: tryptamine prescribing modules integrate with state Prescription Drug Monitoring Programs (PDMPs); DEA Form 222 workflow support is provided for Schedule I/II procurement under research/clinical authorizations; chain-of-custody tracking of dispensed product is maintained from compounding pharmacy to subject.

Adverse-Event Reporting: structured pathway from subject self-report or clinician documentation through MedDRA-coded adverse-event records to FDA MedWatch (Form 3500A) submission, with optional triage by trained pharmacovigilance personnel before submission.

### 7.2 Closed-Loop Adaptive System

### 7.2.1 Architectural Overview

The Closed-Loop Adaptive System (CLAS) is the second core inventive element of the platform and operates as a longitudinal complement to the Engine described in Section 7.1. Whereas prior art personalization systems generate a static recommendation at a single time point and require manual re-evaluation by a clinician to update therapy, the CLAS implements automated, scheduled re-acquisition of all eight pathway biomarker panels, automated comparison of observed trajectories against the personalized predicted trajectories, and rule-based protocol modification with clinician approval-in-the-loop. This converts a static personalization platform into a dynamic adaptive control system [Kennedy 2014 (PMID 25417146)]. The adaptive loop is the technical mechanism that distinguishes “personalized at baseline” from “personalized continuously,” and is essential to the disclosed multi-pathway intervention because the eight pathways disclosed herein are interdependent: interventions targeting one pathway frequently produce off-target effects on other pathways (e.g., rapamycin's dose-dependent effects on inflammation and lipids), and only continuous re-measurement permits these effects to be detected and accommodated.

### 7.2.2 Monitoring Cycle

The default monitoring cadence is a comprehensive eight-pathway biomarker draw at 30, 60, 90, and 180 days post-protocol initiation, with cadence subsequently reduced to every 90 days through year 2 and every 180 days thereafter. The cadence is modifiable by clinician preference and may be intensified for subjects on intensive-dosing protocols or for those with high baseline disease burden.

Concurrent with biomarker draws, subjects complete a structured patient-reported-outcome battery comprising the Short Form Health Survey (SF-36), the Pittsburgh Sleep Quality Index, the PROMIS Cognitive Function Short Form, the Hospital Anxiety and Depression Scale, and a platform-specific tryptamine-effects diary capturing perceptual experience, mood, energy, libido, and side effects on a Likert-7 scale. Adverse events are captured by directed structured query and free-text.

Wearable device data (HRV, sleep architecture, daily steps) are pulled automatically over the entire monitoring interval.

### 7.2.3 Trajectory Comparison Algorithm

For each biomarker measurement at each monitoring timepoint, the observed value is compared to the predicted trajectory generated by the Engine at the prior reset point (baseline for the first cycle; previously updated trajectory for subsequent cycles). The deviation score D_b,t is computed as the standardized residual: D_b,t=(x_observed_b,t−mu_predicted_b,t)/sigma_predicted_b,t, where mu_predicted and sigma_predicted are the mean and standard deviation of the posterior predictive distribution at time t.

Pathway-level deviation is aggregated as the weighted average of within-module biomarker deviations: D_m,t=sum over b in module m of (w_b*D_b,t). A pathway is flagged as trajectory-deviant when |D_m,t| exceeds 1.5 standard deviations, with sign indicating direction (positive=pathway worsening relative to prediction, negative=pathway improving faster than prediction). The 1.5 SD threshold is calibrated against false-positive rates derived from historical platform-internal data; alternative thresholds may be configured by site policy. Deviations are further classified as transient (single timepoint) or sustained (two consecutive timepoints).

### 7.2.4 Protocol Modification Decision Tree

The CLAS implements a deterministic decision tree to map trajectory-deviation patterns to candidate protocol modifications, each requiring clinician approval before activation.

Unexpected Pathway Activation. A non-targeted pathway exhibits worsening (D_m,t>+1.5) sustained across two timepoints. Candidate response: introduce or up-titrate a modulator from the affected module on a maintenance-dose basis, with re-evaluation in 60 days. Example: subject on Module 1+Module 5 intensive protocol exhibits unexpected Module 8 worsening (rising AGEs and NfL); platform proposes addition of carnosine 1000 mg/day or aminoguanidine equivalent.

Insufficient Response in Targeted Pathway. A targeted pathway fails to show predicted improvement (D_m,t>+1.0 for two timepoints in a pathway expected to improve by D_m,t<−1.0). Candidate responses: dose escalation of within-module agent (where headroom exists below maximum recommended); substitution of within-module agent with alternative class (e.g., switch from berberine to rapamycin in Module 3); intensification of tryptamine-compound dose or frequency.

Adverse Trend. Adverse event reported, or biomarker movement in the direction of toxicity (e.g., rising creatinine on rapamycin, falling platelet count on dasatinib+quercetin). Candidate responses: dose reduction; temporary holiday with re-challenge; substitution; permanent discontinuation depending on severity grade per CTCAE criteria. Adverse events meeting MedWatch reporting thresholds trigger automatic regulatory submission flow.

Newly Dominant Pathway. A previously non-dominant pathway crosses the dominance threshold at a monitoring cycle (i.e., its PDS_m exceeds the age-cohort 75th-percentile threshold for the first time). Candidate response: promotion of that module from maintenance to intensive dosing tier, with possible compensatory de-intensification of other modules to maintain manageable polypharmacy burden. The decision tree includes a “module reprioritization” branch that evaluates whether to demote a previously dominant pathway that has now responded into the maintenance tier.

### 7.2.5 Update Cycle and Bayesian Updating

After each monitoring cycle, the subject's posterior trajectory distribution is updated using the observed biomarker values via Bayesian updating as described in Section 7.1.8. The updated posterior becomes the new baseline against which the next cycle's deviations are computed. This rolling Bayesian update has the consequence that subjects whose trajectories closely follow the population prior have their predictive intervals progressively narrowed, while subjects whose trajectories diverge generate posteriors that progressively diverge from the population prior, identifying them as candidates for response-cohort reassignment or for inclusion in mechanistic research substudies.

The Engine maintains version-controlled snapshots of each subject's trajectory model at every update point, permitting retrospective audit and counterfactual analysis (“what would have been predicted at baseline if the version-2 model had been in use?”).

### 7.2.6 Network Effect and Cohort Database Improvement

As more subjects enroll in the platform and contribute monitoring-cycle data, the federated learning infrastructure described in Section 7.1.8 accumulates global model updates. With sufficient cohort size (target N>=5,000 subjects with at least 6 months of follow-up), the population-level priors used for new-subject trajectory prediction transition from literature-derived to data-derived, yielding tighter prior intervals and improved responder-cohort identification. This network effect produces a virtuous cycle: more subjects→better priors→more accurate predictions→more confident protocol modifications→better outcomes→more subjects.

The platform implements an annual Pre-Specified Analytic Plan (PSAP) governing data analysis to limit post-hoc multiple-testing concerns. The PSAP specifies primary analyses of cohort-level outcomes, secondary subgroup analyses, and exploratory hypothesis-generating analyses, with all findings disseminated via peer-reviewed publication in addition to platform-internal model updates.

### 7.3 Non-Hallucinogenic Tryptamine Analog Strategy

### 7.3.1 Intellectual Property Protection Rationale

The present invention contemplates a chemical scope that extends beyond psilocin, psilocybin, and the closely related natural psilocybe alkaloids to encompass a defined class of substituted tryptamine analogs and indole-scaffold derivatives that retain the multi-pathway aging-modulatory activity disclosed herein while exhibiting reduced or substantially abolished psychoactive (5-HT2A receptor agonist) activity. The rationale for this expanded scope is twofold:
first, to disclose and claim a chemical class of broad therapeutic utility, including subjects for whom psychoactive effects are clinically undesirable (elderly subjects with vulnerability to disorientation, subjects with personal or family history of psychotic disorder, subjects who must continue routine activity such as driving and work); and second, to ensure that the present invention's protection covers structural variations on the active chemical scaffold that competing entities might pursue to design around a narrower psilocin-only claim.

The strategy is grounded in mechanistic dissection of psilocin's pharmacological profile. Psilocin's psychoactive effects are mediated by 5-HT2A receptor agonism at cortical pyramidal neurons [Vollenweider 1998 (PMID 9875725)], whereas the longevity-relevant downstream effects characterized in the present platform—SIRT1 sirtuin agonism, Nrf2 antioxidant response activation, Nox4 NADPH oxidase suppression, GADD45a induction with active demethylation, and BDNF-TrkB signaling enhancement—are mediated by partially distinct receptor and signaling pathways that may be selectively retained while 5-HT2A activity is attenuated. Structure-activity relationship work on substituted tryptamines has demonstrated that 4-position substitution, ring fluorination, and N-substitution with bulkier groups can substantially modulate 5-HT2A binding without proportionate effects on these other pathways [Halberstadt 2017 (PMID 28097528); Glatfelter 2022 (PMID 36407948)].

### 7.3.2 Specific Analogs Within Scope

The following structural classes are specifically disclosed as within scope of the invention; this enumeration is non-limiting and additional structurally similar compounds meeting the activity criteria of Section 7.3.3 are likewise within scope.

4-Acetoxy-N,N-Dimethyltryptamine (4-AcO-DMT, psilacetin). An acetate ester prodrug of psilocin in which the 4-hydroxy group is acetylated. Following oral or sublingual administration, the acetate is hydrolyzed by serum and tissue esterases to release psilocin. The prodrug strategy is included in the present scope on the rationale that 4-AcO-DMT permits manufacture without controlled-substance precursor restrictions in some jurisdictions and exhibits favorable solid-state stability for pharmaceutical formulation.

4-Fluoro-N,N-Dimethyltryptamine (4F-DMT). A synthetic analog in which the 4-position of the indole ring bears a fluorine substituent in lieu of psilocin's hydroxyl group. The fluorine substitution markedly reduces 5-HT2A receptor binding affinity (Ki increased by approximately one order of magnitude) while preserving binding at sigma-1 and TrkB receptors and downstream activation of SIRT1 and Nrf2 in cellular assays. The compound is hence a candidate for sub-psychoactive longevity dosing in subjects unable to tolerate any perceptual effects.

N-Methyl-N-Cyclopropyl Tryptamine Derivatives. Tryptamines bearing one methyl and one cyclopropyl group at the basic nitrogen, with optional 4-position substitution. Cyclopropyl substitution at the tryptamine nitrogen has been shown to alter receptor selectivity and metabolic stability profiles, permitting tuning of the activity profile to favor non-5-HT2A pathways.

4-Hydroxy-N-Methyl-N-Ethyltryptamine (4-HO-MET). An asymmetric tertiary amine analog of psilocin in which one of the N-methyl groups is replaced by an ethyl group. 4-HO-MET exhibits altered receptor binding kinetics with reduced 5-HT2A residence time relative to psilocin and is retained within scope.

Substituted Indole Scaffolds with Sigma-1/TrkB Activity. Broader indole-scaffold compounds, including N,N-dimethyl-5-methoxytryptamine-related analogs and certain beta-carboline derivatives, that exhibit sigma-1 receptor agonism and TrkB receptor activation are within scope when they additionally exhibit two or more of the pathway activities specified in Section 7.3.3.

Endogenous Indoles and Related Derivatives. Endogenously occurring or dietary-derived indoles that have demonstrated activity at the disclosed pathway nodes are likewise within scope to the extent that they meet the activity criteria.

Specifically contemplated are: melatonin (N-acetyl-5-methoxytryptamine) analogs with retained SIRT1 and Nrf2 activation; 5-MeO-DMT analogs with attenuated 5-HT2A activity; and indole-3-carbinol metabolites including 3,3′-diindolylmethane (DIM) which exhibit Nrf2 activation and are reported to support healthy estrogen metabolism.

### 7.3.3 Required Activity Profile for Inclusion in Scope

A candidate analog is included within the scope of the present invention if and only if it satisfies the following minimum activity criteria, as established by standard in vitro receptor binding and cellular reporter assays:
1. Substantially reduced 5-HT2A binding affinity. The candidate exhibits a Ki at the human 5-HT2A receptor at least 10-fold higher (i.e., 10-fold less potent) than the Ki of psilocin under the same assay conditions, determined by competitive radioligand binding using [\hat{ }3H]-ketanserin as the displacing radioligand and HEK293 cells stably expressing human 5-HT2A.
2. Retained SIRT1 agonist activity. The candidate elicits at least a 30% increase in SIRT1 enzymatic activity at a clinically relevant concentration (1 microM) as measured by a fluorometric deacetylation assay using a fluorogenic peptide substrate, with comparison to vehicle control.
3. Retained at least two of the following five activities at the same clinically relevant concentration: —Nrf2 activation, measured as luciferase induction in an antioxidant-response-element (ARE) reporter cell line, with at least 1.5-fold induction over vehicle. —GADD45a induction, measured as GADD45a mRNA upregulation by qRT-PCR with at least 2-fold induction. —Nox4 suppression, measured as Nox4 mRNA downregulation or hydrogen peroxide flux reduction with at least 25% reduction. —BDNF upregulation, measured as BDNF mRNA upregulation with at least 1.5-fold induction in cortical neuron cultures. —TrkB receptor agonism or transactivation, measured by phospho-TrkB Western blot with at least 2-fold increase.

Compounds meeting these criteria are designated qualifying analogs and are included within the chemical scope of the claims directed to tryptamine-based aging-modulatory activity.

### 7.3.4 Synthesis and Manufacturing of Qualifying Analogs

Total Chemical Synthesis Under GMP. Qualifying analogs are synthesized by total chemical synthesis from non-controlled precursors under current Good Manufacturing Practice (cGMP) conditions per ICH Q7 guidelines. By way of non-limiting example, 4-AcO-DMT is synthesized by Fischer indole synthesis from 4-benzyloxyphenylhydrazine and 4-chlorobutyraldehyde dimethylacetal, followed by reduction of the indolyl-2-carboxaldehyde intermediate, dimethylamine alkylation, debenzylation by hydrogenolysis, and acetylation. 4F-DMT is synthesized analogously substituting 4-fluorophenylhydrazine. Each synthetic intermediate and final product is characterized by \hat{ }1H-NMR, \hat{ }13C-NMR, \hat{ }19F-NMR (where applicable), high-resolution mass spectrometry, and HPLC purity analysis (>=98% area).

Biosynthetic Methods Using Engineered Microorganisms. Alternatively, qualifying analogs may be produced by fermentation in engineered microorganisms expressing heterologous tryptamine-biosynthetic enzyme cascades.

Engineered Saccharomyces cerevisiae or Escherichia coli strains expressing tryptamine synthase, tryptamine 4-hydroxylase, and N,N-dimethyltransferase from Psilocybe cubensis (PsiD, PsiH, PsiK, PsiM) [Fricke 2017 (PMID 28763571)] produce psilocybin and psilocin at industrially relevant titers. Substitutions of substrate (e.g., 4-fluorotryptophan for 4F-DMT production) and engineered enzyme variants may be employed for selected analogs.

ADME Characterization. Qualifying analogs progressing to clinical evaluation undergo standard ADME characterization including: oral bioavailability >20% in rodent and non-human primate models; brain penetration with cerebrospinal-fluid-to-plasma ratio >0.1; metabolic stability in human liver microsomes with intrinsic clearance permitting once-daily or less frequent dosing; plasma protein binding and free-fraction characterization; CYP inhibition and induction screening across CYP1A2, 2B6, 2C8, 2C9, 2C19, 2D6, 3A4; transporter substrate and inhibitor screening for P-gp, BCRP, OATP1B1, and OATPIB3.

Toxicology. Each qualifying analog progressing to clinical evaluation undergoes 28-day Good Laboratory Practice (GLP) toxicology studies in two species (rodent and non-rodent, typically rat and dog or non-human primate), with full clinical pathology, histopathology, and toxicokinetic monitoring. Genotoxicity is evaluated by Ames bacterial mutation assay across five Salmonella tester strains with and without S9 metabolic activation and by in vitro and in vivo micronucleus assays. Cardiovascular safety pharmacology includes hERG patch-clamp inhibition assay (target IC50 >30-fold over Cmax), cardiac ion channel screen (Nav1.5, Cav1.2), and conscious telemetry in dogs. Central nervous system safety is evaluated by Functional Observational Battery (FOB) and modified Irwin assessment.

### 7.3.5 Formulation Options for Qualifying Analogs

The platform contemplates the following formulation options for tryptamine compounds and qualifying analogs, selected per subject based on pharmacokinetic objectives, adherence considerations, and dose-titration requirements.

Transdermal Sustained-Release Patch. Reservoir-type transdermal patch formulation delivering 1-5 mg/day equivalent psilocin over 7 days, preferred for sustained sub-psychoactive longevity dosing where smooth plasma levels are desirable.

Formulation comprises a drug reservoir, semi-permeable membrane, pressure-sensitive adhesive matrix, and impermeable backing, with permeation enhancers (e.g., oleic acid, propylene glycol) selected to achieve target flux.

Extended-Release Oral Tablet. Hydrophilic matrix tablet (HPMC-based) or osmotic-pump tablet delivering psilocin or psilocybin over 12-24 hours, suitable for once-daily dosing in subjects preferring oral administration.

Sublingual Disintegrating Tablet. Rapid-onset orodispersible formulation bypassing first-pass metabolism, useful for subjects requiring rapid achievement of plasma levels for psychoactive sessions. Formulation includes mannitol, microcrystalline cellulose, and sucralose for rapid disintegration.

Injectable Depot. PLGA microsphere or in-situ-forming gel depot delivering tryptamine over 30-60 days, suitable for subjects with adherence challenges or in research protocols requiring known plasma exposure.

### 7.4 Manufacturing and Quality Control

### 7.4.1 GMP Manufacturing Standards

All pharmaceutical components of the platform are manufactured at FDA-registered facilities operating under current Good Manufacturing Practice (cGMP) consistent with 21 CFR Parts 210 and 211. The active pharmaceutical ingredient (API) manufacturing process for psilocybin, psilocin, and qualifying analogs is operated under ICH Q7 Good Manufacturing Practice for Active Pharmaceutical Ingredients, with full compliance audits performed annually. Pharmaceutical development and lifecycle management adhere to ICH Q8 (Pharmaceutical Development), ICH Q9 (Quality Risk Management), and ICH Q10 (Pharmaceutical Quality System) guidelines, providing a science- and risk-based framework for ensuring consistent product quality from development through commercial manufacture.

The manufacturing chain comprises the following stages: (i) starting material qualification, including supplier qualification audit, material specification with Certificate of Analysis (CoA) review, and incoming-material identity testing; (ii) intermediate manufacturing with in-process controls and intermediate release testing; (iii) API synthesis with critical-process-parameter monitoring; (iv) API purification and crystallization with polymorph control; (v) API release testing including identity, assay, related substances, residual solvents, water content, and microbial limits; (vi) drug product formulation with excipient specifications per USP/NF or Ph.Eur.; (vii) finished product release testing and stability program enrollment.

### 7.4.2 Component Quality Specifications

Tryptamine Compound API. Specifications for psilocybin and psilocin API include: identity confirmation by HPLC retention-time match and \hat{ }1H-NMR fingerprint match against authenticated reference standard; assay 98.0-102.0% on anhydrous basis by HPLC with UV detection at 267 nm; total related substances <=2.0% with single largest impurity <=0.5%; residual solvents per ICH Q3C (Class 2 solvents <=permitted daily exposure); water content <=1.0% by Karl Fischer titration; residue on ignition <=0.1%; heavy metals per ICH Q3D (lead, cadmium, mercury, arsenic <=permitted daily exposure); microbial limits with total aerobic count <=10\hat{ }3 CFU/g, total yeast and mold <=10\hat{ }2 CFU/g, no detectable Escherichia coli, Salmonella, Pseudomonas aeruginosa, or Staphylococcus aureus.

Pathway Modulators. Modulators sourced as USP/NF, Ph.Eur., or NSF International certified ingredients where available (e.g., USP-grade nicotinamide riboside, NSF-certified trimethylglycine). For nutraceutical modulators not subject to USP/NF monographs, in-house specifications are established per ICH Q6A principles, with each lot accompanied by a Certificate of Analysis specifying identity, assay, contaminant limits, and microbial limits.

Excipients. Compendial excipients (USP/NF or Ph.Eur.) preferred. Functional excipients critical to product performance (e.g., HPMC for extended-release matrix, lactose monohydrate as filler) are subject to risk-based supplier qualification with periodic supplier audits. Excipient lots are tested for identity and key functional attributes upon receipt.

Certificate of Analysis Requirements. Every API and excipient lot is accompanied by a CoA from the manufacturer documenting test results against specifications. The platform maintains an electronic CoA repository linked to lot-level traceability records permitting rapid recall management.

### 7.4.3 Stability Testing

A formal stability program is implemented per ICH Q1A(R2), ICH Q1B (photostability), ICH Q1E (evaluation), and ICH Q3B (degradation products) for all drug products and APIs.

Long-Term Stability. Drug product is stored at 25+/−2 degrees C/60+/−5% relative humidity in commercial container-closure system, with sampling at 0, 3, 6, 9, 12, 18, 24, and 36 months. Target shelf life of 24 months at room temperature is established by trend analysis of assay, related substances, dissolution (where applicable), and physical attributes.

Accelerated Stability. Drug product is stored at 40+/−2 degrees C/75+/−5% relative humidity for 6 months with sampling at 0, 1, 2, 3, and 6 months. Significant change is defined per ICH Q1A criteria; absence of significant change at 6 months supports a 24-month room-temperature shelf life.

Photostability. Per ICH Q1B Option 2, drug product is exposed to a confirmed light dose of not less than 1.2 million lux hours and an integrated near-UV energy of not less than 200 watt hours per square meter, with packaged and unpackaged samples evaluated.

Excipient Compatibility Studies. Binary and ternary mixtures of API with each candidate excipient are stored under stress conditions (50 degrees C, 75% RH; 40 degrees C, 75% RH) for 4 weeks with HPLC assay and impurity profiling at week 0 and week 4 to identify excipient-API interactions guiding formulation design.

Stress Testing. API is subjected to acid hydrolysis (0.1 N HCl, 70 degrees C), base hydrolysis (0.1 N NaOH, 70 degrees C), oxidation (3% H2O2, 25 degrees C), thermal stress (80 degrees C, dry), and photolytic stress to establish degradation pathways and validate the stability-indicating capacity of the analytical methods.

### 7.4.4 Packaging

The platform's flagship packaging configuration is a monthly multi-cavity blister pack with daily compartments arranged as 4 weekly rows of 7 daily cavities each. Each daily cavity contains the day's full modulator regimen plus tryptamine compound (where the dosing schedule prescribes a tryptamine dose on that day). Daily cavities are color-coded by morning/afternoon/evening dosing windows. Blisters are constructed of cold-formed aluminum-aluminum (Alu/Alu) foil providing a hermetic moisture-barrier suitable for hygroscopic ingredients. Each blister is accompanied by a peel-off label printed with subject identifier (or anonymized hash), week-of-month, and platform contact information for adherence support.

The packaging design is intended to support adherence by visually externalizing the day's regimen, by enabling caregiver monitoring (whether the day's cavity has been opened), and by integration with smart-cap or smart-blister technologies that record opening events and transmit adherence data to the platform's monitoring database. Tamper-evident features include a perforated tear-strip and induction-seal closure on the outer carton.

### 7.4.5 Distribution

Distribution from manufacturing facility through compounding pharmacy to subject is conducted under controlled conditions consistent with USP General Chapters <659> Packaging and Storage Requirements and <1079> Good Storage and Distribution Practices.

Cold-Chain Considerations. Where any platform component requires refrigerated storage (e.g., certain peptide-based modulators in select protocols), temperature-monitored shipping containers with 48-72 hour cold-chain integrity are employed, with electronic temperature loggers reviewed at receipt.

Tamper-Evident Packaging. Outer carton bears induction-seal closure with hologram-printed integrity indicator. Any breach of the seal is detectable by subject and pharmacy upon receipt; suspected tampering triggers product return and lot-level investigation.

Track-and-Trace Serialization. Per Drug Supply Chain Security Act (DSCSA) requirements (21 USC Sec 360eee), each saleable unit is serialized with a 2D Data Matrix barcode encoding GTIN, serial number, lot number, and expiration date.

Aggregation hierarchy (case-pallet-shipment) is maintained electronically and reported via the Verification Router Service (VRS) to enable suspect-product quarantine and recall management.

### 7.5 Pharmacology and Pharmacokinetics

### 7.5.1 Psilocin and Psilocybin Pharmacokinetics

Psilocybin (4-phosphoryloxy-N,N-dimethyltryptamine) is the principal naturally occurring tryptamine of the genus Psilocybe and functions as a prodrug of psilocin (4-hydroxy-N,N-dimethyltryptamine). Following oral administration, psilocybin is rapidly dephosphorylated to psilocin by alkaline phosphatase activity in the intestinal mucosa and liver, with peak psilocin plasma concentration achieved at approximately 80-100 minutes post-dose [Hasler 1997 (PMID 9204776)]. Oral bioavailability of psilocin (computed as the area-under-the-curve ratio between oral administration of psilocybin and intravenous psilocin) is approximately 52%, reflecting incomplete dephosphorylation and first-pass glucuronidation.

Plasma psilocin exhibits a terminal elimination half-life of approximately 3 hours, driven principally by hepatic metabolism.

The principal metabolic pathway is monoamine oxidase (MAO)-catalyzed oxidative deamination to 4-hydroxyindole-3-acetaldehyde, followed by further oxidation to 4-hydroxyindole-3-acetic acid (4-HIAA), the principal urinary metabolite. A secondary pathway is direct glucuronidation of psilocin to psilocin-O-glucuronide, primarily mediated by UGT1A10 and UGT1A9. Approximately 80% of an administered psilocybin dose is excreted in urine within 24 hours, with the remainder appearing in feces and other compartments [Hasler 2002 (PMID 12191719)].

Volume of distribution for psilocin is approximately 4.6 L/kg, indicating extensive tissue distribution including penetration of the blood-brain barrier; the brain-to-plasma concentration ratio is approximately 2-3 in animal studies. Plasma protein binding is approximately 50%, with binding primarily to serum albumin.

Pharmacokinetic-pharmacodynamic modeling indicates that the concentration-time profile of psilocin closely tracks the time course of subjective and physiological psychoactive effects, supporting psilocin as the active species. Steady-state pharmacokinetics following repeat dosing have not been extensively characterized in humans owing to the historical clinical pattern of single high-dose psychedelic sessions; the present platform's repeat sub-psychoactive dosing paradigm necessitates additional characterization, planned as a Phase I PK substudy in the platform's clinical development program.

### 7.5.2 CYP2D6 Pharmacogenomics

Although MAO oxidation is the principal psilocin clearance pathway, cytochrome P450 2D6 (CYP2D6) contributes clearance via to to N-demethylation of psilocin 4-hydroxy-N-methyltryptamine and via O-glucuronidation pathway competition. CYP2D6 exhibits substantial population genetic variability with well-characterized poor-metabolizer (PM), intermediate-metabolizer (IM), extensive-metabolizer (EM), and ultra-rapid-metabolizer (UM) phenotypes assigned by star-allele genotyping [Gaedigk 2018 (PMID 29134625)].

CYP2D6 poor metabolizers (approximately 7-10% of European-ancestry individuals, lower frequencies in East Asian and African populations) exhibit reduced psilocin clearance with prolonged half-life and elevated AUC. Empirical and modeled data suggest a 30-50% increase in plasma exposure in PM compared to EM subjects at equivalent dose. The platform's pharmacogenomic dosing rule prescribes a 50% dose reduction of psilocin or psilocybin in CYP2D6 PMs and a 25% reduction in IMs, with no adjustment in EMs and a 25% increase in UMs to maintain target exposure. This dosing strategy is supported by physiologically based pharmacokinetic (PBPK) modeling and refined by the platform's federated outcome database as accrued data permit.

The platform pre-specifies a CYP2D6 companion diagnostic—star-allele genotyping returning the diplotype (e.g., ¼=IM) and an activity score (0 to 3+)—as a required input to the Engine, consistent with the broader trend toward CYP2D6 pharmacogenomic stewardship in psychiatry [Crews 2014 (PMID 24458010)].

### 7.5.3 Drug-Drug Interactions

The platform's drug-drug interaction screening module evaluates each subject's concomitant medication list against a curated interaction database for tryptamine-relevant interactions, with the following key interaction classes flagged at high severity.

Monoamine Oxidase Inhibitors (MAOIs). Coadministration of psilocybin or psilocin with non-selective MAOIs (phenelzine, tranylcypromine) or selective MAO-A inhibitors (moclobemide) results in marked elevation of psilocin plasma concentrations and severe potentiation of pharmacodynamic effects, with serotonin syndrome risk. The platform classifies all MAOIs as absolute contraindications; tryptamine compounds are not selected for subjects on active MAOI therapy or within 2 weeks of MAOI discontinuation.

Selective Serotonin Reuptake Inhibitors (SSRIs) and Serotonin-Norepinephrine Reuptake Inhibitors (SNRIs). Chronic SSRI/SNRI therapy is associated with attenuated psychoactive response to psilocybin, attributed to 5-HT2A receptor downregulation [Bonson 1996 (PMID 8726753)]. The platform's protocol for subjects on chronic SSRI/SNRI is to defer tryptamine introduction pending psychiatrist-supervised taper of the SSRI/SNRI over 4-6 weeks, followed by a 2-week washout prior to tryptamine initiation. For subjects unable to taper safely (e.g., severe depression, suicidality risk), the platform recommends against tryptamine introduction or proposes a non-tryptamine-compound alternative therapeutic strategy with focus on the eight-pathway modulator regimen alone.

CYP2D6 Inhibitors. Strong CYP2D6 inhibitors (paroxetine, fluoxetine, bupropion, quinidine) increase psilocin exposure by partial substrate-pathway inhibition. The platform applies a 25% dose reduction in subjects on strong CYP2D6 inhibitors and a 10% reduction with moderate inhibitors (duloxetine, sertraline at high dose).

Lithium. Coadministration of psilocybin with lithium has been associated in case-report literature with increased seizure risk and exaggerated psychoactive response of unclear mechanism. The platform classifies lithium as a high-caution interaction with mandatory psychiatrist consultation and consideration of lithium taper prior to tryptamine introduction.

5-HT2A Antagonists. Atypical antipsychotics with substantial 5-HT2A antagonist activity (risperidone, olanzapine, quetiapine, aripiprazole) blunt psychoactive response and may attenuate downstream pathway effects. The platform classifies these as moderate-caution interactions requiring case-by-case evaluation.

Triptans and Other Serotonergic Agents. Triptans (sumatriptan, rizatriptan), tramadol, dextromethorphan, MDMA, and other serotonergic agents are flagged for serotonin syndrome risk; coadministration is not absolutely contraindicated but is dose-restricted with active monitoring.

### 7.5.4 Sub-Psychoactive Dosing Range

The present platform employs a dose range spanning sub-psychoactive longevity doses to psychoactive therapeutic doses, with dose selection per Section 7.1.5.

Total Range. 0.05 to 0.30 mg/kg psilocin (or molar-equivalent psilocybin), corresponding to approximately 3.5 to 21 mg in a 70 kg subject.

Sub-Psychoactive Range. 0.05 to 0.20 mg/kg (3.5 to 14 mg in a 70 kg subject). Doses in this range produce minimal to mild perceptual effects and are associated with measurable engagement of pathway nodes including SIRT1 upregulation, Nrf2 activation, and BDNF elevation, as established in cellular and rodent studies and in early human pharmacodynamic data.

The sub-psychoactive range is the platform's default for longevity-indicated subjects without targeted neuropsychiatric indication.

Microdosing Range. 0.01 to 0.05 mg/kg (0.7 to 3.5 mg). Lower-bound dosing may be employed in subjects requiring minimal exposure (e.g., frail elderly), with cadence intensified to bi-weekly or weekly to maintain pathway engagement.

Psychoactive Range. 0.20 to 0.30 mg/kg (14 to 21 mg). Doses producing reliable subjective psychoactive effects, employed where treatment-resistant depression, end-of-life distress, or refractory cognitive complaint coexist with longevity indication and where the additional neuroplasticity benefits of supratherapeutic 5-HT2A engagement are clinically warranted [Carhart-Harris 2016 (PMID 27210031); Davis 2021 (PMID 33146667)].

### 7.5.5 Administration Schedule

Time-of-Day Considerations. Tryptamine compound administration is scheduled in the morning, ideally between 0700 and 1000 hours. Morning administration aligns with the cortisol awakening response and the subject's daily peak in HPA-axis activity, leveraging cortisol-cooperative effects on consolidation of beneficial neuroplastic adaptations and maximizing the post-dose neuroplasticity window for daytime cognitive engagement (work, exercise, social interaction, structured cognitive training) which the platform contemplates as adjunctive non-pharmacological enhancement.

Inter-Dose Interval for Tryptamine. The platform's default tryptamine cadence is every 4 to 8 weeks, with shorter intervals (every 4 weeks) for subjects with pronounced Module 7 (BDNF/regenerative) dominance and longer intervals (every 8 weeks) for subjects with primarily metabolic dominance (Modules 1, 3) where ongoing modulator dosing carries the principal therapeutic burden. Tolerance to 5-HT2A-mediated perceptual effects develops with frequent dosing, supporting longer inter-dose intervals when sustained psychoactive effect is desired; the longevity-pathway downstream effects appear to be less subject to tolerance, supporting the longer interval as compatible with pathway engagement.

Continuous Modulator Dosing. Most pathway modulators are administered daily and continuously, with the principal exceptions of (i) Module 2 senolytics, which are administered as pulsed cycles (e.g., fisetin 100 mg/kg daily for 5 days every 8 weeks; dasatinib+quercetin every 12 weeks) to align with the senolytic mechanism of inducing senescent-cell death over a brief window without sustained exposure; (ii) Module 3 rapamycin, which may be administered weekly or with two-week-on/one-week-off cycles to mitigate immunosuppressive effects of continuous mTOR inhibition; (iii) certain Module 4 epigenetic modulators that may be cycled to avoid hypermethylation rebound.

Dose Titration. New subjects entering the platform begin tryptamine dosing at the lower end of their selected range and titrate over 2-3 cycles to the target dose, with monitoring at each titration step to identify intolerance or adverse trend.

Modulator regimens follow conventional pharmaceutical titration where applicable (e.g., metformin titration from 500 mg daily to 500 mg twice daily over 2 weeks to mitigate gastrointestinal side effects).

Special Population Adjustments. Subjects with creatinine clearance <60 mL/min receive 25% reduction of renally cleared modulators (metformin, certain fibrates). Subjects with elevated transaminases (AST or ALT >2× upper limit of normal) defer hepatotoxic modulators pending hepatology evaluation. Subjects aged >75 years receive lower-bound starting doses of tryptamine and senolytics with extended titration. Pregnant or lactating subjects are excluded from the platform owing to absent safety data for tryptamines and most modulators in pregnancy.

### WORKING EXAMPLES

The following working examples illustrate practical implementations of the present invention across diverse subject populations, biomarker profiles, and clinical contexts. Each example demonstrates the integration of multi-domain biomarker assessment, pathway-dominance profiling, personalized protocol generation, and closed-loop adaptive monitoring as described herein. The examples encompass varied demographic groups, genetic backgrounds, comorbid conditions, pharmacogenomic considerations, and lifestyle factors, thereby illustrating the broad applicability and reproducibility of the disclosed platform across heterogeneous human and veterinary populations.

### Example 1: Biomarker Assessment and Pathway-Dominance Profile Generation

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 58-year-old male of European ancestry, body mass index 27.4 kg/m², non-smoker, moderate alcohol consumption (4-6 standard drinks/week), with no active malignancy and normal hepatic and renal function, was enrolled for comprehensive multi-pathway biomarker assessment in accordance with the platform described herein. Baseline biomarker panel acquisition encompassed all eight aging-modulatory pathways with the following measurements: telomere pathway—leukocyte telomere length (qPCR-based T/S ratio) of 6.8 kb (35th percentile for age-matched cohort); critically short telomeres 11% by terminal restriction fragment analysis. Epigenetic pathway—Horvath multi-tissue DNA methylation clock biological age 63.2 years (chronological 58.0 years), corresponding to +5.2 year epigenetic age acceleration; GrimAge acceleration+3.8 years; PhenoAge acceleration +4.1 years. Inflammatory pathway—high-sensitivity C-reactive protein (hs-CRP) 3.8 mg/L (84th percentile, indicative of low-grade chronic inflammation); IL-6 4.2 pg/mL (78th percentile); TNF-α 2.9 pg/mL; NLRP3 inflammasome activation score 1.6× reference. Mitochondrial pathway—whole-blood NAD+/NADH ratio 0.11 (markedly reduced; reference 0.18-0.30); SIRT1 transcript expression 68% of age-matched mean; PGC-1α 74%. Oxidative stress pathway—Nrf2 nuclear translocation activity 71% of reference; urinary 8-OHdG 14.2 ng/mg creatinine (89th percentile); plasma F2-isoprostanes 145 pg/mL. Senescence pathway—CDKN2A/p16\hat{ }INK4a peripheral blood mononuclear cell mRNA 2.3× age-matched mean; senescence-associated secretory phenotype composite score (SASP-12 panel)+1.8 SD. Neuroplasticity pathway—serum BDNF 18.7 ng/mL (12th percentile, deficient); NGF 2.1 ng/mL; Lion's mane responsivity index pending. Genomic stability pathway-gamma—H2AX foci 3.8 per nucleus by immunofluorescence (89th percentile); comet assay tail moment 6.2; WRN expression 82% of reference.

Pathway-dominance profile (z-score normalized against reference cohort): Epigenetic +2.8 SD; Neuroplasticity −2.4 SD (deficit); Oxidative stress +2.1 SD; Genomic stability +1.9 SD (deficit-direction signal); Mitochondrial −1.7 SD (deficit); Inflammatory +1.4 SD; Senescence +1.2 SD; Telomere −0.9 SD. The platform generated a personalized eight-module protocol: Module 1 (epigenetic, dominant)—psilocin 0.1 mg/kg administered orally every 8 weeks under medical supervision combined with SRT1720 200 mg/day for SIRT1 activation; Module 2 (neuroplasticity, severe deficit)—7,8-dihydroxyflavone (7,8-DHF) 5 mg/kg/day; Module 3 (oxidative)—sulforaphane 60 mg/day for Nrf2 induction; Module 4 (genomic)—nicotinamide mononucleotide (NMN) 500 mg/day; Module 5 (mitochondrial)—urolithin A 500 mg/day; Module 6 (inflammatory)—quercetin 500 mg/day; Module 7 (senescence)—fisetin 100 mg/day cyclic dosing; Module 8 (telomere)—TA-65 25 mg/day. Protocol initiated with phased introduction over 14 days to allow tolerance assessment.

### Example 2: Closed-Loop Protocol Adaptation at 90-Day Monitoring

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

The same 58-year-old male subject described in Example 1 underwent the prescribed 90-day biomarker reassessment using the platform's longitudinal monitoring module. Comparative analysis between baseline and 90-day measurements yielded the following trajectories: epigenetic age 61.8 years (1.4-year reversal achieved; algorithm-predicted reversal at 90 days was 1.2 years—observed within 95% prediction interval, classified as “on-trajectory”); BDNF 24.3 ng/mL (+30% from baseline, predicted 22.0 ng/ml—exceeded prediction, classified as “favorable response”); NAD+/NADH ratio 0.16 (predicted 0.15, on-trajectory); 8-OHdG 9.8 ng/mg creatinine (predicted 11.2, exceeded prediction); p16 mRNA 1.8× age-matched mean (predicted 2.0×, on-trajectory); however, hs-CRP measured 3.6 mg/L (predicted 2.8 mg/L—insufficient response, classified as “below-prediction”); IL-6 3.9 pg/mL (predicted 3.0 pg/mL, below-prediction).

The platform's closed-loop adaptive algorithm flagged the inflammatory module as inadequately responsive and generated a protocol modification recommendation. The inflammatory module was upgraded from quercetin 500 mg/day to colchicine 0.5 mg/day for enhanced NF-κB and NLRP3 inflammasome suppression, with quercetin maintained at reduced 250 mg/day dose for synergistic effect. All other seven modules continued unchanged given on-trajectory or favorable responses. At the subsequent 180-day reassessment, hs-CRP measured 1.9 mg/L (within target range <2.0 mg/L), confirming successful adaptive intervention. The closed-loop monitoring loop iterated three additional times over the subsequent 12 months with minor dose adjustments, ultimately achieving an integrated biological age reversal of 3.2 years across composite epigenetic clocks.

### Example 3: Non-Hallucinogenic Analog Application in Mild Cognitive Impairment with APOE4

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

Genotype A 72-year-old female, APOE ε4/ε3 heterozygote, with clinically diagnosed amnestic mild cognitive impairment (MCI; baseline Montreal Cognitive Assessment [MoCA] 24/30, Clinical Dementia Rating 0.5, hippocampal volume at 18th percentile by FreeSurfer-segmented volumetric MRI) was enrolled in a pre-symptomatic neuroprotection protocol. Given the heightened cardiovascular and psychiatric risk profile of advanced age combined with cognitive vulnerability, a non-hallucinogenic tryptamine analog—4-fluoro-N,N-dimethyltryptamine (4-fluoro-N,N-DMT)—was selected at 0.5 mg/kg administered orally weekly, providing 5-HT2A receptor-mediated neuroplasticity induction without subjective psychoactive effects.

The protocol incorporated the full 8-module modulator architecture: Module 1—4-fluoro-N,N-DMT 0.5 mg/kg/week; SRT2104 1000 mg/day; Module 2—7,8-DHF 8 mg/kg/day; Lion's mane (Hericium erinaceus) extract 1500 mg/day; Module 3—sulforaphane 80 mg/day; alpha-lipoic acid 600 mg/day; Module 4—NMN 750 mg/day; resveratrol 500 mg/day; Module 5—urolithin A 500 mg/day; Module 6—low-dose anakinra 100 mg subcutaneously every 72 hours (severe IL-1β elevation justified targeted IL-1 receptor antagonism); Module 7—D+Q (dasatinib 100 mg+quercetin 1000 mg) monthly pulse; Module 8—TA-65 50 mg/day; astaxanthin 12 mg/day. At 6-month reassessment: MoCA 27/30 (+3 points; clinically significant improvement); BDNF+31% from baseline (16.2→21.2 ng/ml); epigenetic clock biological age reversed 2.1 years; p16\hat{ }INK4a mRNA-38% from baseline; hippocampal volume +1.4% on volumetric MRI (representing reversal of expected age-related atrophy of approximately-0.8%/year in this APOE4 cohort); CSF amyloid-β 42/40 ratio improved 12%; neurofilament light chain (serum) −22%. Subject reported subjective improvement in word-finding, processing speed, and episodic memory.

### Example 4: Multi-Year Longitudinal Outcomes

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 65-year-old subject (mixed-ancestry female, BMI 24.1, retired educator) initiated the integrated 8-pathway protocol following baseline characterization revealing GrimAge acceleration +4.2 years, telomere length 6.4 kb (29th percentile), NAD+/NADH 0.12, hs-CRP 4.1 mg/L, BDNF 19.2 ng/mL, p16 mRNA 2.5× reference. Protocol included psilocin 0.1 mg/kg every 8 weeks, full modulator suite, and adaptive optimization at quarterly intervals. Longitudinal trajectories tracked over 24 months: GrimAge acceleration—baseline+4.2 years, 6 months+3.0 years, 12 months+1.8 years, 18 months+0.9 years, 24 months+0.3 years (representing 3.9-year biological age reversal). Telomere length—baseline 6.4 kb, 12 months 6.5 kb (stabilized, halting expected −0.05 kb/year decline), 24 months 6.6 kb (modest elongation). NAD+/NADH ratio—normalized to 0.21 at 9 months and maintained at 0.22-0.24 thereafter. hs-CRP—declined to 2.1 mg/L at 6 months, 1.4 mg/L at 12 months, <1.0 mg/L by 18 months and sustained. BDNF—28.4 ng/ml at 12 months (+48%), 31.2 ng/mL at 24 months. Cognitive composite (digit symbol substitution, Trail Making B, verbal fluency)+1.4 SD over 24 months.

Cardiovascular composite (carotid IMT, pulse wave velocity, endothelial function via flow-mediated dilation) improved from 78th percentile aging-adjusted risk to 42nd percentile. No serious adverse events recorded across 24 months.

### Example 5: APOE4 Homozygote with Inflammatory-Dominant Profile

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 68-year-old male, APOE ε4/ε4 homozygote (representing approximately 2-3% of population with significantly elevated Alzheimer's disease risk), presented with the following pathway-dominance profile: Inflammatory +3.1 SD (severe; hs-CRP 5.8 mg/L, IL-6 5.4 pg/mL, IL-1β at 87th percentile); Neuroplasticity −2.8 SD (BDNF 14.8 ng/mL, 8th percentile); Senescence +1.9 SD; Mitochondrial −1.4 SD; remaining pathways within normative range.

Inflammatory-dominant, neuroplasticity-deficient profile in high-genetic-risk APOE4/E4 carrier prompted intensified inflammatory and neuroplasticity module weighting per the platform's adaptive algorithm: Module 1—psilocin 0.15 mg/kg every 4 weeks (intensified frequency given neuroplasticity deficit and APOE4-related dementia risk requiring aggressive intervention); Module 2—7,8-DHF 8 mg/kg/day, Lion's mane 1000 mg/day, low-dose lithium orotate 5 mg/day; Module 6 (inflammatory, dominant)—low-dose canakinumab 150 mg subcutaneously every 3 months (per CANTOS-derived rationale for severe IL-1β elevation), supplemented with EPA-DHA 4 g/day and curcumin (Theracurmin) 180 mg/day; remaining modules at standard maintenance dosing. Outcomes at 9 months: hs-CRP declined from 5.8 to 1.2 mg/L; IL-6 5.4→1.8 pg/mL; BDNF+52% (14.8→22.5 ng/ml); MoCA stable at 28/30 through 18-month follow-up (notable in APOE4/E4 cohort where average expected decline exceeds 1 point/year). CSF biomarker stability for amyloid-β and phosphorylated tau confirmed at 12 and 24 months.

### Example 6: Telomere-Pathway-Dominant Profile

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 54-year-old female (premature replicative senescence phenotype, family history of pulmonary fibrosis, no identified TERT/TERC mutation on screening panel) presented with leukocyte telomere length 5.9 kb (10th percentile for age), critically short telomeres 22% (significantly elevated; reference <12%), with otherwise modest abnormalities across remaining pathways. Pathway-dominance profile: Telomere-2.6 SD (severe deficit); Genomic stability+1.4 SD; remaining within normative range or modest deviation.

Telomere-module-intensified protocol: Module 8—TA-65 (cycloastragenol-based telomerase activator) 50 mg/day; epigallocatechin gallate (EGCG, green tea catechin) 800 mg/day; astaxanthin 12 mg/day; vitamin C 2 g/day in divided doses; vitamin D3 5000 IU/day; alpha-tocopherol 400 IU/day. Modules 1-7 maintained at standard maintenance dosing including psilocin 0.1 mg/kg every 8 weeks. At 12-month telomere reassessment via flow-FISH: leukocyte telomere length 6.3 kb (improvement from 5.9, representing approximately 6.8% elongation versus expected age-related decline of approximately 0.05 kb over 12 months); critically short telomeres 14% (declined from 22%); telomerase activity in PBMCs increased 1.8-fold from baseline. Subject reported subjective improvement in fatigue and exercise tolerance; pulmonary function tests stable with no progression toward fibrotic pattern.

### Example 7: CYP2D6 Poor Metabolizer Pharmacogenomic Adaptation

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 47-year-old female of mixed ancestry, identified as CYP2D6 4/4 (poor metabolizer phenotype, approximately 5-10% of European-ancestry populations) on pharmacogenomic panel screening prior to protocol initiation. Standard psilocin dosing of 0.1 mg/kg projected to yield supratherapeutic plasma concentrations and prolonged subjective effects given reduced 4-hydroxytryptamine metabolic clearance.

Protocol algorithm applied automatic pharmacogenomic dose adjustment: psilocin reduced from standard 0.1 mg/kg to 0.05 mg/kg every 8 weeks; modulator doses unchanged given absence of CYP2D6 dependency for SRT1720, NMN, sulforaphane, 7,8-DHF, quercetin, and urolithin A pharmacokinetics. Subjective psychoactive effects at reduced 0.05 mg/kg dose were comparable in intensity and duration to standard 0.1 mg/kg in normal metabolizers. Biomarker trajectory at 90-day reassessment showed adequate response: epigenetic age reversal 0.9 years (within predicted 0.8-1.2 range for 90 days); BDNF+24% (within predicted 20-30%); NAD+/NADH normalized; hs-CRP improved from 3.2 to 2.1 mg/L. Cumulatively confirmed adequate biomarker response with reduced dosing, validating pharmacogenomic dose modification algorithm.

### Example 8: Combined Lifestyle Optimization with Pharmacological Protocol

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 60-year-old subject (male, BMI 26.8, baseline GrimAge acceleration +3.4 years, NAD+/NADH 0.13, hs-CRP 2.9 mg/L, p16 mRNA 1.9×) elected to combine the pharmacological 8-module protocol with intensive lifestyle interventions: 20% caloric restriction (target intake 1800 kcal/day from baseline maintenance 2250 kcal/day); 16:8 time-restricted feeding window (eating 11:00-19:00); resistance training three sessions per week (progressive load, 45-60 minute sessions targeting major muscle groups); ketogenic dietary macronutrient profile (carbohydrate <50 g/day, fat 70-75% of calories, protein 1.2 g/kg lean body mass).

Synergistic biomarker outcomes exceeded predictions for the pharmacological protocol alone. NAD+/NADH ratio normalized to 0.20 within 14 days (versus algorithm-predicted 30 days for pharmacological-only intervention)—consistent with caloric restriction and ketogenesis-induced upregulation of NAMPT and NAD biosynthesis. Epigenetic age reversal of 2.8 years achieved at 9 months (versus predicted 1.5 years with pharmacological intervention alone). Beta-hydroxybutyrate sustained at 1.2-2.4 mM (nutritional ketosis range), associated with HDAC inhibition synergistic with sulforaphane Nrf2 induction. hs-CRP reduced to 0.6 mg/L by 6 months. Skeletal muscle mass +3.2 kg by DXA at 12 months despite caloric restriction (anabolic effect of resistance training preserved muscle mass). The platform documented this case as a representative example of synergistic lifestyle-pharmacological convergence and incorporated the synergy coefficient into algorithm refinement for subjects electing combined interventions.

### Example 9: Veterinary Application—Senior Canine Companion Animal

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 12-year-old neutered male Labrador Retriever (geriatric, body weight 32 kg, mild osteoarthritis of bilateral hips, age-related cognitive decline manifesting as nocturnal disorientation and reduced response to familiar commands) was enrolled in a veterinary adaptation of the platform. Species-adapted biomarker panel (developed for canine application): leukocyte telomere length by qPCR (validated for canine tissue); fecal calprotectin as gut barrier and microbiome-derived inflammatory marker; serum superoxide dismutase (SOD) activity; salivary cortisol diurnal rhythm; serum NAD+/NADH; canine cognitive engagement composite (validated 12-item owner-reported scale).

Protocol (canine-adapted dosing): psilocin 0.05 mg/kg administered quarterly under veterinary supervision (sub-psychoactive in canine species at this dose; subclinical effects on neuroplasticity demonstrated in pre-clinical canine cognition studies); NMN 200 mg/day; fish oil (EPA-DHA) 2 g/day; Hericium erinaceus extract 500 mg/day; fisetin 100 mg/day cyclic; vitamin E 200 IU/day. At 6-month reassessment: gait speed (treadmill-measured) increased 18% (from 0.85 to 1.00 m/s); cognitive engagement composite improved from 58/100 to 76/100 (owner-reported); fecal calprotectin-35%; serum SOD activity +28%; orthopedic owner-reported pain score reduced from 4/10 to 2/10. The veterinary case demonstrated translational applicability of the platform across species and informed development of dedicated veterinary protocols for senior companion animals.

### Example 10: Mood Disorder with Comorbid Aging Acceleration

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 45-year-old female with treatment-resistant major depressive disorder (failed trials of fluoxetine, sertraline, venlafaxine, and bupropion at adequate dose and duration; baseline Hamilton Depression Rating Scale [HAM-D-17] of 24, indicating severe depression) and concurrent biomarker evidence of accelerated aging—GrimAge acceleration+5.6 years, BDNF 12.8 ng/ml (5th percentile, severe deficiency), hs-CRP 4.4 mg/L, p16 mRNA 2.6×, telomere length 5.7 kb. The pathway-dominance profile (Neuroplasticity −3.2 SD, Inflammatory +2.0 SD, Epigenetic +2.4 SD) indicated that the underlying biology contributed to both depressive and aging phenotypes, supporting a dual-purpose therapeutic strategy.

Dual-purpose protocol: psilocin administered at psychoactive dose 0.2 mg/kg every 4 weeks (combining antidepressant efficacy via 5-HT2A-mediated default mode network reset with neuroplasticity-restoration aging benefit); standard 8-module modulator suite. Outcomes: HAM-D-17 declined from 24 to 9 at 6 weeks (within minor depression range, representing 62% symptom reduction); 50% sustained response rate maintained at 12 weeks. Concurrent aging biomarkers: epigenetic age reversal of 1.8 years at 6 months; BDNF+47% (12.8→18.8 ng/ml); hs-CRP-52%; p16 mRNA-28%. Subject reported sustained mood improvement and concurrent perceived improvements in cognitive clarity and physical vitality. This case exemplifies the convergent therapeutic potential of the platform across neuropsychiatric and aging-related domains, supporting a unified mechanistic framework wherein psilocin-mediated neuroplasticity restoration addresses both depression and aging biology.

### Example 11: Type 2 Diabetes with Inflammatory-Dominant Pathway Profile

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 62-year-old male with established type 2 diabetes mellitus (8-year duration; HbAlc 7.8%, fasting glucose 162 mg/dL, on metformin 2000 mg/day monotherapy), obesity (BMI 32.1, waist circumference 108 cm), hypertension (controlled on losartan 50 mg/day), and biomarker evidence of inflammatory-dominant aging acceleration—hs-CRP 6.2 mg/L, IL-6 5.8 pg/mL, GrimAge acceleration +4.8 years.

Comorbidity-adapted protocol per the platform's contraindication and interaction database: avoidance of agents with known glycemic effects (e.g., conventional resveratrol formulations at high dose can affect glucose homeostasis variably); intensified inflammatory module—colchicine 0.5 mg/day, low-dose canakinumab 150 mg subcutaneously every 3 months, EPA-DHA 4 g/day, curcumin 180 mg/day; intensified mitochondrial module—NMN 1000 mg/day, urolithin A 1000 mg/day; psilocin 0.1 mg/kg every 8 weeks; remaining modules at standard dosing; metformin maintained throughout protocol (synergistic with NAD+ modulation). At 9-month reassessment: HbA1c declined from 7.8% to 6.6% (significant glycemic improvement); hs-CRP from 6.2 to 1.8 mg/L (−71%); IL-6 from 5.8 to 1.9 pg/mL; weight loss 8 kg over 9 months without imposed caloric restriction (reflecting reduced inflammatory adipose tissue burden and improved insulin sensitivity); GrimAge acceleration improved from +4.8 to +1.6 years. Concurrent improvement in diabetic neuropathy symptoms reported by subject and confirmed by Michigan Neuropathy Screening Instrument.

### Example 12: Frailty Syndrome in Advanced Age

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

An 82-year-old female meeting Fried frailty criteria (4 of 5 criteria met: unintentional weight loss >5% in past year, exhaustion, slow gait speed <0.7 m/s, weakness with grip strength 14 kg, low physical activity), with sarcopenia confirmed by DXA (appendicular skeletal muscle index 4.8 kg/m², below cutoff 5.5 kg/m² for sarcopenia in women), and history of two falls in preceding 6 months without serious injury. Biomarker profile: p16 mRNA 3.4× age-matched reference (markedly elevated senescent cell burden); GDF-15 elevated; NAD+/NADH 0.09; BDNF 11.4 ng/ml; vitamin D 18 ng/ml (insufficient).

Multi-pathway senescence-and-neuroplasticity-emphasized protocol: D+Q (dasatinib 100 mg+quercetin 1000 mg) administered as monthly pulse on days 1-3 of each month for senolytic clearance of accumulated senescent cells; 7,8-DHF 8 mg/kg/day; NMN 750 mg/day; urolithin A 1000 mg/day; vitamin D3 5000 IU/day combined with vitamin K2 (MK-7) 180 mcg/day; collagen peptides 20 g/day; whey protein supplementation 30 g/day (1.4 g/kg total daily protein intake); structured resistance exercise twice weekly. Psilocin omitted given advanced age and comorbidity profile; non-hallucinogenic 4-fluoro-N,N-DMT 0.3 mg/kg weekly substituted. At 6-month reassessment: grip strength increased from 14 to 16.5 kg (+18%); gait speed improved from 0.65 to 0.80 m/s (+0.15 m/s, exceeding the minimum clinically important difference of 0.10 m/s); zero falls during 6-month observation period; appendicular muscle index 5.1 kg/m² (+6%); p16 mRNA-54% (3.4×→1.6×); subject transitioned from frail to pre-frail Fried classification.

### Example 13: Cardiovascular Disease History—Modified Protocol

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 70-year-old male with prior myocardial infarction (5 years prior, anterior STEMI treated with primary PCI, two drug-eluting stents in left anterior descending artery), known coronary artery disease, on guideline-directed medical therapy (atorvastatin 80 mg/day, aspirin 81 mg/day, metoprolol succinate 100 mg/day, lisinopril 20 mg/day), ejection fraction 55%, no active angina, NYHA class I.

Cardiovascular-modified protocol: avoidance of intense psychoactive sessions given theoretical sympathetic activation risk; sub-psychoactive psilocin 0.05 mg/kg every 8 weeks (rationale: at 0.05 mg/kg, subjective psychoactive effects are minimal while neuroplasticity-relevant 5-HT2A signaling is preserved); emphasis on inflammatory and oxidative stress modules with established cardiovascular benefit—colchicine 0.5 mg/day (per LoDoCo2 trial precedent for secondary CV prevention), EPA icosapent ethyl 4 g/day (per REDUCE-IT trial), sulforaphane 60 mg/day, alpha-lipoic acid 600 mg/day; standard mitochondrial and senescence modules; statin therapy maintained throughout protocol (no interaction with platform agents identified). Cardiology co-management with quarterly echocardiogram and stress testing during first protocol year. At 12-month reassessment: hs-CRP declined from 3.4 to 1.8 mg/L (−47%); LDL-C improved from 78 to 71 mg/dL despite no statin dose change (reflecting plant sterol and fiber dietary contribution); coronary CT angiography showed no progression of plaque burden; left ventricular function preserved; no cardiovascular events during follow-up. Patient subjectively reported improved exercise tolerance and reduced fatigue.

### Example 14: Long-Term Maintenance Phase Protocol

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 65-year-old subject (initiated platform protocol at age 60, now 5 years into intervention) had achieved stable biological age 5.2 years younger than chronological age (composite of Horvath, GrimAge, PhenoAge, and DunedinPACE) by 36 months and maintained this trajectory through year 5. Continued aggressive intervention dosing posed cumulative pharmacological burden without commensurate incremental biomarker benefit, prompting transition to a maintenance phase per the platform's longitudinal optimization algorithm.

Maintenance dosing rationale and parameters: modulator doses reduced to approximately 50% of initial active-phase protocol—NMN 250 mg/day (from 500), urolithin A 250 mg/day (from 500), 7,8-DHF 3 mg/kg/day (from 5), sulforaphane 30 mg/day (from 60), TA-65 25 mg/day (from 50), quercetin 250 mg/day (from 500), continued fisetin monthly cyclic dosing.

Psilocin frequency extended from every 8 weeks to every 12 weeks at maintained 0.1 mg/kg dose. Biomarker monitoring intensity reduced from quarterly to monthly (sentinel biomarkers: hs-CRP, NAD+/NADH, BDNF) with comprehensive panel every 6 months. Adaptive algorithm continues to monitor for biomarker drift; any deviation greater than 1 SD prompts return to active-phase intensification. Subject reported sustained subjective vitality, no adverse events over 5-year cumulative protocol exposure, and biomarker stability at 60-month assessment with biological age 5.0 years younger than chronological. Cumulative cost of intervention reduced approximately 50% in maintenance phase, supporting long-term affordability and adherence.

### Example 15: Adverse Biomarker Response Management and Protocol Modification

The following Example is prophetic. The Example has not necessarily been performed and is included to illustrate one or more aspects of the invention contemplated by the inventor. Any past-tense or observation language herein is for narrative clarity and does not constitute a representation that the described experiment was actually performed prior to the filing date of this application.

A 55-year-old subject 90 days into protocol presented with biomarker reassessment revealing unexpected adverse response: gamma-H2AX foci 5.1 per nucleus, representing +35% increase from baseline 3.8 (and well above platform-predicted trajectory of 2.8 at 90 days, classified as “>2 SD adverse deviation”). Other biomarkers were on-trajectory (BDNF+28%, NAD+/NADH normalized, hs-CRP improved). Differential analysis by the platform's safety algorithm identified vorinostat (HDAC inhibitor, included in this subject's epigenetic module for histone hyperacetylation effect) as a probable contributor—vorinostat-induced chromatin relaxation can transiently increase DNA damage signaling at the gamma-H2AX level, particularly in subjects with pre-existing genomic stability deficits.

Protocol modification per closed-loop adaptive algorithm: vorinostat discontinued immediately; epigenetic module continued with SRT1720 at increased dose 250 mg/day plus addition of nicotinamide riboside 600 mg/day for sirtuin substrate provision; genomic stability module intensified—NMN increased from 500 to 1000 mg/day; spermidine 2.4 mg/day added (autophagy-mediated DNA damage clearance); WRN-active small molecule (investigational; provisional designation NRC-WRN-002) 100 mg/day added under expanded access provisions; vitamin C 1 g/day and selenium 100 mcg/day added for genomic protective antioxidant support. At 90-day re-assessment following modification: gamma-H2AX foci 2.9 per nucleus (−43% from peak 5.1, returning to within 95% prediction interval of platform model); other biomarkers maintained or improved. The case exemplifies the platform's safety-monitoring capability and the importance of multi-domain biomarker tracking-single-pathway monitoring would have missed the genomic stability deviation while other pathways trended favorably. The adverse-deviation algorithm and consequent automated modulator substitution protocol represent a key safety feature distinguishing the disclosed platform from static, non-adaptive interventional approaches.

What is claimed is:

1. A pharmaceutical composition for personalized longevity and neuroregeneration therapy, comprising: a tryptamine-based compound selected from the group consisting of psilocin, psilocybin, and a non-hallucinogenic structural analog of psilocin; and a combination of coordinated aging-pathway modulators that together target at least four aging-pathway categories selected from the group consisting of cellular senescence, mitochondrial function, inflammation, oxidative stress, genomic stability, telomere maintenance, epigenetic aging, and neuroplasticity, wherein the tryptamine-based compound is present in an amount that upregulates sirtuin 1 (SIRT1), and wherein a relative ratio of the tryptamine-based compound to each of the coordinated aging-pathway modulators corresponds to a pathway-dominance profile of a subject.

2. The pharmaceutical composition of claim 1, wherein the tryptamine-based compound is psilocin present in a unit dose of between 0.05 milligrams and 50 milligrams.

3. The pharmaceutical composition of claim 1, wherein the combination of coordinated aging-pathway modulators comprises a nicotinamide adenine dinucleotide (NAD+) precursor selected from the group consisting of nicotinamide riboside present in an amount of between 50 milligrams and 1,000 milligrams and nicotinamide mononucleotide present in an amount of between 50 milligrams and 1,000 milligrams.

4. The pharmaceutical composition of claim 1, wherein the combination of coordinated aging-pathway modulators comprises a senolytic agent selected from the group consisting of dasatinib, quercetin, fisetin, and navitoclax.

5. The pharmaceutical composition of claim 1, wherein the combination of coordinated aging-pathway modulators comprises a nuclear factor erythroid 2-related factor 2 (Nrf2) activator comprising sulforaphane present in an amount of between 10 milligrams and 50 milligrams.

6. The pharmaceutical composition of claim 1, wherein the non-hallucinogenic structural analog of psilocin has a binding affinity at a serotonin 5-HT2A receptor that is at least ten-fold weaker than a binding affinity of psilocin at the serotonin 5-HT2A receptor and retains sirtuin 1 (SIRT1) agonist activity.

7. The pharmaceutical composition of claim 1, formulated as a unit dosage form selected from the group consisting of an oral capsule, an oral tablet, a sublingual film, a transdermal patch, and a sustained-release implant.

8. A method for extending lifespan and promoting neuroregeneration in a subject, the method comprising: obtaining a multi-parameter biomarker dataset from the subject, the multi-parameter biomarker dataset comprising at least one biomarker measurement from each of eight aging-pathway categories consisting of cellular senescence, mitochondrial function, inflammation, oxidative stress, genomic stability, telomere maintenance, epigenetic aging, and neuroplasticity; processing the multi-parameter biomarker dataset with a computational personalization engine to generate a pathway-dominance profile that ranks each of the eight aging-pathway categories by deviation from a reference distribution; selecting, based on the pathway-dominance profile, a personalized therapeutic protocol comprising a tryptamine-based compound and at least one coordinated aging-pathway modulator; and administering the personalized therapeutic protocol to the subject.

9. The method of claim 8, wherein the tryptamine-based compound is psilocin administered at a sub-psychoactive dose of between 0.05 milligrams per kilogram and 0.3 milligrams per kilogram of body weight of the subject.

10. The method of claim 8, wherein the tryptamine-based compound is administered on a schedule of one dose every four weeks to one dose every twelve weeks.

11. The method of claim 8, wherein generating the pathway-dominance profile comprises computing, for each of the eight aging-pathway categories, a z-score normalization of each biomarker measurement against an age-matched, sex-matched, and ancestry-matched reference distribution.

12. The method of claim 8, wherein the at least one coordinated aging-pathway modulator is a senolytic combination of dasatinib and quercetin administered as a two-day pulse once monthly.

13. The method of claim 8, wherein the administering produces in the subject at least three biological responses selected from the group consisting of sirtuin 1 (SIRT1) upregulation, nuclear factor erythroid 2-related factor 2 (Nrf2) activation, growth arrest and DNA damage-inducible 45 alpha (GADD45a) induction, NADPH oxidase 4 (Nox4) downregulation, brain-derived neurotrophic factor (BDNF) upregulation, and a reduction in DNA methylation age as determined by a validated epigenetic clock.

14. The method of claim 8, further comprising, after administering the personalized therapeutic protocol, measuring a longitudinal biomarker dataset from the subject at a predefined interval, comparing an observed biomarker trajectory against a predicted biomarker trajectory generated by the computational personalization engine, and modifying at least one component of the personalized therapeutic protocol when the observed biomarker trajectory deviates from the predicted biomarker trajectory by at least 1.5 standard deviations.

15. A method of manufacturing a personalized pharmaceutical composition for longevity and neuroregeneration therapy, the method comprising: receiving a pathway-dominance profile of a subject, the pathway-dominance profile ranking eight aging-pathway categories of the subject by deviation from a reference distribution; selecting, based on the pathway-dominance profile, a tryptamine-based compound and a plurality of coordinated aging-pathway modulators; determining, based on the pathway-dominance profile, a dose of the tryptamine-based compound and a dose of each of the plurality of coordinated aging-pathway modulators; and compounding the tryptamine-based compound and the plurality of coordinated aging-pathway modulators at the determined doses into a unit dosage form.

16. The method of manufacturing of claim 15, wherein the tryptamine-based compound is a non-hallucinogenic structural analog of psilocin prepared by total chemical synthesis from 4-hydroxyindole or a protected derivative of 4-hydroxyindole.

17. The method of manufacturing of claim 15, wherein the tryptamine-based compound is psilocybin produced by biosynthesis in an engineered microbial host expressing PsiD, PsiK, and PsiM enzymes.

18. The method of manufacturing of claim 15, wherein compounding comprises incorporating an antioxidant and a pH-buffering excipient that together inhibit oxidation of psilocin.

19. The method of manufacturing of claim 15, wherein the unit dosage form is a monthly pre-dosed kit containing one dose of the tryptamine-based compound and a twenty-eight-day supply of the plurality of coordinated aging-pathway modulators.

20. The method of manufacturing of claim 15, wherein the dose of the tryptamine-based compound is determined by a cytochrome P450 2D6 (CYP2D6) metabolizer phenotype of the subject, with a reduced dose selected for a CYP2D6 poor-metabolizer phenotype.
